SEQUENCE LISTING (148 sequences)

SEQ ID NO: 1 - protein, 501 aa, Homo sapiens
QPSVSPGEPSPPSIHPGKSDLIVRVGDEIRLLCTDPGFVKWTFEILDETNENKQNEWITEKAEATNTGKYTCTNKHGLSNSIYVFVRDPAKLFLVDRSLYGKEDNDTLVRCPLTDPEVTNYSLKGCQGKPLPKDLRFIPDPKAGIMIKSVKRAYHRLCLHCSVDQEGKSVLSEKFILKVRPAFKAVPVVSVSKASYLLREGEEFTVTCTIKDVSSSVYSTWKRENSQTKLQEKYNSWHHGDFNYERQATLTISSARVNDSGVFMCYANNTFGSANVTTTLEVVDKGFINIFPMINTTVFVNDGENVDLIVEYEAFPKPEHQQWIYMNRTFTDKWEDYPKSENESNIRYVSELHLTRLKGTEGGTYTFLVSNSDVNAAIAFNVYVNTKPEILTYDRLVNGMLQCVAAGFPEPTIDWYFCPGTEQRCSASVLPVDVQTLNSSGPPFGKLVVQSSIDSSAFKHNGTVECKAYNDVGKTSAYFNFAFKGNNKEQIHPHTHHHHHH

SEQ ID NO: 2 - protein, 123 aa, Artificial Sequence
  Description of Artificial Sequence Synthetic polypeptide
EVQLVQSGAEVKKPGESLKISCKGSGYSFTNYWIGWVRQMPGKGLEWMAIINPRDSDTRYRPSFQGQVTISADKSISTAYLQWSSLKASDTAMYYCARHGRGYEGYEGAFDIWGQGTLVTVSS

SEQ ID NO: 3 - protein, 5 aa, Artificial Sequence
  Description of Artificial Sequence Synthetic peptide
NYWIG

SEQ ID NO: 4 - protein, 17 aa, Artificial Sequence
  Description of Artificial Sequence Synthetic peptide
IINPRDSDTRYRPSFQG

SEQ ID NO: 5 - protein, 14 aa, Artificial Sequence
  Description of Artificial Sequence Synthetic peptide
HGRGYEGYEGAFDI

SEQ ID NO: 6 - protein, 107 aa, Artificial Sequence
  Description of Artificial Sequence Synthetic polypeptide
DIQMTQSPSSLSASVGDRVTITCRSSQGIRSDLGWYQQKPGKAPKLLIYDASNLETGVPSRFSGSGSGTDFTLTISSLQPEDFATYYCQQANGFPLTFGGGTKVEIK

SEQ ID NO: 7 - protein, 11 aa, Artificial Sequence
  Description of Artificial Sequence Synthetic peptide
RSSQGIRSDLG

SEQ ID NO: 8 - protein, 7 aa, Artificial Sequence
  Description of Artificial Sequence Synthetic peptide
DASNLET

SEQ ID NO: 9 - protein, 9 aa, Artificial Sequence
  Description of Artificial Sequence Synthetic peptide
QQANGFPLT

SEQ ID NO: 10 - protein, 330 aa, Homo sapiens [sequence omitted]

SEQ ID NO: 11 - protein, 330 aa, Artificial Sequence
  Description of Artificial Sequence Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 330 aa, Artificial Sequence
  Description of Artificial Sequence Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 330 aa, Artificial Sequence
  Description of Artificial Sequence Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNAYTQKSLSLSPGK

SEQ ID NO: 14 - protein, 330 aa, Artificial Sequence
  Description of Artificial Sequence Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 330 aa, Artificial Sequence
  Description of Artificial Sequence Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNAYTQKSLSLSPGK

SEQ ID NO: 16 - protein, 453 aa, Artificial Sequence
  Description of Artificial Sequence Synthetic polypeptide
EVQLVQSGAEVKKPGESLKISCKGSGYSFTNYWIGWVRQMPGKGLEWMAIINPRDSDTRYRPSFQGQVTISADKSISTAYLQWSSLKASDTAMYYCARHGRGYEGYEGAFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - protein, 453 aa, Artificial Sequence
  Description of Artificial Sequence Synthetic polypeptide
EVQLVQSGAEVKKPGESLKISCKGSGYSFTNYWIGWVRQMPGKGLEWMAIINPRDSDTRYRPSFQGQVTISADKSISTAYLQWSSLKASDTAMYYCARHGRGYEGYEGAFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 453 aa, Artificial Sequence
  Description of Artificial Sequence Synthetic polypeptide
EVQLVQSGAEVKKPGESLKISCKGSGYSFTNYWIGWVRQMPGKGLEWMAIINPRDSDTRYRPSFQGQVTISADKSISTAYLQWSSLKASDTAMYYCARHGRGYEGYEGAFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - protein, 453 aa, Artificial Sequence
  Description of Artificial Sequence Synthetic polypeptide
EVQLVQSGAEVKKPGESLKISCKGSGYSFTNYWIGWVRQMPGKGLEWMAIINPRDSDTRYRPSFQGQVTISADKSISTAYLQWSSLKASDTAMYYCARHGRGYEGYEGAFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNAYTQKSLSLSPGK

SEQ ID NO: 20 - protein, 107 aa, Artificial Sequence [sequence omitted]

SEQ ID NO: 21 - protein, 214 aa, Artificial Sequence
  Description of Artificial Sequence Synthetic polypeptide
DIQMTQSPSSLSASVGDRVTITCRSSQGIRSDLGWYQQKPGKAPKLLIYDASNLETGVPSRFSGSGSGTDFTLTISSLQPEDFATYYCQQANGFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 22 - protein, 123 aa, Artificial Sequence
  Description of Artificial Sequence Synthetic polypeptide
QVQLVQSGAAVKKPGESLKISCKGSGYRFTSYWIGWVRQMPGKGLEWMGIIYPGDSDTRYSPSFQGQVTISAGKSISTAYLQWSSLKASDTAMYYCARHGRGYNGYEGAFDIWGQGTMVTVSS

SEQ ID NO: 23 - protein, 5 aa, Artificial Sequence
  Description of Artificial Sequence Synthetic peptide
SYWIG

SEQ ID NO: 24 - protein, 17 aa, Artificial Sequence
  Description of Artificial Sequence Synthetic peptide
IIYPGDSDTRYSPSFQG

SEQ ID NO: 25 - protein, 14 aa, Artificial Sequence
  Description of Artificial Sequence Synthetic peptide
HGRGYNGYEGAFDI

SEQ ID NO: 26 - protein, 107 aa, Artificial Sequence [sequence omitted]

SEQ ID NO: 27 - protein, 11 aa, Artificial Sequence
  Description of Artificial Sequence Synthetic peptide
RASQGISSALA

SEQ ID NO: 28 - protein, 7 aa, Artificial Sequence
  Description of Artificial Sequence Synthetic peptide
DASSLES

SEQ ID NO: 29 - protein, 10 aa, Artificial Sequence
  Description of Artificial Sequence Synthetic peptide
CQQFNSYPLT

SEQ ID NO: 30 - protein, 123 aa, Artificial Sequence
  Description of Artificial Sequence Synthetic polypeptide
QVQLVQSGAAVKKPGESLKISCKGSGYRFTTYWIGWVRQMPGKGLEWMGIIYPGDSDTRYSPSFQGQVTISAGKSISTAYLQWSSLKASDTAMYYCARHGRGYNGYEGAFDIWGQGTMVTVSS

SEQ ID NO: 31 - protein, 107 aa, Artificial Sequence
  Description of Artificial Sequence Synthetic polypeptide
AIQLTQSPSSLSASVGDRVTITCRASQGVSSALAWYQQKPGKAPKLLIYDASSLESGVPSRFSGSGSGTDFTLTISSLQPEDFATYYCQQFNSYPLTFGGGTKVEIK

SEQ ID NO: 32 - protein, 123 aa, Artificial Sequence; identical to SEQ ID NO: 30
  Description of Artificial Sequence Synthetic polypeptide

SEQ ID NO: 33 - protein, 107 aa, Artificial Sequence
  Description of Artificial Sequence Synthetic polypeptide
DIQLTQSPSSLSASVGDRVTITCRASQGIRTDLGWYQQKPGKAPKLLIYDASSLESGVPSRFSGSGSGTDFTLTISSLQPEDFATYYCQQFNSYPLTFGGGTKVEIK

SEQ ID NO: 34 - protein, 123 aa, Artificial Sequence; identical to SEQ ID NO: 30
  Description of Artificial Sequence Synthetic polypeptide

SEQ ID NO: 35 - protein, 107 aa, Artificial Sequence
  Description of Artificial Sequence Synthetic polypeptide
AIRMTQSPSSLSASVGDRVTITCRASQGIRNDLAWYQQKPGKTPKLLIYDASSLESGVPSRFSGSGSGTDFTLTISSLQPEDFATYYCQQFNSYPLTFGGGTKVEIK

SEQ ID NO: 36 - protein, 123 aa, Artificial Sequence; identical to SEQ ID NO: 30
  Description of Artificial Sequence Synthetic polypeptide

SEQ ID NO: 37 - protein, 107 aa, Artificial Sequence
  Description of Artificial Sequence Synthetic polypeptide
AIQMTQSPSSLSASVGDRVTITCRASQGIRNDLGWYQQKPGKAPKLLIYDASSLESGVPSRFSGSGSGTDFTLTISSLQPEDFATYYCQQFNSYPLTFGGGTKVDIK

SEQ ID NO: 38 - protein, 123 aa, Artificial Sequence; identical to SEQ ID NO: 30
  Description of Artificial Sequence Synthetic polypeptide

SEQ ID NO: 39 - protein, 107 aa, Artificial Sequence
  Description of Artificial Sequence Synthetic polypeptide
NIQMTQSPSSLSASVGDRVTITCRASQAISDYLAWFQQKPGKAPKLLIYDASNLETGVPSRFSGSGSGTDFTLTISSLQPEDFATYYCQQLNSYPLTFGGGTKVEIK

SEQ ID NO: 40 - protein, 123 aa, Artificial Sequence; identical to SEQ ID NO: 30
  Description of Artificial Sequence Synthetic polypeptide

SEQ ID NO: 41 - protein, 107 aa, Artificial Sequence
  Description of Artificial Sequence Synthetic polypeptide
AIRMTQSPSSLSASVGDRVIIACRASQGIGGALAWYQQKPGNAPKVLVYDASTLESGVPSRFSGGGSGTDFTLTISSLQPEDFATYYCQQFNSYPLTFGGGTKLEIK

SEQ ID NO: 42 - protein, 123 aa, Artificial Sequence; identical to SEQ ID NO: 30
  Description of Artificial Sequence Synthetic polypeptide

SEQ ID NO: 43 - protein, 107 aa, Artificial Sequence
  Description of Artificial Sequence Synthetic polypeptide
DIAMTQSPPSLSAFVGDRVTITCRASQGIISSLAWYQQKPGKAPKLLIYDASSLESGVPSRFSGSGSGTDFTLTIRSLQPEDFATYYCQQFNSYPLTFGGGTKLEIK

SEQ ID NO: 44 - protein, 123 aa, Artificial Sequence; identical to SEQ ID NO: 30
  Description of Artificial Sequence Synthetic polypeptide

SEQ ID NO: 45 - protein, 107 aa, Artificial Sequence
  Description of Artificial Sequence Synthetic polypeptide
DIQMTQSPSSLSASVGDRVTITCRASQGISSALAWYQQKAGKAPKVLISDASSLESGVPSRFSGSGSGTDFTLSISSLQPEDFATYYCQQFNGYPLTFGGGTKVDIK

SEQ ID NO: 46 - protein, 123 aa, Artificial Sequence; identical to SEQ ID NO: 30
  Description of Artificial Sequence Synthetic polypeptide

SEQ ID NO: 47 - protein, 107 aa, Artificial Sequence
  Description of Artificial Sequence Synthetic polypeptide
AIRMTQSPSSLSASVGDRVTITCQASQGIRNDLGWYQQKPGKAPKLLIYDASNLETGVPSRFSGSGSGTDFTFTISSLQPEDIATYYCQQFNSYPLTFGGGTKLEIK

SEQ ID NO: 48 - protein, 123 aa, Artificial Sequence; identical to SEQ ID NO: 30
  Description of Artificial Sequence Synthetic polypeptide

SEQ ID NO: 49 - protein, 107 aa, Artificial Sequence
  Description of Artificial Sequence Synthetic polypeptide
NIQMTQSPSSLSTSVGDRVTITCRASQGIGTSLAWYQQKPGKPPKLLIYDASSLESGVPSRLSGSGSGTDFTLTISSLQPEDFATYYCQQSNSYPITFGQGTRLEIK

SEQ ID NO: 50 - protein, 123 aa, Artificial Sequence; identical to SEQ ID NO: 30
  Description of Artificial Sequence Synthetic polypeptide

SEQ ID NO: 51 - protein, 107 aa, Artificial Sequence
  Description of Artificial Sequence Synthetic polypeptide
AIQLTQSPSSLSASVGDRVTITCRASQSIGDYLTWYQQKPGKAPKVLIYGASSLQSGVPPRFSGSGSGTDFTLTVSSLQPEDFATYYCQQLNSYPLTFGGGTKLEIK

SEQ ID NO: 52 - protein, 123 aa, Artificial Sequence; identical to SEQ ID NO: 30
  Description of Artificial Sequence Synthetic polypeptide

SEQ ID NO: 53 - protein, 107 aa, Artificial Sequence
  Description of Artificial Sequence Synthetic polypeptide
DIQLTQSPSSLSASVGDRVTITCRASQGVRSTLAWYQQKPGKAPKLLIYDASILESGVPSRFSGSGSGTDFTLTISSLQPEDFATYYCQQFNGYPLTFGQGTRLEIK

SEQ ID NO: 54 - protein, 123 aa, Artificial Sequence; identical to SEQ ID NO: 30
  Description of Artificial Sequence Synthetic polypeptide

SEQ ID NO: 55 - protein, 107 aa, Artificial Sequence
  Description of Artificial Sequence Synthetic polypeptide
DIVMTQSPSSLSASVGDRVTITCRASQGIRNDLGWYQQKPGKAPKLLIYDASSLESGVPSRFSGSGSGTDFTLTISSLQPEDFATYYCQQFNSYPLTFGGGTKLEIK

SEQ ID NO: 56 - protein, 123 aa, Artificial Sequence; identical to SEQ ID NO: 30
  Description of Artificial Sequence Synthetic polypeptide

SEQ ID NO: 57 - protein, 107 aa, Artificial Sequence
  Description of Artificial Sequence Synthetic polypeptide
DIQLTQSPSSLSASVGDRVTITCRASQGISSFLAWYQQKPGKAPKLLIYDASTLQSGVPSRFSGSASGTDFTLTISSLQPEDFATYYCQQLNGYPLTFGGGTKVEIK

SEQ ID NO: 58 - protein, 123 aa, Artificial Sequence; identical to SEQ ID NO: 30
  Description of Artificial Sequence Synthetic polypeptide

SEQ ID NO: 59 - protein, 107 aa, Artificial Sequence
  Description of Artificial Sequence Synthetic polypeptide
AIQLTQSPSSLSASVGDRVTITCRASQGIGSALAWYQQKPGIGPKLLIYDASTLESGVPARFSGSGSRTDFTLTITSLQPEDFATYYCQQFNGYPLTFGGGTKLEIK

SEQ ID NO: 60 - protein, 123 aa, Artificial Sequence; identical to SEQ ID NO: 30
  Description of Artificial Sequence Synthetic polypeptide

SEQ ID NO: 61 - protein, 107 aa, Artificial Sequence
  Description of Artificial Sequence Synthetic polypeptide
AIQLTQSPSSLSASVGDRVTITCRASQGITSALAWYQEKPGKAPNLLIYDASSLESGVPSRFSGSGYGTDFTLTISSLQPEDFATYYCQQLNSYPLTFGGGTKVDIK

SEQ ID NO: 62 - protein, 125 aa, Artificial Sequence
  Description of Artificial Sequence Synthetic polypeptide
QVQLVQSGAEVKKPGASVKVSCKASGYTFTDYYIHWVRQAPGQGLEWMGWMNPHSGDTGYAQKFQGRVTMTRDTSTSTVYMELSSLRSEDTAVYYCARHGRGYNGYEGAFDIWGQGTLVTVSSAS

SEQ ID NO: 63 - protein, 107 aa, Artificial Sequence
  Description of Artificial Sequence Synthetic polypeptide
DIQMTQSPSSLSASVGDRVTITCRASQGIGNELGWYQQKPGKAPKLLIYAASNLQSGVPSRFSGSGSGTDFTLTISSLQPEDFATYYCQQYDNLPLTFGQGTKVEIK

SEQ ID NO: 64 - protein, 125 aa, Artificial Sequence
  Description of Artificial Sequence Synthetic polypeptide
QVQLVQSGAEVKKPGASVKVSCKASGYTFTGYYLHWVRQAPGQGLEWMGWINPNSGDTNYAQNFQGRVTMTRDTSTSTVYMELSSLRSEDTAVYYCARHGRGYNGYEGAFDIWGQGTLVTVSSAS

SEQ ID NO: 65 - protein, 107 aa, Artificial Sequence
  Description of Artificial Sequence Synthetic polypeptide
DIQMTQSPSSLSASVGDRVTITCRASQGIRNDLGWYQQKPGKAPKLLIYDASSLESGVPSRFSGSGSGTDFTLTISSLQPEDFATYYCQQLNGYPLTFGGGTKVEIK

SEQ ID NO: 66 - protein, 125 aa, Artificial Sequence
  Description of Artificial Sequence Synthetic polypeptide
QVQLVQSGAEVKKPGASVKVSCKASGYTFTGYYLHWVRQAPGQGLEWMGWINPNSGGTNYAQKFQGRVTMTRDTSTSTVYMELSSLRSEDTAVYYCARHGRGYEGYEGAFDIWGQGTLVTVSSAS

SEQ ID NO: 67 - protein, 107 aa, Artificial Sequence
  Description of Artificial Sequence Synthetic polypeptide
DIQMTQSPSSLSASVGDRVTITCRASQGIRNDLGWYQQKPGKAPKLLIYDASELETGVPSRFSGSGSGTDFTLTISSLQPEDFATYYCQQLNGYPITFGQGTKVEIK

SEQ ID NO: 68 - protein, 125 aa, Artificial Sequence
  Description of Artificial Sequence Synthetic polypeptide
QVQLVQSGAEVKKPGASVKVSCKASGYTFTSYYIHWVRQAPGQGLEWMGWLNPSGGGTSYAQKFQGRVTMTRDTSTSTVYMELSSLRSEDTAVYYCARHGRGYDGYEGAFDIWGQGTLVTVSSAS

SEQ ID NO: 69 - protein, 107 aa, Artificial Sequence
  Description of Artificial Sequence Synthetic polypeptide
DIQMTQSPSSLSASVGDRVTITCRASQGIRNDLGWYQQKPGKAPKLLIYDASNLETGVPSRFSGSGSGTDFTLTISSLQPEDFATYYCQQLNGYPLTFGGGTKVEIK

SEQ ID NO: 70 - protein, 125 aa, Artificial Sequence
  Description of Artificial Sequence Synthetic polypeptide
QVQLVQSGAEVKKPGASVKVSCKASGYTFSTYYMHWVRQAPGQGLEWMGIINPSGGSTSYAQKFQGRVTMTRDTSTSTVYMKLSSLRSEDTAVYYCARHGRGYEGYEGAFDIWGQGTLVTVSSAS

SEQ ID NO: 71 - protein, 107 aa, Artificial Sequence
  Description of Artificial Sequence Synthetic polypeptide
DIQMTQSPSSLSASVGDRVTITCRASQGIRDDLGWYQQKPGKAPKLLIYDASNLETGVPSRFSGSGSGTDFTLTISSLQPEDFATYYCQQANGFPLTFGGGTKVEIK

SEQ ID NO: 72 - protein, 125 aa, Artificial Sequence
  Description of Artificial Sequence Synthetic polypeptide
QVQLVQSGAEVKKPGASVKVSCKASGYTFTGYYIHWVRQAPGQGLEWMGIINPSGGNTNYAQNFQGRVTMTRDTSTSTVYMELSSLRSEDTAVYYCARHGRGYNAYEGAFDIWGQGTLVTVSSAS

SEQ ID NO: 73 - protein, 107 aa, Artificial Sequence
  Description of Artificial Sequence Synthetic polypeptide
DIQMTQSPSSLSASVGDRVTITCRASQGIRNDLGWYQQKPGKAPKLLIYDASNLETGVPSRFSGSGSGTDFTLTISSLQPEDFATYYCQQVNGYPLTFGGGTKVEIK

SEQ ID NO: 74 - protein, 125 aa, Artificial Sequence
  Description of Artificial Sequence Synthetic polypeptide
QVQLVQSGAEVKKPGASVKVSCKASGGTFSSYAISWVRQAPGQGLEWMGVINPTVGGANYAQKFQGRVTMTRDTSTSTVYMELSSLRSEDTAVYYCARHGRGYNEYEGAFDIWGQGTLVTVSSAS

SEQ ID NO: 75 - protein, 107 aa, Artificial Sequence
  Description of Artificial Sequence Synthetic polypeptide
DIQMTQSPSSLSASVGDRVTITCQASQDISDYLNWYQQKPGKAPKLLIYDASNLETGVPSRFSGSGSGTDFTLTISSLQPEDFATYYCQQGNSFPLTFGGGTKLEIK

SEQ ID NO: 76 - protein, 125 aa, Artificial Sequence
  Description of Artificial Sequence Synthetic polypeptide
QVQLVQSGAEVKKLGASVKVSCKASGYTFSSYYMHWVRQAPGQGLEWMGVINPNGAGTNFAQKFQGRVTMTRDTSTSTVYMELSSLRSEDTAVYYCARHGRGYEGYEGAFDIWGQGTLVTVSSAS

SEQ ID NO: 77 - protein, 107 aa, Artificial Sequence; identical to SEQ ID NO: 65
  Description of Artificial Sequence Synthetic polypeptide

SEQ ID NO: 78 - protein, 125 aa, Artificial Sequence
  Description of Artificial Sequence Synthetic polypeptide
QVQLVQSGAEVKKPGASVKVSCKASGYTFTTYYMHWVRQAPGQGLEWMGWINPTGGGTNYAQNFQGRVTMTRDTSTSTVYMELSSLRSEDTAVYYCARHGRGYEGYEGAFDIWGQGTLVTVSSAS

SEQ ID NO: 79 - protein, 107 aa, Artificial Sequence
  Description of Artificial Sequence Synthetic polypeptide
DIQMTQSPSSLSASVGDRVTITCRASQGIRNDVSWYQQKPGKAPKLLIYDASNLETGVPSRFSGSGSGTDFTLTISSLQPEDFATYYCQQLSGYPITFGQGTKLEIK

SEQ ID NO: 80 - protein, 125 aa, Artificial Sequence
  Description of Artificial Sequence Synthetic polypeptide
QVQLVQSGAEVKKPGASVKVSCKASGYTFTSYYIHWVRQAPGQGLEWMGMINPSGGSTNYAQKFQGRVTMTRDTSTSTVYMELSSLRSEDTAVYYCARHGRGYNDYEGAFDIWGQGTLVTVSSAS

SEQ ID NO: 81 - protein, 107 aa, Artificial Sequence
  Description of Artificial Sequence Synthetic polypeptide
DIQMTQSPSSLSASVGDRVTITCRASQSISDWLAWYQQKPGKAPKLLIYEASNLEGGVPSRFSGSGSGTDFTLTISSLQPEDFATYYCQQANSFPYTFGQGTKVEIK

SEQ ID NO: 82 - protein, 125 aa, Artificial Sequence
  Description of Artificial Sequence Synthetic polypeptide
QVQLVQSGAEVKKPGASVKVSCKASGYIFSAYYIHWVRQAPGQGLEWMGIINPSGGSTRYAQKFQGRVTMTRDTSTSTVYMELSSLRSEDTAVYYCARHGRGYGGYEGAFDIWDQGTLVTVSSAS

SEQ ID NO: 83 - protein, 107 aa, Artificial Sequence
  Description of Artificial Sequence Synthetic polypeptide
DIQMTQSPSSLSASVGDRVTITCRASQGIGDYVAWYQQKPGKAPKLLIYDASNLETGVPSRFSGSGSGTDFTLTISSLQPEDFATYYCQQLNGYPITFGQGTRLEIK

SEQ ID NO: 84 - protein, 125 aa, Artificial Sequence
  Description of Artificial Sequence Synthetic polypeptide
EVQLVQSGAEVKKPGESLKISCKGSGYRFTSYWIGWVRQMPGKGLEWMGIIYPDDSDTRYSPSFQGQVTISVDKSNSTAYLQWSSLKASDTAMYYCARHGRGYNGYEGAFDIWGQGTLVTVSSAS

SEQ ID NO: 85 - protein, 107 aa, Artificial Sequence
  Description of Artificial Sequence Synthetic polypeptide
DIQMTQSPSSLSASVGDRVTITCRASQGISSYLAWYQQKPGKAPKLLIYDASNLETGVPSRFSGSGSGTYFTLTISSLQPEDFATYYCQQGASFPITFGQGTKVEIK

SEQ ID NO: 86 - protein, 125 aa, Artificial Sequence
  Description of Artificial Sequence Synthetic polypeptide
EVQLVQSGAEVKKPGESLKISCKGSGSSFPNSWIAWVRQMPGKGLEWMGIIYPSDSDTRYSPSFQGQVTISADKSISTAYLQWSSLEASDTAMYYCARHGRGYNGYEGAFDIWGQGTLVTVSSAS

SEQ ID NO: 87 - protein, 107 aa, Artificial Sequence
  Description of Artificial Sequence Synthetic polypeptide
DIQMTQSPSSLSASVGDRVTITCRASQGIRNYLAWYQQKPGKAPKLLIYDASSLQSGVPSRFSGSGSGTDFTLTISSLQPEDFATYYCQQLNSYPLTFGGGTKVEIK

SEQ ID NO: 88 - protein, 125 aa, Artificial Sequence
  Description of Artificial Sequence Synthetic polypeptide
EVQLVQSGAEVKKPGESLKISCKGSGYSFDSYWIGWVRQMPGKGLEWMGIMYPGDSDTRYSPSFQGQVTISADKSISTAYLQWSSLKASDTAMYYCARHGRGYNAYEGAFDIWGQGTLVTVSSAS

SEQ ID NO: 89 - protein, 107 aa, Artificial Sequence
  Description of Artificial Sequence Synthetic polypeptide
DIQMTQSPSSLSASVGDRVTITCRASQSINNWLAWYQQKPGKAPKLLIYDAFILQSGVPSRFSGSGSGTDFTLTISSLQPEDFATYYCLQLNSYPLTFGPGTKVDIK

SEQ ID NO: 90 - protein, 124 aa, Artificial Sequence
  Description of Artificial Sequence Synthetic polypeptide
EVQLVQSGAEVKKPGESLKISCKGSGYSFTNWIAWVRQMPGKGLEWMGIIYPGDSETRYSPSFQGQVTISADKSISTAYLQWSSLKASDTAMYYCARHGRGYYGYEGAFDIWGQGTLVTVSSAS

SEQ ID NO: 91 - protein, 107 aa, Artificial Sequence
  Description of Artificial Sequence Synthetic polypeptide
DIQMTQSPSSLSASVGDRVTITCRASQGISDNLNWYQQKPGKAPKLLIYDASNLETGVPSRFSGSGSGTDFTLTISSLQPEDFATYYCQQAISFPLTFGQGTKVEIK

SEQ ID NO: 92 - protein, 125 aa, Artificial Sequence
  Description of Artificial Sequence Synthetic polypeptide
EVQLVQSGAEVKKPGESLKISCKGSGYNFTSYWIGWVRQMPGKGLEWMGVIYPDDSETRYSPSFQGQVTISADKSISTAYLQWSSLKASDTAMYYCARHGRGYNGYEGAFDIWGQGTLVTVSSAS

SEQ ID NO: 93 - protein, 107 aa, Artificial Sequence
  Description of Artificial Sequence Synthetic polypeptide
DIQMTQSPSSLSASVGDRVTITCRASRDIRDDLGWYQQKPGKAPKLLIYDASNLETGVPSRFSGSGSGTDFTLTISSLQPEDFATYYCQQANSFPLTFGGGTKVEIK

SEQ ID NO: 94 - protein, 124 aa, Artificial Sequence
  Description of Artificial Sequence Synthetic polypeptide
EVQLVQSGAEVKKPGESLKISCKGSGYTFNTYIGWVRQMPGKGLEWMGIIYPGDSGTRYSPSFQGQVTISADKAISTAYLQWSSLKASDTAMYYCARHSRGYNGYEGAFDIWGQGTLVTVSSAS

SEQ ID NO: 95 - protein, 107 aa, Artificial Sequence
  Description of Artificial Sequence Synthetic polypeptide
DIQMTQSPSSLSASVGDRVTITCRASQGISNYLAWYQQKPGKAPKLLIYDASNLETGVPSRFSGSGSGTDFTLTISSLQPEDFATYYCQQANSFPVTFGQGTKVEIK

SEQ ID NO: 96 - protein, 125 aa, Artificial Sequence
  Description of Artificial Sequence Synthetic polypeptide
EVQLVQSGAEVKKPGESLKISCKGSGYNFTTYWIGWVRQMPGKGLEWMGIIHPADSDTRYNPSFQGQVTISADKSISTAYLQWSSLKASDTAMYYCARHGRGYNGYEGAFDIWGQGTLVTVSSAS

SEQ ID NO: 97 - protein, 107 aa, Artificial Sequence
  Description of Artificial Sequence Synthetic polypeptide
DIQMTQSPSSLSASVGDRVTITCRVSQGISSYLAWYQQKPGKAPKLLIYDASNLETGVPSRFSGSGSGTDFTLTISSLQPEDFATYYCQQANSFPLTFGGGTKVEIK

SEQ ID NO: 98 - protein, 125 aa, Artificial Sequence
  Description of Artificial Sequence Synthetic polypeptide
EVQLVQSGAEVKKPGESLKISCKGSGYRFSNYWIAWVRQMPGKGLEWMGIIYPDNSDTRYSPSFQGQVTISADKSISTAYLQWSSLKASDTAMYYCARHGRGYDGYEGAFDIWGQGTLVTVSSAS

SEQ ID NO: 99 - protein, 107 aa, Artificial Sequence
  Description of Artificial Sequence Synthetic polypeptide
DIQMTQSPSSLSASVGDRVTITCRASQGIRSDLAWYQQKPGKAPKLLIYGASSLQSGVPSRFSGSGSGTDFTLTISSLQPEDFATYYCQQANSFPLSFGQGTKVEIK

SEQ ID NO: 100 - protein, 124 aa, Artificial Sequence
  Description of Artificial Sequence Synthetic polypeptide
EVQLVQSGAEVKKPGESLKISCKGSGYRFASYWIGWVRQMPGKGLEWMGITYPGDSETRYNPSQGQVTISADKSISTAYLQWSSLKASDTAMYYCARHGRGYGGYEGAFDIWGQGTLVTVSSAS

SEQ ID NO: 101 - protein, 107 aa, Artificial Sequence
  Description of Artificial Sequence Synthetic polypeptide
DIQMTQSPSSLSASVGDRVTITCRASQGIRNDLGWYQQKPGKAPKLLIYDASNLETGVPSRFSGSGSGTDFTLTISSLQPEDFATYYCQQANSFPLTFGGGTKVEIK

SEQ ID NO: 102 - protein, 125 aa, Artificial Sequence
  Description of Artificial Sequence Synthetic polypeptide
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARHGRGYNGYEGAFDIWGQGTLVTVSSAS

SEQ ID NO: 103 - protein, 107 aa, Artificial Sequence
  Description of Artificial Sequence Synthetic polypeptide
DIQMTQSPSSLSASVGDRVTITCRASQSISNWLAWYQQKPGKAPKLLIYDASNLETGVPSRFSGSGSGTDFTLTISSLQPEDFATYYCQQTNSFPLTFGQGTRLEIK

SEQ ID NO: 104 - protein, 123 aa, Artificial Sequence; identical to SEQ ID NO: 30
  Description of Artificial Sequence Synthetic polypeptide

SEQ ID NO: 105 - protein, 107 aa, Artificial Sequence
  Description of Artificial Sequence Synthetic polypeptide
DIQLTQSPSSLSASVGDRVTITCRASQGVISALAWYQQKPGKAPKLLIYDASSLESGVPSRFSGSGSGTDFTLTISSLQPEDFATYYCQQFNSYPLTFGGGTKVEIK

SEQ ID NO: 106 - protein, 123 aa, Artificial Sequence; identical to SEQ ID NO: 30
  Description of Artificial Sequence Synthetic polypeptide

SEQ ID NO: 107 - protein, 107 aa, Artificial Sequence
  Description of Artificial Sequence Synthetic polypeptide
DIQLTQSPSSLSASVGDRVTITCRASQGIRSALAWYQQKPGKAPKLLIYDASSLESGVPSRFSGSGSGTDFTLTISSLQPEDFATYYCQQFNSYPLTFGGGTKVEIK

SEQ ID NO: 108 - protein, 123 aa, Artificial Sequence; identical to SEQ ID NO: 30
  Description of Artificial Sequence Synthetic polypeptide

SEQ ID NO: 109 - protein, 107 aa, Artificial Sequence
  Description of Artificial Sequence Synthetic polypeptide
DIQLTQSPSSLSASVGDRVTITCRASQGVGSALAWYQQKPGKAPKLLIYDASSLESGVPSRFSGSGSGTDFTLTISSLQPEDFATYYCQQFNSYPLTFGGGTKVEIK

SEQ ID NO: 110 - protein, 123 aa, Artificial Sequence; identical to SEQ ID NO: 30
  Description of Artificial Sequence Synthetic polypeptide

SEQ ID NO: 111 - protein, 107 aa, Artificial Sequence
  Description of Artificial Sequence Synthetic polypeptide
DIQLTQSPSSLSASVGDRVTITCRASQGVISALAWYQQKPGKAPKLLIYDASILESGVPSRFSGSGSGTDFTLTISSLQPEDFATYYCQQFNSYPLTFGGGTKVEIK

SEQ ID NO: 112 - protein, 123 aa, Artificial Sequence; identical to SEQ ID NO: 30
  Description of Artificial Sequence Synthetic polypeptide

SEQ ID NO: 113 - protein, 107 aa, Artificial Sequence
  Description of Artificial Sequence Synthetic polypeptide
DIQLTQSPSSLSASVGDRVTITCRASQGIRSALAWYQQKPGKAPKLLIYDASILESGVPSRFSGSGSGTDFTLTISSLQPEDFATYYCQQFNSYPLTFGGGTKVEIK

SEQ ID NO: 114 - protein, 123 aa, Artificial Sequence; identical to SEQ ID NO: 30
  Description of Artificial Sequence Synthetic polypeptide

SEQ ID NO: 115 - protein, 107 aa, Artificial Sequence
  Description of Artificial Sequence Synthetic polypeptide
DIQLTQSPSSLSASVGDRVTITCRASQGVGSALAWYQQKPGKAPKLLIYDASILESGVPSRFSGSGSGTDFTLTISSLQPEDFATYYCQQFNSYPLTFGGGTKVEIK

SEQ ID NO: 116 - protein, 123 aa, Artificial Sequence; identical to SEQ ID NO: 30
  Description of Artificial Sequence Synthetic polypeptide

SEQ ID NO: 117 - protein, 107 aa, Artificial Sequence
  Description of Artificial Sequence Synthetic polypeptide
DIQLTQSPSSLSASVGDRVTITCRASQGISSALAWYQQKPGKAPKLLIYDASILESGVPSRFSGSGSGTDFTLTISSLQPEDFATYYCQQFNSYPLTFGGGTKVEIK

SEQ ID NO: 118 - protein, 123 aa, Artificial Sequence; identical to SEQ ID NO: 30
  Description of Artificial Sequence Synthetic polypeptide

SEQ ID NO: 119 - protein, 107 aa, Artificial Sequence
  Description of Artificial Sequence Synthetic polypeptide
DIQLTQSPSSLSASVGDRVTITCRASQGVISALAWYQQKPGKAPKLLIYDASTLESGVPSRFSGSGSGTDFTLTISSLQPEDFATYYCQQFNSYPLTFGGGTKVEIK

SEQ ID NO: 120 - protein, 123 aa, Artificial Sequence; identical to SEQ ID NO: 30
  Description of Artificial Sequence Synthetic polypeptide

SEQ ID NO: 121 - protein, 107 aa, Artificial Sequence
  Description of Artificial Sequence Synthetic polypeptide
DIQLTQSPSSLSASVGDRVTITCRASQGIRSALAWYQQKPGKAPKLLIYDASTLESGVPSRFSGSGSGTDFTLTISSLQPEDFATYYCQQFNSYPLTFGGGTKVEIK

SEQ ID NO: 122 - protein, 123 aa, Artificial Sequence; identical to SEQ ID NO: 30
  Description of Artificial Sequence Synthetic polypeptide

SEQ ID NO: 123 - protein, 107 aa, Artificial Sequence
  Description of Artificial Sequence Synthetic polypeptide
DIQLTQSPSSLSASVGDRVTITCRASQGVGSALAWYQQKPGKAPKLLIYDASTLESGVPSRFSGSGSGTDFTLTISSLQPEDFATYYCQQFNSYPLTFGGGTKVEIK

SEQ ID NO: 124 - protein, 123 aa, Artificial Sequence; identical to SEQ ID NO: 30
  Description of Artificial Sequence Synthetic polypeptide

SEQ ID NO: 125 - protein, 107 aa, Artificial Sequence; identical to SEQ ID NO: 123
  Description of Artificial Sequence Synthetic polypeptide

SEQ ID NO: 126 - protein, 123 aa, Artificial Sequence
  Description of Artificial Sequence Synthetic polypeptide
EVQLVQSGAEVKKPGESLKISCKGSGYSFTNYWIGWVRQMPGKGLEWMGIIYPGDSDIRYSPSLQGQVTISVDTSTSTAYLQWNSLKPSDTAMYYCARHGRGYNGYEGAFDIWGQGTLVTVSS

SEQ ID NO: 127 - protein, 107 aa, Artificial Sequence
  Description of Artificial Sequence Synthetic polypeptide
DIQMTQSPSSLSASVGDRVTITCRASQGIGDSLAWYQQKPGKAPKLLIYDASNLETGVPSRFSGSGSGTDFTLTISSLQPEDFATYYCQQLNGYPITFGQGTKVEIK

SEQ ID NO: 128 - protein, 123 aa, Artificial Sequence; identical to SEQ ID NO: 2
  Description of Artificial Sequence Synthetic polypeptide

SEQ ID NO: 129 - protein, 107 aa, Artificial Sequence
  Description of Artificial Sequence Synthetic polypeptide
DIQMTQSPSSLSASVGDRVTITCRASQGIRNDLGWYQQKPGKAPKLLIYDASSLESGVPSRFSGSGSGTDFTLTISSLQPEDFATYYCQQLNGYPITFGQGTKVEIK

SEQ ID NO: 130 - protein, 123 aa, Artificial Sequence
  Description of Artificial Sequence Synthetic polypeptide
EVQLVQSGAEVKKPGESLKISCKGSGYSFTNYWIGWVRQMPGKGLEWMGIIYPGDSLTRYSPSFQGQVTISADKSISTAYLQWSSLKASDTAMYYCARHGRGYNGYEGAFDIWGQGTLVTVSS

SEQ ID NO: 131 - protein, 107 aa, Artificial Sequence; identical to SEQ ID NO: 129
  Description of Artificial Sequence Synthetic polypeptide

SEQ ID NO: 132 - protein, 123 aa, Artificial Sequence
  Description of Artificial Sequence Synthetic polypeptide
EVQLVQSGAEVKKPGESLKISCKGSGYSFTNYWIGWVRQMPGKGLEWMGIIYPGDSDTRYSPSFQGQVTISADKSISTAYLQWSSLKASDTAMYYCARHGRGYNGYEGAFDIWGQGTLVTVSS

SEQ ID NO: 133 - protein, 107 aa, Artificial Sequence; identical to SEQ ID NO: 127
  Description of Artificial Sequence Synthetic polypeptide

SEQ ID NO: 134 - protein, 123 aa, Artificial Sequence
  Description of Artificial Sequence Synthetic polypeptide
EVQLVQSGAEVKKPGESLKISCKGSGYRFTTSWIGWVRQMPGKGLEWMGIIYPGDSDTRYSPSFQGQVTISADKSISTAYLQWSSLKASDTAMYYCARHGLGYNGYEGAFDIWGQGTLVTVSS

SEQ ID NO: 135 - protein, 107 aa, Artificial Sequence
  Description of Artificial Sequence Synthetic polypeptide
DIQMTQSPSSLSASVGDRVTITCRASQGIGSALAWYQQKPGKAPKLLIYDASTLESGVPSRFSGSGSGTDFTLTISSLQPEDFATYYCQQFNGYPLTFGQGTRLEIK

SEQ ID NO: 136 - protein, 123 aa, Artificial Sequence; identical to SEQ ID NO: 30
  Description of Artificial Sequence Synthetic polypeptide

SEQ ID NO: 137 - protein, 107 aa, Artificial Sequence; identical to SEQ ID NO: 135
  Description of Artificial Sequence Synthetic polypeptide

SEQ ID NO: 138 - protein, 123 aa, Artificial Sequence; identical to SEQ ID NO: 30
  Description of Artificial Sequence Synthetic polypeptide

SEQ ID NO: 139 - protein, 107 aa, Artificial Sequence
  Description of Artificial Sequence Synthetic polypeptide
DIQMTQSPSSLSASVGDRVTITCRASRGISDYLAWYQQKPGKAPKLLIYDASNLETGVPSRFSGSGSGTDFTLTISSLQPEDFATYYCQQANSFPITFGQGTRLEIK

SEQ ID NO: 140 - protein, 123 aa, Artificial Sequence; identical to SEQ ID NO: 134
  Description of Artificial Sequence Synthetic polypeptide

SEQ ID NO: 141 - protein, 107 aa, Artificial Sequence
  Description of Artificial Sequence Synthetic polypeptide
DIQMTQSPSSLSASVGDRVTITCRASQGIGSALAWYQQKPGKAPKLLIYDASTLESGVPSRFSGSGSGTDFTLTISSLQPEDFATYYCQQLNGYPLTFGQGTRLEIK

SEQ ID NO: 142 - protein, 123 aa, Artificial Sequence; identical to SEQ ID NO: 134
  Description of Artificial Sequence Synthetic polypeptide

SEQ ID NO: 143 - protein, 107 aa, Artificial Sequence
  Description of Artificial Sequence Synthetic polypeptide
DIQMTQSPSSLSASVGDRVTITCRASQGIGSALAWYQQKPGKAPKLLIYDASNLETGVPSRFSGSGSGTDFTLTISSLQPEDFATYYCQQLNGYPLTFGQGTRLEIK

SEQ ID NO: 144 - protein, 293 aa, Escherichia coli
KEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASRVARMASDEFPSMCPADGRVRGITHNKILWDSSTLGAILMRRTISS

SEQ ID NO: 145 - protein, 293 aa, Shigella dysenteriae
KEFTLDFSTAKTYVDSLNVIRSAIGTPLQTISSGGTSLLMIDSGTGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASRVARMASDEFPSMCPADGRVRGITHNKILWDSSTLGAILMRRTISS

SEQ ID NO: 146 - protein, 297 aa, Escherichia coli
DEFTVDFSSQKSYVDSLNSIRSAISTPLGNISQGGVSVSVINHVLGGNYISLNVRGLDPYSERFNHLRLIMERNNLYVAGFINTETNIFYRFSDFSHISVPDVITVSMTTDSSYSSLQRIADLERTGMQIGRHSLVGSYLDLMEFRGRSMTRASSRAMLRFVTVIAEALRFRQIQRGFRPALSEASPLYTMTAQDVDLTLNWGRISNVLPEYRGEEGVRIGRISFNSLSAILGSVAVILNCHSTGSYSVRSVSQKQKTECQIVGDRAAIKVNNVLWEANTIAALLNRKPQDLTEPNQ

SEQ ID NO: 147 - protein, 31 aa, Homo sapiens
EKAEATNTGKYTCTNKHGLSNSIYVFVRDPA

SEQ ID NO: 148 - protein, 21 aa, Homo sapiens
RCPLTDPEVTNYSLKGCQGKP

(19) United States
(12) Patent Application Publication
(10) Pub. No.: US 2022/0177575 A1
(43) Pub. Date: Jun. 9, 2022

(54) ANTI-CD117 ANTIBODIES AND USES THEREOF

(71) Applicant: Magenta Therapeutics, Inc., Cambridge, MA (US)

(72) Inventors: Rahul Palchaudhuri, Somerville, MA (US); Anthony Boitano, Newton, MA (US); Michael Cooke, Boston, MA (US); Bradley R. Pearse, Watertown, MA (US)

(21) Appl. No.: 17/452,045

(22) Filed: Oct. 22, 2021

Related U.S. Application Data
(63) Continuation of application No. PCT/US2020/029648, filed on Apr. 23, 2020.
(60) Provisional application No. 62/841,733, filed on May 1, 2019.
(60) Provisional application No. 62/838,264, filed on Apr. 24, 2019.

(51) Int. Cl. C07K 16/28; C07K 14/705

(52) U.S. Cl. CPC C07K 16/2803; C07K 14/70596; C07K 2317/76; C07K 2317/34; C07K 2317/21

(57) ABSTRACT

The present disclosure provides compositions and methods useful for the depletion of CD117+ cells and for the treatment of various hematopoietic diseases, metabolic disorders, cancers, e.g., acute myeloid leukemia (AML) and autoimmune diseases, among others. Described herein are antibodies, antigen-binding fragments, and conjugates thereof that can be applied to effect the treatment of these conditions, for instance, by depleting a population of CD117+ cells in a patient, such as a human. The compositions and methods described herein can be used to treat a disorder directly, for instance, by depleting a population of CD117+ cancer cells or autoimmune cells. The compositions and methods described herein can also be used to prepare a patient for hematopoietic stem cell transplant therapy and to improve the engraftment of hematopoietic stem cell transplants by selectively depleting endogenous hematopoietic stem cells prior to the transplant procedure.

### RELATED APPLICATIONS

This application claims priority to U.S. Provisional Application No. 62/838,264, filed on Apr. 24, 2019 and U.S. Provisional Application No. 62/841,733, filed on May 1, 2019. The content of each of the foregoing priority applications is incorporated by reference herein.

### SEQUENCE LISTING

The instant application contains a Sequence Listing which has been submitted electronically in ASCII format and is hereby incorporated by reference in its entirety. Said ASCII copy, created on Apr. 20, 2020, is named M103034_2150WO_SL.txt and is 186,529 bytes in size.

### FIELD

The present disclosure relates anti-CD117 antibodies, antibody drug conjugates (ADCs), and antigen-binding fragments thereof, as well as methods of treating patients suffering from various pathologies, such as blood diseases, metabolic disorders, cancers, and autoimmune diseases, among others, by administration of an antibody, or antibody drug conjugate (ADC), capable of binding an antigen expressed by a hematopoietic cell, such as a hematopoietic stem cell.

### BACKGROUND

Despite advances in the medicinal arts, there remains a demand for treating pathologies of the hematopoietic system, such as diseases of a particular blood cell, metabolic disorders, cancers, and autoimmune conditions, among others. While hematopoietic stem cells have significant therapeutic potential, a limitation that has hindered their use in the clinic has been the difficulty associated with ensuring engraftment of hematopoietic stem cell transplants in a host.

There is currently a need for compositions that target specific endogenous stem cells that can be used as conditioning agents to promote the engraftment of exogenous hematopoietic stem cell grafts such that the multi-potency and hematopoietic functionality of these cells is preserved in the patient following transplantation.

CD117 (also referred to as c-kit or Stem Cell Factor Receptor (SCRF)) is a single transmembrane, receptor tyrosine kinase that binds the ligand Stem Cell Factor (SCF). SCF induces homodimerization of cKIT which activates its tyrosine kinase activity and signals through both the PI3-AKT and MAPK pathways (Kindblom et al., Am J. Path. 1998 152(5):1259).

CD117 was initially discovered as an oncogene and has been studied in the field of oncology (see, for example, Stankov et al. (2014) Curr Pharm Des. 20(17):2849-80). An antibody drug conjugate (KTN0158) directed to CD117 is currently under investigation for the treatment of refractory gastrointestinal stromal tumors (GIST) (e.g., “KTN0158, a humanized anti-KIT monoclonal antibody, demonstrates biologic activity against both normal and malignant canine mast cells” London et al. (2016) Clin Cancer Res DOI: 10.1158/1078-0432.CCR-16-2152).

CD117 is highly expressed on hematopoietic stem cells (HSCs). This expression pattern makes CD117 a potential target for conditioning across a broad range of diseases. There remains, however, a need for anti-CD117 based therapy that is effective for conditioning a patient for transplantation, such as a bone marrow transplantation.

### SUMMARY

Described herein are antibodies, and antigen binding portions thereof, that specifically bind human CD117 (also known as c-kit), as well as compositions and methods of using said antibodies. In particular, the antibodies and fragments described herein can be used in anti-CD117 antibody drug conjugates (ADCs).

In one embodiment, the present disclosure provides compositions and methods for the direct treatment of various disorders of the hematopoietic system, metabolic disorders, cancers, and autoimmune diseases, among others. The present disclosure additionally features methods for conditioning a patient, such as a human patient, prior to receiving hematopoietic stem cell transplant therapy so as to promote the engraftment of hematopoietic stem cell grafts. The patient may be one that is suffering from one or more blood disorders, such as a hemoglobinopathy or other hematopoietic pathology, and is thus in need of hematopoietic stem cell transplantation. As described herein, hematopoietic stem cells are capable of differentiating into a multitude of cell types in the hematopoietic lineage, and can be administered to a patient in order to populate or re-populate a cell type that is deficient in the patient. The present disclosure features methods of treating a patient with antibodies and antibody drug conjugates (ADCs) capable of binding proteins expressed by hematopoietic cells, such as CD117 (including, for example, GNNK+ CD117), so as to (i) directly treat a disease such as a blood disorder, metabolic disease, cancer, or autoimmune disease, among others described herein, by selectively depleting a population of cells that express CD117, such as an aberrant blood cell, cancer cell, or autoimmune cell, and/or (ii) deplete a population of endogenous hematopoietic stem cells within the patient. The former activity enables the direct treatment of a wide range of disorders associated with a cell of the hematopoietic lineage, as CD117 may be expressed by a cancerous cell, such as a leukemic cell, an autoimmune lymphocyte, such as a T-cell that expresses a T-cell receptor that cross-reacts with a self antigen, among other cell types. The latter activity, the selective depletion of hematopoietic stem cells, in turn creates a vacancy that can subsequently be filled by transplantation of an exogenous (for instance, an autologous, allogeneic, or syngeneic) hematopoietic stem cell graft. The present disclosure thus provides methods of treating a variety of hematopoietic conditions, such as sickle cell anemia, thalassemia, Fanconi anemia, Wiskott-Aldrich syndrome, adenosine deaminase deficiency-severe combined immunodeficiency, metachromatic leukodystrophy, Diamond-Blackfan anemia and Schwachman-Diamond syndrome, human immunodeficiency virus infection, and acquired immune deficiency syndrome, as well as cancers and autoimmune diseases, among others.

In one aspect, the present disclosure provides an isolated anti-CD117 antibody, or antigen-binding fragment thereof, which binds to human CD117 at least at two of the following amino acid residues T67, K69, T71, S81, Y83, T114, T119, or K129 of CD117 listed in SEQ ID NO: 1. In one embodiment, the isolated anti-CD117 antibody, or antigen-binding fragment thereof, binds to at least three of the following amino acid residues T67, K69, T71, S81, Y83, T114, T119, or K129 of CD117 listed in SEQ ID NO: 1. In another embodiment, the isolated anti-CD117 antibody, or antigen-binding fragment thereof, binds to at least four of the following amino acid residues T67, K69, T71, S81, Y83, T114, T119, or K129 of CD117 listed in SEQ ID NO: 1. In one embodiment, the isolated anti-CD117 antibody, or antigen-binding fragment thereof, binds to at least five of the following amino acid residues T67, K69, T71, S81, Y83, T114, T119, or K129 of CD117 listed in SEQ ID NO: 1. In one embodiment, the isolated anti-CD117 antibody, or antigen-binding fragment thereof, binds to at least six of the following amino acid residues T67, K69, T71, S81, Y83, T114, T119, or K129 of CD117 listed in SEQ ID NO: 1. In one embodiment, the isolated anti-CD117 antibody, or antigen-binding fragment thereof, binds to at least seven of the following amino acid residues T67, K69, T71, S81, Y83, T114, T119, or K129 of CD117 listed in SEQ ID NO: 1. In one embodiment, the isolated anti-CD117 antibody, or antigen-binding fragment thereof, binds to each of the following amino acid residues T67, K69, T71, S81, Y83, T114, T119, and K129 of CD117 listed in SEQ ID NO: 1. In one embodiment, the isolated anti-CD117 antibody, or antigen-binding fragment thereof, comprises a heavy chain variable region having an amino acid sequence that is about 80% identical to SEQ ID NO: 2 and a light chain variable region having an amino acid sequence that is about 80% identical to the SEQ ID NO: 6. In another embodiment, the isolated anti-CD117 antibody, or antigen-binding fragment thereof, comprises a heavy chain variable region having an amino acid sequence that is at least 80% identical to SEQ ID NO: 2 and a light chain variable region having an amino acid sequence that is at least 80% identical to the SEQ ID NO: 6. In one embodiment, the isolated anti-CD117 antibody, or antigen-binding fragment thereof, comprises a heavy chain variable region having an amino acid sequence that is about 85% identical to SEQ ID NO: 2 and a light chain variable region having an amino acid sequence that is about 85% identical to the SEQ ID NO: 6. In another embodiment, the isolated anti-CD117 antibody, or antigen-binding fragment thereof, comprises a heavy chain variable region having an amino acid sequence that is at least 85% identical to SEQ ID NO: 2 and a light chain variable region having an amino acid sequence that is at least 85% identical to the SEQ ID NO: 6. In one embodiment, the isolated anti-CD117 antibody, or antigen-binding fragment thereof, comprises a heavy chain variable region having an amino acid sequence that is about 90% identical to SEQ ID NO: 2 and a light chain variable region having an amino acid sequence that is about 90% identical to the SEQ ID NO: 6. In another embodiment, the isolated anti-CD117 antibody, or antigen-binding fragment thereof, comprises a heavy chain variable region having an amino acid sequence that is at least 90% identical to SEQ ID NO: 2 and a light chain variable region having an amino acid sequence that is at least 90% identical to the SEQ ID NO: 6. In one embodiment, the isolated anti-CD117 antibody, or antigen-binding fragment thereof, comprises a heavy chain variable region having an amino acid sequence that is about 95% identical to SEQ ID NO: 2 and a light chain variable region having an amino acid sequence that is about 95% identical to the SEQ ID NO: 6. In another embodiment, the isolated anti-CD117 antibody, or antigen-binding fragment thereof, comprises a heavy chain variable region having an amino acid sequence that is at least 95% identical to SEQ ID NO: 2 and a light chain variable region having an amino acid sequence that is at least 95% identical to the SEQ ID NO: 6. In one embodiment, the isolated anti-CD117 antibody, or antigen-binding fragment thereof, comprises a heavy chain variable region having an amino acid sequence that is about 96% identical to SEQ ID NO: 2 and a light chain variable region having an amino acid sequence that is about 96% identical to the SEQ ID NO: 6. In another embodiment, the isolated anti-CD117 antibody, or antigen-binding fragment thereof, comprises a heavy chain variable region having an amino acid sequence that is at least 96% identical to SEQ ID NO: 2 and a light chain variable region having an amino acid sequence that is at least 96% identical to the SEQ ID NO: 6. In one embodiment, the isolated anti-CD117 antibody, or antigen-binding fragment thereof, comprises a heavy chain variable region having an amino acid sequence that is about 97% identical to SEQ ID NO: 2 and a light chain variable region having an amino acid sequence that is about 97% identical to the SEQ ID NO: 6. In another embodiment, the isolated anti-CD117 antibody, or antigen-binding fragment thereof, comprises a heavy chain variable region having an amino acid sequence that is at least 97% identical to SEQ ID NO: 2 and a light chain variable region having an amino acid sequence that is at least 97% identical to the SEQ ID NO: 6. In one embodiment, the isolated anti-CD117 antibody, or antigen-binding fragment thereof, comprises a heavy chain variable region having an amino acid sequence that is about 98% identical to SEQ ID NO: 2 and a light chain variable region having an amino acid sequence that is about 98% identical to the SEQ ID NO: 6. In another embodiment, the isolated anti-CD117 antibody, or antigen-binding fragment thereof, comprises a heavy chain variable region having an amino acid sequence that is at least 98% identical to SEQ ID NO: 2 and a light chain variable region having an amino acid sequence that is at least 98% identical to the SEQ ID NO: 6. In one embodiment, the isolated anti-CD117 antibody, or antigen-binding fragment thereof, comprises a heavy chain variable region having an amino acid sequence that is about 99% identical to SEQ ID NO: 2 and a light chain variable region having an amino acid sequence that is about 99% identical to the SEQ ID NO: 6. In another embodiment, the isolated anti-CD117 antibody, or antigen-binding fragment thereof, comprises a heavy chain variable region having an amino acid sequence that is at least 99% identical to SEQ ID NO: 2 and a light chain variable region having an amino acid sequence that is at least 99% identical to the SEQ ID NO: 6.

In another aspect, the present disclosure pertains to an antibody, or antigen binding fragment thereof, capable of binding CD117 which binds to an epitope in CD117, comprising at least two, at least three, at least four, at least five, at least six, at least seven, or all of the amino acid residues T67, K69, T71, S81, Y83, T114, T119, and K129 of CD117 of SEQ ID NO: 1.

In one aspect, the present disclosure provides an isolated anti-CD117 antibody, or antigen-binding fragment thereof, capable of binding CD117 that binds to an epitope having amino acid residues within at least amino acids 67-83 and 114-129 of SEQ ID NO: 1.

In another aspect, the present disclosure provides an isolated anti-CD117 antibody, or antigen-binding fragment thereof, capable of binding CD117 that binds to an epitope in CD117 comprising two, three, four, five, six, seven, or all of the amino acid residues T67, K69, T71, S81, Y83, T114, T119, and K129 of CD117 of SEQ ID NO: 1.

In certain embodiments, the anti-CD117 antibody, or antigen-binding fragment thereof, does not comprise the CDRs or the variable regions set forth in SEQ ID NO: 2 and SEQ ID NO: 6. In another embodiment, the anti-CD117 antibody, or antigen-binding fragment thereof, does not comprise the CDRs or the variable regions set forth in SEQ ID NO: 22 and SEQ ID NO: 26. In certain embodiments, the anti-CD117 antibody, or antigen-binding fragment thereof, does not comprise the CDRs or the variable regions set forth in SEQ ID NO: 30 and SEQ ID NO: 31. In certain embodiments, the anti-CD117 antibody, or antigen-binding fragment thereof, does not comprise the CDRs or the variable regions set forth in SEQ ID NO: 32 and SEQ ID NO: 33. In certain embodiments, the anti-CD117 antibody, or antigen-binding fragment thereof, does not comprise the CDRs or the variable regions set forth in SEQ ID NO: 34 and SEQ ID NO: 35. In certain embodiments, the anti-CD117 antibody, or antigen-binding fragment thereof, does not comprise the CDRs or the variable regions set forth in SEQ ID NO: 36 and SEQ ID NO: 37. In certain embodiments, the anti-CD117 antibody, or antigen-binding fragment thereof, does not comprise the CDRs or the variable regions set forth in SEQ ID NO: 38 and SEQ ID NO: 39. In certain embodiments, the anti-CD117 antibody, or antigen-binding fragment thereof, does not comprise the CDRs or the variable regions set forth in SEQ ID NO: 40 and SEQ ID NO: 41. In certain embodiments, the anti-CD117 antibody, or antigen-binding fragment thereof, does not comprise the CDRs or the variable regions set forth in SEQ ID NO: 42 and SEQ ID NO: 43. In certain embodiments, the anti-CD117 antibody, or antigen-binding fragment thereof, does not comprise the CDRs or the variable regions set forth in SEQ ID NO: 44 and SEQ ID NO: 45. In certain embodiments, the anti-CD117 antibody, or antigen-binding fragment thereof, does not comprise the CDRs or the variable regions set forth in SEQ ID NO: 46 and SEQ ID NO: 47. In certain embodiments, the anti-CD117 antibody, or antigen-binding fragment thereof, does not comprise the CDRs or the variable regions set forth in SEQ ID NO: 48 and SEQ ID NO: 49. In certain embodiments, the anti-CD117 antibody, or antigen-binding fragment thereof, does not comprise the CDRs or the variable regions set forth in SEQ ID NO: 50 and SEQ ID NO: 51. In certain embodiments, the anti-CD117 antibody, or antigen-binding fragment thereof, does not comprise the CDRs or the variable regions set forth in SEQ ID NO: 52 and SEQ ID NO: 53. In certain embodiments, the anti-CD117 antibody, or antigen-binding fragment thereof, does not comprise the CDRs or the variable regions set forth in SEQ ID NO: 54 and SEQ ID NO: 55. In certain embodiments, the anti-CD117 antibody, or antigen-binding fragment thereof, does not comprise the CDRs or the variable regions set forth in SEQ ID NO: 56 and SEQ ID NO: 57. In certain embodiments, the anti-CD117 antibody, or antigen-binding fragment thereof, does not comprise the CDRs or the variable regions set forth in SEQ ID NO: 58 and SEQ ID NO: 59. In certain embodiments, the anti-CD117 antibody, or antigen-binding fragment thereof, does not comprise the CDRs or the variable regions set forth in SEQ ID NO: 60 and SEQ ID NO: 61. In certain embodiments, the anti-CD117 antibody, or antigen-binding fragment thereof, does not comprise the CDRs or the variable regions set forth in SEQ ID NO: 62 and SEQ ID NO: 63. In certain embodiments, the anti-CD117 antibody, or antigen-binding fragment thereof, does not comprise the CDRs or the variable regions set forth in SEQ ID NO: 64 and SEQ ID NO: 65. In certain embodiments, the anti-CD117 antibody, or antigen-binding fragment thereof, does not comprise the CDRs or the variable regions set forth in SEQ ID NO: 66 and SEQ ID NO: 67. In certain embodiments, the anti-CD117 antibody, or antigen-binding fragment thereof, does not comprise the CDRs or the variable regions set forth in SEQ ID NO: 68 and SEQ ID NO: 69. In certain embodiments, the anti-CD117 antibody, or antigen-binding fragment thereof, does not comprise the CDRs or the variable regions set forth in SEQ ID NO: 70 and SEQ ID NO: 71. In certain embodiments, the anti-CD117 antibody, or antigen-binding fragment thereof, does not comprise the CDRs or the variable regions set forth in SEQ ID NO: 72 and SEQ ID NO: 73. In certain embodiments, the anti-CD117 antibody, or antigen-binding fragment thereof, does not comprise the CDRs or the variable regions set forth in SEQ ID NO: 74 and SEQ ID NO: 75. In certain embodiments, the anti-CD117 antibody, or antigen-binding fragment thereof, does not comprise the CDRs or the variable regions set forth in SEQ ID NO: 76 and SEQ ID NO: 77. In certain embodiments, the anti-CD117 antibody, or antigen-binding fragment thereof, does not comprise the CDRs or the variable regions set forth in SEQ ID NO: 78 and SEQ ID NO: 79. In certain embodiments, the anti-CD117 antibody, or antigen-binding fragment thereof, does not comprise the CDRs or the variable regions set forth in SEQ ID NO: 80 and SEQ ID NO: 81. In certain embodiments, the anti-CD117 antibody, or antigen-binding fragment thereof, does not comprise the CDRs or the variable regions set forth in SEQ ID NO: 82 and SEQ ID NO: 83. In certain embodiments, the anti-CD117 antibody, or antigen-binding fragment thereof, does not comprise the CDRs or the variable regions set forth in SEQ ID NO: 84 and SEQ ID NO: 85. In certain embodiments, the anti-CD117 antibody, or antigen-binding fragment thereof, does not comprise the CDRs or the variable regions set forth in SEQ ID NO: 86 and SEQ ID NO: 87. In certain embodiments, the anti-CD117 antibody, or antigen-binding fragment thereof, does not comprise the CDRs or the variable regions set forth in SEQ ID NO: 88 and SEQ ID NO: 89. In certain embodiments, the anti-CD117 antibody, or antigen-binding fragment thereof, does not comprise the CDRs or the variable regions set forth in SEQ ID NO: 90 and SEQ ID NO: 91. In certain embodiments, the anti-CD117 antibody, or antigen-binding fragment thereof, does not comprise the CDRs or the variable regions set forth in SEQ ID NO: 92 and SEQ ID NO: 93. In certain embodiments, the anti-CD117 antibody, or antigen-binding fragment thereof, does not comprise the CDRs or the variable regions set forth in SEQ ID NO: 94 and SEQ ID NO: 95. In certain embodiments, the anti-CD117 antibody, or antigen-binding fragment thereof, does not comprise the CDRs or the variable regions set forth in SEQ ID NO: 96 and SEQ ID NO: 97. In certain embodiments, the anti-CD117 antibody, or antigen-binding fragment thereof, does not comprise the CDRs or the variable regions set forth in SEQ ID NO: 98 and SEQ ID NO: 99. In certain embodiments, the anti-CD117 antibody, or antigen-binding fragment thereof, does not comprise the CDRs or the variable regions set forth in SEQ ID NO: 100 and SEQ ID NO: 101. In certain embodiments, the anti-CD117 antibody, or antigen-binding fragment thereof, does not comprise the CDRs or the variable regions set forth in SEQ ID NO: 102 and SEQ ID NO: 103. In certain embodiments, the anti-CD117 antibody, or antigen-binding fragment thereof, does not comprise the CDRs or the variable regions set forth in SEQ ID NO: 104 and SEQ ID NO: 105. In certain embodiments, the anti-CD117 antibody, or antigen-binding fragment thereof, does not comprise the CDRs or the variable regions set forth in SEQ ID NO: 106 and SEQ ID NO: 107. In certain embodiments, the anti-CD117 antibody, or antigen-binding fragment thereof, does not comprise the CDRs or the variable regions set forth in SEQ ID NO: 108 and SEQ ID NO: 109. In certain embodiments, the anti-CD117 antibody, or antigen-binding fragment thereof, does not comprise the CDRs or the variable regions set forth in SEQ ID NO: 110 and SEQ ID NO: 111. In certain embodiments, the anti-CD117 antibody, or antigen-binding fragment thereof, does not comprise the CDRs or the variable regions set forth in SEQ ID NO: 112 and SEQ ID NO: 113. In certain embodiments, the anti-CD117 antibody, or antigen-binding fragment thereof, does not comprise the CDRs or the variable regions set forth in SEQ ID NO: 114 and SEQ ID NO: 115. In certain embodiments, the anti-CD117 antibody, or antigen-binding fragment thereof, does not comprise the CDRs or the variable regions set forth in SEQ ID NO: 116 and SEQ ID NO: 117. In certain embodiments, the anti-CD117 antibody, or antigen-binding fragment thereof, does not comprise the CDRs or the variable regions set forth in SEQ ID NO: 118 and SEQ ID NO: 119. In certain embodiments, the anti-CD117 antibody, or antigen-binding fragment thereof, does not comprise the CDRs or the variable regions set forth in SEQ ID NO: 120 and SEQ ID NO: 121. In certain embodiments, the anti-CD117 antibody, or antigen-binding fragment thereof, does not comprise the CDRs or the variable regions set forth in SEQ ID NO: 122 and SEQ ID NO: 123. In certain embodiments, the anti-CD117 antibody, or antigen-binding fragment thereof, does not comprise the CDRs or the variable regions set forth in SEQ ID NO: 124 and SEQ ID NO: 125. In certain embodiments, the anti-CD117 antibody, or antigen-binding fragment thereof, does not comprise the CDRs or the variable regions set forth in SEQ ID NO: 126 and SEQ ID NO: 127. In certain embodiments, the anti-CD117 antibody, or antigen-binding fragment thereof, does not comprise the CDRs or the variable regions set forth in SEQ ID NO: 128 and SEQ ID NO: 129. In certain embodiments, the anti-CD117 antibody, or antigen-binding fragment thereof, does not comprise the CDRs or the variable regions set forth in SEQ ID NO: 130 and SEQ ID NO: 131. In certain embodiments, the anti-CD117 antibody, or antigen-binding fragment thereof, does not comprise the CDRs or the variable regions set forth in SEQ ID NO: 132 and SEQ ID NO: 133. In certain embodiments, the anti-CD117 antibody, or antigen-binding fragment thereof, does not comprise the CDRs or the variable regions set forth in SEQ ID NO: 134 and SEQ ID NO: 135. In certain embodiments, the anti-CD117 antibody, or antigen-binding fragment thereof, does not comprise the CDRs or the variable regions set forth in SEQ ID NO: 136 and SEQ ID NO: 137. In certain embodiments, the anti-CD117 antibody, or antigen-binding fragment thereof, does not comprise the CDRs or the variable regions set forth in SEQ ID NO: 138 and SEQ ID NO: 139. In certain embodiments, the anti-CD117 antibody, or antigen-binding fragment thereof, does not comprise the CDRs or the variable regions set forth in SEQ ID NO: 140 and SEQ ID NO: 141. In certain embodiments, the anti-CD117 antibody, or antigen-binding fragment thereof, does not comprise the CDRs or the variable regions set forth in SEQ ID NO: 142 and SEQ ID NO: 143. In yet another embodiment, the anti-CD117 antibody, or antigen-binding fragment thereof, is antagonistic.

In another aspect, the present disclosure provides a pharmaceutical composition comprising an isolated anti-CD117 antibody, or antigen-binding fragment thereof, wherein, when bound to CD117, the antibody, or antigen-binding fragment thereof, binds to at least two of the following amino acid residues T67, K69, T71, S81, Y83, T114, T119, or K129 of CD117 listed in SEQ ID NO: 1 and blocks or inhibits the biological activity of CD117. In one embodiment, the antibody, or antigen-binding fragment thereof, binds to at least three of the following amino acid residues T67, K69, T71, S81, Y83, T114, T119, or K129 of CD117 listed in SEQ ID NO: 1 and blocks or inhibits the biological activity of CD117. In another embodiment, the antibody, or antigen-binding fragment thereof, binds to at least four of the following amino acid residues T67, K69, T71, S81, Y83, T114, T119, or K129 of CD117 listed in SEQ ID NO: 1 and blocks or inhibits the biological activity of CD117. In some embodiments, the antibody, or antigen-binding fragment thereof, binds to at least five of the following amino acid residues T67, K69, T71, S81, Y83, T114, T119, or K129 of CD117 listed in SEQ ID NO: 1 and blocks or inhibits the biological activity of CD117. In another embodiment, the antibody, or antigen-binding fragment thereof, binds to at least six of the following amino acid residues T67, K69, T71, S81, Y83, T114, T119, or K129 of CD117 listed in SEQ ID NO: 1 and blocks or inhibits the biological activity of CD117. In yet another embodiment, the antibody, or antigen-binding fragment thereof, binds to at least seven of the following amino acid residues T67, K69, T71, S81, Y83, T114, T119, or K129 of CD117 listed in SEQ ID NO: 1 and blocks or inhibits the biological activity of CD117. In other embodiments, the antibody, or antigen-binding fragment thereof, binds to each of the following amino acid residues T67, K69, T71, S81, Y83, T114, T119, and K129 of CD117 listed in SEQ ID NO: 1 and blocks or inhibits the biological activity of CD117.

In one aspect, the present disclosure provides an isolated anti-CD117 antibody, or antigen-binding fragment thereof, which binds to human CD117 at least at two of the following amino acid residues T67, K69, T71, S81, Y83, T114, T119, or K129 of CD117 listed in SEQ ID NO: 1, wherein the isolated anti-CD117 antibody, or antigen-binding fragment thereof, does not have a heavy chain variable region comprising a CDR1 domain comprising the amino acid sequence as set forth in SEQ ID NO: 3, a CDR2 domain comprising the amino acid sequence as set forth in SEQ ID NO:4, and a CDR3 domain comprising the amino acid sequence as set forth in SEQ ID NO: 5; and a light chain variable region comprising a CDR1 domain comprising the amino acid sequence as set forth in SEQ ID NO: 7, a CDR2 domain comprising the amino acid sequence as set forth in SEQ ID NO:8, and a CDR3 domain comprising the amino acid sequence as set forth in SEQ ID NO: 9. In one embodiment, the isolated anti-CD117 antibody, or antigen-binding fragment thereof, binds to at least three of the following amino acid residues T67, K69, T71, S81, Y83, T114, T119, or K129 of CD117 listed in SEQ ID NO: 1, wherein the isolated anti-CD117 antibody, or antigen-binding fragment thereof, does not have a heavy chain variable region comprising a CDR1 domain comprising the amino acid sequence as set forth in SEQ ID NO: 3, a CDR2 domain comprising the amino acid sequence as set forth in SEQ ID NO:4, and a CDR3 domain comprising the amino acid sequence as set forth in SEQ ID NO: 5; and a light chain variable region comprising a CDR1 domain comprising the amino acid sequence as set forth in SEQ ID NO: 7, a CDR2 domain comprising the amino acid sequence as set forth in SEQ ID NO:8, and a CDR3 domain comprising the amino acid sequence as set forth in SEQ ID NO: 9. In another embodiment, the isolated anti-CD117 antibody, or antigen-binding fragment thereof, binds to at least four of the following amino acid residues T67, K69, T71, S81, Y83, T114, T119, or K129 of CD117 listed in SEQ ID NO: 1, wherein the isolated anti-CD117 antibody, or antigen-binding fragment thereof, does not have a heavy chain variable region comprising a CDR1 domain comprising the amino acid sequence as set forth in SEQ ID NO: 3, a CDR2 domain comprising the amino acid sequence as set forth in SEQ ID NO:4, and a CDR3 domain comprising the amino acid sequence as set forth in SEQ ID NO: 5; and a light chain variable region comprising a CDR1 domain comprising the amino acid sequence as set forth in SEQ ID NO: 7, a CDR2 domain comprising the amino acid sequence as set forth in SEQ ID NO:8, and a CDR3 domain comprising the amino acid sequence as set forth in SEQ ID NO: 9. In one embodiment, the isolated anti-CD117 antibody, or antigen-binding fragment thereof, binds to at least five of the following amino acid residues T67, K69, T71, S81, Y83, T114, T119, or K129 of CD117 listed in SEQ ID NO: 1, wherein the isolated anti-CD117 antibody, or antigen-binding fragment thereof, does not have a heavy chain variable region comprising a CDR1 domain comprising the amino acid sequence as set forth in SEQ ID NO: 3, a CDR2 domain comprising the amino acid sequence as set forth in SEQ ID NO:4, and a CDR3 domain comprising the amino acid sequence as set forth in SEQ ID NO: 5; and a light chain variable region comprising a CDR1 domain comprising the amino acid sequence as set forth in SEQ ID NO: 7, a CDR2 domain comprising the amino acid sequence as set forth in SEQ ID NO:8, and a CDR3 domain comprising the amino acid sequence as set forth in SEQ ID NO: 9. In one embodiment, the isolated anti-CD117 antibody, or antigen-binding fragment thereof, binds to at least six of the following amino acid residues T67, K69, T71, S81, Y83, T114, T119, or K129 of CD117 listed in SEQ ID NO: 1, wherein the isolated anti-CD117 antibody, or antigen-binding fragment thereof, does not have a heavy chain variable region comprising a CDR1 domain comprising the amino acid sequence as set forth in SEQ ID NO: 3, a CDR2 domain comprising the amino acid sequence as set forth in SEQ ID NO:4, and a CDR3 domain comprising the amino acid sequence as set forth in SEQ ID NO: 5; and a light chain variable region comprising a CDR1 domain comprising the amino acid sequence as set forth in SEQ ID NO: 7, a CDR2 domain comprising the amino acid sequence as set forth in SEQ ID NO:8, and a CDR3 domain comprising the amino acid sequence as set forth in SEQ ID NO: 9. In one embodiment, the isolated anti-CD117 antibody, or antigen-binding fragment thereof, binds to at least seven of the following amino acid residues T67, K69, T71, S81, Y83, T114, T119, or K129 of CD117 listed in SEQ ID NO: 1, wherein the isolated anti-CD117 antibody, or antigen-binding fragment thereof, does not have a heavy chain variable region comprising a CDR1 domain comprising the amino acid sequence as set forth in SEQ ID NO: 3, a CDR2 domain comprising the amino acid sequence as set forth in SEQ ID NO:4, and a CDR3 domain comprising the amino acid sequence as set forth in SEQ ID NO: 5; and a light chain variable region comprising a CDR1 domain comprising the amino acid sequence as set forth in SEQ ID NO: 7, a CDR2 domain comprising the amino acid sequence as set forth in SEQ ID NO:8, and a CDR3 domain comprising the amino acid sequence as set forth in SEQ ID NO: 9. In one embodiment, the isolated anti-CD117 antibody, or antigen-binding fragment thereof, binds to each of the following amino acid residues T67, K69, T71, S81, Y83, T114, T119, and K129 of CD117 listed in SEQ ID NO: 1, wherein the isolated anti-CD117 antibody, or antigen-binding fragment thereof, does not have a heavy chain variable region comprising a CDR1 domain comprising the amino acid sequence as set forth in SEQ ID NO: 3, a CDR2 domain comprising the amino acid sequence as set forth in SEQ ID NO:4, and a CDR3 domain comprising the amino acid sequence as set forth in SEQ ID NO: 5; and a light chain variable region comprising a CDR1 domain comprising the amino acid sequence as set forth in SEQ ID NO: 7, a CDR2 domain comprising the amino acid sequence as set forth in SEQ ID NO:8, and a CDR3 domain comprising the amino acid sequence as set forth in SEQ ID NO: 9.

In one aspect, the present disclosure provides an isolated anti-CD117 antibody, or antigen-binding fragment thereof, which binds to human CD117 at least at two of the following amino acid residues T67, K69, T71, S81, Y83, T114, T119, or K129 of CD117 listed in SEQ ID NO: 1, wherein the isolated anti-CD117 antibody, or antigen-binding fragment thereof, does not have a heavy chain variable region comprising the amino acid sequence as set forth in SEQ ID NO: 2, and a light chain variable region comprising the amino acid sequence as set forth in SEQ ID NO: 6.

In another aspect, the present disclosure provides a method of depleting a population of CD117+ cells in a human patient in need of a hematopoietic stem cell transplant by administering to the patient an effective amount of an isolated anti-CD117 antibody, or antigen-binding fragment thereof, or anti-CD117 ADC, wherein the isolated anti-CD117 antibody, or antigen-binding fragment thereof, or anti-CD117 ADC comprises an antibody, or antigen-binding fragment thereof, which binds to human CD117 at least at two of the following amino acid residues T67, K69, T71, S81, Y83, T114, T119, or K129 of CD117 listed in SEQ ID NO: 1. In one embodiment, the isolated anti-CD117 antibody, or antigen-binding fragment thereof, or anti-CD117 ADC, binds to at least three of the following amino acid residues T67, K69, T71, S81, Y83, T114, T119, or K129 of CD117 listed in SEQ ID NO: 1. In another embodiment, the isolated anti-CD117 antibody, or antigen-binding fragment thereof, or anti-CD117 ADC binds to at least four of the following amino acid residues T67, K69, T71, S81, Y83, T114, T119, or K129 of CD117 listed in SEQ ID NO: 1. In one embodiment, the isolated anti-CD117 antibody, or antigen-binding fragment thereof, or anti-CD117 ADC binds to at least five of the following amino acid residues T67, K69, T71, S81, Y83, T114, T119, or K129 of CD117 listed in SEQ ID NO: 1. In one embodiment, the isolated anti-CD117 antibody, or antigen-binding fragment thereof, or anti-CD117 ADC binds to at least six of the following amino acid residues T67, K69, T71, S81, Y83, T114, T119, or K129 of CD117 listed in SEQ ID NO: 1. In one embodiment, the isolated anti-CD117 antibody, or antigen-binding fragment thereof, or anti-CD117 ADC binds to at least seven of the following amino acid residues T67, K69, T71, S81, Y83, T114, T119, or K129 of CD117 listed in SEQ ID NO: 1. In one embodiment, the isolated anti-CD117 antibody, or antigen-binding fragment thereof, or anti-CD117 ADC binds to each of the following amino acid residues T67, K69, T71, S81, Y83, T114, T119, and K129 of CD117 listed in SEQ ID NO: 1.

In another aspect, the present disclosure provides a method comprising administering to a human patient an isolated anti-CD117 antibody, or antigen-binding fragment thereof, or anti-CD117 ADC, wherein the isolated anti-CD117 antibody, or antigen-binding fragment thereof, or anti-CD117 ADC comprises an antibody, or antigen-binding fragment thereof, which binds to human CD117 at least at two of the following amino acid residues T67, K69, T71, S81, Y83, T114, T119, or K129 of CD117 listed in SEQ ID NO: 1 and subsequently administering to the patient a transplant comprising hematopoietic stem cells. In one embodiment, the isolated anti-CD117 antibody, or antigen-binding fragment thereof, or anti-CD117 ADC, binds to at least three of the following amino acid residues T67, K69, T71, S81, Y83, T114, T119, or K129 of CD117 listed in SEQ ID NO: 1. In another embodiment, the isolated anti-CD117 antibody, or antigen-binding fragment thereof, or anti-CD117 ADC binds to at least four of the following amino acid residues T67, K69, T71, S81, Y83, T114, T119, or K129 of CD117 listed in SEQ ID NO: 1. In one embodiment, the isolated anti-CD117 antibody, or antigen-binding fragment thereof, or anti-CD117 ADC binds to at least five of the following amino acid residues T67, K69, T71, S81, Y83, T114, T119, or K129 of CD117 listed in SEQ ID NO: 1. In one embodiment, the isolated anti-CD117 antibody, or antigen-binding fragment thereof, or anti-CD117 ADC binds to at least six of the following amino acid residues T67, K69, T71, S81, Y83, T114, T119, or K129 of CD117 listed in SEQ ID NO: 1. In one embodiment, the isolated anti-CD117 antibody, or antigen-binding fragment thereof, or anti-CD117 ADC binds to at least seven of the following amino acid residues T67, K69, T71, S81, Y83, T114, T119, or K129 of CD117 listed in SEQ ID NO: 1. In one embodiment, the isolated anti-CD117 antibody, or antigen-binding fragment thereof, or anti-CD117 ADC binds to each of the following amino acid residues T67, K69, T71, S81, Y83, T114, T119, and K129 of CD117 listed in SEQ ID NO: 1.

### BRIEF DESCRIPTION OF THE FIGURES

FIGS. 1A and 1B provide the variable heavy (VH) and variable light (VL) chain region of the amino acid sequences of Ab85 and CK6. (A) Depicts the alignment of the variable heavy (VH) chain regions of Ab85 (SEQ ID NO: 2) and CK6 (SEQ ID NO: 22). (B) Depicts the alignment of the variable light (VL) chain regions of Ab85 (SEQ ID NO: 6) and CK6 (SEQ ID NO: 26).

FIG. 2 graphically depicts the results of an epitope mapping analysis characterizing the molecular interface between CD117 and Ab85 (SEQ ID NOS 147-148, respectively, in order of appearance).

### DETAILED DESCRIPTION

Described herein are isolated anti-CD117 human antibodies that bind to human CD117. The antibodies provided herein have many characteristics making them advantageous for therapy, including methods of conditioning human patients for stem cell transplantation. For example, antibodies disclosed herein, in certain embodiments, have high affinity and a low off rate for human CD117, as well as the ability to internalize in cells expressing CD117. Further, certain of the antibodies presented herein have improved biophysical stability. These features also make the anti-CD117 antibodies disclosed herein advantageous for use in conjugates (antibody drug conjugates (ADCs)) for delivering cytotoxins to CD117 expressing cells.

The present disclosure provides anti-CD117 antibodies, specifically isolated human anti-CD117 antibodies that bind to the ectodomain of human CD117. The binding regions of the isolated anti-CD117 antibodies identified herein are described below.

The anti-CD117 antibodies and ADCs described herein can be used in methods of treating a variety of disorders, such as diseases of a cell type in the hematopoietic lineage, cancers, autoimmune diseases, metabolic disorders, and stem cell disorders, among others. The compositions and methods described herein may (i) directly deplete a population of cells that give rise to a pathology, such as a population of cancer cells (e.g., leukemia cells) and autoimmune cells (e.g., autoreactive T-cells), and/or (ii) deplete a population of endogenous hematopoietic stem cells so as to promote the engraftment of transplanted hematopoietic stem cells by providing a niche to which the transplanted cells may home. The foregoing activities can be achieved by administration of an ADC, antibody, or antigen-binding fragment thereof, capable of binding an antigen expressed by an endogenous disease-causing cell or a hematopoietic stem cell. In the case of direct treatment of a disease, this administration can cause a reduction in the quantity of the cells that give rise to the pathology of interest. In the case of preparing a patient for hematopoietic stem cell transplant therapy, this administration can cause the selective depletion of a population of endogenous hematopoietic stem cells, thereby creating a vacancy in the hematopoietic tissue, such as the bone marrow, that can subsequently be filled by transplanted, exogenous hematopoietic stem cells. The present disclosure is based in part on the discovery that ADCs, antibodies, or antigen-binding fragments thereof, capable of binding CD117 (such as GNNK+ D117) can be administered to a patient to affect both of the above activities. ADCs, antibodies, or antigen-binding fragments thereof, that bind CD117 can be administered to a patient suffering from a cancer or autoimmune disease to directly deplete a population of cancerous cells or autoimmune cells, and can also be administered to a patient in need of hematopoietic stem cell transplant therapy in order to promote the survival and engraftment potential of transplanted hematopoietic stem cells.

Engraftment of hematopoietic stem cell transplants due to the administration of anti-CD117 ADCs, antibodies, or antigen-binding fragments thereof, can manifest in a variety of empirical measurements. For instance, engraftment of transplanted hematopoietic stem cells can be evaluated by assessing the quantity of competitive repopulating units (CRU) present within the bone marrow of a patient following administration of an ADC, antibody or antigen-binding fragment thereof capable of binding CD117 and subsequent administration of a hematopoietic stem cell transplant. Additionally, one can observe engraftment of a hematopoietic stem cell transplant by incorporating a reporter gene, such as an enzyme that catalyzes a chemical reaction yielding a fluorescent, chromophoric, or luminescent product, into a vector with which the donor hematopoietic stem cells have been transfected and subsequently monitoring the corresponding signal in a tissue into which the hematopoietic stem cells have homed, such as the bone marrow. One can also observe hematopoietic stem cell engraftment by evaluation of the quantity and survival of hematopoietic stem and progenitor cells, for instance, as determined by fluorescence activated cell sorting (FACS) analysis methods known in the art. Engraftment can also be determined by measuring white blood cell counts in peripheral blood during a post-transplant period, and/or by measuring recovery of marrow cells by donor cells in a bone marrow aspirate sample.

The sections that follow provide a description of ADCs, antibodies, or antigen-binding fragments thereof, that can be administered to a patient, such as a patient suffering from a cancer (such as acute myelogenous leukemia or myelodysplastic syndrome) or autoimmune disease, or a patient in need of hematopoietic stem cell transplant therapy in order to promote engraftment of hematopoietic stem cell grafts, as well as methods of administering such therapeutics to a patient (e.g., prior to hematopoietic stem cell transplantation).

### Definitions

As used herein, the term “about” refers to a value that is within 10% above or below the value being described. For example, the term “about 5 nM” indicates a range of from 4.5 nM to 5.5 nM.

As used herein, the term “amatoxin” refers to a member of the amatoxin family of peptides produced by Amanita phalloides mushrooms, or a variant or derivative thereof, such as a variant or derivative thereof capable of inhibiting RNA polymerase II activity. Amatoxins useful in conjunction with the compositions and methods described herein include compounds according to, but are not limited to, formula (III), including α-amanitin, β-amanitin, γ-amanitin, ε-amanitin, amanin, amaninamide, amanullin, amanullinic acid, or proamanullin. As described herein, amatoxins may be conjugated to an antibody, or antigen-binding fragment thereof, for instance, by way of a linker moiety (L) (thus forming an ADC). Exemplary methods of amatoxin conjugation and linkers useful for such processes are described below. Exemplary linker-containing amatoxins useful for conjugation to an antibody, or antigen-binding fragment, in accordance with the compositions and methods are also described herein.

Formula (III) is as follows:

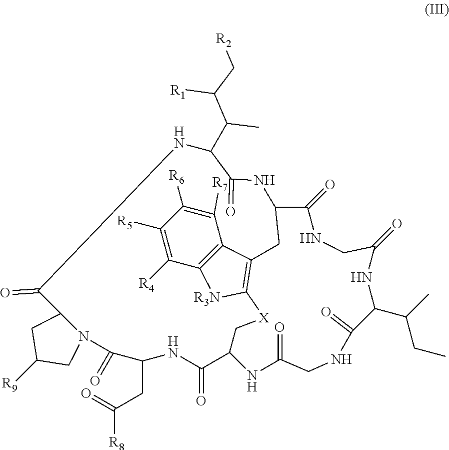

wherein R₁ is H, OH, or OR_{A};

R₂ is H, OH, or OR_{B};

R_{A} and R_{B}, when present, together with the oxygen atoms to which they are bound, combine to form an optionally substituted 5-membered heterocycloalkyl group;

R₃ is H or R_{D};

R₄ is H, OH, OR_{D}, or R_{D};

R₅ is H, OH, OR_{D}, or R_{D};

R₆ is H, OH, OR_{D}, or R_{D};

R₇ is H, OH, OR_{D}, or R_{D};

R₈ is OH, NH₂, or OR_{D};

R₉ is H, OH, or OR_{D};

X is —S—, —S(O)—, or —SO₂—; and

R_{D} is optionally substituted alkyl (e.g., C₁-C₆ alkyl), optionally substituted heteroalkyl (e.g., C₁-C₆ heteroalkyl), optionally substituted alkenyl (e.g., C₂-C₆ alkenyl), optionally substituted heteroalkenyl (e.g., C₂-C₆ heteroalkenyl), optionally substituted alkynyl (e.g., C₂-C₆ alkynyl), optionally substituted heteroalkynyl (e.g., C₂-C₆ heteroalkynyl), optionally substituted cycloalkyl, optionally substituted heterocycloalkyl, optionally substituted aryl, or optionally substituted heteroaryl.

For instance, amatoxins useful in conjunction with the compositions and methods described herein include compounds according to formula (IIIA), below:

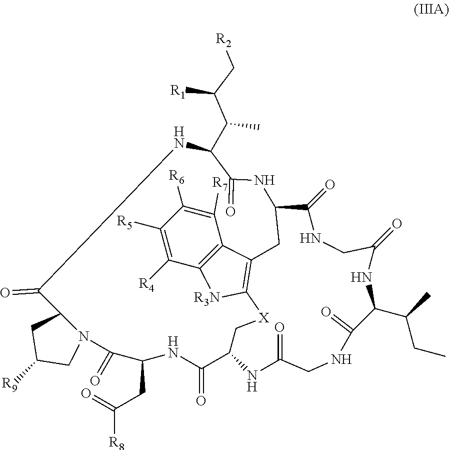

wherein R₁ is H, OH, or OR_{A};

R₂ is H, OH, or OR_{B};

R_{A} and R_{B}, when present, together with the oxygen atoms to which they are bound, combine to form an optionally substituted 5-membered heterocycloalkyl group;

R₃ is H or R_{D};

R₄ is H, OH, OR_{D}, or R_{D};

R₅ is H, OH, OR_{D}, or R_{D};

R₆ is H, OH, OR_{D}, or R_{D};

R₇ is H, OH, OR_{D}, or R_{D};

R₈ is OH, NH₂, or OR_{D};

R₉ is H, OH, or OR_{D};

X is —S—, —S(O)—, or —SO₂—; and

R_{D} is optionally substituted alkyl (e.g., C₁-C₆ alkyl), optionally substituted heteroalkyl (e.g., O₆ heteroalkyl), optionally substituted alkenyl (e.g., C₂-C₆ alkenyl), optionally substituted heteroalkenyl (e.g., C₂-C₆ heteroalkenyl), optionally substituted alkynyl (e.g., C₂-C₆ alkynyl), optionally substituted heteroalkynyl (e.g., C₂-C₆ heteroalkynyl), optionally substituted cycloalkyl, optionally substituted heterocycloalkyl, optionally substituted aryl, or optionally substituted heteroaryl.

Amatoxins useful in conjunction with the compositions and methods described herein also include compounds according to formula (IIIB), below:

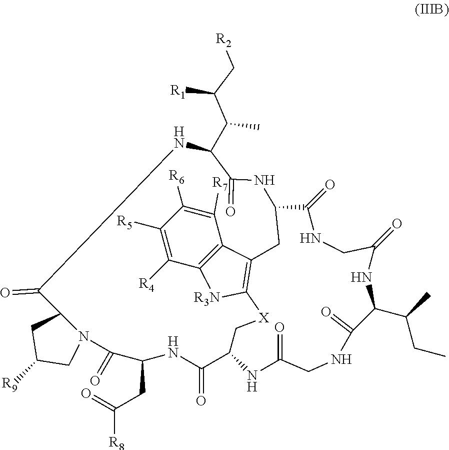

wherein R₁ is H, OH, or OR_{A};

R₂ is H, OH, or OR_{B};

R_{A} and R_{B}, when present, together with the oxygen atoms to which they are bound, combine to form an optionally substituted 5-membered heterocycloalkyl group;

R₃ is H or R_{D};

R₄ is H, OH, OR_{D}, or R_{D};

R₅ is H, OH, OR_{D}, or R_{D};

R₆ is H, OH, OR_{D}, or R_{D};

R₇ is H, OH, OR_{D}, or R_{D};

R₈ is OH, NH₂, or OR_{D};

R₉ is H, OH, or OR_{D};

X is —S—, —S(O)—, or —SO₂—; and

R_{D} is optionally substituted alkyl (e.g., C₁-C₆ alkyl), optionally substituted heteroalkyl (e.g., C₁-C₆ heteroalkyl), optionally substituted alkenyl (e.g., C₂-C₆ alkenyl), optionally substituted heteroalkenyl (e.g., C₂-C₆ heteroalkenyl), optionally substituted alkynyl (e.g., C₂-C₆ alkynyl), optionally substituted heteroalkynyl (e.g., C₂-C₆ heteroalkynyl), optionally substituted cycloalkyl, optionally substituted heterocycloalkyl, optionally substituted aryl, or optionally substituted heteroaryl.

For instance, in one embodiment, amatoxins useful in conjunction with the compositions and methods described herein include compounds according to formula (IIIC)

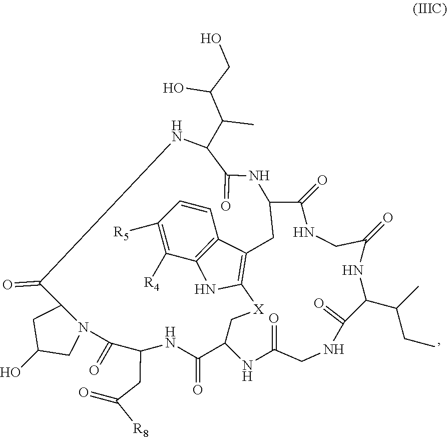

wherein R₄, R₅, X, and R₈ are each as defined above.

As described herein, amatoxins may be conjugated to an antibody, or antigen-binding fragment thereof, for instance, by way of a linker moiety (L) (thus forming an ADC). Exemplary methods of amatoxin conjugation and linkers useful for such processes are described below, including Table 1. Exemplary linker-containing amatoxins useful for conjugation to an antibody, or antigen-binding fragment, in accordance with the compositions and methods described herein are shown in structural formulas (I), (IA), (IB), (II), (IIA), or (IIB), recited herein.

As used herein, the term “antibody” refers to an immunoglobulin molecule that specifically binds to, or is immunologically reactive with, a particular antigen. An antibody includes, but is not limited to, monoclonal antibodies, polyclonal antibodies, multispecific antibodies (e.g., bispecific antibodies), genetically engineered, and otherwise modified forms of antibodies, including but not limited to de-immunized antibodies, chimeric antibodies, humanized antibodies, heteroconjugate antibodies (e.g., bi- tri- and quad-specific antibodies, diabodies, triabodies, and tetrabodies), and antibody fragments (i.e., antigen binding fragments of antibodies), including, for example, Fab′, F(ab′)₂, Fab, Fv, rIgG, and scFv fragments, so long as they exhibit the desired antigen-binding activity.

The antibodies of the present disclosure are generally isolated or recombinant. “Isolated,” when used herein refers to a polypeptide, e.g., an antibody, that has been identified and separated and/or recovered from a cell or cell culture from which it was expressed. Ordinarily, an isolated antibody will be prepared by at least one purification step. Thus, an “isolated antibody,” refers to an antibody which is substantially free of other antibodies having different antigenic specificities. For instance, an isolated antibody that specifically binds to CD117 is substantially free of antibodies that specifically bind antigens other than CD117.

The term “monoclonal antibody” (mAb) as used herein refers to an antibody that is derived from a single clone, including any eukaryotic, prokaryotic, or phage clone, by any means available or known in the art, and is not limited to antibodies produced through hybridoma technology. Monoclonal antibodies useful with the present disclosure can be prepared using a wide variety of techniques known in the art including the use of hybridoma, recombinant, and phage display technologies, or a combination thereof. The term “monoclonal antibody” is meant to include both intact molecules, as well as antibody fragments (including, for example, Fab and F(ab′)₂ fragments) that are capable of specifically binding to a target protein. As used herein, the Fab and F(ab′)₂ fragments refer to antibody fragments that lack the Fc fragment of an intact antibody. Examples of these antibody fragments are described herein.

Generally, antibodies comprise heavy and light chains containing antigen binding regions. Each heavy chain is comprised of a heavy chain variable region (abbreviated herein as HCVR or VH) and a heavy chain constant region. The heavy chain constant region is comprised of three domains, CH1, CH2 and CH3. Each light chain is comprised of a light chain variable region (abbreviated herein as LCVR or VL) and a light chain constant region. The light chain constant region is comprised of one domain, CL. The VH, and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxyl-terminus in the following order: FR1, CDR1, FR2, CDR2, FR3, CDR3, FR4. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (Clq) of the classical complement system.

An “intact” or “full length” antibody, as used herein, refers to an antibody having two heavy (H) chain polypeptides and two light (L) chain polypeptides interconnected by disulfide bonds. Each heavy chain is comprised of a heavy chain variable region (abbreviated herein as HCVR or VH) and a heavy chain constant region. The heavy chain constant region is comprised of three domains, CH1, CH2 and CH3. Each light chain is comprised of a light chain variable region (abbreviated herein as LCVR or VL) and a light chain constant region. The light chain constant region is comprised of one domain, CL. The VH, and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxyl-terminus in the following order: FR1, CDR1, FR2, CDR2, FR3, CDR3, FR4. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (Clq) of the classical complement system.

The terms “Fc”, “Fc region,” and “Fc domain,” as used herein refer to the portion of an immunoglobulin, e.g., an IgG molecule that correlates to a crystallizable fragment obtained by papain digestion of an IgG molecule. The Fc region comprises the C-terminal half of two heavy chains of an IgG molecule that are linked by disulfide bonds. It has no antigen binding activity but contains the carbohydrate moiety and binding sites for complement and Fc receptors, including the FcRn receptor (see below). For example, an Fc region contains the second constant domain CH2 (e.g., residues at EU positions 231-340 of IgG1) and the third constant domain CH3 (e.g., residues at EU positions 341-447 of human IgG1). As used herein, the Fc domain includes the “lower hinge region” (e.g., residues at EU positions 233-239 of IgG1).

Fc can refer to this region in isolation, or this region in the context of an antibody, antibody fragment, or Fc fusion protein. Polymorphisms have been observed at a number of positions in Fc domains, including but not limited to EU positions 270, 272, 312, 315, 356, and 358, and thus slight differences between the sequences presented in the instant application and sequences known in the art can exist. Thus, a “wild type IgG Fc domain” or “WT IgG Fc domain” refers to any naturally occurring IgG Fc region (i.e., any allele). The sequences of the heavy chains of human IgG1, IgG2, IgG3 and IgG4 can be found in a number of sequence databases, for example, at the Uniprot database (www.uniprot.org) under accession numbers P01857 (IGHG1_HUMAN), P01859 (IGHG2_HUMAN), P01860 (IGHG3_HUMAN), and P01861 (IGHG1_HUMAN), respectively. An example of a “WT” Fc region is provided in SEQ ID NO: 10 (which provides a heavy chain constant region containing an Fc region).

The terms “modified Fc region” or “variant Fc region” as used herein refers to an IgG Fc domain comprising one or more amino acid substitutions, deletions, insertions or modifications introduced at any position within the Fc region. In certain aspects a variant IgG Fc domain comprises one or more amino acid substitutions resulting in decreased or ablated binding affinity for an Fc gamma R and/or C1q as compared to the wild type Fc domain not comprising the one or more amino acid substitutions. Further, Fc binding interactions are essential for a variety of effector functions and downstream signaling events including, but not limited to, antibody dependent cell-mediated cytotoxicity (ADCC) and complement dependent cytotoxicity (CDC). Accordingly, in certain aspects, an antibody comprising a variant Fc domain (e.g., an antibody, fusion protein or conjugate) can exhibit altered binding affinity for at least one or more Fc ligands (e.g., Fc gamma Rs) relative to a corresponding antibody otherwise having the same amino acid sequence but not comprising the one or more amino acid substitution, deletion, insertion or modifications such as, for example, an unmodified Fc region containing naturally occurring amino acid residues at the corresponding position in the Fc region.

Variant Fc domains are defined according to the amino acid modifications that compose them. For all amino acid substitutions discussed herein in regard to the Fc region, numbering is always according to the EU index as in Kabat. Thus, for example, D265C is an Fc variant with the aspartic acid (D) at EU position 265 substituted with cysteine (C) relative to the parent Fc domain. It is noted that the order in which substitutions are provided is arbitrary. Likewise, e.g., D265C/L234A/L235A defines a variant Fc variant with substitutions at EU positions 265 (D to C), 234 (L to A), and 235 (L to A) relative to the parent Fc domain. A variant can also be designated according to its final amino acid composition in the mutated EU amino acid positions. For example, the L234A/L235A mutant can be referred to as “LALA”. As a further example, the E233P.L234V.L235A.delG236 (deletion of 236) mutant can be referred to as “EPLVLAdelG”. As yet another example, the I253A.H310A.H435A mutant can be referred to as “IHH”. It is noted that the order in which substitutions are provided is arbitrary.

The terms “Fc gamma receptor” or “Fc gamma R” as used herein refer to any member of the family of proteins that bind the IgG antibody Fc region and are encoded by the Fc gamma R genes. In humans this family includes but is not limited to Fcgamma RI (CD64), including isoforms Fc gamma RIa, Fc gamma RIb, and Fc gamma RIc; Fc gamma RII (CD32), including isoforms Fc gamma RIIa (including allotypes H131 and R131), Fc gamma RIIb (including Fc gamma RIIb-1 and Fc gamma RIIb-2), and Fc gamma RIIc; and Fc gamma RIII (CD16), including isoforms Fc gamma RIIIa (including allotypes V158 and F158) and Fc gamma RIIIb (including allotypes Fc gamma RIII b-NA1 and Fc gamma RIIIb-NA2), as well as any undiscovered human Fc gamma Rs or Fc gamma R isoforms or allotypes. An Fc gamma R can be from any organism, including but not limited to humans, mice, rats, rabbits, and monkeys. Mouse Fc gamma Rs include but are not limited to Fc gamma RI (CD64), Fc gamma RII (CD32), Fc gamma RIII (CD16), and Fc gamma RIII-2 (CD16-2), as well as any undiscovered mouse Fc gamma Rs or Fc gamma R isoforms or allotypes.

The term “effector function” as used herein refers to a biochemical event that results from the interaction of an Fc domain with an Fc receptor. Effector functions include but are not limited to ADCC, ADCP, and CDC. By “effector cell” as used herein is meant a cell of the immune system that expresses or one or more Fc receptors and mediates one or more effector functions. Effector cells include but are not limited to monocytes, macrophages, neutrophils, dendritic cells, eosinophils, mast cells, platelets, B cells, large granular lymphocytes, Langerhans' cells, natural killer (NK) cells, and gamma delta T cells, and can be from any organism included but not limited to humans, mice, rats, rabbits, and monkeys.

The term “silent”, “silenced”, or “silencing” as used herein refers to an antibody having a modified Fc region described herein that has decreased binding to an Fc gamma receptor (FcγR) relative to binding of an identical antibody comprising an unmodified Fc region to the FcγR (e.g., a decrease in binding to a FcγR by at least 70%, at least 80%, at least 90%, at least 95%, at least 98%, at least 99%, or 100% relative to binding of the identical antibody comprising an unmodified Fc region to the FcγR as measured by, e.g., BLI). In some embodiments, the Fc silenced antibody has no detectable binding to an FcγR. Binding of an antibody having a modified Fc region to an FcγR can be determined using a variety of techniques known in the art, for example but not limited to, equilibrium methods (e.g., enzyme-linked immunoabsorbent assay (ELISA); KinExA, Rathanaswami et al. Analytical Biochemistry, Vol. 373:52-60, 2008; or radioimmunoassay (RIA)), or by a surface plasmon resonance assay or other mechanism of kinetics-based assay (e.g., BIACORE™ analysis or Octet™ analysis (forteBIO)), and other methods such as indirect binding assays, competitive binding assays fluorescence resonance energy transfer (FRET), gel electrophoresis and chromatography (e.g., gel filtration). These and other methods may utilize a label on one or more of the components being examined and/or employ a variety of detection methods including but not limited to chromogenic, fluorescent, luminescent, or isotopic labels. A detailed description of binding affinities and kinetics can be found in Paul, W. E., ed., Fundamental Immunology, 4th Ed., Lippincott-Raven, Philadelphia (1999), which focuses on antibody-immunogen interactions. One example of a competitive binding assay is a radioimmunoassay comprising the incubation of labeled antigen with the antibody of interest in the presence of increasing amounts of unlabeled antigen, and the detection of the antibody bound to the labeled antigen. The affinity of the antibody of interest for a particular antigen and the binding off-rates can be determined from the data by scatchard plot analysis. Competition with a second antibody can also be determined using radioimmunoassays. In this case, the antigen is incubated with antibody of interest conjugated to a labeled compound in the presence of increasing amounts of an unlabeled second antibody.

As used herein, the term “identical antibody comprising an unmodified Fc region” refers to an antibody that lacks the recited amino acid substitutions (e.g., D265C, H435A, L234A, and/or L235A), but otherwise has the same amino acid sequence as the Fc modified antibody to which it is being compared.

The terms “antibody-dependent cell-mediated cytotoxicity” or “ADCC” refer to a form of cytotoxicity in which a polypeptide comprising an Fc domain, e.g., an antibody, bound onto Fc receptors (FcRs) present on certain cytotoxic cells (e.g., primarily NK cells, neutrophils, and macrophages) and enables these cytotoxic effector cells to bind specifically to an antigen-bearing “target cell” and subsequently kill the target cell with cytotoxins. (Hogarth et al., Nature review Drug Discovery 2012, 11:313) It is contemplated that, in addition to antibodies and fragments thereof, other polypeptides comprising Fc domains, e.g., Fc fusion proteins and Fc conjugate proteins, having the capacity to bind specifically to an antigen-bearing target cell will be able to effect cell-mediated cytotoxicity.

For simplicity, the cell-mediated cytotoxicity resulting from the activity of a polypeptide comprising an Fc domain is also referred to herein as ADCC activity. The ability of any particular polypeptide of the present disclosure to mediate lysis of the target cell by ADCC can be assayed. To assess ADCC activity, a polypeptide of interest (e.g., an antibody) is added to target cells in combination with immune effector cells, resulting in cytolysis of the target cell. Cytolysis is generally detected by the release of label (e.g., radioactive substrates, fluorescent dyes or natural intracellular proteins) from the lysed cells. Useful effector cells for such assays include peripheral blood mononuclear cells (PBMC) and Natural Killer (NK) cells. Specific examples of in vitro ADCC assays are described in Bruggemann et al., J. Exp. Med. 166:1351 (1987); Wilkinson et al., J. Immunol. Methods 258:183 (2001); Patel et al., J. Immunol. Methods 184:29 (1995). Alternatively, or additionally, ADCC activity of the antibody of interest can be assessed in vivo, e.g., in an animal model such as that disclosed in Clynes et al., Proc. Natl. Acad. Sci. USA 95:652 (1998).

The term “antigen-binding fragment,” as used herein, refers to one or more portions of an antibody that retain the ability to specifically bind to a target antigen. The antigen-binding function of an antibody can be performed by fragments of a full-length antibody. The antibody fragments can be, for example, a Fab, F(ab)₂, scFv, diabody, a triabody, an affibody, a nanobody, an aptamer, or a domain antibody. Examples of binding fragments encompassed of the term “antigen-binding fragment” of an antibody include, but are not limited to: (i) a Fab fragment, a monovalent fragment consisting of the V_{L}, V_{H}, C_{L}, and C_{H}1 domains; (ii) a F(ab′)₂ fragment, a bivalent fragment containing two Fab fragments linked by a disulfide bridge at the hinge region; (iii) a Fd fragment consisting of the V_{H} and C_{H}1 domains; (iv) a Fv fragment consisting of the V_{L} and V_{H} domains of a single arm of an antibody, (v) a dAb including V_{H} and V_{L} domains; (vi) a dAb fragment that consists of a V_{H} domain (see, e.g., Ward et al., Nature 341:544-546, 1989); (vii) a dAb which consists of a V_{H} or a V_{L} domain; (viii) an isolated complementarity determining region (CDR); and (ix) a combination of two or more (e.g., two, three, four, five, or six) isolated CDRs which may optionally be joined by a synthetic linker. Furthermore, although the two domains of the Fv fragment, V_{L} and V_{H}, are coded for by separate genes, they can be joined, using recombinant methods, by a linker that enables them to be made as a single protein chain in which the V_{L} and V_{H} regions pair to form monovalent molecules (known as single chain Fv (scFv); see, for example, Bird et al., Science 242:423-426, 1988 and Huston et al., Proc. Natl. Acad. Sci. USA 85:5879-5883, 1988). These antibody fragments can be obtained using conventional techniques known to those of skill in the art, and the fragments can be screened for utility in the same manner as intact antibodies. Antigen-binding fragments can be produced by recombinant DNA techniques, enzymatic or chemical cleavage of intact immunoglobulins, or, in certain cases, by chemical peptide synthesis procedures known in the art.

As used herein, the term “anti-CD117 antibody” or “an antibody that binds to CD117” refers to an antibody that is capable of binding CD117 with sufficient affinity such that the antibody is useful as a diagnostic and/or therapeutic agent in targeting CD117.

The term “antagonist” or “inhibitor”, as used herein, refer to a modulator that, when contacted with a molecule of interest causes a decrease in the magnitude of a certain activity or function of the molecule compared to the magnitude of the activity or function observed in the absence of the antagonist. Particular antagonists of interest include those that block or modulate the biological or immunological activity of human CD117. An “antagonist” anti-CD117 antibody inhibits SCF-dependent proliferation and is able to cross block SCF binding to CD117. Antagonists and inhibitors of human CD117 may include, but are not limited to, proteins, nucleic acids, carbohydrates, or any other molecules, which bind to human CD117. An example of an antagonist antibody is Ab85 (or an antibody having the binding regions of Ab85).

The term “inhibit binding to CD117” refers to the ability of an antibody, or antigen-binding fragment thereof, to prevent the association of CD117 with SCF (stem cell factor) and/or to prevent the binding and/or dimerization of CD117 to a further molecule of CD117. Such prevention of the association of CD117 with SCF and/or inhibition of binding and/or dimerization of CD117 to a further molecule of CD117 would result in diminishing or abolishing the biological activity mediated by the association of CD117 with SCF or by the binding and/or dimerization of CD117 to a further molecule of CD117.

The term “regulate” and “modulate” are used interchangeably, and, as used herein, refers to a change or an alteration in the activity of a molecule of interest (e.g., the biological activity of human CD117). Modulation may be an increase or a decrease in the magnitude of a certain activity or function of the molecule of interest. Exemplary activities and functions of a molecule include, but are not limited to, binding characteristics, enzymatic activity, cell receptor activation, and signal transduction.

Correspondingly, the term “modulator,” as used herein, is a compound capable of changing or altering an activity or function of a molecule of interest (e.g., the biological activity of human CD117). For example, a modulator may cause an increase or decrease in the magnitude of a certain activity or function of a molecule compared to the magnitude of the activity or function observed in the absence of the modulator. In certain embodiments, a modulator is an inhibitor, which decreases the magnitude of at least one activity or function of a molecule.

As used herein, the term “bispecific antibody” refers to an antibody, for example, a monoclonal, e.g., a de-immunized, a human or humanized antibody, that is capable of binding two different epitopes that can be on the same or different antigens. For instance, one of the binding specificities can be directed towards an epitope on a hematopoietic stem cell surface antigen, such as CD117 (e.g., GNNK+ CD117), and the other can specifically bind an epitope on a different hematopoietic stem cell surface antigen or another cell surface protein, such as a receptor or receptor subunit involved in a signal transduction pathway that potentiates cell growth, among others. In some embodiments, the binding specificities can be directed towards unique, non-overlapping epitopes on the same target antigen (i.e., a biparatopic antibody).

The term “de-immunized” or “de-immunization”, as used herein, relates to modification of an original wild type construct (or parent antibody) by rendering said wild type construct non-immunogenic or less immunogenic in humans. De-immunized antibodies contain part(s), e.g., a framework region(s) and/or a CDR(s), of non-human origin. As used herein, the term “deimmunized antibody” refers to an antibody that is de-immunized by mutation not to activate the immune system of a subject (for example, Nanus et al., J. Urology 170: S84-S89, 2003; WO98/52976; WO00/34317).

As used herein, the term “complementarity determining region” (CDR) refers to a hypervariable region found both in the light chain and the heavy chain variable domains of an antibody. The more highly conserved portions of variable domains are referred to as framework regions (FRs). The amino acid positions that delineate a hypervariable region of an antibody can vary, depending on the context and the various definitions known in the art. Some positions within a variable domain may be viewed as hybrid hypervariable positions in that these positions can be deemed to be within a hypervariable region under one set of criteria while being deemed to be outside a hypervariable region under a different set of criteria. One or more of these positions can also be found in extended hypervariable regions. The antibodies described herein may contain modifications in these hybrid hypervariable positions. The variable domains of native heavy and light chains each contain four framework regions that primarily adopt a β-sheet configuration, connected by three CDRs, which form loops that connect, and in some cases form part of, the β-sheet structure. The CDRs in each chain are held together in close proximity by the framework regions in the order FR1-CDR1-FR2-CDR2-FR3-CDR3-FR4 and, with the CDRs from the other antibody chains, contribute to the formation of the target binding site of antibodies (see Kabat et al., Sequences of Proteins of Immunological Interest, National Institute of Health, Bethesda, Md., 1987). In certain embodiments, numbering of immunoglobulin amino acid residues is performed according to the immunoglobulin amino acid residue numbering system of Kabat et al., unless otherwise indicated (although any antibody numbering scheme, including, but not limited to IMGT and Chothia, can be utilized).

As used herein, the terms “condition” and “conditioning” refer to processes by which a patient is prepared for receipt of a transplant, e.g., a transplant containing hematopoietic stem cells. Such procedures promote the engraftment of a hematopoietic stem cell transplant (for instance, as inferred from a sustained increase in the quantity of viable hematopoietic stem cells within a blood sample isolated from a patient following a conditioning procedure and subsequent hematopoietic stem cell transplantation. According to the methods described herein, a patient may be conditioned for hematopoietic stem cell transplant therapy by administration to the patient of an ADC, an antibody or antigen-binding fragment thereof capable of binding an antigen expressed by hematopoietic stem cells, such as CD117 (e.g., GNNK+ CD117). As described herein, the antibody may be covalently conjugated to a cytotoxin so as to form an antibody drug conjugate (ADC). Administration of an antibody, antigen-binding fragment thereof, or ADC capable of binding one or more of the foregoing antigens to a patient in need of hematopoietic stem cell transplant therapy can promote the engraftment of a hematopoietic stem cell graft, for example, by selectively depleting endogenous hematopoietic stem cells, thereby creating a vacancy filled by an exogenous hematopoietic stem cell transplant.

As used herein, the term “conjugate” or “antibody drug conjugate” or “ADC” refers to an antibody which is linked to a cytotoxin. An ADC is formed by the chemical bonding of a reactive functional group of one molecule, such as an antibody or antigen-binding fragment thereof, with an appropriately reactive functional group of another molecule, such as a cytotoxin described herein. Conjugates may include a linker between the two molecules bound to one another, e.g., between an antibody and a cytotoxin. Examples of linkers that can be used for the formation of a conjugate include peptide-containing linkers, such as those that contain naturally occurring or non-naturally occurring amino acids, such as D-amino acids. Linkers can be prepared using a variety of strategies described herein and known in the art. Depending on the reactive components therein, a linker may be cleaved, for example, by enzymatic hydrolysis, photolysis, hydrolysis under acidic conditions, hydrolysis under basic conditions, oxidation, disulfide reduction, nucleophilic cleavage, or organometallic cleavage (see, for example, Leriche et al., Bioorg. Med. Chem., 20:571-582, 2012). The foregoing conjugates are also referred to interchangeably herein as a “drug antibody conjugate”, an “antibody drug conjugate” and an “ADC”.

As used herein, the term “coupling reaction” refers to a chemical reaction in which two or more substituents suitable for reaction with one another react so as to form a chemical moiety that joins (e.g., covalently) the molecular fragments bound to each substituent. Coupling reactions include those in which a reactive substituent bound to a fragment that is a cytotoxin, such as a cytotoxin known in the art or described herein, reacts with a suitably reactive substituent bound to a fragment that is an antibody, or antigen-binding fragment thereof, such as an antibody, or antigen-binding fragment thereof, specific for CD117 (such as GNNK+ CD117) known in the art or described herein. Examples of suitably reactive substituents include a nucleophile/electrophile pair (e.g., a thiol/haloalkyl pair, an amine/carbonyl pair, or a thiol/α,β-unsaturated carbonyl pair, among others), a diene/dienophile pair (e.g., an azide/alkyne pair, among others), and the like. Coupling reactions include, without limitation, thiol alkylation, hydroxyl alkylation, amine alkylation, amine condensation, amidation, esterification, disulfide formation, cycloaddition (e.g., [4+2] Diels-Alder cycloaddition, [3+2] Huisgen cycloaddition, among others), nucleophilic aromatic substitution, electrophilic aromatic substitution, and other reactive modalities known in the art or described herein.

As used herein, “CRU (competitive repopulating unit)” refers to a unit of measure of long-term engrafting stem cells, which can be detected after in-vivo transplantation.

As used herein, “drug-to-antibody ratio” or “DAR” refers to the number of cytotoxins, e.g., amatoxin, attached to the antibody of an ADC. The DAR of an ADC can range from 1 to 8, although higher loads are also possible depending on the number of linkage sites on an antibody. Thus, in certain embodiments, an ADC described herein has a DAR of 1, 2, 3, 4, 5, 6, 7, or 8.

As used herein, the term “donor” refers to a human or animal from which one or more cells are isolated prior to administration of the cells, or progeny thereof, into a recipient. The one or more cells may be, for example, a population of hematopoietic stem cells.

As used herein, the term “diabody” refers to a bivalent antibody containing two polypeptide chains, in which each polypeptide chain includes V_{H} and V_{L} domains joined by a linker that is too short (e.g., a linker composed of five amino acids) to allow for intramolecular association of V_{H} and V_{L} domains on the same peptide chain. This configuration forces each domain to pair with a complementary domain on another polypeptide chain so as to form a homodimeric structure. Accordingly, the term “triabody” refers to trivalent antibodies containing three peptide chains, each of which contains one V_{H} domain and one V_{L} domain joined by a linker that is exceedingly short (e.g., a linker composed of 1-2 amino acids) to permit intramolecular association of V_{H} and V_{L} domains within the same peptide chain. In order to fold into their native structures, peptides configured in this way typically trimerize so as to position the V_{H} and V_{L} domains of neighboring peptide chains spatially proximal to one another (see, for example, Holliger et al., Proc. Natl. Acad. Sci. USA 90:6444-48, 1993).

As used herein, a “dual variable domain immunoglobulin” (“DVD-Ig”) refers to an antibody that combines the target-binding variable domains of two monoclonal antibodies via linkers to create a tetravalent, dual-targeting single agent (see, for example, Gu et al., Meth. Enzymol., 502:25-41, 2012).

As used herein, the term “endogenous” describes a substance, such as a molecule, cell, tissue, or organ (e.g., a hematopoietic stem cell or a cell of hematopoietic lineage, such as a megakaryocyte, thrombocyte, platelet, erythrocyte, mast cell, myeoblast, basophil, neutrophil, eosinophil, microglial cell, granulocyte, monocyte, osteoclast, antigen-presenting cell, macrophage, dendritic cell, natural killer cell, T-lymphocyte, or B-lymphocyte) that is found naturally in a particular organism, such as a human patient.

As used herein, the term “engraftment potential” is used to refer to the ability of hematopoietic stem and progenitor cells to repopulate a tissue, whether such cells are naturally circulating or are provided by transplantation. The term encompasses all events surrounding or leading up to engraftment, such as tissue homing of cells and colonization of cells within the tissue of interest. The engraftment efficiency or rate of engraftment can be evaluated or quantified using any clinically acceptable parameter as known to those of skill in the art and can include, for example, assessment of competitive repopulating units (CRU); incorporation or expression of a marker in tissue(s) into which stem cells have homed, colonized, or become engrafted; or by evaluation of the progress of a subject through disease progression, survival of hematopoietic stem and progenitor cells, or survival of a recipient. Engraftment can also be determined by measuring white blood cell counts in peripheral blood during a post-transplant period. Engraftment can also be assessed by measuring recovery of marrow cells by donor cells in a bone marrow aspirate sample.

As used herein, the term “epitope” includes any polypeptide determinant capable of specific binding to a binding protein, e.g., an antibody or antigen binding portion thereof. In certain embodiments, epitope determinants include chemically active surface groupings of molecules such as amino acids, sugar side chains, phosphoryl, or sulfonyl, and, in certain embodiments, may have specific three dimensional structural characteristics, and/or specific charge characteristics. In various embodiments, an epitope may be a linear or sequential epitope, i.e., a linear sequence of amino acids, of the primary structure of the antigen, i.e., CD117. Alternatively, in other embodiments, an epitope may be a conformational epitope having a specific three-dimensional shape when the antigen assumes its secondary structure. For example, the conformational epitope may comprise non-linear, i.e., non-sequential, amino acids of the antigen.

In a particular embodiment, an epitope is a region of an antigen that is bound by a binding protein, e.g., antibody or antigen binding portion thereof. In certain embodiments, a binding protein, e.g., antibody or antigen binding portion thereof, is said to specifically bind an antigen when it preferentially recognizes its target antigen in a complex mixture of proteins and/or macromolecules. In a particular embodiment, an epitope of the antigen, i.e., CD117, includes those amino acid residues within about 4 angstroms (Å) of the binding protein, e.g., antibody or antigen binding portion thereof, when the binding protein is bound to the antigen.

As used herein, the term “exogenous” describes a substance, such as a molecule, cell, tissue, or organ (e.g., a hematopoietic stem cell or a cell of hematopoietic lineage, such as a megakaryocyte, thrombocyte, platelet, erythrocyte, mast cell, myeoblast, basophil, neutrophil, eosinophil, microglial cell, granulocyte, monocyte, osteoclast, antigen-presenting cell, macrophage, dendritic cell, natural killer cell, T-lymphocyte, or B-lymphocyte) that is not found naturally in a particular organism, such as a human patient. A substance that is exogenous to a recipient organism, e.g., a recipient patient, may be naturally present in a donor organism, e.g., a donor subject, from which the substance is derived. For example, an allogeneic cell transplant contains cells that are exogenous to the recipient, but native to the donor. Exogenous substances include those that are provided from an external source to an organism or to cultured matter extracted therefrom.

As used herein, the term “framework region” or “FW region” includes amino acid residues that are adjacent to the CDRs of an antibody or antigen-binding fragment thereof. FW region residues may be present in, for example, human antibodies, humanized antibodies, monoclonal antibodies, antibody fragments, Fab fragments, single chain antibody fragments, scFv fragments, antibody domains, and bispecific antibodies, among others.

Also provided are “conservative sequence modifications” of the sequences set forth in the SEQ ID NOs described herein, i.e., nucleotide and amino acid sequence modifications which do not abrogate the binding of the antibody encoded by the nucleotide sequence or containing the amino acid sequence, to the antigen. Such conservative sequence modifications include conservative nucleotide and amino acid substitutions, as well as, nucleotide and amino acid additions and deletions. For example, modifications can be introduced into SEQ ID NOs described herein by standard techniques known in the art, such as site-directed mutagenesis and PCR-mediated mutagenesis. Conservative sequence modifications include conservative amino acid substitutions, in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, a predicted nonessential amino acid residue in an anti-CD117 antibody is preferably replaced with another amino acid residue from the same side chain family. Methods of identifying nucleotide and amino acid conservative substitutions that do not eliminate antigen binding are well-known in the art (see, e.g., Brummell et al., Biochem. 32:1180-1187 (1993); Kobayashi et al. Protein Eng. 12(10):879-884 (1999); and Burks et al. Proc. Natl. Acad. Sci. USA 94:412-417 (1997)).

As used herein, the term “half-life” refers to the time it takes for the plasma concentration of the antibody drug in the body to be reduced by one half or 50% in a subject, e.g., a human subject. This 50% reduction in serum concentration reflects the amount of drug circulating.

As used herein, the term “hematopoietic stem cells” (“HSCs”) refers to immature blood cells having the capacity to self-renew and to differentiate into mature blood cells containing diverse lineages including but not limited to granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), monocytes (e.g., monocytes, macrophages), dendritic cells, microglia, osteoclasts, and lymphocytes (e.g., NK cells, B-cells and T-cells). Such cells may include CD34⁺ cells. CD34⁺ cells are immature cells that express the CD34 cell surface marker. In humans, CD34⁺ cells are believed to include a subpopulation of cells with the stem cell properties defined above, whereas in mice, HSCs are CD34-. In addition, HSCs also refer to long term repopulating HSCs (LT-HSC) and short term repopulating HSCs (ST-HSC). LT-HSCs and ST-HSCs are differentiated, based on functional potential and on cell surface marker expression. For example, human HSCs are CD34⁺, CD38−, CD45RA−, CD90+, CD49F+, and lin− (negative for mature lineage markers including CD2, CD3, CD4, CD7, CD8, CD10, CD11B, CD19, CD20, CD56, CD235A). In mice, bone marrow LT-HSCs are CD34−, SCA-1+, C-kit+, CD135−, Slamfl/CD150+, CD48−, and lin− (negative for mature lineage markers including Ter119, CD11b, Gr1, CD3, CD4, CD8, B220, IL7ra), whereas ST-HSCs are CD34⁺, SCA-1+, C-kit+, CD135−, Slamfl/CD150+, and lin− (negative for mature lineage markers including Ter119, CD11b, Gr1, CD3, CD4, CD8, B220, IL7ra). In addition, ST-HSCs are less quiescent and more proliferative than LT-HSCs under homeostatic conditions. However, LT-HSC have greater self renewal potential (i.e., they survive throughout adulthood, and can be serially transplanted through successive recipients), whereas ST-HSCs have limited self renewal (i.e., they survive for only a limited period of time, and do not possess serial transplantation potential). Any of these HSCs can be used in the methods described herein. ST-HSCs are particularly useful because they are highly proliferative and thus, can more quickly give rise to differentiated progeny.

As used herein, the term “hematopoietic stem cell functional potential” refers to the functional properties of hematopoietic stem cells which include 1) multi-potency (which refers to the ability to differentiate into multiple different blood lineages including, but not limited to, granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), monocytes (e.g., monocytes, macrophages), dendritic cells, microglia, osteoclasts, and lymphocytes (e.g., NK cells, B-cells and T-cells), 2) self-renewal (which refers to the ability of hematopoietic stem cells to give rise to daughter cells that have equivalent potential as the mother cell, and further that this ability can repeatedly occur throughout the lifetime of an individual without exhaustion), and 3) the ability of hematopoietic stem cells or progeny thereof to be reintroduced into a transplant recipient whereupon they home to the hematopoietic stem cell niche and re-establish productive and sustained hematopoiesis.

As used herein, the term “human antibody” is intended to include antibodies having variable and constant regions derived from human germline immunoglobulin sequences. A human antibody may include amino acid residues not encoded by human germline immunoglobulin sequences (e.g., mutations introduced by random or site-specific mutagenesis in vitro or during gene rearrangement or by somatic mutation in vivo). However, the term “human antibody”, as used herein, is not intended to include antibodies in which CDR sequences derived from the germline of another mammalian species, such as a mouse, have been grafted onto human framework sequences. A human antibody can be produced in a human cell (for example, by recombinant expression) or by a non-human animal or a prokaryotic or eukaryotic cell that is capable of expressing functionally rearranged human immunoglobulin (such as heavy chain and/or light chain) genes. When a human antibody is a single chain antibody, it can include a linker peptide that is not found in native human antibodies. For example, an Fv can contain a linker peptide, such as two to about eight glycine or other amino acid residues, which connects the variable region of the heavy chain and the variable region of the light chain. Such linker peptides are considered to be of human origin. Human antibodies can be made by a variety of methods known in the art including phage display methods using antibody libraries derived from human immunoglobulin sequences. Human antibodies can also be produced using transgenic mice that are incapable of expressing functional endogenous immunoglobulins, but which can express human immunoglobulin genes (see, for example, PCT Publication Nos. WO 1998/24893; WO 1992/01047; WO 1996/34096; WO 1996/33735; U.S. Pat. Nos. 5,413,923; 5,625,126; 5,633,425; 5,569,825; 5,661,016; 5,545,806; 5,814,318; 5,885,793; 5,916,771; and 5,939,598).

“Humanized” forms of non-human (e.g., murine or rat) antibodies are immunoglobulins contain minimal sequences derived from non-human immunoglobulin. In general, a humanized antibody will comprise substantially all of at least one, and typically two, variable domains, in which all or substantially all of the CDR regions correspond to those of a non-human immunoglobulin and all or substantially all of the FR regions are those of a human immunoglobulin sequence. A humanized antibody can also comprise at least a portion of an immunoglobulin constant region (Fc), typically that of a human immunoglobulin consensus sequence. Methods of antibody humanization are known in the art and have been described, for example, in Riechmann et al., Nature 332:323-7, 1988; U.S. Pat. Nos. 5,530,101; 5,585,089; 5,693,761; 5,693,762; and U.S. Pat. No. 6,180,370 to Queen et al.; EP239400; PCT publication WO 91/09967; U.S. Pat. No. 5,225,539; EP592106; EP519596; Padlan, 1991, Mol. Immunol., 28:489-498; Studnicka et al., 1994, Prot. Eng. 7:805-814; Roguska et al., 1994, Proc. Natl. Acad. Sci. 91:969-973; and U.S. Pat. No. 5,565,332.

As used herein, patients that are “in need of” a hematopoietic stem cell transplant include patients that exhibit a defect or deficiency in one or more blood cell types, as well as patients having a stem cell disorder, autoimmune disease, cancer, or other pathology described herein. Hematopoietic stem cells generally exhibit 1) multi-potency, and can thus differentiate into multiple different blood lineages including, but not limited to, granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), monocytes (e.g., monocytes, macrophages), dendritic cells, microglia, osteoclasts, and lymphocytes (e.g., NK cells, B-cells and T-cells), 2) self-renewal, and can thus give rise to daughter cells that have equivalent potential as the mother cell, and 3) the ability to be reintroduced into a transplant recipient whereupon they home to the hematopoietic stem cell niche and re-establish productive and sustained hematopoiesis. Hematopoietic stem cells can thus be administered to a patient defective or deficient in one or more cell types of the hematopoietic lineage in order to re-constitute the defective or deficient population of cells in vivo. For example, the patient may be suffering from cancer, and the deficiency may be caused by administration of a chemotherapeutic agent or other medicament that depletes, either selectively or non-specifically, the cancerous cell population. Additionally or alternatively, the patient may be suffering from a hemoglobinopathy (e.g., a non-malignant hemoglobinopathy), such as sickle cell anemia, thalassemia, Fanconi anemia, aplastic anemia, and Wiskott-Aldrich syndrome. The subject may be one that is suffering from adenosine deaminase severe combined immunodeficiency (ADA SCID), HIV/AIDS, metachromatic leukodystrophy, Diamond-Blackfan anemia, and Schwachman-Diamond syndrome. The subject may have or be affected by an inherited blood disorder (e.g., sickle cell anemia) or an autoimmune disorder. Additionally or alternatively, the subject may have or be affected by a malignancy, such as neuroblastoma or a hematologic cancer. For instance, the subject may have a leukemia, lymphoma, or myeloma. In some embodiments, the subject has acute myeloid leukemia, acute lymphoid leukemia, chronic myeloid leukemia, chronic lymphoid leukemia, multiple myeloma, diffuse large B-cell lymphoma, or non-Hodgkin's lymphoma. In some embodiments, the subject has myelodysplastic syndrome. In some embodiments, the subject has an autoimmune disease, such as scleroderma, multiple sclerosis, ulcerative colitis, Crohn's disease, Type 1 diabetes, or another autoimmune pathology described herein. In some embodiments, the subject is in need of chimeric antigen receptor T-cell (CART) therapy. In some embodiments, the subject has or is otherwise affected by a metabolic storage disorder. The subject may suffer or otherwise be affected by a metabolic disorder selected from the group consisting of glycogen storage diseases, mucopolysaccharidoses, Gaucher's Disease, Hurlers Disease, sphingolipidoses, metachromatic leukodystrophy, or any other diseases or disorders which may benefit from the treatments and therapies disclosed herein and including, without limitation, severe combined immunodeficiency, Wiscott-Aldrich syndrome, hyper immunoglobulin M (IgM) syndrome, Chediak-Higashi disease, hereditary lymphohistiocytosis, osteopetrosis, osteogenesis imperfecta, storage diseases, thalassemia major, sickle cell disease, systemic sclerosis, systemic lupus erythematosus, multiple sclerosis, juvenile rheumatoid arthritis and those diseases, or disorders described in “Bone Marrow Transplantation for Non-Malignant Disease,” ASH Education Book, 1:319-338 (2000), the disclosure of which is incorporated herein by reference in its entirety as it pertains to pathologies that may be treated by administration of hematopoietic stem cell transplant therapy. Additionally or alternatively, a patient “in need of” a hematopoietic stem cell transplant may one that is or is not suffering from one of the foregoing pathologies, but nonetheless exhibits a reduced level (e.g., as compared to that of an otherwise healthy subject) of one or more endogenous cell types within the hematopoietic lineage, such as megakaryocytes, thrombocytes, platelets, erythrocytes, mast cells, myeoblasts, basophils, neutrophils, eosinophils, microglia, granulocytes, monocytes, osteoclasts, antigen-presenting cells, macrophages, dendritic cells, natural killer cells, T-lymphocytes, and B-lymphocytes. One of skill in the art can readily determine whether one's level of one or more of the foregoing cell types, or other blood cell type, is reduced with respect to an otherwise healthy subject, for instance, by way of flow cytometry and fluorescence activated cell sorting (FACS) methods, among other procedures, known in the art.

As used herein, the term “recipient” refers to a patient that receives a transplant, such as a transplant containing a population of hematopoietic stem cells. The transplanted cells administered to a recipient may be, e.g., autologous, syngeneic, or allogeneic cells.

As used herein, the term “sample” refers to a specimen (e.g., blood, blood component (e.g., serum or plasma), urine, saliva, amniotic fluid, cerebrospinal fluid, tissue (e.g., placental or dermal), pancreatic fluid, chorionic villus sample, and cells) taken from a subject.

As used herein, the term “scFv” refers to a single chain Fv antibody in which the variable domains of the heavy chain and the light chain from an antibody have been joined to form one chain.

scFv fragments contain a single polypeptide chain that includes the variable region of an antibody light chain (V_{L}) (e.g., CDR-L1, CDR-L2, and/or CDR-L3) and the variable region of an antibody heavy chain (V_{H}) (e.g., CDR-H1, CDR-H2, and/or CDR-H3) separated by a linker. The linker that joins the V_{L} and V_{H} regions of a scFv fragment can be a peptide linker composed of proteinogenic amino acids. Alternative linkers can be used to so as to increase the resistance of the scFv fragment to proteolytic degradation (for example, linkers containing D-amino acids), in order to enhance the solubility of the scFv fragment (for example, hydrophilic linkers such as polyethylene glycol-containing linkers or polypeptides containing repeating glycine and serine residues), to improve the biophysical stability of the molecule (for example, a linker containing cysteine residues that form intramolecular or intermolecular disulfide bonds), or to attenuate the immunogenicity of the scFv fragment (for example, linkers containing glycosylation sites). It will also be understood by one of ordinary skill in the art that the variable regions of the scFv molecules described herein can be modified such that they vary in amino acid sequence from the antibody molecule from which they were derived. For example, nucleotide or amino acid substitutions leading to conservative substitutions or changes at amino acid residues can be made (e.g., in CDR and/or framework residues) so as to preserve or enhance the ability of the scFv to bind to the antigen recognized by the corresponding antibody.

The terms “specific binding” or “specifically binding”, as used herein, refers to the ability of an antibody (or ADC) to recognize and bind to a specific protein structure (epitope) rather than to proteins generally. If an antibody is specific for epitope “A”, the presence of a molecule containing epitope A (or free, unlabeled A), in a reaction containing labeled “A” and the antibody, will reduce the amount of labeled A bound to the antibody. By way of example, an antibody “binds specifically” to a target if the antibody, when labeled, can be competed away from its target by the corresponding non-labeled antibody. In one embodiment, an antibody specifically binds to a target, e.g., CD117, if the antibody has a K_{D} for the target of at least about 10⁻⁴ M, 10⁻⁵ M, 10⁻⁶ M, 10⁻⁷ M, 10⁻⁸ M, 10⁻⁹ M, 10⁻¹⁰ M, 10⁻¹¹ M, 10⁻¹² M, or less (less meaning a number that is less than 10⁻¹², e.g. 10⁻¹³). In one embodiment, the term “specific binding to CD117” or “specifically binds to CD117,” as used herein, refers to an antibody (or ADC) that binds to CD117 and has a dissociation constant (K_{D}) of 1.0×10⁻⁷ M or less, as determined by surface plasmon resonance. In one embodiment, K_{D} (M) is determined according to standard bio-layer interferometery (BLI). In one embodiment, K_{off} (1/s) is determined according to standard bio-layer interferometery (BLI). It shall be understood, however, that the antibody may be capable of specifically binding to two or more antigens which are related in sequence. For example, in one embodiment, an antibody can specifically bind to both human and a non-human (e.g., mouse or non-human primate) orthologs of CD117.

As used herein, the terms “subject” and “patient” refer to an organism, such as a human, that receives treatment for a particular disease or condition as described herein. For instance, a patient, such as a human patient, may receive treatment prior to hematopoietic stem cell transplant therapy in order to promote the engraftment of exogenous hematopoietic stem cells.

As used herein, the phrase “substantially cleared from the blood” refers to a point in time following administration of a therapeutic agent (such as an anti-CD117 antibody, antigen-binding fragment thereof, or anti-CD117 ADC) to a patient when the concentration of the therapeutic agent in a blood sample isolated from the patient is such that the therapeutic agent is not detectable by conventional means (for instance, such that the therapeutic agent is not detectable above the noise threshold of the device or assay used to detect the therapeutic agent). A variety of techniques known in the art can be used to detect antibodies, antigen-binding fragments, and ADCs, such as ELISA-based detection assays known in the art or described herein. Additional assays that can be used to detect antibodies, or antibody fragments, include immunoprecipitation techniques and immunoblot assays, among others known in the art.

As used herein, the phrase “stem cell disorder” broadly refers to any disease, disorder, or condition that may be treated or cured by conditioning a subject's target tissues, and/or by ablating an endogenous stem cell population in a target tissue (e.g., ablating an endogenous hematopoietic stem or progenitor cell population from a subject's bone marrow tissue) and/or by engrafting or transplanting stem cells in a subject's target tissues. For example, Type I diabetes has been shown to be cured by hematopoietic stem cell transplant and may benefit from conditioning in accordance with the compositions and methods described herein. Additional disorders that can be treated using the compositions and methods described herein include, without limitation, sickle cell anemia, thalassemias, Fanconi anemia, aplastic anemia, Wiskott-Aldrich syndrome, ADA SCID, HIV/AIDS, metachromatic leukodystrophy, Diamond-Blackfan anemia, and Schwachman-Diamond syndrome. Additional diseases that may be treated using the patient conditioning and/or hematopoietic stem cell transplant methods described herein influde inherited blood disorders (e.g., sickle cell anemia) and autoimmune disorders, such as scleroderma, multiple sclerosis, ulcerative colitis, and Crohn's disease. Additional diseases that may be treated using the conditioning and/or transplantation methods described herein include a malignancy, such as a neuroblastoma or a hematologic cancer, such as leukemia, lymphoma, and myeloma. For instance, the cancer may be acute myeloid leukemia, acute lymphoid leukemia, chronic myeloid leukemia, chronic lymphoid leukemia, multiple myeloma, diffuse large B-cell lymphoma, or non-Hodgkin's lymphoma. Additional diseases treatable using the conditioning and/or transplantation methods described herein include myelodysplastic syndrome. In some embodiments, the subject has or is otherwise affected by a metabolic storage disorder. For example, the subject may suffer or otherwise be affected by a metabolic disorder selected from the group consisting of glycogen storage diseases, mucopolysaccharidoses, Gaucher's Disease, Hurlers Disease, sphingolipidoses, metachromatic leukodystrophy, or any other diseases or disorders which may benefit from the treatments and therapies disclosed herein and including, without limitation, severe combined immunodeficiency, Wiscott-Aldrich syndrome, hyper immunoglobulin M (IgM) syndrome, Chediak-Higashi disease, hereditary lymphohistiocytosis, osteopetrosis, osteogenesis imperfecta, storage diseases, thalassemia major, sickle cell disease, systemic sclerosis, systemic lupus erythematosus, multiple sclerosis, juvenile rheumatoid arthritis and those diseases, or disorders described in “Bone Marrow Transplantation for Non-Malignant Disease,” ASH Education Book, 1:319-338 (2000), the disclosure of which is incorporated herein by reference in its entirety as it pertains to pathologies that may be treated by administration of hematopoietic stem cell transplant therapy.

As used herein, the term “transfection” refers to any of a wide variety of techniques commonly used for the introduction of exogenous DNA into a prokaryotic or eukaryotic host cell, such as electroporation, lipofection, calcium-phosphate precipitation, DEAE-dextran transfection and the like.

As used herein, the terms “treat” or “treatment” refers to reducing the severity and/or frequency of disease symptoms, eliminating disease symptoms and/or the underlying cause of said symptoms, reducing the frequency or likelihood of disease symptoms and/or their underlying cause, and improving or remediating damage caused, directly or indirectly, by disease, any improvement of any consequence of disease, such as prolonged survival, less morbidity, and/or a lessening of side effects which are the byproducts of an alternative therapeutic modality; as is readily appreciated in the art, full eradication of disease is a preferred but albeit not a requirement for a treatment act. Beneficial or desired clinical results include, but are not limited to, promoting the engraftment of exogenous hematopoietic cells in a patient following antibody conditioning therapy as described herein and subsequent hematopoietic stem cell transplant therapy. Additional beneficial results include an increase in the cell count or relative concentration of hematopoietic stem cells in a patient in need of a hematopoietic stem cell transplant following conditioning therapy and subsequent administration of an exogenous hematopoietic stem cell graft to the patient. Beneficial results of therapy described herein may also include an increase in the cell count or relative concentration of one or more cells of hematopoietic lineage, such as a megakaryocyte, thrombocyte, platelet, erythrocyte, mast cell, myeoblast, basophil, neutrophil, eosinophil, microglial cell, granulocyte, monocyte, osteoclast, antigen-presenting cell, macrophage, dendritic cell, natural killer cell, T-lymphocyte, or B-lymphocyte, following conditioning therapy and subsequent hematopoietic stem cell transplant therapy. Additional beneficial results may include the reduction in quantity of a disease-causing cell population, such as a population of cancer cells (e.g., CD117+ leukemic cells) or autoimmune cells (e.g., CD117+ autoimmune lymphocytes, such as a CD117+ T-cell that expresses a T-cell receptor that cross-reacts with a self antigen). Insofar as the methods of the present disclosure are directed to preventing disorders, it is understood that the term “prevent” does not require that the disease state be completely thwarted. Rather, as used herein, the term preventing refers to the ability of the skilled artisan to identify a population that is susceptible to disorders, such that administration of the compounds of the present disclosure may occur prior to onset of a disease. The term does not imply that the disease state is completely avoided.

As used herein, the terms “variant” and “derivative” are used interchangeably and refer to naturally-occurring, synthetic, and semi-synthetic analogues of a compound, peptide, protein, or other substance described herein. A variant or derivative of a compound, peptide, protein, or other substance described herein may retain or improve upon the biological activity of the original material.

As used herein, the term “vector” includes a nucleic acid vector, such as a plasmid, a DNA vector, a plasmid, a RNA vector, virus, or other suitable replicon. Expression vectors described herein may contain a polynucleotide sequence as well as, for example, additional sequence elements used for the expression of proteins and/or the integration of these polynucleotide sequences into the genome of a mammalian cell. Certain vectors that can be used for the expression of antibodies and antibody fragments of the present disclosure include plasmids that contain regulatory sequences, such as promoter and enhancer regions, which direct gene transcription. Other useful vectors for expression of antibodies and antibody fragments contain polynucleotide sequences that enhance the rate of translation of these genes or improve the stability or nuclear export of the mRNA that results from gene transcription. These sequence elements may include, for example, 5′ and 3′ untranslated regions and a polyadenylation signal site in order to direct efficient transcription of the gene carried on the expression vector. The expression vectors described herein may also contain a polynucleotide encoding a marker for selection of cells that contain such a vector. Examples of a suitable marker include genes that encode resistance to antibiotics, such as ampicillin, chloramphenicol, kanamycin, and nourseothricin.

The term “acyl” as used herein refers to —C(═O)R, wherein R is hydrogen (“aldehyde”), alkyl, alkenyl, alkynyl, carbocyclyl, aryl, heteroaryl, or heterocyclyl, as defined herein. Non-limiting examples include formyl, acetyl, propanoyl, benzoyl, and acryloyl.

As used herein, the term “alkyl” refers to a straight- or branched-chain alkyl group having, for example, from 1 to 20 carbon atoms in the chain. Examples of alkyl groups include methyl, ethyl, n-propyl, isopropyl, butyl, isobutyl, sec-butyl, tert-butyl, pentyl, isopentyl, tert-pentyl, hexyl, isohexyl, and the like.

As used herein, the term “alkylene” refers to a straight- or branched-chain divalent alkyl group. The divalent positions may be on the same or different atoms within the alkyl chain. Examples of alkylene include methylene, ethylene, propylene, isopropylene, and the like.

As used herein, the term “heteroalkyl” refers to a straight or branched-chain alkyl group having, for example, from 1 to 20 carbon atoms in the chain, and further containing one or more heteroatoms (e.g., oxygen, nitrogen, or sulfur, among others) in the chain.

As used herein, the term “heteroalkylene” refers to a straight- or branched-chain divalent heteroalkyl group. The divalent positions may be on the same or different atoms within the heteroalkyl chain. The divalent positions may be one or more heteroatoms.

As used herein, the term “alkenyl” refers to a straight- or branched-chain alkenyl group having, for example, from 2 to 20 carbon atoms in the chain. Examples of alkenyl groups include vinyl, propenyl, isopropenyl, butenyl, tert-butylenyl, hexenyl, and the like.

As used herein, the term “alkenylene” refers to a straight- or branched-chain divalent alkenyl group. The divalent positions may be on the same or different atoms within the alkenyl chain. Examples of alkenylene include ethenylene, propenylene, isopropenylene, butenylene, and the like.

As used herein, the term “heteroalkenyl” refers to a straight- or branched-chain alkenyl group having, for example, from 2 to 20 carbon atoms in the chain, and further containing one or more heteroatoms (e.g., oxygen, nitrogen, or sulfur, among others) in the chain.

As used herein, the term “heteroalkenylene” refers to a straight- or branched-chain divalent heteroalkenyl group. The divalent positions may be on the same or different atoms within the heteroalkenyl chain. The divalent positions may be one or more heteroatoms.

As used herein, the term “alkynyl” refers to a straight- or branched-chain alkynyl group having, for example, from 2 to 20 carbon atoms in the chain. Examples of alkynyl groups include propargyl, butynyl, pentynyl, hexynyl, and the like.

As used herein, the term “alkynylene” refers to a straight- or branched-chain divalent alkynyl group. The divalent positions may be on the same or different atoms within the alkynyl chain.

As used herein, the term “heteroalkynyl” refers to a straight- or branched-chain alkynyl group having, for example, from 2 to 20 carbon atoms in the chain, and further containing one or more heteroatoms (e.g., oxygen, nitrogen, or sulfur, among others) in the chain.

As used herein, the term “heteroalkynylene” refers to a straight- or branched-chain divalent heteroalkynyl group. The divalent positions may be on the same or different atoms within the heteroalkynyl chain. The divalent positions may be one or more heteroatoms.

As used herein, the term “cycloalkyl” refers to a monocyclic, or fused, bridged, or spiro polycyclic ring structure that is saturated and has, for example, from 3 to 12 carbon ring atoms. Examples of cycloalkyl groups include cyclopropyl, cyclobutyl, cyclopentyl, cyclohexyl, cycloheptyl, cyclooctyl, bicyclo[3.1.0]hexane, and the like.

As used herein, the term “cycloalkylene” refers to a divalent cycloalkyl group. The divalent positions may be on the same or different atoms within the ring structure. Examples of cycloalkylene include cyclopropylene, cyclobutylene, cyclopentylene, cyclohexylene, and the like.

As used herein, the term “heterocyloalkyl” refers to a monocyclic, or fused, bridged, or spiro polycyclic ring structure that is saturated and has, for example, from 3 to 12 ring atoms per ring structure selected from carbon atoms and heteroatoms selected from, e.g., nitrogen, oxygen, and sulfur, among others. The ring structure may contain, for example, one or more oxo groups on carbon, nitrogen, or sulfur ring members. Examples of heterocycloalkyls include by way of example and not limitation dihydroypyridyl, tetrahydropyridyl (piperidyl), tetrahydrothiophenyl, piperidinyl, 4-piperidonyl, pyrrolidinyl, 2-pyrrolidonyl, tetrahydrofuranyl, tetrahydropyranyl, bis-tetrahydropyranyl, tetrahydroquinolinyl, tetrahydroisoquinolinyl, decahydroquinolinyl, octahydroisoquinolinyl, piperazinyl, quinuclidinyl, and morpholinyl.

As used herein, the term “heterocycloalkylene” refers to a divalent heterocyclolalkyl group. The divalent positions may be on the same or different atoms within the ring structure.

As used herein, the term “aryl” refers to a monocyclic or multicyclic aromatic ring system containing, for example, from 6 to 19 carbon atoms. Aryl groups include, but are not limited to, phenyl, fluorenyl, naphthyl, and the like. The divalent positions may be one or more heteroatoms.

As used herein, the term “arylene” refers to a divalent aryl group. The divalent positions may be on the same or different atoms.

As used herein, the term “heteroaryl” refers to a monocyclic heteroaromatic, or a bicyclic or a tricyclic fused-ring heteroaromatic group in which one or more ring atoms is a heteroatom, e.g., nitrogen, oxygen, or sulfur. Heteroaryl groups include pyridyl, pyrrolyl, furyl, thienyl, imidazolyl, oxazolyl, isoxazolyl, thiazolyl, isothiazolyl, pyrazolyl, 1,2,3-triazolyl, 1,2,4-triazolyl, 1,2,3-oxadiazolyl, 1,2,4-oxadia-zolyl, 1,2,5-oxadiazolyl, 1,3,4-oxadiazolyl, 1,3,4-triazinyl, 1,2,3-triazinyl, benzofuryl, [2,3-dihydro]benzofuryl, isobenzofuryl, benzothienyl, benzotriazolyl, isobenzothienyl, indolyl, isoindolyl, 3H-indolyl, benzimidazolyl, imidazo[1,2-a]pyridyl, benzothiazolyl, benzoxazolyl, quinolizinyl, quinazolinyl, pthalazinyl, quinoxalinyl, cinnolinyl, napthyridinyl, pyrido[3,4-b]pyridyl, pyrido[3,2-b]pyridyl, pyrido[4,3-b]pyridyl, quinolyl, isoquinolyl, tetrazolyl, 5,6,7,8-tetrahydroquinolyl, 5,6,7,8-tetrahydroisoquinolyl, purinyl, pteridinyl, carbazolyl, xanthenyl, benzoquinolyl, and the like.

As used herein, the term “heteroarylene” refers to a divalent heteroaryl group. The divalent positions may be on the same or different atoms. The divalent positions may be one or more heteroatoms.

Unless otherwise constrained by the definition of the individual substituent, the foregoing chemical moieties, such as “alkyl”, “alkylene”, “heteroalkyl”, “heteroalkylene”, “alkenyl”, “alkenylene”, “heteroalkenyl”, “heteroalkenylene”, “alkynyl”, “alkynylene”, “heteroalkynyl”, “heteroalkynylene”, “cycloalkyl”, “cycloalkylene”, “heterocyclolalkyl”, heterocycloalkylene”, “aryl,” “arylene”, “heteroaryl”, and “heteroarylene” groups can optionally be substituted with, for example, from 1 to 5 substituents selected from the group consisting of alkyl, alkenyl, alkynyl, cycloalkyl, heterocycloalkyl, alkyl aryl, alkyl heteroaryl, alkyl cycloalkyl, alkyl heterocycloalkyl, amino, ammonium, acyl, acyloxy, acylamino, aminocarbonyl, alkoxycarbonyl, ureido, carbamate, aryl, heteroaryl, sulfinyl, sulfonyl, alkoxy, sulfanyl, halogen, carboxy, trihalomethyl, cyano, hydroxy, mercapto, nitro, and the like. Typical substituents include, but are not limited to, —X, —R, —OH, —OR, —SH, —SR, NH₂, —NHR, —N(R)₂, —N⁺(R)₃, —CX₃, —CN, —OCN, —SCN, —NCO, —NCS, —NO, —NO₂, —N₃, —NC(═O)H, —NC(═O)R, —C(═O)H, —C(═O)R, —C(═O)NH₂, —C(═O)N(R)₂, —SO₃—, —SO₃H, —S(═O)₂R, —OS(═O)₂OR, —S(═O)₂NH₂, —S(═O)₂N(R)₂, —S(═O)R, —OP(═O)(OH)₂, —OP(═O)(OR)₂, —P(═O)(OR)₂, —PO₃, —PO₃H₂, —C(═O)X, —C(═S)R, —CO₂H, —CO₂R, —CO₂—, —C(═S)OR, —C(═O)SR, —C(═S)SR, —C(═O)NH₂, —C(═O)N(R)₂, —C(═S)NH₂, —C(═S)N(R)₂, —C(═NH)NH₂, and —C(═NR)N(R)₂; wherein each X is independently selected for each occasion from F, Cl, Br, and I; and each R is independently selected for each occasion from alkyl, aryl, heterocycloalkyl or heteroaryl, protecting group and prodrug moiety. Wherever a group is described as “optionally substituted,” that group can be substituted with one or more of the above substituents, independently for each occasion. The substitution may include situations in which neighboring substituents have undergone ring closure, such as ring closure of vicinal functional substituents, to form, for instance, lactams, lactones, cyclic anhydrides, acetals, hemiacetals, thioacetals, aminals, and hemiaminals, formed by ring closure, for example, to furnish a protecting group.

It is to be understood that certain radical naming conventions can include either a mono-radical or a di-radical, depending on the context. For example, where a substituent requires two points of attachment to the rest of the molecule, it is understood that the substituent is a di-radical. For example, a substituent identified as alkyl that requires two points of attachment includes di-radicals such as —CH₂—, —CH₂CH₂—, —CH₂CH(CH₃)CH₂—, and the like. Other radical naming conventions clearly indicate that the radical is a di-radical such as “alkylene,” “alkenylene,” “arylene,” “heterocycloalkylene,” and the like.

Wherever a substituent is depicted as a di-radical (i.e., has two points of attachment to the rest of the molecule), it is to be understood that the substituent can be attached in any directional configuration unless otherwise indicated.

“Isomerism” means compounds that have identical molecular formulae but differ in the sequence of bonding of their atoms or in the arrangement of their atoms in space. Isomers that differ in the arrangement of their atoms in space are termed “stereoisomers.” Stereoisomers that are not mirror images of one another are termed “diastereoisomers,” and stereoisomers that are non-superimposable mirror images of each other are termed “enantiomers,” or sometimes “optical isomers.”

A carbon atom bonded to four non-identical substituents is termed a “chiral center.” “Chiral isomer” means a compound with at least one chiral center. Compounds with more than one chiral center may exist either as an individual diastereomer or as a mixture of diastereomers, termed “diastereomeric mixture.” When one chiral center is present, a stereoisomer may be characterized by the absolute configuration (R or S) of that chiral center. Absolute configuration refers to the arrangement in space of the substituents attached to the chiral center. The substituents attached to the chiral center under consideration are ranked in accordance with the Sequence Rule of Cahn, Ingold and Prelog. (Cahn et al., Angew. Chem. Inter. Edit. 1966, 5, 385; errata 511; Cahn et al., Angew. Chem. 1966, 78, 413; Cahn and Ingold, J. Chem. Soc. 1951 (London), 612; Cahn et al., Experientia 1956, 12, 81; Cahn, J. Chem. Educ. 1964, 41, 116). A mixture containing equal amounts of individual enantiomeric forms of opposite chirality is termed a “racemic mixture.”

The compounds disclosed in this description and in the claims may comprise one or more asymmetric centers, and different diastereomers and/or enantiomers of each of the compounds may exist. The description of any compound in this description and in the claims is meant to include all enantiomers, diastereomers, and mixtures thereof, unless stated otherwise. In addition, the description of any compound in this description and in the claims is meant to include both the individual enantiomers, as well as any mixture, racemic or otherwise, of the enantiomers, unless stated otherwise. When the structure of a compound is depicted as a specific enantiomer, it is to be understood that the disclosure of the present application is not limited to that specific enantiomer. Accordingly, enantiomers, optical isomers, and diastereomers of each of the structural formulae of the present disclosure are contemplated herein. In the present specification, the structural formula of the compound represents a certain isomer for convenience in some cases, but the present disclosure includes all isomers, such as geometrical isomers, optical isomers based on an asymmetrical carbon, stereoisomers, tautomers, and the like, it being understood that not all isomers may have the same level of activity. The compounds may occur in different tautomeric forms. The compounds according to the disclosure are meant to include all tautomeric forms, unless stated otherwise. When the structure of a compound is depicted as a specific tautomer, it is to be understood that the disclosure of the present application is not limited to that specific tautomer.

The compounds of any formula described herein include the compounds themselves, as well as their salts, and their solvates, if applicable. A salt, for example, can be formed between an anion and a positively charged group (e.g., amino) on a compound of the disclosure. Suitable anions include chloride, bromide, iodide, sulfate, bisulfate, sulfamate, nitrate, phosphate, citrate, methanesulfonate, trifluoroacetate, glutamate, glucuronate, glutarate, malate, maleate, succinate, fumarate, tartrate, tosylate, salicylate, lactate, naphthalenesulfonate, and acetate (e.g., trifluoroacetate). The term “pharmaceutically acceptable anion” refers to an anion suitable for forming a pharmaceutically acceptable salt. Likewise, a salt can also be formed between a cation and a negatively charged group (e.g., carboxylate) on a compound of the disclosure. Suitable cations include sodium ion, potassium ion, magnesium ion, calcium ion, and an ammonium cation such as tetramethylammonium ion. Examples of some suitable substituted ammonium ions are those derived from: ethylamine, diethylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. The compounds of the disclosure also include those salts containing quaternary nitrogen atoms.

Examples of suitable inorganic anions include, but are not limited to, those derived from the following inorganic acids: hydrochloric, hydrobromic, hydroiodic, sulfuric, sulfurous, nitric, nitrous, phosphoric, and phosphorous. Examples of suitable organic anions include, but are not limited to, those derived from the following organic acids: 2-acetyoxybenzoic, acetic, ascorbic, aspartic, benzoic, camphorsulfonic, cinnamic, citric, edetic, ethanedisulfonic, ethanesulfonic, fumaric, glucheptonic, gluconic, glutamic, glycolic, hydroxymaleic, hydroxynaphthalene carboxylic, isethionic, lactic, lactobionic, lauric, maleic, malic, methanesulfonic, mucic, oleic, oxalic, palmitic, pamoic, pantothenic, phenylacetic, phenylsulfonic, propionic, pyruvic, salicylic, stearic, succinic, sulfanilic, tartaric, toluenesulfonic, and valeric. Examples of suitable polymeric organic anions include, but are not limited to, those derived from the following polymeric acids: tannic acid, carboxymethyl cellulose.

Additionally, the compounds of the present disclosure, for example, the salts of the compounds, can exist in either hydrated or unhydrated (the anhydrous) form or as solvates with other solvent molecules. Non-limiting examples of hydrates include monohydrates, dihydrates, etc. Non-limiting examples of solvates include ethanol solvates, acetone solvates, etc. “Solvate” means solvent addition forms that contain either stoichiometric or non-stoichiometric amounts of solvent. Some compounds have a tendency to trap a fixed molar ratio of solvent molecules in the crystalline solid state, thus forming a solvate. If the solvent is water the solvate formed is a hydrate; and if the solvent is alcohol, the solvate formed is an alcoholate. Hydrates are formed by the combination of one or more molecules of water with one molecule of the substance in which the water retains its molecular state as H2O. A hydrate refers to, for example, a mono-hydrate, a di-hydrate, a tri-hydrate, etc.

In addition, a crystal polymorphism may be present for the compounds or salts thereof represented by the formulae disclosed herein. It is noted that any crystal form, crystal form mixture, or anhydride or hydrate thereof, is included in the scope of the present disclosure.

### Anti-CD117 Antibodies

The present disclosure is based in part on the discovery of novel anti-CD117 antibodies and antigen binding portions thereof that are useful for therapeutic purposes. The present disclosure is also based in part on the discovery that antibodies, or antigen-binding fragments thereof, capable of binding CD117, such as GNNK+ CD117, can be used as therapeutic agents alone (or as ADCs) to (i) treat cancers (such as acute myelogenous leukemia or myelodysplastic syndrome) and autoimmune diseases characterized by CD117+ cells and (ii) promote the engraftment of transplanted hematopoietic stem cells in a patient in need of transplant therapy. These therapeutic activities can be caused, for instance, by the binding of anti-CD117 antibodies, or antigen-binding fragments thereof, to CD117 (e.g., GNNK+ CD117) expressed on the surface of a cell, such as a cancer cell, autoimmune cell, or hematopoietic stem cell and subsequently inducing cell death. The depletion of endogenous hematopoietic stem cells can provide a niche toward which transplanted hematopoietic stem cells can home, and subsequently establish productive hematopoiesis. In this way, transplanted hematopoietic stem cells may successfully engraft in a patient, such as human patient suffering from a stem cell disorder described herein.

Antibodies and antigen-binding fragments capable of binding human CD117 (also referred to as c-Kit, mRNA NCBI Reference Sequence: NM_000222.2, Protein NCBI Reference Sequence: NP_000213.1), including those capable of binding GNNK+ CD117, can be used in conjunction with the compositions and methods described herein in order to condition a patient for hematopoietic stem cell transplant therapy. Polymorphisms affecting the coding region or extracellular domain of CD117 in a significant percentage of the population are not currently well-known in non-oncology indications. There are at least four isoforms of CD117 that have been identified, with the potential of additional isoforms expressed in tumor cells. Two of the CD117 isoforms are located on the intracellular domain of the protein, and two are present in the external juxtamembrane region. The two extracellular isoforms, GNNK+ and GNNK−, differ in the presence (GNNK+) or absence (GNNK−) of a 4 amino acid sequence. These isoforms are reported to have the same affinity for the ligand (SCF), but ligand binding to the GNNK− isoform was reported to increase internalization and degradation. The GNNK+ isoform can be used as an immunogen in order to generate antibodies capable of binding CD117, as antibodies generated against this isoform will be inclusive of the GNNK+ and GNNK− proteins.

In some embodiments, the antibody, or antigen-binding fragment thereof binds human CD117 at an epitope located within amino acid residues of SEQ ID NO: 1. SEQ ID NO: 1 corresponds to the the human CD117 antigen and has the following amino acid sequence:

(SEQ ID NO: 1)
QPSVSPGEPSPPSIHPGKSDLIVRVGDEIRLLCTD
PGFVKWTFEILDETNENKQNEWITEKAEATNTGKY
TCTNKHGLSNSIYVFVRDPAKLFLVDRSLYGKEDN
DTLVRCPLTDPEVTNYSLKGCQGKPLPKDLRFIPD
PKAGIMIKSVKRAYHRLCLHCSVDQEGKSVLSEKF
ILKVRPAFKAVPVVSVSKASYLLREGEEFTVTCTI
KDVSSSVYSTWKRENSQTKLQEKYNSWHHGDFNYE
RQATLTISSARVNDSGVFMCYANNTFGSANVTTTL
EVVDKGFINIFPMINTTVFVNDGENVDLIVEYEAF
PKPEHQQWIYMNRTFTDKWEDYPKSENESNIRYVS
ELHLTRLKGTEGGTYTFLVSNSDVNAAIAFNVYVN
TKPEILTYDRLVNGMLQCVAAGFPEPTIDWYFCPG
TEQRCSASVLPVDVQTLNSSGPPFGKLVVQSSIDS
SAFKHNGTVECKAYNDVGKTSAYFNFAFKGNNKEQ
IHPHTHHHHHH.

As described in PCT/US2018/057180 (the disclosure of which is incorporated herein by reference in its entirety), an scFV phage display library screen of human antibodies was performed to identify novel anti-CD117 antibodies, and fragments thereof, having therapeutic use. Antibody 85 (Ab85) among others, was identified in this screen.

The heavy chain variable region (VH) amino acid sequence of Ab85 is provided below as SEQ ID NO: 2. The VH CDR amino acid sequences of Ab85 are underlined below and are as follows:

(VH CDR1; SEQ ID NO: 3)
NYVVIG;
(VH CDR2; SEQ ID NO: 4)
IINPRDSDTRYRPSFQG;
and
(VH CDR3; SEQ ID NO: 5)
HGRGYEGYEGAFDI.

Ab85 VH sequence

(SEQ ID NO: 2)
EVQLVQSGAEVKKPGESLKISCKGSGYSFTNYWIGWVRQMPGK
GLEWMAIINPRDSDTRYRPSFQGQVTISADKSISTAYLQWSSL
KASDTAMYYCARHGRGYEGYEGAFDIWGQGTLVTVSS

The light chain variable region (VL) amino acid sequence of Ab85 is provided below as SEQ ID NO 6. The VL CDR amino acid sequences of Ab85 are underlined below and are as follows:

(VL CDR1; SEQ ID NO: 7)
RSSQGIRSDLG;
(VL CDR2; SEQ ID NO: 8)
DASNLET;
and
(VL CDR3; SEQ ID NO: 9)
QQANGFPLT.

Ab85 VL sequence

(SEQ ID NO: 6)
DIQMTQSPSSLSASVGDRVTITCRSSQGIRSDLGVVYQQKPGK
APKLLIYDASNLETGVPSRFSGSGSGTDFTLTISSLQPEDFAT
YYCQQANGFPLTFGGGTKVEIK

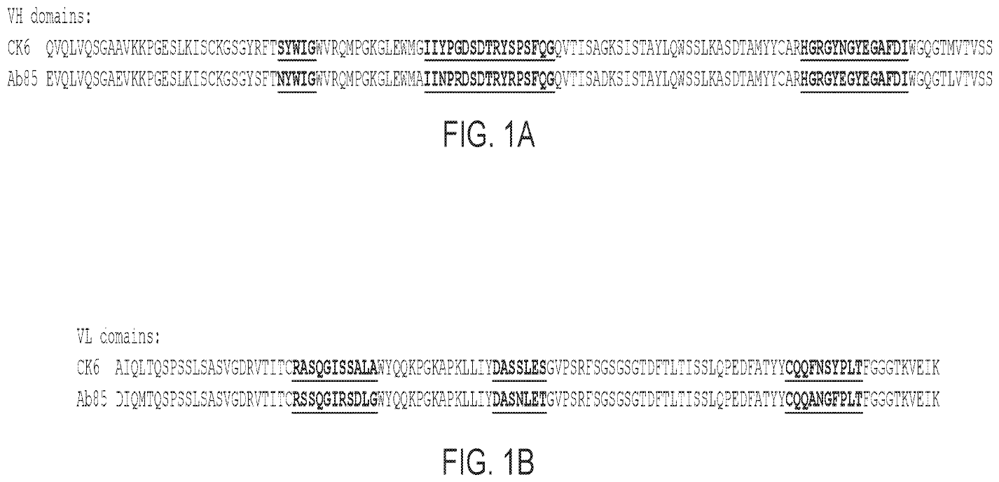

Human antibody Ab85 was derived from antibody CK6, which is an antagonist anti-CD117 antibody. A comparison of the amino acid sequences of the variable regions of Ab85 and Ab249 versus CK6 is shown in FIGS. 1A to 1B (where the CDRs are also designated). The Ab85 antibody has improved properties, e.g., improved binding characteristics, over CK6.

CK6 includes a potential deamidation site in the CDR3 domain of the heavy chain variable region. While advantageous to remove for future production of the antibody, the position of the asparagine presents a significant challenge. The potential deamidation site was successfully removed, however, in the Ab85 heavy chain CDR3 such that the antibody (having Ab85 heavy and light chain CDRs) was able to maintain a high affinity level specificity for human CD117 and the ability to internalize. Further, Ab85 has an improved off rate relative to its parent.

Thus, in certain embodiments, an anti-CD117 antibody comprises a heavy chain comprising a CDR set (CDR1, CDR2, and CDR3) as set forth in SEQ ID Nos: 3, 4, and 5, and a light chain comprising a CDR set as set forth in SEQ ID Nos: 7, 8, and 9 and internalizes in cells expressing CD117.

The anti-CD117 antibodies or binding fragments described herein may also include modifications and/or mutations that alter the properties of the antibodies and/or fragments, such as those that increase half-life, increase or decrease ADCC, etc., as is known in the art.

In one embodiment, the anti-CD117 antibody, or binding fragment thereof, comprises a variant (or modified) Fc region, wherein said variant Fc region comprises at least one amino acid modification relative to a wild-type Fc region, such that said molecule has an altered affinity for an FcgammaR. Certain amino acid positions within the Fc region are known through crystallography studies to make a direct contact with FcγR. Specifically, amino acids 234-239 (hinge region), amino acids 265-269 (B/C loop), amino acids 297-299 (C′/E loop), and amino acids 327-332 (F/G) loop. (see Sondermann et al., 2000 Nature, 406: 267-273). For example, amino acid substitutions at amino acid positions 234 and 235 of the Fc region have been identified as decreasing affinity of an IgG antibody for binding to an Fc receptor, particularly an Fc gamma receptor (FcγR). In one embodiment, an anti-CD117 antibody described herein comprises an Fc region comprising an amino acid substitution at L234 and/or L235, e.g., L234A and L235A (EU index). In some embodiments, the anti-CD117 antibodies described herein may comprise variant Fc regions comprising modification of at least one residue that makes a direct contact with an FcγR based on structural and crystallographic analysis. In one embodiment, the Fc region of the anti-CD117 antibody (or Fc containing fragment thereof) comprises an amino acid substitution at amino acid 265 according to the EU index as in Kabat et al., Sequences of Proteins of Immunological Interest, 5th Ed. Public Health Service, NH1, MD (1991), expressly incorporated herein by references. The “EU index as in Kabat” or “EU index” refers to the numbering of the human IgG1 EU antibody and is used herein in reference to Fc amino acid positions unless otherwise indicated.

In one embodiment, the Fc region comprises a D265A mutation. In one embodiment, the Fc region comprises a D265C mutation.

In some embodiments, the Fc region of the anti-CD117 antibody (or fragment thereof) comprises an amino acid substitution at amino acid 234 according to the EU index as in Kabat. In one embodiment, the Fc region comprises a L234A mutation. In some embodiments, the Fc region of the anti-CD117 antibody (or fragment thereof) comprises an amino acid substitution at amino acid 235 according to the EU index as in Kabat. In one embodiment, the Fc region comprises a L235A mutation. In yet another embodiment, the Fc region comprises a L234A and L235A mutation. In a further embodiment, the Fc region comprises a D265C, L234A, and L235A mutation.

In some embodiments, the Fc region of the anti-CD117 antibody (or fragment thereof) comprises an amino acid substitution at amino acid 239 according to the EU index as in Kabat. In one embodiment, the Fc region comprises a S239C mutation.

In one embodiment, the Fc region comprises a mutation at an amino acid position of D265, V205, H435, I253, and/or H310. For example, specific mutations at these positions include D265C, V205C, H435A, I253A, and/or H310A.

In one embodiment, the Fc region comprises a L234A mutation. In some embodiments, the Fc region of the anti-CD117 antibody (or fragment thereof) comprises an amino acid substitution at amino acid 235 according to the EU index as in Kabat. In one embodiment, the Fc region comprises a L235A mutation. In yet another embodiment, the Fc region comprises a L234A and L235A mutation. In a further embodiment, the Fc region comprises a D265C, L234A, and L235A mutation. In yet a further embodiment, the Fc region comprises a D265C, L234A, L235A, and H435A mutation. In a further embodiment, the Fc region comprises a D265C and H435A mutation.

In yet another embodiment, the Fc region comprises a L234A and L235A mutation (also referred to herein as “L234A.L235A” or as “LALA”). In another embodiment, the Fc region comprises a L234A and L235A mutation, wherein the Fc region does not include a P329G mutation. In a further embodiment, the Fc region comprises a D265C, L234A, and L235A mutation (also referred to herein as “D265C.L234A.L235A”). In another embodiment, the Fc region comprises a D265C, L234A, and L235A mutation, wherein the Fc region does not include a P329G mutation. In yet a further embodiment, the Fc region comprises a D265C, L234A, L235A, and H435A mutation (also referred to herein as “D265C.L234A.L235A.H435A”). In another embodiment, the Fc region comprises a D265C, L234A, L235A, and H435A mutation, wherein the Fc region does not include a P329G mutation. In a further embodiment, the Fc region comprises a D265C and H435A mutation (also referred to herein as “D265C.H435A”). In yet another embodiment, the Fc region comprises a D265A, S239C, L234A, and L235A mutation (also referred to herein as “D265A.S239C.L234A.L235A”). In yet another embodiment, the Fc region comprises a D265A, S239C, L234A, and L235A mutation, wherein the Fc region does not include a P329G mutation. In another embodiment, the Fc region comprises a D265C, N297G, and H435A mutation (also referred to herein as “D265C.N297G.H435A”). In another embodiment, the Fc region comprises a D265C, N297Q, and H435A mutation (also referred to herein as “D265C.N297Q.H435A”). In another embodiment, the Fc region comprises a E233P, L234V, L235A and delG236 (deletion of 236) mutation (also referred to herein as “E233P.L234V.L235A.delG236” or as “EPLVLAdelG”). In another embodiment, the Fc region comprises a E233P, L234V, L235A and delG236 (deletion of 236) mutation, wherein the Fc region does not include a P329G mutation. In another embodiment, the Fc region comprises a E233P, L234V, L235A, delG236 (deletion of 236) and H435A mutation (also referred to herein as “E233P.L234V.L235A.delG236.H435A” or as “EPLVLAdelG.H435A”). In another embodiment, the Fc region comprises a E233P, L234V, L235A, delG236 (deletion of 236) and H435A mutation, wherein the Fc region does not include a P329G mutation. In another embodiment, the Fc region comprises a L234A, L235A, S239C and D265A mutation. In another embodiment, the Fc region comprises a L234A, L235A, S239C and D265A mutation, wherein the Fc region does not include a P329G mutation. In another embodiment, the Fc region comprises a H435A, L234A, L235A, and D265C mutation. In another embodiment, the Fc region comprises a H435A, L234A, L235A, and D265C mutation, wherein the Fc region does not include a P329G mutation.

In some embodiments, the antibody has a modified Fc region such that, the anti-CD117 antibody decreases an effector function in an in vitro effector function assay with a decrease in binding to an Fc receptor (Fc R) relative to binding of an identical antibody comprising an unmodified Fc region to the FcR. In some embodiments, the antibody has a modified Fc region such that, the anti-CD117 antibody decreases an effector function in an in vitro effector function assay with a decrease in binding to an Fc gamma receptor (FcγR) relative to binding of an identical antibody comprising an unmodified Fc region to the FcγR. In some embodiments, the FcγR is FcγR1. In some embodiments, the FcγR is FcγR2A. In some embodiments, the FcγR is FcγR2B. In other embodiments, the FcγR is FcγR2C. In some embodiments, the FcγR is FcγR3A. In some embodiments, the FcγR is FcγR3B. In other embodiments, the decrease in binding is at least a 70% decrease, at least a 80% decrease, at least a 90% decrease, at least a 95% decrease, at least a 98% decrease, at least a 99% decrease, or a 100% decrease in antibody binding to a FcγR relative to binding of the identical antibody comprising an unmodified Fc region to the FcγR. In other embodiments, the decrease in binding is at least a 70% to a 100% decrease, at least a 80% to a 100% decrease, at least a 90% to a 100% decrease, at least a 95% to a 100% decrease, or at least a 98% to a 100% decrease, in antibody binding to a FcγR relative to binding of the identical antibody comprising an unmodified Fc region to the FcγR.

In some embodiments, the anti-CD117 antibody has a modified Fc region such that, the antibody decreases cytokine release in an in vitro cytokine release assay with a decrease in cytokine release of at least 50% relative to cytokine release of an identical antibody comprising an unmodified Fc region. In some embodiments, the decrease in cytokine release is at least a 70% decrease, at least a 80% decrease, at least a 90% decrease, at least a 95% decrease, at least a 98% decrease, at least a 99% decrease, or a 100% decrease in cytokine release relative to cytokine release of the identical antibody comprising an unmodified Fc region. In some embodiments, the decrease in cytokine release is at least a 70% to a 100% decrease, at least an 80% to a 100% decrease, at least a 90% to a 100% decrease, at least a 95% to a 100% decrease in cytokine release relative to cytokine release of the identical antibody comprising an unmodified Fc region. In certain embodiments, cytokine release is by immune cells.

In some embodiments, the anti-CD117 antibody has a modified Fc region such that, the antibody decreases mast cell degranulation in an in vitro mast cell degranulation assay with a decrease in mast cell degranulation of at least 50% relative to mast cell degranulation of an identical antibody comprising an unmodified Fc region. In some embodiments, the decrease in mast cell degranulation is at least a 70% decrease, at least a 80% decrease, at least a 90% decrease, at least a 95% decrease, at least a 98% decrease, at least a 99% decrease, or a 100% decrease in mast cell degranulation relative to mast cell degranulation of the identical antibody comprising an unmodified Fc region. In some embodiments, the decrease in mast cell degranulation is at least a 70% to a 100% decrease, at least a 80% to a 100% decrease, at least a 90% to a 100% decrease, or at least a 95% to a 100% decrease, in mast cell degranulation relative to mast cell degranulation of the identical antibody comprising an unmodified Fc region.

In some embodiments, the anti-CD117 antibody has a modified Fc region such that, the antibody decreases or prevents antibody dependent cell phagocytosis (ADCP) in an in vitro antibody dependent cell phagocytosis assay, with a decrease in ADCP of at least 50% relative to ADCP of an identical antibody comprising an unmodified Fc region. In some embodiments, the decrease in ADCP is at least a 70% decrease, at least a 80% decrease, at least a 90% decrease, at least a 95% decrease, at least a 98% decrease, at least a 99% decrease, or a 100% decrease in cytokine release relative to cytokine release of the identical antibody comprising an unmodified Fc region.

In some embodiments, the anti-CD117 antibody, as described herein, comprises an Fc region comprising one of the following modifications or combinations of modifications: D265A, D265C, D265C/H435A, D265C/LALA, D265C/LALA/H435A, D265A/S239C/L234A/L235A/H435A, D265A/S239C/L234A/L235A, D265C/N297G, D265C/N297G/H435A, D265C (EPLVLAdelG *), D265C (EPLVLAdelG)/H435A, D265C/N297Q/H435A, D265C/N297Q, EPLVLAdelG/H435A, EPLVLAdelG/D265C, EPLVLAdelG/D265A, N297A, N297G, or N297Q. In some embodiments, the anti-CD117 antibody herein comprises an Fc region comprising one of the following modifications or combinations of modifications: D265A, D265C, D265C/H435A, D265C/LALA, D265C/LALA/H435A, D265C/N297G, D265C/N297G/H435A, D265C (IgG2*), D265C (IgG2)/H435A, D265C/N297Q/H435A, D265C/N297Q, EPLVLAdelG/H435A, N297A, N297G, or N297Q.

Binding or affinity between a modified Fc region and a Fc gamma receptor can be determined using a variety of techniques known in the art, for example but not limited to, equilibrium methods (e.g., enzyme-linked immunoabsorbent assay (ELISA); KinExA, Rathanaswami et al. Analytical Biochemistry, Vol. 373:52-60, 2008; or radioimmunoassay (RIA)), or by a surface plasmon resonance assay or other mechanism of kinetics-based assay (e.g., BIACORE® analysis or Octet™ analysis (forteBIO)), and other methods such as indirect binding assays, competitive binding assays fluorescence resonance energy transfer (FRET), gel electrophoresis and chromatography (e.g., gel filtration). These and other methods may utilize a label on one or more of the components being examined and/or employ a variety of detection methods including but not limited to chromogenic, fluorescent, luminescent, or isotopic labels. A detailed description of binding affinities and kinetics can be found in Paul, W. E., ed., Fundamental Immunology, 4th Ed., Lippincott-Raven, Philadelphia (1999), which focuses on antibody-immunogen interactions. One example of a competitive binding assay is a radioimmunoassay comprising the incubation of labeled antigen with the antibody of interest in the presence of increasing amounts of unlabeled antigen, and the detection of the antibody bound to the labeled antigen. The affinity of the antibody of interest for a particular antigen and the binding off-rates can be determined from the data by scatchard plot analysis. Competition with a second antibody can also be determined using radioimmunoassays. In this case, the antigen is incubated with antibody of interest conjugated to a labeled compound in the presence of increasing amounts of an unlabeled second antibody.

In one embodiment, an antibody having the Fc modifications described herein (e.g., D265C, L234A, L235A, and/or H435A) has at least a 70% decrease, at least a 80% decrease, at least a 90% decrease, at least a 95% decrease, at least a 98% decrease, at least a 99% decrease, or a 100% decrease in binding to a Fc gamma receptor relative to binding of the identical antibody comprising an unmodified Fc region to the Fc gamma receptor (e.g., as assessed by biolayer interferometry (BLI)).

Without wishing to be bound by any theory, it is believed that Fc region binding interactions with a Fc gamma receptor are essential for a variety of effector functions and downstream signaling events including, but not limited to, antibody dependent cell-mediated cytotoxicity (ADCC) and complement dependent cytotoxicity (CDC). Accordingly, in certain aspects, an antibody comprising a modified Fc region (e.g., comprising a L234A, L235A, and/or a D265C mutation) has substantially reduced or abolished effector functions. Effector functions can be assayed using a variety of methods known in the art, e.g., by measuring cellular responses (e.g., mast cell degranulation or cytokine release) in response to the antibody of interest. For example, using standard methods in the art, the Fc-modified antibodies can be assayed for their ability to trigger mast cell degranulation in or for their ability to trigger cytokine release, e.g. by human peripheral blood mononuclear cells.

In certain aspects a variant IgG Fc domain comprises one or more amino acid substitutions resulting in decreased or ablated binding affinity for an FcgammaR and/or Clq as compared to the wild type Fc domain not comprising the one or more amino acid substitutions. Fc binding interactions are essential for a variety of effector functions and downstream signaling events including, but not limited to, antibody dependent cell-mediated cytotoxicity (ADCC) and complement dependent cytotoxicity (CDC). Accordingly, in certain aspects, an antibody comprising a modified Fc region (e.g., comprising a L234A, L235A, and a D265C mutation) has substantially reduced or abolished effector functions.

Affinity to an Fc region can be determined using a variety of techniques known in the art, for example but not limited to, equilibrium methods (e.g., enzyme-linked immunoabsorbent assay (ELISA); KinExA, Rathanaswami et al. Analytical Biochemistry, Vol. 373:52-60, 2008; or radioimmunoassay (RIA)), or by a surface plasmon resonance assay or other mechanism of kinetics-based assay (e.g., BIACORE™ analysis or Octet™ analysis (forteBIO)), and other methods such as indirect binding assays, competitive binding assays fluorescence resonance energy transfer (FRET), gel electrophoresis and chromatography (e.g., gel filtration). These and other methods may utilize a label on one or more of the components being examined and/or employ a variety of detection methods including but not limited to chromogenic, fluorescent, luminescent, or isotopic labels. A detailed description of binding affinities and kinetics can be found in Paul, W. E., ed., Fundamental Immunology, 4th Ed., Lippincott-Raven, Philadelphia (1999), which focuses on antibody-immunogen interactions. One example of a competitive binding assay is a radioimmunoassay comprising the incubation of labeled antigen with the antibody of interest in the presence of increasing amounts of unlabeled antigen, and the detection of the antibody bound to the labeled antigen. The affinity of the antibody of interest for a particular antigen and the binding off-rates can be determined from the data by scatchard plot analysis. Competition with a second antibody can also be determined using radioimmunoassays. In this case, the antigen is incubated with antibody of interest conjugated to a labeled compound in the presence of increasing amounts of an unlabeled second antibody.

In one embodiment, an anti-CD117 antibody described herein comprises an Fc region comprising L235A, L235A, and D265C (EU index). The antibodies of the present disclosure may be further engineered to further modulate antibody half-life by introducing additional Fc mutations, such as those described for example in (Dall'Acqua et al. (2006) J Biol Chem 281: 23514-24), (Zalevsky et al. (2010) Nat Biotechnol 28: 157-9), (Hinton et al. (2004) J Biol Chem 279: 6213-6), (Hinton et al. (2006) J Immunol 176: 346-56), (Shields et al. (2001) J Biol Chem 276: 6591-604), (Petkova et al. (2006) Int Immunol 18: 1759-69), (Datta-Mannan et al. (2007) Drug Metab Dispos 35: 86-94), (Vaccaro et al. (2005) Nat Biotechnol 23: 1283-8), (Yeung et al. (2010) Cancer Res 70: 3269-77) and (Kim et al. (1999) Eur J Immunol 29: 2819-25), and include positions 250, 252, 253, 254, 256, 257, 307, 376, 380, 428, 434 and 435. Exemplary mutations that may be made singularly or in combination are T250Q, M252Y, I253A, S254T, T256E, P2571, T307A, D376V, E380A, M428L, H433K, N434S, N434A, N434H, N434F, H435A and H435R mutations.

Thus, in one embodiment, the Fc region comprises a mutation resulting in a decrease in half life. An antibody having a short half life may be advantageous in certain instances where the antibody is expected to function as a short-lived therapeutic, e.g., the conditioning step described herein where the antibody is administered followed by HSCs. Ideally, the antibody would be substantially cleared prior to delivery of the HSCs, which also generally express CD117 but are not the target of the anti-CD117 antibody, unlike the endogenous stem cells. In one embodiment, the Fc region comprises a mutation at position 435 (EU index according to Kabat). In one embodiment, the mutation is an H435A mutation.

In one embodiment, the anti-CD117 antibody described herein has a half life of equal to or less than about 24 hours, equal to or less than about 22 hours, equal to or less than about 20 hours, equal to or less than about 18 hours, equal to or less than about 16 hours, equal to or less than about 14 hours, equal to or less than about 13 hours, equal to or less than about 12 hours, or equal to or less than about 11 hours. In one embodiment, the half life of the antibody is about 11 hours to about 24 hours; about 12 hours to about 22 hours; about 10 hours to about 20 hours; about 8 hours to about 18 hours; or about 14 hours to about 24 hours.

In some aspects, the Fc region comprises two or more mutations that confer reduced half-life and greatly diminish or completely abolish an effector function of the antibody. In some embodiments, the Fc region comprises a mutation resulting in a decrease in half-life and a mutation of at least one residue that can make direct contact with an FcγR (e.g., as based on structural and crystallographic analysis). In one embodiment, the Fc region comprises a H435A mutation, a L234A mutation, and a L235A mutation. In one embodiment, the Fc region comprises a H435A mutation and a D265C mutation. In one embodiment, the Fc region comprises a H435A mutation, a L234A mutation, a L235A mutation, and a D265C mutation. In one embodiment, the Fc region comprises a S239C mutation.

In some embodiments, the anti-CD117 antibody or antigen-binding fragment thereof is conjugated to a cytotoxin (e.g., calicheamicin) by way of a cysteine residue in the Fc domain of the antibody or antigen-binding fragment thereof. In some embodiments, the cysteine residue is introduced by way of a mutation in the Fc domain of the antibody or antigen-binding fragment thereof. For instance, the cysteine residue may be selected from the group consisting of Cys118, Cys239 (e.g., S239C), and Cys265. In one embodiment, the Fc region of the anti-CD117 antibody (or fragment thereof) comprises an amino acid substitution at amino acid 265 according to the EU index as in Kabat. In one embodiment, the Fc region comprises a D265C mutation. In one embodiment, the Fc region comprises a D265C and H435A mutation. In one embodiment, the Fc region comprises a D265C, a L234A, and a L235A mutation. In one embodiment, the Fc region comprises a D265C, a L234A, a L235A, and a H435A mutation. In one embodiment, the Fc region of the anti-CD117 antibody, or antigen-binding fragment thereof, comprises an amino acid substitution at amino acid 239 according to the EU index as in Kabat. In one embodiment, the Fc region comprises a S239C mutation. In one embodiment, the Fc region comprises a L234A mutation, a L235A mutation, a S239C mutation and a D265A mutation. In another embodiment, the Fc region comprises a S239C and H435A mutation. In another embodiment, the Fc region comprises a L234A mutation, a L235A mutation, and S239C mutation. In yet another embodiment, the Fc region comprises a H435A mutation, a L234A mutation, a L235A mutation, and S239C mutation. In yet another embodiment, the Fc region comprises a H435A mutation, a L234A mutation, a L235A mutation, a S239C mutation and D265A mutation.

Notably, Fc amino acid positions are in reference to the EU numbering index unless otherwise indicated.

In some embodiments of these aspects, the cysteine residue is naturally occurring in the Fc domain of the anti-CD117 antibody or antigen-binding fragment thereof. For instance, the Fc domain may be an IgG Fc domain, such as a human IgG1 Fc domain, and the cysteine residue may be selected from the group consisting of Cys261, Csy321, Cys367, and Cys425.

The variant Fc domains described herein are defined according to the amino acid modifications that compose them. For all amino acid substitutions discussed herein in regard to the Fc region, numbering is always according to the EU index. Thus, for example, D265C is an Fc variant with the aspartic acid (D) at EU position 265 substituted with cysteine (C) relative to the parent Fc domain. Likewise, e.g., D265C/L234A/L235A defines a variant Fc variant with substitutions at EU positions 265 (D to C), 234 (L to A), and 235 (L to A) relative to the parent Fc domain. A variant can also be designated according to its final amino acid composition in the mutated EU amino acid positions. For example, the L234A/L235A mutant can be referred to as LALA. It is noted that the order in which substitutions are provided is arbitrary.

Anti-CD117 antibodies that can be used in conjunction with the patient conditioning methods described herein include, for instance, antibodies produced and released from ATCC Accession No. 10716 (deposited as BA7.3C.9), such as the SR-1 antibody, which is described, for example, in U.S. Pat. No. 5,489,516, the disclosure of which is incorporated herein by reference as it pertains to anti-CD117 antibodies.

In one embodiment, an anti-CD117 antibody described herein comprises an Fc region comprising L235A, L235A, D265C, and H435A (EU index).

Additional anti-CD117 antibodies that can be used in conjunction with the patient conditioning methods described herein include those described in U.S. Pat. No. 7,915,391, which describes, e.g., humanized SR-1 antibodies; U.S. Pat. No. 5,808,002, which describes, e.g., the anti-CD117 A3C6E2 antibody, as well as those described in, for example, WO 2015/050959, which describes anti-CD117 antibodies that bind epitopes containing Pro317, Asn320, Glu329, Va1331, Asp332, Lus358, Glue360, Glue376, His378, and/or Thr380 of human CD117; and US 2012/0288506 (also published as U.S. Pat. No. 8,552,157), which describes, e.g., the anti-CD117 antibody CK6, having the CDR sequences of:

a CDR-H1 having the amino acid sequence SYWIG (SEQ ID NO: 23);

a CDR-H2 having the amino acid sequence IIYPGDSDTRYSPSFQG (SEQ ID NO: 24);

a CDR-H3 having the amino acid sequence HGRGYNGYEGAFDI (SEQ ID NO: 25);

a CDR-L1 having the amino acid sequence RASQGISSALA (SEQ ID NO: 27);

a CDR-L2 having the amino acid sequence DASSLES (SEQ ID NO: 28); and

a CDR-L3 having the amino acid sequence CQQFNSYPLT (SEQ ID NO: 29)

The heavy chain variable region amino acid sequence of CK6 is provided in SEQ ID NO: 22):

QVQLVQSGAAVKKPGESLKISCKGSGYRFTSYWIGVVVRQMPGK
GLEWMGIIYPGDSDTRYSPSFQGQVTISAGKSISTAYLQWSSLK
ASDTAMYYCARHGRGYNGYEGAFDIWGQGTMVTVSS
(SEQ ID NO: 22; CDRs are underlined are in bold).

The light chain amino acid variable sequence of CK6 is provided in SEQ ID NO: 26:

AIQLTQSPSSLSASVGDRVTITCRASQGISSALAWYQQKPG
KAPKLLIYDASSLESGVPSRFSGSGSGTDFTLTISSLQPED
FATYYCQQFNSYPLTFGGGTKVEIK
(SEQ ID NO: 26; CDRs are underlined and in bold).

Additional anti-CD117 antibodies and antigen-binding fragments thereof that may be used in conjunction with the compositions and methods described herein include those described in US 2015/0320880, such as the clones 9P3, NEG024, NEG027, NEG085, NEG086, and 20376.

The disclosures of each of the foregoing publications are incorporated herein by reference as they pertain to anti-CD117 antibodies. Antibodies and antigen-binding fragments that may be used in conjunction with the compositions and methods described herein include the above-described antibodies and antigen-binding fragments thereof, as well as humanized variants of those non-human antibodies and antigen-binding fragments described above and antibodies or antigen-binding fragments that bind the same epitope as those described above, as assessed, for instance, by way of a competitive CD117 binding assay.

Exemplary antigen-binding fragments of the foregoing antibodies include a dual-variable immunoglobulin domain, a single-chain Fv molecule (scFv), a diabody, a triabody, a nanobody, an antibody-like protein scaffold, a Fv fragment, a Fab fragment, a F(ab)₂ molecule, and a tandem di-scFv, among others.

Antibodies may be produced using recombinant methods and compositions, e.g., as described in U.S. Pat. No. 4,816,567. In one embodiment, isolated nucleic acid encoding an anti-CD117 antibody described herein is provided. Such nucleic acid may encode an amino acid sequence comprising the VL and/or an amino acid sequence comprising the VH of the antibody (e.g., the light and/or heavy chains of the antibody). In a further embodiment, one or more vectors (e.g., expression vectors) comprising such nucleic acid are provided. In a further embodiment, a host cell comprising such nucleic acid is provided. In one such embodiment, a host cell comprises (e.g., has been transformed with): (1) a vector comprising a nucleic acid that encodes an amino acid sequence comprising the VL of the antibody and an amino acid sequence comprising the VH of the antibody, or (2) a first vector comprising a nucleic acid that encodes an amino acid sequence comprising the VL of the antibody and a second vector comprising a nucleic acid that encodes an amino acid sequence comprising the VH of the antibody. In one embodiment, the host cell is eukaryotic, e.g. a Chinese Hamster Ovary (CHO) cell or lymphoid cell (e.g., Y0, NS0, Sp20 cell). In one embodiment, a method of making an anti-CLL-1 antibody is provided, wherein the method comprises culturing a host cell comprising a nucleic acid encoding the antibody, as provided above, under conditions suitable for expression of the antibody, and optionally recovering the antibody from the host cell (or host cell culture medium).

For recombinant production of an anti-CD117 antibody, nucleic acid encoding an antibody, e.g., as described above, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Vol. 248 (B. K. C. Lo, ed., Humana Press, Totowa, N.J., 2003), pp. 245-254, describing expression of antibody fragments in E. coli.) After expression, the antibody may be isolated from the bacterial cell paste in a soluble fraction and can be further purified.

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR− CHO cells (Urlaub et al., Proc. Natl. Acad. Sci. USA 77:4216 (1980)); and myeloma cell lines such as Y0, NS0 and Sp2/0. For a review of certain mammalian host cell lines suitable for antibody production, see, e.g., Yazaki and Wu, Methods in Molecular Biology, Vol. 248 (B. K. C. Lo, ed., Humana Press, Totowa, N.J.), pp. 255-268 (2003).

In one embodiment, the anti-CD117 antibody, or antigen binding fragment thereof, comprises variable regions having an amino acid sequence that is at least about 80%, about 85%, about 90%, about 91%, about 92%, about 93%, about 94%, about 95%, about 96%, about 97%, about 98% about 99% or more identical to the SEQ ID Nos disclosed herein. Alternatively, the anti-CD117 antibody, or antigen binding fragment thereof, comprises CDRs comprising the SEQ ID Nos disclosed herein with framework regions of the variable regions described herein having an amino acid sequence that is at least about 80%, about 85%, about 90%, about 91%, about 92%, about 93%, about 94%, about 95%, about 96%, about 97%, about 98% about 99% or more identical to the SEQ ID NOs disclosed herein.

In one embodiment, the anti-CD117 antibody, or antigen binding fragment thereof, comprises a heavy chain variable region and a heavy chain constant region having an amino acid sequence that is disclosed herein. In another embodiment, the anti-CD117 antibody, or antigen binding fragment thereof, comprises a light chain variable region and a light chain constant region having an amino acid sequence that is disclosed herein. In yet another embodiment, the anti-CD117 antibody, or antigen binding fragment thereof, comprises a heavy chain variable region, a light chain variable region, a heavy chain constant region and a light chain constant region having an amino acid sequence that is disclosed herein.

### Methods of Identifying Anti-CD117 Antibodies

Methods for high throughput screening of antibody, or antibody fragment, libraries for molecules capable of binding CD117 (e.g., GNNK+ CD117) can be used to identify and affinity mature antibodies useful for treating cancers, autoimmune diseases, and conditioning a patient (e.g., a human patient) in need of hematopoietic stem cell therapy as described herein. Such methods include in vitro display techniques known in the art, such as phage display, bacterial display, yeast display, mammalian cell display, ribosome display, mRNA display, and cDNA display, among others. The use of phage display to isolate ligands that bind biologically relevant molecules has been reviewed, for example, in Felici et al., Biotechnol. Annual Rev. 1:149-183, 1995; Katz, Annual Rev. Biophys. Biomol. Struct. 26:27-45, 1997; and Hoogenboom et al., Immunotechnology 4:1-20, 1998, the disclosures of each of which are incorporated herein by reference as they pertain to in vitro display techniques. Randomized combinatorial peptide libraries have been constructed to select for polypeptides that bind cell surface antigens as described in Kay, Perspect. Drug Discovery Des. 2:251-268, 1995 and Kay et al., Mol. Divers. 1:139-140, 1996, the disclosures of each of which are incorporated herein by reference as they pertain to the discovery of antigen-binding molecules. Proteins, such as multimeric proteins, have been successfully phage-displayed as functional molecules (see, for example, EP 0349578; EP 4527839; and EP 0589877, as well as Chiswell and McCafferty, Trends Biotechnol. 10:80-84 1992, the disclosures of each of which are incorporated herein by reference as they pertain to the use of in vitro display techniques for the discovery of antigen-binding molecules). In addition, functional antibody fragments, such as Fab and scFv fragments, have been expressed in in vitro display formats (see, for example, McCafferty et al., Nature 348:552-554, 1990; Barbas et al., Proc. Natl. Acad. Sci. USA 88:7978-7982, 1991; and Clackson et al., Nature 352:624-628, 1991, the disclosures of each of which are incorporated herein by reference as they pertain to in vitro display platforms for the discovery of antigen-binding molecules). These techniques, among others, can be used to identify and improve the affinity of antibodies that bind CD117 (e.g., GNNK+ CD117) that can in turn be used to deplete endogenous hematopoietic stem cells in a patient (e.g., a human patient) in need of hematopoietic stem cell transplant therapy.

In addition to in vitro display techniques, computational modeling techniques can be used to design and identify antibodies, or antibody fragments, in silico that bind CD117 (e.g., GNNK+ CD117). For example, using computational modeling techniques, one of skill in the art can screen libraries of antibodies, or antibody fragments, in silico for molecules capable of binding specific epitopes, such as extracellular epitopes of this antigen. The antibodies, or antigen-binding fragments thereof, identified by these computational techniques can be used in conjunction with the therapeutic methods described herein, such as the cancer and autoimmune disease treatment methods described herein and the patient conditioning procedures described herein.

Additional techniques can be used to identify antibodies, or antigen-binding fragments thereof, that bind CD117 (e.g., GNNK+ CD117) on the surface of a cell (e.g., a cancer cell, autoimmune cell, or hematopoietic stem cell) and that are internalized by the cell, for instance, by receptor-mediated endocytosis. For example, the in vitro display techniques described above can be adapted to screen for antibodies, or antigen-binding fragments thereof, that bind CD117 (e.g., GNNK+ CD117) on the surface of a cancer cell, autoimmune cell, or hematopoietic stem cell and that are subsequently internalized. Phage display represents one such technique that can be used in conjunction with this screening paradigm. To identify antibodies, or fragments thereof, that bind CD117 (e.g., GNNK+CD117) and are subsequently internalized by cancer cells, autoimmune cells, or hematopoietic stem cells, one of skill in the art can adapt the phage display techniques described, for example, in Williams et al., Leukemia 19:1432-1438, 2005, the disclosure of which is incorporated herein by reference in its entirety. For example, using mutagenesis methods known in the art, recombinant phage libraries can be produced that encode antibodies, antibody fragments, such as scFv fragments, Fab fragments, diabodies, triabodies, and ¹⁰Fn3 domains, among others, that contain randomized amino acid cassettes (e.g., in one or more, or all, of the CDRs or equivalent regions thereof or an antibody or antibody fragment). The framework regions, hinge, Fc domain, and other regions of the antibodies or antibody fragments may be designed such that they are non-immunogenic in humans, for instance, by virtue of having human germline antibody sequences or sequences that exhibit only minor variations relative to human germline antibodies.

Using phage display techniques described herein or known in the art, phage libraries containing randomized antibodies, or antibody fragments, covalently bound to the phage particles can be incubated with CD117 (e.g., GNNK+ CD117) antigen, for instance, by first incubating the phage library with blocking agents (such as, for instance, milk protein, bovine serum albumin, and/or IgG so as to remove phage encoding antibodies, or fragments thereof, that exhibit non-specific protein binding and phage that encode antibodies or fragments thereof that bind Fc domains, and then incubating the phage library with a population of hematopoietic stem cells. The phage library can be incubated with the target cells, such as cancer cells, autoimmune cells, or hematopoietic stem cells for a time sufficient to allow CD117-specific antibodies, or antigen-binding fragments thereof, (e.g., GNNK+ CD117-specific antibodies, or antigen-binding fragments thereof) to bind cell-surface CD117 (e.g., sell-surface GNNK+ CD117) antigen and to subsequently be internalized by the cancer cells, autoimmune cells, or hematopoietic stem cells (e.g., from 30 minutes to 6 hours at 4° C., such as 1 hour at 4° C.). Phage containing antibodies, or fragments thereof, that do not exhibit sufficient affinity for one or more of these antigens so as to permit binding to, and internalization by, cancer cells, autoimmune cells, or hematopoietic stem cells can subsequently be removed by washing the cells, for instance, with cold (4° C.) 0.1 M glycine buffer at pH 2.8. Phage bound to antibodies, or fragments thereof, or that have been internalized by the cancer cells, autoimmune cells, or hematopoietic stem cells can be identified, for instance, by lysing the cells and recovering internalized phage from the cell culture medium. The phage can then be amplified in bacterial cells, for example, by incubating bacterial cells with recovered phage in 2×YT medium using methods known in the art. Phage recovered from this medium can then be characterized, for instance, by determining the nucleic acid sequence of the gene(s) encoding the antibodies, or fragments thereof, inserted within the phage genome. The encoded antibodies, or fragments thereof, or can subsequently be prepared de novo by chemical synthesis (for instance, of antibody fragments, such as scFv fragments) or by recombinant expression (for instance, of full-length antibodies).

An exemplary method for in vitro evolution of anti-CD117 (e.g., anti-GNNK+ CD117) antibodies for use with the compositions and methods described herein is phage display. Phage display libraries can be created by making a designed series of mutations or variations within a coding sequence for the CDRs of an antibody or the analogous regions of an antibody-like scaffold (e.g., the BC, CD, and DE loops of 10Fn3 domains). The template antibody-encoding sequence into which these mutations are introduced may be, for example, a naive human germline sequence. These mutations can be performed using standard mutagenesis techniques known in the art. Each mutant sequence thus encodes an antibody corresponding to the template save for one or more amino acid variations. Retroviral and phage display vectors can be engineered using standard vector construction techniques known in the art. P3 phage display vectors along with compatible protein expression vectors can be used to generate phage display vectors for antibody diversification.

The mutated DNA provides sequence diversity, and each transformant phage displays one variant of the initial template amino acid sequence encoded by the DNA, leading to a phage population (library) displaying a vast number of different but structurally related amino acid sequences. Due to the well-defined structure of antibody hypervariable regions, the amino acid variations introduced in a phage display screen are expected to alter the binding properties of the binding peptide or domain without significantly altering its overall molecular structure.

In a typical screen, a phage library may be contacted with and allowed to bind one of the foregoing antigens or an epitope thereof. To facilitate separation of binders and non-binders, it is convenient to immobilize the target on a solid support. Phage bearing a CD117-binding moiety can form a complex with the target on the solid support, whereas non-binding phage remain in solution and can be washed away with excess buffer. Bound phage can then liberated from the target by changing the buffer to an extreme pH (pH 2 or pH 10), changing the ionic strength of the buffer, adding denaturants, or other known means.

The recovered phage can then be amplified through infection of bacterial cells, and the screening process can be repeated with the new pool that is now depleted in non-binding antibodies and enriched for antibodies that bind CD117 (e.g., GNNK+ CD117). The recovery of even a few binding phage is sufficient to amplify the phage for a subsequent iteration of screening. After a few rounds of selection, the gene sequences encoding the antibodies or antigen-binding fragments thereof derived from selected phage clones in the binding pool are determined by conventional methods, thus revealing the peptide sequence that imparts binding affinity of the phage to the target. During the panning process, the sequence diversity of the population diminishes with each round of selection until desirable peptide-binding antibodies remain. The sequences may converge on a small number of related antibodies or antigen-binding fragments thereof. An increase in the number of phage recovered at each round of selection is an indication that convergence of the library has occurred in a screen.

Another method for identifying antibodies includes using humanizing non-human antibodies that bind a cell surface target antigen (e.g., CD117), for instance, according to the following procedure. Consensus human antibody heavy chain and light chain sequences are known in the art (see e.g., the “VBASE” human germline sequence database; Kabat et al. Sequences of Proteins of Immunological Interest, Fifth Edition, U.S. Department of Health and Human Services, NIH Publication No. 91-3242, 1991; Tomlinson et al., J. Mol. Biol. 227:776-798, 1992; and Cox et al. Eur. J. Immunol. 24:827-836, 1994, the disclosures of each of which are incorporated herein by reference as they pertain to consensus human antibody heavy chain and light chain sequences. Using established procedures, one of skill in the art can identify the variable domain framework residues and CDRs of a consensus antibody sequence (e.g., by sequence alignment). One can substitute one or more CDRs of the heavy chain and/or light chain variable domains of consensus human antibody with one or more corresponding CDRs of a non-human antibody that binds a cell surface antigen (e.g., CD117) as described herein in order to produce a humanized antibody. This CDR exchange can be performed using gene editing techniques described herein or known in the art.

To produce humanized antibodies, one can recombinantly express a polynucleotide encoding the above consensus sequence in which one or more variable region CDRs have been replaced with one or more variable region CDR sequences of a non-human antibody that binds CD117 (e.g., GNNK+ CD117). As the affinity of the antibody for the hematopoietic stem cell antigen is determined primarily by the CDR sequences, the resulting humanized antibody is expected to exhibit an affinity for the hematopoietic stem cell antigen that is about the same as that of the non-human antibody from which the humanized antibody was derived. Methods of determining the affinity of an antibody for a target antigen include, for instance, ELISA-based techniques described herein and known in the art, as well as surface plasmon resonance, fluorescence anisotropy, and isothermal titration calorimetry, among others.

The internalizing capacity of the prepared antibodies, or fragments thereof, can be assessed, for instance, using radionuclide internalization assays known in the art. For example, antibodies, or fragments thereof, identified using in vitro display techniques described herein or known in the art can be functionalized by incorporation of a radioactive isotope, such as ¹⁸F, ⁷⁵Br, ⁷⁷Br, ¹²²I, ¹²³I, ¹²⁴I, ¹²⁵I, ¹²⁹I, ¹³¹I, ²¹¹At, ⁶⁷Ga, ¹¹¹In, ⁹⁹Tc, ¹⁶⁹Yb, ¹⁸⁶Re, ⁶⁴Cu, ⁶⁷Cu, ¹⁷⁷Lu, ⁷⁷As, ⁷²As, ⁸⁶Y, ⁹⁰Y, ⁸⁹Zr, ²¹²Bi, ²¹³Bi, or ²²⁵Ac. For instance, radioactive halogens, such as ¹⁸F, ⁷⁵Br, ⁷⁷Br, ¹²²I, ¹²³I, ¹²⁴I, ¹²⁵I, ¹²⁹I, ¹³¹I, ²¹¹At, can be incorporated into antibodies, or fragments thereof, using beads, such as polystyrene beads, containing electrophilic halogen reagents (e.g., Iodination Beads, Thermo Fisher Scientific, Inc., Cambridge, Mass.). Radiolabeled antibodies, or fragments thereof, can be incubated with cancer cells, autoimmune cells, or hematopoietic stem cells for a time sufficient to permit internalization (e.g., from 30 minutes to 6 hours at 4° C., such as 1 hour at 4° C.). The cells can then be washed to remove non-internalized antibodies, or fragments thereof, (e.g., using cold (4° C.) 0.1 M glycine buffer at pH 2.8). Internalized antibodies, or fragments thereof, can be identified by detecting the emitted radiation (e.g., γ-radiation) of the resulting cancer cells, autoimmune cells, or hematopoietic stem cells in comparison with the emitted radiation (e.g., γ-radiation) of the recovered wash buffer.

### CD117 Epitopes

In another aspect, the present disclosure pertains to an antibody, or an antigen binding fragment thereof, capable of binding CD117, which binds to an epitope in CD117 comprising at least two, at least three, at least four, at least five, at least six, at least seven, or all eight of the amino acid residues of T67, K69, T71, S81, Y83, T114, T119, and K129 of SEQ ID NO:1. In one embodiment, the antibody, or antigen binding fragment thereof, capable of binding CD117, binds to an epitope in CD117 comprising at least two of the amino acid residues of T67, K69, T71, S81, Y83, T114, T119, and K129 of SEQ ID NO:1. In one embodiment, the antibody, or antigen binding fragment thereof, capable of binding CD117, binds to an epitope in CD117 comprising at least three of the amino acid residues of T67, K69, T71, S81, Y83, T114, T119, and K129 of SEQ ID NO:1. In one embodiment, the antibody, or antigen binding fragment thereof, capable of binding CD117, binds to an epitope in CD117 comprising at least four of the amino acid residues of T67, K69, T71, S81, Y83, T114, T119, and K129 of SEQ ID NO:1. In one embodiment, the antibody, or antigen binding fragment thereof, capable of binding CD117, binds to an epitope in CD117 comprising at least five of the amino acid residues of T67, K69, T71, S81, Y83, T114, T119, and K129 of SEQ ID NO:1. In one embodiment, the antibody, or antigen binding fragment thereof, capable of binding CD117, binds to an epitope in CD117 comprising at least six of the amino acid residues of T67, K69, T71, S81, Y83, T114, T119, and K129 of SEQ ID NO:1. In one embodiment, the antibody, or antigen binding fragment thereof, capable of binding CD117, binds to an epitope in CD117 comprising at least seven of the amino acid residues of T67, K69, T71, S81, Y83, T114, T119, and K129 of SEQ ID NO:1. In another embodiment, the binding protein, e.g., antibody, or antigen binding fragment thereof, capable of binding CD117, binds to an epitope in CD117 comprising all of amino acid residues T67, K69, T71, S81, Y83, T114, T119, and K129 of SEQ ID NO:1.

In another aspect, the present disclosure pertains to an antibody, or antigen binding fragment thereof, capable of binding CD117 that binds to an epitope in CD117, wherein the epitope comprises amino acid residues selected from the group consisting of at least two, at least three, at least four, at least five, at least six, at least seven, or each of eight of the amino acid residues T67, K69, T71, S81, Y83, T114, T119, and K129 of SEQ ID NO:1.

In another aspect, the present disclosure pertains to an antibody, or antigen binding fragment thereof, capable of binding CD117 that binds to an epitope having residues within at least amino acids 67-83 and 114-129 of SEQ ID NO:1.

As described in the Protein Data Bank (PDB) under the reference number P10721 (see https://www.rcsb.org/pdb/protein/P10721 (as of Apr. 24, 2019), the extracellular portion of CD117 comprises five domains referred to as Ig-like C2-type 1 domain (a.k.a. D1), Ig-like C2-type 2 domain (a.k.a. D2), Ig-like C2-type 3 domain (a.k.a. D3), Ig-like C2-type 4 domain (a.k.a. D4), and Ig-like C2-type 5 domain (a.k.a. D5). In another aspect, the present disclosure pertains to an antibody, or antigen binding fragment thereof, capable of binding CD117 that binds to an epitope within the Ig-like C2-type 1 domain and Ig-like C2-type 2 domain (a.k.a. D2) of CD117. In some embodiments, the antibody, or antigen binding fragment thereof, binds to epitope comprising amino acid residues of the Ig-like C2-type 1 domain and at least one, at least two or all three of the amino acid residues T114, T119, or K129. In another embodiment, the antibody, or antigen binding fragment thereof, capable of binding CD117 binds to epitope comprising amino acid residues of the Ig-like C2-type 2 domain and at least one, at least two, at least three, at least four, or all five of the amino acid residues T67, K69, T71, S81, and Y83.

In another aspect, the antibodies described herein, have antagonistic activity without substantial agonist activity for CD117. Thus, included in the present disclosure are antibodies that bind to the epitope recognized by antibody Ab85. In a particular embodiment, the present disclosure includes an isolated antibody, or antigen-binding fragment thereof, wherein said antibody, or antigen binding fragment thereof, binds human CD117 such that CD117 with said antibody, or antigen binding fragment thereof, bound to an epitope defined by the topographic regions T67-Y83 and/or T114-K129 of SEQ ID NO:1 prevents the association of CD117 with SCF (stem cell factor) or is inhibited from dimerizing with a further human CD117. In another aspect, the present disclosure pertains to an antibody, or antigen-binding fragment thereof, capable of binding human CD117 that binds to an epitope in human CD117 comprising two, three, four, five, six, seven, or all of the amino acid residues of T67, K69, T71, S81, Y83, T114, T119, and K129 of SEQ ID NO:1.

### Antibody Drug Conjugates (ADCs)

### Cytotoxins

Anti-CD117 antibodies, and antigen-binding fragments thereof, described herein can be conjugated (linked) to a cytotoxin. In particular, the anti-CD117 ADCs include an antibody (or an antigen-binding fragment thereof) conjugated (i.e., covalently attached by a linker) to a cytotoxic moiety (or cytotoxin). In various embodiments, the cytotoxic moiety exhibits reduced or no cytotoxicity when bound in a conjugate, but resumes cytotoxicity after cleavage from the linker. In various embodiments, the cytotoxic moiety maintains cytotoxicity without cleavage from the linker. In some embodiments, the cytotoxic molecule is conjugated to a cell internalizing antibody, or antigen-binding fragment thereof as disclosed herein, such that following the cellular uptake of the antibody, or fragment thereof, the cytotoxin may access its intracellular target and, e.g., mediate T cell death.

Antibodies, and antigen-binding fragments thereof, described herein (e.g., antibodies, and antigen-binding fragments thereof, that recognize and bind CD117) can be conjugated (or linked) to a cytotoxin.

ADCs of the present disclosure therefore may be of the general formula Ab-(Z-L-D)ₙ wherein an antibody or antigen-binding fragment thereof (Ab) is conjugated (covalently linked) to linker (L), through a chemical moiety (Z), to a cytotoxic moiety (“drug,” D). “n” represents the number of drugs linked to the antibody, and generally ranges from 1 to 8.

Accordingly, the antibody or antigen-binding fragment thereof may be conjugated to a number of drug moieties as indicated by integer n, which represents the average number of cytotoxins per antibody, which may range, e.g., from about 1 to about 20. In some embodiments, n is from 1 to 4. In some embodiments, n is 1. In some embodiments, n is 2. The average number of drug moieties per antibody in preparations of ADC from conjugation reactions may be characterized by conventional means such as mass spectroscopy, ELISA assay, and HPLC. The quantitative distribution of ADC in terms of n may also be determined. In some instances, separation, purification, and characterization of homogeneous ADC where n is a certain value from ADC with other drug loadings may be achieved by means such as reverse phase HPLC or electrophoresis.

For some anti-CD117 ADCs, the average number of cytotoxins per antibody may be limited by the number of attachment sites on the antibody. For example, where the attachment is a cysteine thiol, an antibody may have only one or several cysteine thiol groups, or may have only one or several sufficiently reactive thiol groups through which a linker and chemical moiety may be attached. Generally, antibodies do not contain many free and reactive cysteine thiol groups which may be linked to a drug moiety; primarily, cysteine thiol residues in antibodies exist as disulfide bridges. In certain embodiments, an antibody may be reduced with a reducing agent such as dithiothreitol (DTT) or tricarbonylethylphosphine (TCEP), under partial or total reducing conditions, to generate reactive cysteine thiol groups.

In certain embodiments, fewer than the theoretical maximum of drug moieties are conjugated to an antibody during a conjugation reaction. An antibody may contain, for example, lysine residues that do not react with the drug-linker intermediate or linker reagent, as discussed below. Only the most reactive lysine groups may react with an amine-reactive linker reagent. In certain embodiments, an antibody is subjected to denaturing conditions to reveal reactive nucleophilic groups such as lysine or cysteine.

The loading (drug/antibody ratio) of an ADC may be controlled in different ways, e.g., by: (i) limiting the molar excess of drug-linker intermediate or linker reagent relative to antibody, (ii) limiting the conjugation reaction time or temperature, (iii) partial or limiting reductive conditions for cysteine thiol modification, (iv) engineering by recombinant techniques the amino acid sequence of the antibody such that the number and position of cysteine residues is modified for control of the number and/or position of linker-drug attachments.

In some embodiments, the cytotoxic molecule is conjugated to a cell internalizing antibody, or antigen-binding fragment thereof as disclosed herein such that following the cellular uptake of the antibody, or fragment thereof, the cytotoxin may access its intracellular target and mediate hematopoietic cell death. Any number of cytotoxins can be conjugated to the anti-CD117 antibody, e.g., 1, 2, 3, 4, 5, 6, 7, or 8.

Cytotoxins suitable for use with the compositions and methods described herein include DNA-intercalating agents, (e.g., anthracyclines), agents capable of disrupting the mitotic spindle apparatus (e.g., vinca alkaloids, maytansine, maytansinoids, and derivatives thereof), RNA polymerase inhibitors (e.g., an amatoxin, such as α-amanitin, and derivatives thereof), and agents capable of disrupting protein biosynthesis (e.g., agents that exhibit rRNA N-glycosidase activity, such as saporin and ricin A-chain), among others known in the art.

In some embodiments, the cytotoxin is a microtubule-binding agent (for instance, maytansine or a maytansinoid), an amatoxin, Pseudomonas exotoxin A, deBouganin, diphtheria toxin, saporin, an auristatin, an anthracycline, a calicheamicin, irinotecan, SN-38, a duocarmycin, a pyrrolobenzodiazepine, a pyrrolobenzodiazepine dimer, an indolinobenzodiazepine, an indolinobenzodiazepine dimer, an indolinobenzodiazepine pseudodimer, or a variant thereof, or another cytotoxic compound described herein or known in the art.

Anti-CD117 antibodies, and antigen-binding fragments thereof, described herein can be conjugated to a cytotoxin that is a microtubule binding agent. As used herein, the term “microtubule-binding agent” refers to a compound which acts by disrupting the microtubular network that is essential for mitotic and interphase cellular function in a cell. Examples of microtubule-binding agents include, but are not limited to, maytasine, maytansinoids, and derivatives thereof, such as those described herein or known in the art, vinca alkaloids, such as vinblastine, vinblastine sulfate, vincristine, vincristine sulfate, vindesine, and vinorelbine, taxanes, such as docetaxel and paclitaxel, macrolides, such as discodermolides, cochicine, and epothilones, and derivatives thereof, such as epothilone B or a derivative thereof.

### Maytansinoids

Antibodies and antigen-binding fragments thereof described herein can be conjugated to a cytotoxin that is a microtubule binding agent. In some embodiments, the microtubule binding agent is a maytansine, a maytansinoid or a maytansinoid analog. Maytansinoids are mitototic inhibitors which bind microtubules and act by inhibiting tubulin polymerization. Maytansine was first isolated from the east African shrub Maytenus serrata (U.S. Pat. No. 3,896,111). Subsequently, it was discovered that certain microbes also produce maytansinoids, such as maytansinol and C-3 maytansinol esters (U.S. Pat. No. 4,151,042). Synthetic maytansinol and derivatives and analogues thereof are disclosed, for example, in U.S. Pat. Nos. 4,137,230; 4,248,870; 4,256,746; 4,260,608; 4,265,814; 4,294,757; 4,307,016; 4,308,268; 4,308,269; 4,309,428; 4,313,946; 4,315,929; 4,317,821; 4,322,348; 4,331,598; 4,361,650; 4,364,866; 4,424,219; 4,450,254; 4,362,663; and 4,371,533. Maytansinoid drug moieties are attractive drug moieties in antibody drug conjugates because they are: (i) relatively accessible to prepare by fermentation or chemical modification, derivatization of fermentation products, (ii) amenable to derivatization with functional groups suitable for conjugation through the non-disulfide linkers to antibodies, (iii) stable in plasma, and (iv) effective against a variety of tumor cell lines.

Examples of suitable maytansinoids include esters of maytansinol, synthetic maytansinol, and maytansinol analogs and derivatives. Included herein are any cytotoxins that inhibit microtubule formation and that are highly toxic to mammalian cells, as are maytansinoids, maytansinol, and maytansinol analogs, and derivatives.

Examples of suitable maytansinol esters include those having a modified aromatic ring and those having modifications at other positions. Such suitable maytansinoids are disclosed in U.S. Pat. Nos. 4,137,230; 4,151,042; 4,248,870; 4,256,746; 4,260,608; 4,265,814; 4,294,757; 4,307,016; 4,308,268; 4,308,269; 4,309,428; 4,313,946; 4,315,929; 4,317,821; 4,322,348; 4,331,598; 4,361,650; 4,362,663; 4,364,866; 4,424,219; 4,450,254; 4,322,348; 4,362,663; 4,371,533; 5,208,020; 5,416,064; 5,475,092;

5,585,499; 5,846,545; 6,333,410; 7,276,497; and 7,473,796, the disclosures of each of which are incorporated herein by reference as they pertain to maytansinoids and derivatives thereof.

In some embodiments, the antibody-drug conjugates (ADCs) of the present disclosure utilize the thiol-containing maytansinoid (DM1), formally termed N^{2′}-deacetyl-N^{2′}-(3-mercapto-1-oxopropyl)-maytansine, as the cytotoxic agent. DM1 is represented by the structural formula (V):

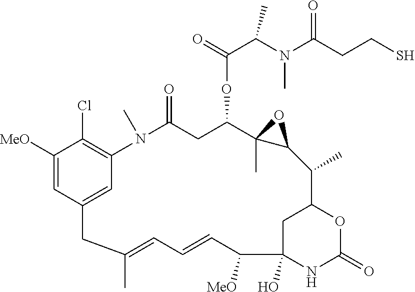 [SMILES: COc1cc2cc(c1Cl)N(C)C(=O)C[C@H](OC(=O)[C@H](C)N(C)C(=O)CCS)[C@]1(C)O[C@H]1[C@H](C)C1C[C@@](O)(NC(=O)O1)[C@H](OC)/C=C/C=C(\C)C2]

In another embodiment, the conjugates of the present disclosure utilize the thiol-containing maytansinoid N^{2′}-deacetyl-N^{2′}(4-methyl-4-mercapto-1-oxopentyl)-maytansine (e.g., DM4) as the cytotoxic agent. DM4 is represented by the structural formula (VI):

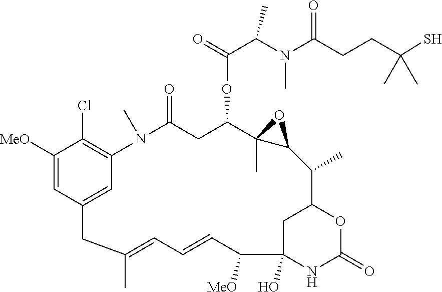 [SMILES: COc1cc2cc(c1Cl)N(C)C(=O)C[C@H](OC(=O)[C@H](C)N(C)C(=O)CCC(C)(C)S)[C@]1(C)O[C@H]1[C@H](C)C1C[C@@](O)(NC(=O)O1)[C@H](OC)/C=C/C=C(\C)C2]

Another maytansinoid comprising a side chain that contains a sterically hindered thiol bond is N^{2′}-deacetyl-N-^{2′}(4-mercapto-1-oxopentyl)-maytansine (termed DM3), represented by the structural formula (VII):

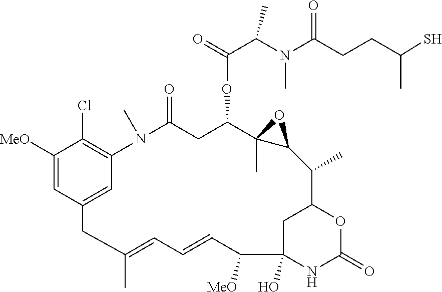 [SMILES: COc1cc2cc(c1Cl)N(C)C(=O)C[C@H](OC(=O)[C@H](C)N(C)C(=O)CCC(C)S)[C@]1(C)O[C@H]1[C@H](C)C1C[C@@](O)(NC(=O)O1)[C@H](OC)/C=C/C=C(\C)C2]

Each of the maytansinoids taught in U.S. Pat. Nos. 5,208,020 and 7,276,497, can also be used in the conjugate of the present disclosure. In this regard, the entire disclosure of U.S. Pat. Nos. 5,208,020 and 7,276,697 is incorporated herein by reference.

Many positions on maytansinoids can serve as the position to chemically link the linking moiety. For example, the C-3 position having a hydroxyl group, the C-14 position modified with hydroxymethyl, the C-15 position modified with hydroxy and the C-20 position having a hydroxy group are all expected to be useful. In some embodiments, the C-3 position serves as the position to chemically link the linking moiety, and in some particular embodiments, the C-3 position of maytansinol serves as the position to chemically link the linking moiety. There are many linking groups known in the art for making antibody-maytansinoid conjugates, including, for example, those disclosed in U.S. Pat. Nos. 5,208,020, 6,441,163, and EP Patent No. 0425235 B1; Chari et al., Cancer Research 52:127-131 (1992); and U.S. 2005/0169933 A1, the disclosures of which are hereby expressly incorporated by reference. Additional linking groups are described and exemplified herein.

The present disclosure also includes various isomers and mixtures of maytansinoids and conjugates. Certain compounds and conjugates of the present disclosure may exist in various stereoisomeric, enantiomeric, and diastereomeric forms. Several descriptions for producing such antibody-maytansinoid conjugates are provided in U.S. Pat. Nos. 5,208,020, 5,416,064 6,333,410, 6,441,163, 6,716,821, and 7,368,565, each of which is incorporated herein in its entirety.

A therapeutically effective number of maytansinoid molecules bound per antibody molecule can be determined by measuring spectrophotometrically the ratio of the absorbance at 252 nm and 280 nm. In certain embodiments, an average of 3 to 4 maytansinoid molecules conjugated per antibody molecule may enhance the cytotoxicity of target cells without negatively affecting the function or solubility of the antibody, although one molecule of toxin/antibody can enhance cytotoxicity over antibody alone. The average number of maytansinoid molecules/antibody or antigen binding fragment thereof can be, for example, 1-10 or 2-5.

### Anthracyclines

In other embodiments, the antibodies and antigen-binding fragments thereof described herein can be conjugated to a cytotoxin that is an anthracycline molecule. Anthracyclines are antibiotic compounds that exhibit cytotoxic activity. Studies have indicated that anthracyclines may operate to kill cells by a number of different mechanisms including: 1) intercalation of the drug molecules into the DNA of the cell thereby inhibiting DNA-dependent nucleic acid synthesis; 2) production by the drug of free radicals which then react with cellular macromolecules to cause damage to the cells or 3) interactions of the drug molecules with the cell membrane [see, e.g., C. Peterson et al., “Transport And Storage Of Anthracycline In Experimental Systems And Human Leukemia” in Anthracycline Antibiotics In Cancer Therapy; N. R. Bachur, “Free Radical Damage” id. at pp. 97-102]. Because of their cytotoxic potential, anthracyclines have been used in the treatment of numerous cancers such as leukemia, breast carcinoma, lung carcinoma, ovarian adenocarcinoma and sarcomas [see e.g., P. H-Wiernik, in Anthracycline: Current Status And New Developments p 11]. Commonly used anthracyclines include doxorubicin, epirubicin, idarubicin and daunomycin. In some embodiments, the cytotoxin is an anthracycline selected from the group consisting of daunorubicin, doxorubicin, epirubicin, and idarubicin. Representative examples of anthracyclines include, but are not limited to daunorubicin (Cerubidine; Bedford Laboratories), doxorubicin (Adriamycin; Bedford Laboratories; also referred to as doxorubicin hydrochloride, hydroxy-daunorubicin, and Rubex), epirubicin (Ellence; Pfizer), and idarubicin (Idamycin; Pfizer Inc.).

The anthracycline analog, doxorubicin (ADRIAMYCINO) is thought to interact with DNA by intercalation and inhibition of the progression of the enzyme topoisomerase II, which unwinds DNA for transcription. Doxorubicin stabilizes the topoisomerase II complex after it has broken the DNA chain for replication, preventing the DNA double helix from being resealed and thereby stopping the process of replication. Doxorubicin and daunorubicin (DAUNOMYCIN) are prototype cytotoxic natural product anthracycline chemotherapeutics (Sessa et al., (2007) Cardiovasc. Toxicol. 7:75-79).

One non-limiting example of a suitable anthracycline for use herein is PNU-159682 (“PNU”). PNU exhibits greater than 3000-fold cytotoxicity relative to the parent nemorubicin (Quintieri et al., Clinical Cancer Research 2005, 11, 1608-1617). PNU is represented by structural formula:

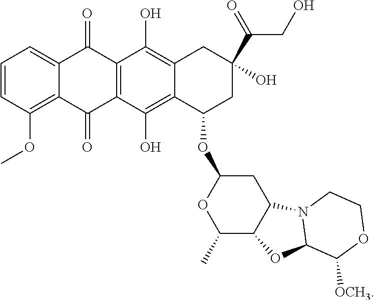

Multiple positions on anthracyclines such as PNU can serve as the position to covalently bond the linking moiety and, hence the anti-CD117 antibodies or antigen-binding fragments thereof as described herein. For example, linkers may be introduced through modifications to the hydroxymethyl ketone side chain.

In some embodiments, the cytotoxin is a PNU derivative represented by structural formula:

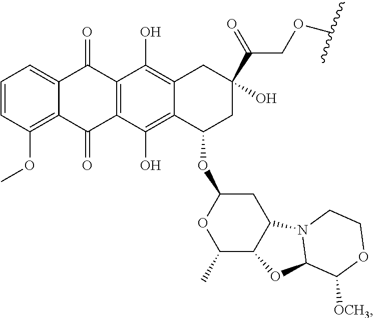

wherein the wavy line indicates the point of covalent attachment to the linker of the ADC as described herein.

In some embodiments, the cytotoxin is a PNU derivative represented by structural formula:

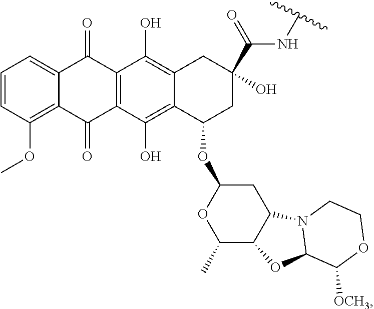

wherein the wavy line indicates the point of covalent attachment to the linker of the ADC as described herein.

### Benzodiazepine Cytotoxins (e.g., Pyrrolobenzodiazepines (PBDs))

In other embodiments, the anti-CD117 antibodies or antigen-binding fragments thereof described herein can be conjugated to a cytotoxin that is a pyrrolobenzodiazepine (PBD) or a cytotoxin that comprises a PBD. PBDs are natural products produced by certain actinomycetes and have been shown to be sequence selective DNA alkylating compounds. PBD cytotoxins include, but are not limited to, anthramycin, dimeric PBDs, and those disclosed in, for example, Hartley, J. A. (2011). “The development of pyrrolobenzodiazepines as antitumour agents.” Expert Opin. Inv. Drug, 20(6), 733-744; and Antonow, D.; Thurston, D. E. (2011) “Synthesis of DNA-interactive pyrrolo[2,1-c][1,4]benzodiazepines (PBDs).” Chem. Rev. 111: 2815-2864.

In some embodiments, the cytotoxin may be a pyrrolobenzodiazepine dimer represented by the formula:

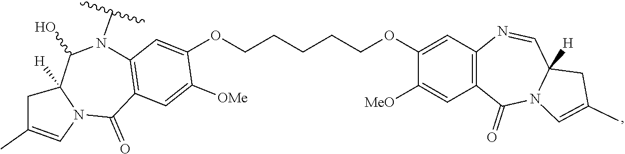

wherein the wavy line indicates the attachment point of the linker.

In some embodiments, the cytotoxin is conjugated to the antibody, or the antigen-binding fragment thereof, by way of a maleimidocaproyl linker.

In some embodiments, the linker comprises one or more of a peptide, oligosaccharide, —(CH₂)ₚ—, —(CH₂CH₂O)_{q}—, —(C═O)(CH₂)ᵣ—, —(C═O)(CH₂CH₂O)ₜ—, —(NHCH₂CH₂)ᵤ—, -PAB, Val-Cit-PAB, Val-Ala-PAB, Val-Lys(Ac)-PAB, Phe-Lys-PAB, Phe-Lys(Ac)-PAB, D-Val-Leu-Lys, Gly-Gly-Arg, Ala-Ala-Asn-PAB, or Ala-PAB, wherein each of p, q, r, t, and u are integers from 1-12, selected independently for each occurrence.

In some embodiments, the linker has the structure of formula:

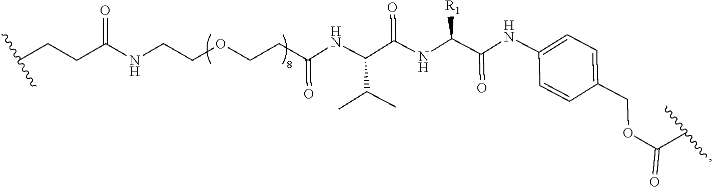

wherein R₁ is CH₃ (Ala) or (CH₂)₃NH(CO)NH₂ (Cit).

In some embodiments, the linker, prior to conjugation to the antibody and including the reactive substituent Z′, taken together as L-Z′, has the structure:

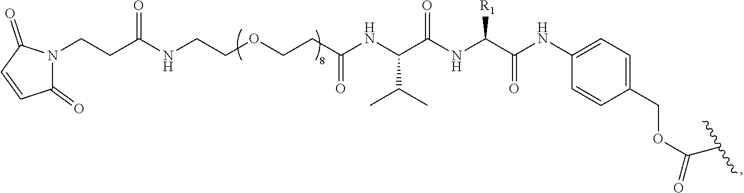

wherein the wavy line indicates the attachment point to the cytotoxin (e.g., a PBD). In certain embodiments, R₁ is CH₃.

In some embodiments, the cytotoxin-linker conjugate, prior to conjugation to the antibody and including the reactive substituent Z′, taken together as Cy-L-Z′, has the structure of formula:

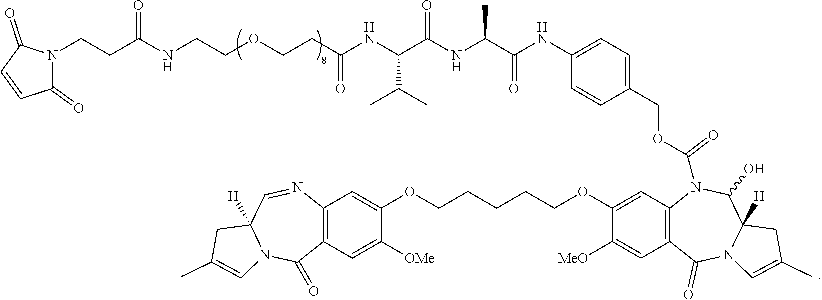

This particular cytotoxin-linker conjugate is known as tesirine (SG3249), and has been described in, for example, Howard et al., ACS Med. Chem. Lett. 2016, 7(11), 983-987, the disclosure of which is incorporated by reference herein in its entirety.

In some embodiments, the cytotoxin may be a pyrrolobenzodiazepine dimer represented by formula:

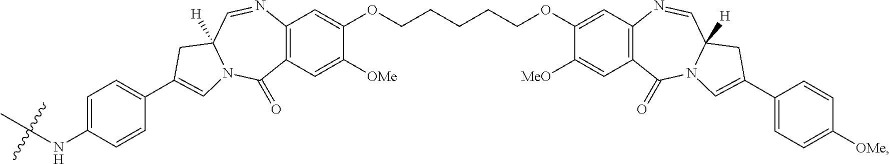 [SMILES: *Nc1ccc(C2=CN3C(=O)c4cc(OC)c(OCCCCCOc5cc6c(cc5OC)C(=O)N5C=C(c7ccc(OC)cc7)C[C@H]5C=N6)cc4N=C[C@@H]3C2)cc1]

wherein the wavy line indicates the attachment point of the linker.

In some embodiments, the cytotoxin-linker conjugate, prior to conjugation to the antibody and including the reactive substituent Z′, taken together as Cy-L-Z′, has the structure of formula:

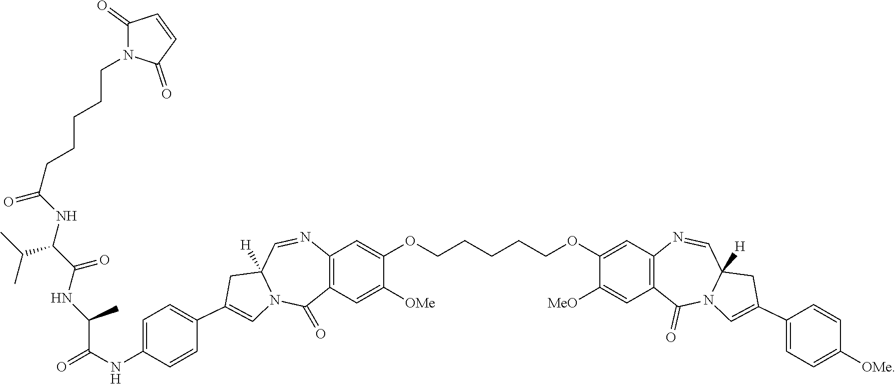 [SMILES: COc1ccc(C2=CN3C(=O)c4cc(OC)c(OCCCCCOc5cc6c(cc5OC)C(=O)N5C=C(c7ccc(NC(=O)[C@H](C)NC(=O)[C@@H](NC(=O)CCCCCN8C(=O)C=CC8=O)C(C)C)cc7)C[C@H]5C=N6)cc4N=C[C@@H]3C2)cc1]

This particular cytotoxin-linker conjugate is known as talirine, and has been described, for example, in connection with the ADC Vadastuximab talirine (SGN-CD33A), Mantaj et al., Angewandte Chemie International Edition English 2017,56, 462-488, the disclosure of which is incorporated by reference herein in its entirety.

In some embodiments, the cytotoxin may be an indolinobenzodiazepine pseudodimer having the structure of formula:

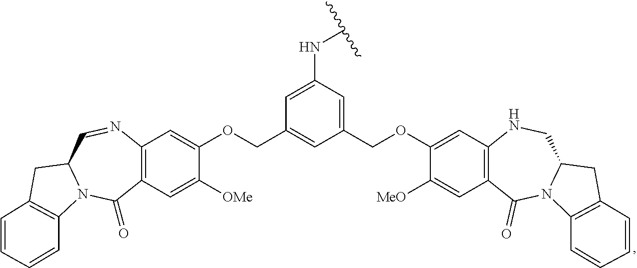 [SMILES: *Nc1cc(COc2cc3c(cc2OC)C(=O)N2c4ccccc4C[C@H]2C=N3)cc(COc2cc3c(cc2OC)C(=O)N2c4ccccc4C[C@H]2CN3)c1]

wherein the wavy line indicates the attachment point of the linker.

In some embodiments, the cytotoxin-linker conjugate, prior to conjugation to the antibody and including the reactive substituent Z′, taken together as Cy-L-Z′, has the structure of formula:

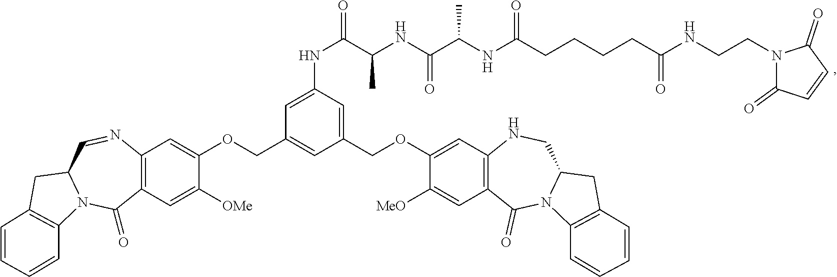 [SMILES: COc1cc2c(cc1OCc1cc(COc3cc4c(cc3OC)C(=O)N3c5ccccc5C[C@H]3CN4)cc(NC(=O)[C@H](C)NC(=O)[C@H](C)NC(=O)CCCCC(=O)NCCN3C(=O)C=CC3=O)c1)N=C[C@@H]1Cc3ccccc3N1C2=O]

which comprises the ADC IMGN632, disclosed in, for example, International Patent Application Publication No. WO2017004026, which is incorporated by reference herein.

Calicheamicin

In other embodiments, the antibodies and antigen-binding fragments thereof described herein can be conjugated to a cytotoxin that is an enediyne antitumor antibiotic (e.g., calicheamicins, ozogamicin). The calicheamicin family of antibiotics are capable of producing double-stranded DNA breaks at sub-picomolar concentrations. For the preparation of conjugates of the calicheamicin family, see U.S. Pat. Nos. 5,712,374; 5,714,586; 5,739,116; 5,767,285; 5,770,701; 5,770,710; 5,773,001; and 5,877,296 (all to American Cyanamid Company). Structural analogues of calicheamicin which may be used include, but are not limited to, those disclosed in, for example, Hinman et al., Cancer Research 53:3336-3342 (1993), Lode et al., Cancer Research 58:2925-2928 (1998), and the aforementioned U.S. patents to American Cyanamid.

An exemplary calicheamicin is designated γ₁, which is herein referenced simply as gamma, and has the structural formula:

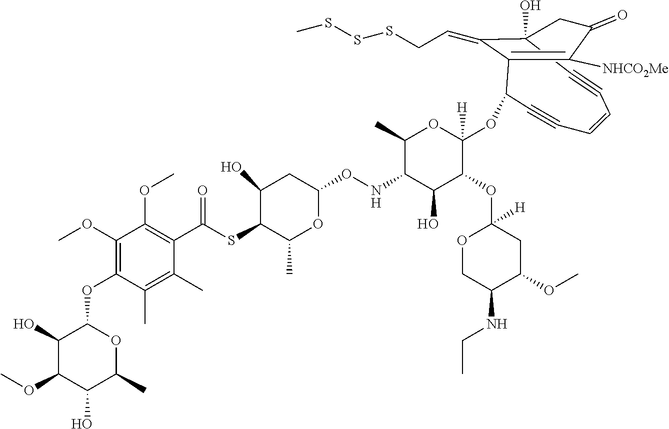

In some embodiments, the calicheamicin may be a gamma-calicheamicin derivative or an N-acetyl gamma-calicheamicin derivative. Structural analogues of calicheamicin which may be used include, but are not limited to, those disclosed in, for example, Hinman et al., Cancer Research 53:3336-3342 (1993), Lode et al., Cancer Research 58:2925-2928 (1998), and the aforementioned U.S. patents. Calicheamicins contain a methyltrisulfide moiety that can be reacted with appropriate thiols to form disulfides, at the same time introducing a functional group that is useful in attaching a calicheamicin derivative to an anti-CD117 antibody or antigen-binding fragment thereof as described herein, via a linker. For the preparation of conjugates of the calicheamicin family, see U.S. Pat. Nos. 5,712,374; 5,714,586; 5,739,116; 5,767,285; 5,770,701; 5,770,710; 5,773,001; and 5,877,296 (all to American Cyanamid Company). Structural analogues of calicheamicin which may be used include, but are not limited to, those disclosed in, for example, Hinman et al., Cancer Research 53:3336-3342 (1993), Lode et al., Cancer Research 58:2925-2928 (1998), and the aforementioned U.S. patents to American Cyanamid.

In one embodiment, the cytotoxin of the ADC as disclosed herein may be a calicheamicin disulfide derivative represented by the formula:

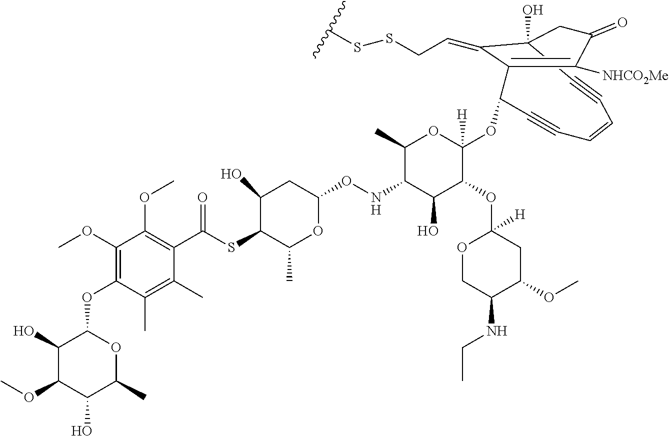

wherein the wavy line indicates the attachment point of the linker.

### Ribosome Inactivating Proteins (RIPs)

In some embodiments, the cytotoxin conjugated to an anti-CD117 antibody is a ribosome-inactivating protein (RIP). Ribosome inactivating proteins are protein synthesis inhibitors that act on ribosomes, usually irreversibly. RIPs are found in plants, as well as bacteria. Examples of RIPs include, but are not limited to, saporin, ricin, abrin, gelonin, Pseudomonas exotoxin (or exotoxin A), trichosanthin, luffin, agglutinin and the diphtheria toxin.

Another example of an RIP that may be used in the ADCs and methods disclosed herein are a Shiga toxin (Stx) or a Shiga-like toxins (SLT). Shiga toxin (Stx) is a potent bacterial toxin found in Shigella dysenteriae 1 and in some serogroups (including serotypes O157:H7, and O104:H4) of Escherichia coli (called Stx1 in E. coli). In addition to Stx1, some E. coli strains produce a second type of Stx (Stx2) that has the same mode of action as Stx/Stx1 but is antigenically distinct. The toxins are named after Kiyoshi Shiga, who first described the bacterial origin of dysentery caused by Shigella dysenteriae. SLT is a historical term for similar or identical toxins produced by Escherichia coli. Because subtypes of each toxin have been identified, the prototype toxin for each group is now designated Stx1a or Stx2a. Stx1a and Stx2a exhibit differences in cytotoxicity to various cell types, bind dissimilarly to receptor analogs or mimics, induce differential chemokine responses, and have several distinctive structural characteristics.

A member of the Shiga toxin family refers to any member of a family of naturally occurring protein toxins which are structurally and functionally related, notably, toxins isolated from S. dysenteriae and E. coli (Johannes L, Romer W, Nat Rev Microbiol 8: 105-16 (2010)). For example, the Shiga toxin family encompasses true Shiga toxin (Stx) isolated from S. dysenteriae serotype 1, Shiga-like toxin 1 variants (SLT1 or Stx1 or SLT-1 or Slt-I) isolated from serotypes of enterohemorrhagic E. coli, and Shiga-like toxin 2 variants (SLT2 or Stx2 or SLT-2) isolated from serotypes of enterohemorrhagic E. coli. SLT1 differs by only one residue from Stx, and both have been referred to as Verocytotoxins or Verotoxins (VTs) (O'Brien A et al., Curr Top Microbiol Immunol 180: 65-94 (1992)). Although SLT1 and SLT2 variants are only about 53-60% similar to each other at the amino acid sequence level, they share mechanisms of enzymatic activity and cytotoxicity common to the members of the Shiga toxin family (Johannes, Nat Rev Microbiol 8: 105-16 (2010)).

Members of the Shiga toxin family have two subunits; A subunit and a B subunit. The B subunit of the toxin binds to a component of the cell membrane known as glycolipid globotriaosylceramide (Gb3). Binding of the subunit B to Gb3 causes induction of narrow tubular membrane invaginations, which drives formation of inward membrane tubules for the bacterial uptake into the cell. The Shiga toxin (a non-pore forming toxin) is transferred to the cytosol via Golgi network and ER. From the Golgi toxin is trafficked to the ER. Shiga toxins act to inhibit protein synthesis within target cells by a mechanism similar to that of ricin (Sandvig and van Deurs (2000) EMBO J 19(220:5943). After entering a cell the A subunit of the toxin cleaves a specific adenine nucleobase from the 28S RNA of the 60S subunit of the ribosome, thereby halting protein synthesis (Donohue-Rolfe et al. (2010) Reviews of Infectious Diseases 13 Suppl. 4(7): S293-297).

As used herein, reference to Shiga family toxin refers to any member of the Shiga toxin family of naturally occurring protein toxins (e.g., toxins isolated from S. dysenteriae and E. coli) which are structurally and functionally related. For example, the Shiga toxin family encompasses true Shiga toxin (Stx) isolated from S. dysenteriae serotype 1, Shiga-like toxin 1 variants (SLT1 or Stx1 or SLT-1 or Slt-I) isolated from serotypes of enterohemorrhagic E. coli, and Shiga-like toxin 2 variants (SLT2 or Stx2 or SLT-2) isolated from serotypes of enterohemorrhagic E. coli. As used herein, “subunit A from a Shiga family toxin” or “Shiga family toxin subunit A” refers to a subunit A from any member of the Shiga toxin family, including Shiga toxins or Shiga-like toxins.

In one embodiment, an anti-CD117 ADC comprises an anti-CD117 antibody conjugated to a Shiga family toxin subunit A, or a portion of a Shiga family toxin subunit A having cytotoxic activity, i.e., ribosome inhibiting activity. Shiga toxin subunit A cytotoxic activities include, for example, ribosome inactivation, protein synthesis inhibition, N-glycosidase activity, polynucleotide:adenosine glycosidase activity, RNAase activity, and DNAase activity. Non-limiting examples of assays for Shiga toxin effector activity measure protein synthesis inhibitory activity, depurination activity, inhibition of cell growth, cytotoxicity, supercoiled DNA relaxation activity, and nuclease activity.

In certain embodiments, an anti-CD117 antibody, or an antigen binding fragment thereof, is conjugated to Shiga family toxin A subunit, or a fragment thereof having ribosome inhibiting activity. An example of a Shiga family toxin subunit A is Shiga-like toxin 1 subunit A (SLT-1A), the amino acid sequence of which is provided below

(SEQ ID NO: 144)
KEFTLDFSTAKTYVDSLNVIRSAIGTPLQTISSGG
TSLLMIDSGSGDNLFAVDVRGIDPEEGRFNNLRLI
VERNNLYVTGFVNRTNNVFYRFADFSHVTFPGTTA
VTLSGDSSYTTLQRVAGISRTGMQINRHSLTTSYL
DLMSHSGTSLTQSVARAMLRFVTVTAEALRFRQIQ
RGFRTTLDDLSGRSYVMTAEDVDLTLNWGRLSSVL
PDYHGQDSVRVGRISFGSINAILGSVALILNCHHH
ASRVARMASDEFPSMCPADGRVRGITHNKILWDSS
TLGAILMRRTISS.

Another example of a Shiga family toxin subunit A is Shiga toxin subunit A (StxA), the amino acid sequence of which is provided below

(SEQ ID NO: 145)
KEFTLDFSTAKTYVDSLNVIRSAIGTPLQTISSGG
TSLLMIDSGTGDNLFAVDVRGIDPEEGRFNNLRLI
VERNNLYVTGFVNRTNNVFYRFADFSHVTFPGTTA
VTLSGDSSYTTLQRVAGISRTGMQINRHSLTTSYL
DLMSHSGTSLTQSVARAMLRFVTVTAEALRFRQIQ
RGFRTTLDDLSGRSYVMTAEDVDLTLNWGRLSSVL
PDYHGQDSVRVGRISFGSINAILGSVALILNCHHH
ASRVARMASDEFPSMCPADGRVRGITHNKILWDSS
TLGAILMRRTISS.

Another example of a Shiga family toxin subunit A is Shiga-like toxin 2 subunit A (SLT-2A), the amino acid sequence of which is provided below

(SEQ ID NO: 146)
DEFTVDFSSQKSYVDSLNSIRSAISTPLGNISQGG
VSVSVINHVLGGNYISLNVRGLDPYSERFNHLRLI
MERNNLYVAGFINTETNIFYRFSDFSHISVPDVIT
VSMTTDSSYSSLQRIADLERTGMQIGRHSLVGSYL
DLMEFRGRSMTRASSRAMLRFVTVIAEALRFRQIQ
RGFRPALSEASPLYTMTAQDVDLTLNWGRISNVLP
EYRGEEGVRIGRISFNSLSAILGSVAVILNCHSTG
SYSVRSVSQKQKTECQIVGDRAAIKVNNVLWEANT
IAALLNRKPQDLTEPNQ.

In certain circumstances, naturally occurring Shiga family toxin subunits A may comprise precursor forms containing signal sequences of about 22 amino acids at their amino-terminals which are removed to produce mature Shiga family toxin A subunits and are recognizable to the skilled worker. Cytotoxic fragments or truncated versions of Shiga family toxin subunit A may also be used in the ADCs and methods disclosed herein.

In certain embodiments, a Shiga family toxin subunit A differs from a naturally occurring Shiga toxin A subunit by up to 1, 2, 3, 4, 5, 6, 7, 8, 9, 10, 15, 20, 25, 30, 35, 40 or more amino acid residues (but by no more than that which retains at least about 85%, at least about 90%, at least about 95%, at least about 96%, at least about 97%, at least about 98%, at least about 99%, or more amino acid sequence identity). In some embodiments, the Shiga family toxin subunit A differs from a naturally occurring Shiga family toxin A subunit by up to 1, 2, 3, 4, 5, 6, 7, 8, 9, 10, 15, 20, 25, 30, 35, 40 or more amino acid residues (but by no more than that which retains at least about 85%, at least about 90%, at least about 95%, at least about 96%, at least about 97%, at least about 98%, at least about 99% or more amino acid sequence identity). Thus, a polypeptide region derived from an A Subunit of a member of the Shiga toxin family may comprise additions, deletions, truncations, or other alterations from the original sequence as long as at least about 85%, at least about 90%, at least about 95%, at least about 96%, at least about 97%, at least about 98%, at least about 99% or more amino acid sequence identity is maintained to a naturally occurring Shiga family toxin subunit A.

Accordingly, in certain embodiments, the Shiga family toxin subunit A comprises or consists essentially of amino acid sequences having at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, at least about 99.5%, at least about 99.6%, at least about 99.7%, at least about 99.8%, at least about 99.9% or more overall sequence identity to a naturally occurring Shiga family toxin subunit A, such as SLT-1A (SEQ ID NO: 144), StxA (SEQ ID NO:145), and/or SLT-2A (SEQ ID NO:146).

Suitable Shiga toxins and RIP s suitable as cytotoxins are disclosed in, for example, US20180057544, which is incorporated by reference herein in its entirety.

### Auristatins

Anti-CD117 antibodies, and antigen-binding fragments thereof, described herein can be conjugated to a cytotoxin that is an auristatin (U.S. Pat. Nos. 5,635,483; 5,780,588). Auristatins are anti-mitotic agents that interfere with microtubule dynamics, GTP hydrolysis, and nuclear and cellular division (Woyke et al (2001) Antimicrob. Agents and Chemother. 45(12):3580-3584) and have anticancer (U.S. Pat. No. 5,663,149) and antifungal activity (Pettit et al (1998) Antimicrob. Agents Chemother. 42:2961-2965). (U.S. Pat. Nos. 5,635,483; 5,780,588). The auristatin drug moiety may be attached to the antibody through the N- (amino) terminus or the C- (carboxyl) terminus of the peptidic drug moiety (WO 02/088172).

Exemplary auristatin embodiments include the N-terminus linked monomethylauristatin drug moieties DE and DF, disclosed in Senter et al, Proceedings of the American Association for Cancer Research, Volume 45, Abstract Number 623, presented Mar. 28, 2004, the disclosure of which is expressly incorporated by reference in its entirety.

An exemplary auristatin embodiment is MMAE:

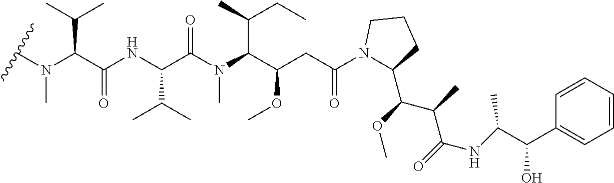 [SMILES: *N(C)[C@H](C(=O)N[C@H](C(=O)N(C)[C@@H]([C@@H](C)CC)[C@@H](CC(=O)N1CCC[C@H]1[C@H](OC)[C@@H](C)C(=O)N[C@H](C)[C@@H](O)c1ccccc1)OC)C(C)C)C(C)C]

wherein the wavy line indicates the point of covalent attachment to the linker of an antibody-linker conjugate (-L-Z-Ab, as described herein).

Another exemplary auristatin embodiment is MMAF:

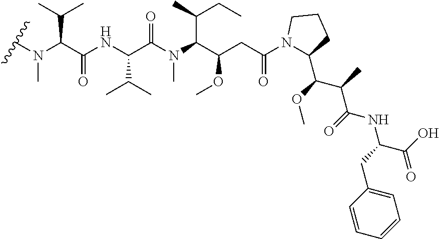 [SMILES: *N(C)[C@H](C(=O)N[C@H](C(=O)N(C)[C@@H]([C@@H](C)CC)[C@@H](CC(=O)N1CCC[C@H]1[C@H](OC)[C@@H](C)C(=O)N[C@@H](Cc1ccccc1)C(=O)O)OC)C(C)C)C(C)C]

wherein the wavy line indicates the point of covalent attachment to the linker of an antibody-linker conjugate (-L-Z-Ab, as described herein), as disclosed in US 2005/0238649.

Auristatins may be prepared according to the methods of: U.S. Pat. Nos. 5,635,483; 5,780,588; Pettit et al (1989) J. Am. Chem. Soc. 111:5463-5465; Pettit et al (1998) Anti-Cancer Drug Design 13:243-277; Pettit, G. R., et al. Synthesis, 1996, 719-725; Pettit et al (1996) J. Chem. Soc. Perkin Trans. 15:859-863; and Doronina (2003) Nat. Biotechnol. 21(7):778-784.

### Amatoxins

In some embodiments, the cytotoxin of the antibody-drug conjugate is an RNA polymerase inhibitor. In some embodiments, the RNA polymerase inhibitor is an amatoxin or derivative thereof.

In some embodiments, the cytotoxin is an amatoxin or derivative thereof, such as α-amanitin, β-amanitin, γ-amanitin, ε-amanitin, amanin, amaninamide, amanullin, amanullinic acid, and proamanullin. Structures of the various naturally occurring amatoxins are represented by formula III, and are disclosed in, e.g., Zanotti et al., Int. J. Peptide Protein Res. 30, 1987, 450-459. In one embodiment, the cytotoxin is an amanitin.

In some embodiments, the cytotoxin is an amatoxin of formula III, IIIA, IIIB, or IIIC. For instance, the antibodies, or antigen-binding fragments, described herein may be bound to an amatoxin so as to form a conjugate represented by the formula Ab-Z-L-Am, wherein Ab is the antibody, or antigen-binding fragment thereof, L is a linker, Z is a chemical moiety and Am is an amatoxin. Many positions on amatoxins or derivatives thereof can serve as the position to covalently bond the linking moiety L, and, hence the antibodies or antigen-binding fragments thereof. In some embodiments, Am-L-Z is represented by formula (I)

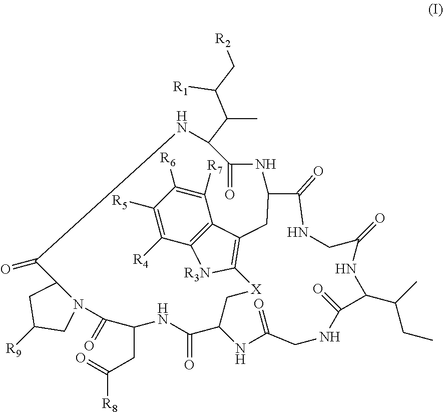

wherein R₁ is H, OH, OR_{A}, or OR_{C};

R₂ is H, OH, OR_{B}, or OR_{C};

R_{A} and R_{B}, when present, together with the oxygen atoms to which they are bound, combine to

form an optionally substituted 5-membered heterocycloalkyl group;

R₃ is H, R_{C}, or R_{D};

R₄ is H, OH, OR_{C}, OR_{D}, R_{C}, or R_{D};

R₅ is H, OH, OR_{C}, OR_{D}, R_{C}, or R_{D};

R₆ is H, OH, OR_{C}, OR_{D}, R_{C}, or R_{D};

R₇ is H, OH, OR_{C}, OR_{D}, R_{C}, or R_{D};

R₈ is OH, NH₂, OR_{C}, OR_{D}, NHR_{C}, or NR_{C}R_{D};

R₉ is H, OH, OR_{C}, or OR_{D};

X is —S—, —S(O)—, or —SO₂—;

R_{C} is -L-Z;

R_{D} is optionally substituted alkyl (e.g., C₁-C₆ alkyl), optionally substituted heteroalkyl (e.g., C₁-C₆ heteroalkyl), optionally substituted alkenyl (e.g., C₂-C₆ alkenyl), optionally substituted heteroalkenyl (e.g., C₂-C₆ heteroalkenyl), optionally substituted alkynyl (e.g., C₂-C₆ alkynyl), optionally substituted heteroalkynyl (e.g., C₂-C₆ heteroalkynyl), optionally substituted cycloalkyl, optionally substituted heterocycloalkyl, optionally substituted aryl, or optionally substituted heteroaryl;

L is a linker, such as optionally substituted alkylene (e.g., C₁-C₆ alkylene), optionally substituted heteroalkylene (C₁-C₆ heteroalkylene), optionally substituted alkenylene (e.g., C₂-C₆ alkenylene), optionally substituted heteroalkenylene (e.g., C₂-C₆ heteroalkenylene), optionally substituted alkynylene (e.g., C₂-C₆ alkynylene), optionally substituted heteroalkynylene (e.g., C₂-C₆ heteroalkynylene), optionally substituted cycloalkylene, optionally substituted heterocycloalkylene, optionally substituted arylene, optionally substituted heteroarylene, a peptide, a dipeptide, —(C═O)—, a disulfide, a thioether, a hydrazone, or a combination thereof; and

Z is a chemical moiety formed from a coupling reaction between a reactive substituent present on L and a reactive substituent present within an antibody, or antigen-binding fragment thereof, that binds CD117 (such as GNNK+ CD117).

In some embodiments, Am contains exactly one R_{C} substituent.

In some embodiments, the linker comprises a —(CH)₂ₙ— unit, where n is an integer from 2-6. In some embodiments, the linker includes —((CH₂)ₙ where n is 6. In some embodiments, L-Z is

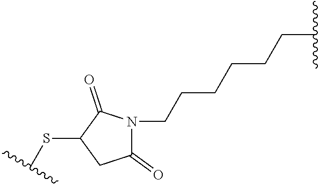

where S is a sulfur atom which represents the reactive substituent present within an antibody, or antigen-binding fragment thereof, that binds CD117 (e.g., from the —SH group of a cysteine residue).

In some embodiments, L-Z is

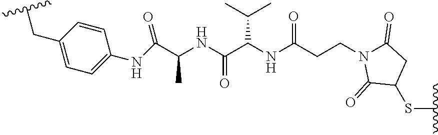

where S is a sulfur atom which represents the reactive substituent present within an antibody, or antigen-binding fragment thereof, that binds CD117 (e.g., from the —SH group of a cysteine residue).

In some embodiments, Am-L-Z-Ab is one of:

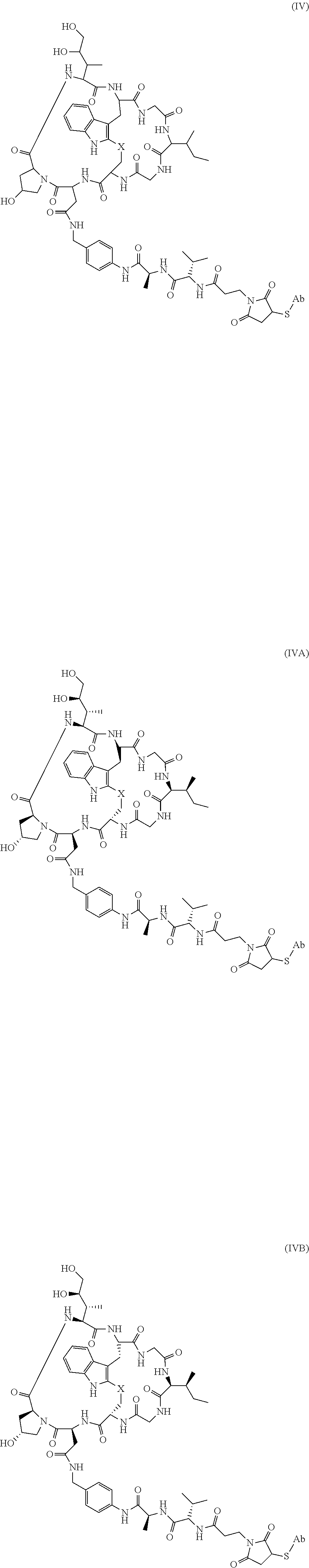

wherein X is —S—, —S(O)—, or —SO₂—, and the Ab is shown to indicate the point of Ab attachment.

In some embodiments, Am-L-Z-Ab is

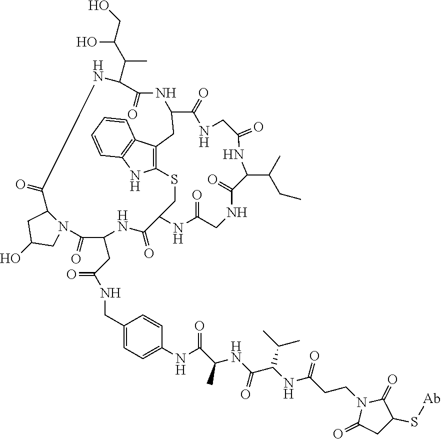

In some embodiments, Am-L-Z-Ab is

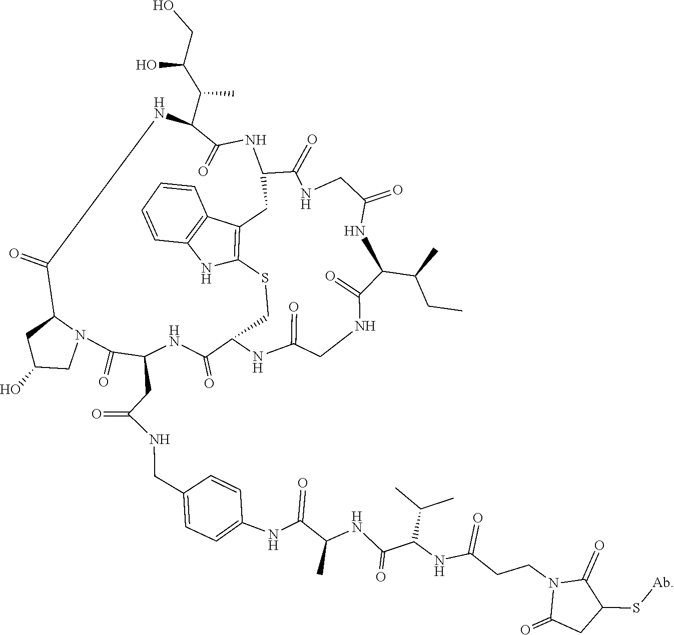

In some embodiments, Am-L-Z-Ab is

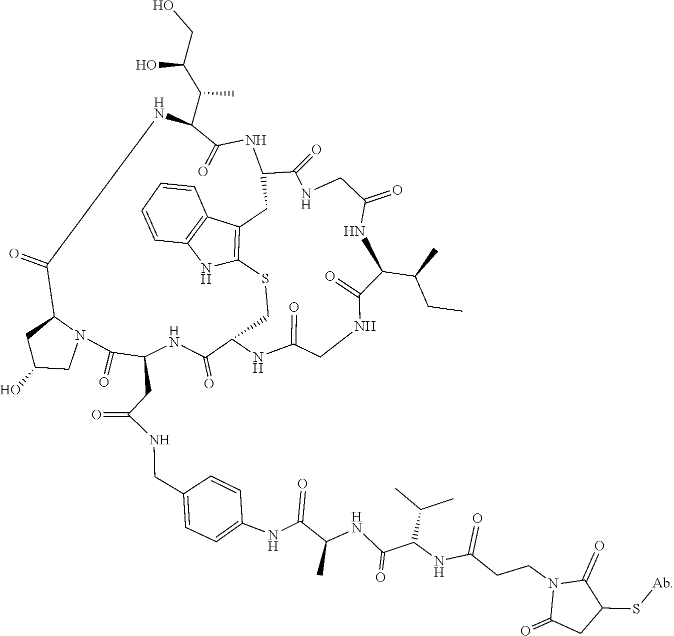

In some embodiments, Am-L-Z=Ab is represented by formula (IA)

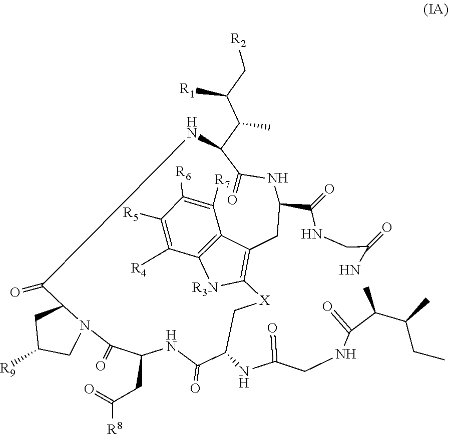

wherein R₁ is H, OH, OR_{A}, or OR_{C};

R₂ is H, OH, OR_{B}, or OR_{C};

R_{A} and R_{B}, when present, together with the oxygen atoms to which they are bound, combine to form an optionally substituted 5-membered heterocycloalkyl group;

R₃ is H, R_{C}, or R_{D};

R₄ is H, OH, OR_{C}, OR_{D}, R_{C}, or R_{D};

R₅ is H, OH, OR_{C}, OR_{D}, R_{C}, or R_{D};

R₆ is H, OH, OR_{C}, OR_{D}, R_{C}, or R_{D};

R₇ is H, OH, OR_{C}, OR_{D}, R_{C}, or R_{D};

R₈ is OH, NH₂, OR_{C}, OR_{D}, NHR_{C}, or NR_{C}R_{D};

R₉ is H, OH, OR_{C}, or OR_{D};

X is —S—, —S(O)—, or —SO₂—;

R_{C} is -L-Z;

R_{D} is optionally substituted alkyl (e.g., C₁-C₆ alkyl), optionally substituted heteroalkyl (e.g., C₁-C₆ heteroalkyl), optionally substituted alkenyl (e.g., C₂-C₆ alkenyl), optionally substituted heteroalkenyl (e.g., C₂-C₆ heteroalkenyl), optionally substituted alkynyl (e.g., C₂-C₆ alkynyl), optionally substituted heteroalkynyl (e.g., C₂-C₆ heteroalkynyl), optionally substituted cycloalkyl, optionally substituted heterocycloalkyl, optionally substituted aryl, or optionally substituted heteroaryl;

L is a linker, such as optionally substituted alkylene (e.g., C₁-C₆ alkylene), optionally substituted heteroalkylene (C₁-C₆ heteroalkylene), optionally substituted alkenylene (e.g., C₂-C₆ alkenylene), optionally substituted heteroalkenylene (e.g., C₂-C₆ heteroalkenylene), optionally substituted alkynylene (e.g., C₂-C₆ alkynylene), optionally substituted heteroalkynylene (e.g., C₂-C₆ heteroalkynylene), optionally substituted cycloalkylene, optionally substituted heterocycloalkylene, optionally substituted arylene, optionally substituted heteroarylene, a peptide, a dipeptide, —(C═O)—, a disulfide, a thioether, a hydrazone, or a combination thereof;

Z is a chemical moiety formed from a coupling reaction between a reactive substituent present on L and a reactive substituent present within an antibody, or antigen-binding fragment thereof, that binds CD117 (such as GNNK+ CD117); and

wherein Am contains exactly one R_{C} substituent.

In some embodiments, the linker includes —((CH₂)ₙ where n is 6. In some embodiments, L-Z is

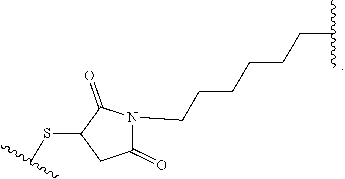 [SMILES: *CCCCCCN1C(=O)CC(S*)C1=O]

In some embodiments, L-Z is

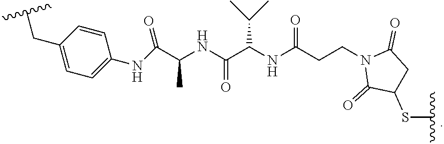

In some embodiments, Am-L-Z is represented by formula (IB)

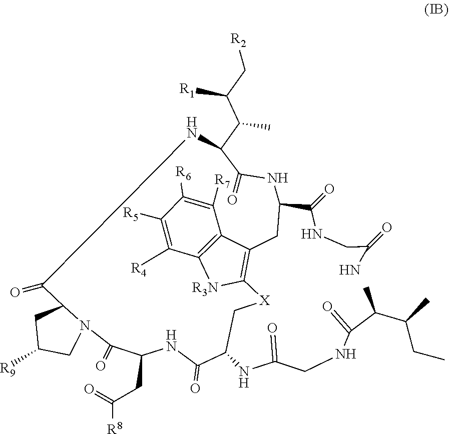

wherein R₁ is H, OH, OR_{A}, or OR_{C};

R₂ is H, OH, OR_{B}, or OR_{C};

R_{A} and R_{B}, when present, together with the oxygen atoms to which they are bound, combine to form an optionally substituted 5-membered heterocycloalkyl group;

R₃ is H, R_{C}, or R_{D};

R₄ is H, OH, OR_{C}, OR_{D}, R_{C}, or R_{D};

R₅ is H, OH, OR_{C}, OR_{D}, R_{C}, or R_{D};

R₆ is H, OH, OR_{C}, OR_{D}, R_{C}, or R_{D};

R₇ is H, OH, OR_{C}, OR_{D}, R_{C}, or R_{D};

R₈ is OH, NH₂, OR_{C}, OR_{D}, NHR_{C}, or NR_{C}R_{D};

R₆ is H, OH, OR_{C}, or OR_{D};

X is —S—, —S(O)—, or —SO₂—;

R_{C} is -L-Z;

R_{D} is optionally substituted alkyl (e.g., C₁-C₆ alkyl), optionally substituted heteroalkyl (e.g., C₁-C₆ heteroalkyl), optionally substituted alkenyl (e.g., C₂-C₆ alkenyl), optionally substituted heteroalkenyl (e.g., C₂-C₆ heteroalkenyl), optionally substituted alkynyl (e.g., C₂-C₆ alkynyl), optionally substituted heteroalkynyl (e.g., C₂-C₆ heteroalkynyl), optionally substituted cycloalkyl, optionally substituted heterocycloalkyl, optionally substituted aryl, or optionally substituted heteroaryl; L is a linker, such as optionally substituted alkylene (e.g., C₁-C₆ alkylene), optionally substituted heteroalkylene (C₁-C₆ heteroalkylene), optionally substituted alkenylene (e.g., C₂-C₆ alkenylene), optionally substituted heteroalkenylene (e.g., C₂-C₆ heteroalkenylene), optionally substituted alkynylene (e.g., C₂-C₆ alkynylene), optionally substituted heteroalkynylene (e.g., C₂-C₆ heteroalkynylene), optionally substituted cycloalkylene, optionally substituted heterocycloalkylene, optionally substituted arylene, optionally substituted heteroarylene, a peptide, a dipeptide, —(C═O)—, a disulfide, a thioether, a hydrazone, or a combination thereof;

Z is a chemical moiety formed from a coupling reaction between a reactive substituent present on L and a reactive substituent present within an antibody, or antigen-binding fragment thereof, that binds CD117 (such as GNNK+ CD117); and
wherein Am contains exactly one R_{C} substituent.

In some embodiments, the linker L and the chemical moiety Z, taken together as L-Z, is

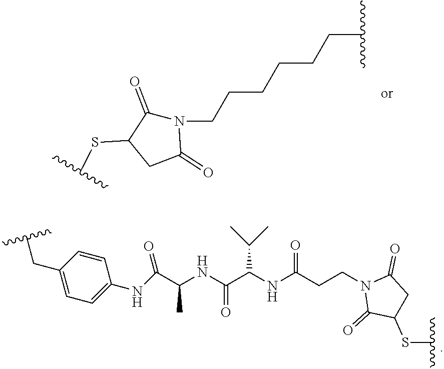

In some embodiments, L-Z is

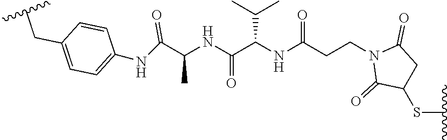 [SMILES: *Cc1ccc(NC(=O)[C@H](C)NC(=O)[C@@H](NC(=O)CCN2C(=O)CC(S*)C2=O)C(C)C)cc1]

In some embodiments, R_{A} and R_{B}, together with the oxygen atoms to which they are bound, combine to form to form a 5-membered heterocycloalkyl group of formula:

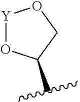

wherein Y is —(C═O)—, —(C═S)—, —(C≡NR_{E})—, or —(CR_{E}R_{E′})—; and

R_{E} and R_{E′} are each independently optionally substituted C₁-C₆ alkylene-R_{C}, optionally substituted C₁-C₆ heteroalkylene-R_{C}, optionally substituted C₂-C₆ alkenylene-R_{C}, optionally substituted C₂-C₆ heteroalkenylene-R_{C}, optionally substituted C₂-C₆ alkynylene-R_{C}, optionally substituted C₂-C₆ heteroalkynylene-R_{C}, optionally substituted cycloalkylene-R_{C}, optionally substituted heterocycloalkylene-R_{C}, optionally substituted arylene-R_{C}, or optionally substituted heteroarylene-R_{C}.

In some embodiments, Am-L-Z is represented by formula (IA) or formula (IB),

wherein R₁ is H, OH, OR_{A}, or OR_{C};

R₂ is H, OH, OR_{B}, or OR_{C};

R_{A} and R_{B}, when present, together with the oxygen atoms to which they are bound, combine to form:

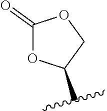 [SMILES: *[C@@H]1COC(=O)O1]

R₃ is H or R_{C};

R₄ is H, OH, OR_{C}, OR_{D}, R_{C}, or R_{D};

R₅ is H, OH, OR_{C}, OR_{D}, R_{C}, or R_{D};

R₆ is H, OH, OR_{C}, OR_{D}, R_{C}, or R_{D};

R₇ is H, OH, OR_{C}, OR_{D}, R_{C}, or R_{D};

R₈ is OH, NH₂, OR_{C}, or NHR_{C};

R₉ is H or OH;

X is —S—, —S(O)—, or —SO₂—; and

wherein R_{C} and R_{D} are each as defined above.

In some embodiments, Am-L-Z is represented by formula (IA) or formula (IB),

wherein R₁ is H, OH, OR_{A}, or OR_{C};

R₂ is H, OH, OR_{B}, or OR_{C};

R_{A} and R_{B}, when present, together with the oxygen atoms to which they are bound, combine to form:

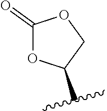 [SMILES: *[C@@H]1COC(=O)O1]

R₃ is H or R_{C};

R₄ and R₅ are each independently H, OH, OR_{C}, R_{C}, or OR_{D};

R₆ and R₇ are each H;

R₈ is OH, NH₂, OR_{C}, or NHR_{C};

R₉ is H or OH;

X is —S—, —S(O)—, or —SO₂—; and

wherein R_{C} is as defined above.

In some embodiments, Am-L-Z is represented by formula (IA) or formula (IB),

wherein R₁ is H, OH, or OR_{A};

R₂ is H, OH, or OR_{B};

R_{A} and R_{B}, when present, together with the oxygen atoms to which they are bound, combine to form:

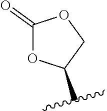 [SMILES: *[C@@H]1COC(=O)O1]

R₃, R₄, R₆, and R₇ are each H;

R₅ is OR_{C};

R₈ is OH or NH₂;

R₉ is H or OH;

X is —S—, —S(O)—, or —SO₂—; and

wherein R_{C} is as defined above. Such amatoxin conjugates are described, for example, in US Patent Application Publication No. 2016/0002298, the disclosure of which is incorporated herein by reference in its entirety.

In some embodiments, Am-L-Z is represented by formula (IA) or formula (IB),

wherein R₁ and R₂ are each independently H or OH;

R₃ is R_{C};

R₄, R₆, and R₇ are each H;

R₅ is H, OH, or OC₁-C₆ alkyl;

R₈ is OH or NH₂;

R₉ is H or OH; and

wherein R_{C} is as defined above. Such amatoxin conjugates are described, for example, in US Patent Application Publication No. 2014/0294865, the disclosure of which is incorporated herein by reference in its entirety.

In some embodiments, Am-L-Z is represented by formula (IA) or formula (IB),

wherein R₁ and R₂ are each independently H or OH;

R₃, R₆, and R₇ are each H;

R₄ and R₅ are each independently H, OH, OR_{C}, or R_{C};

R₈ is OH or NH₂;

R₉ is H or OH; and

wherein R_{C} is as defined above. Such amatoxin conjugates are described, for example, in US Patent Application Publication No. 2015/0218220, the disclosure of which is incorporated herein by reference in its entirety.

In some embodiments, Am-L-Z is represented by formula (IA) or formula (IB),

wherein R₁ and R₂ are each independently H or OH;

R₃, R₆, and R₇ are each H;

R₄ and R₅ are each independently H or OH;

R₈ is OH, NH₂, OR_{C}, or NHR_{C};

R₉ is H or OH; and

wherein R_{C} is as defined above. Such amatoxin conjugates are described, for example, in U.S. Pat. Nos. 9,233,173 and 9,399,681, as well as in US 2016/0089450, the disclosures of each of which are incorporated herein by reference in their entirety.

In some embodiments, Am-L-Z′ is

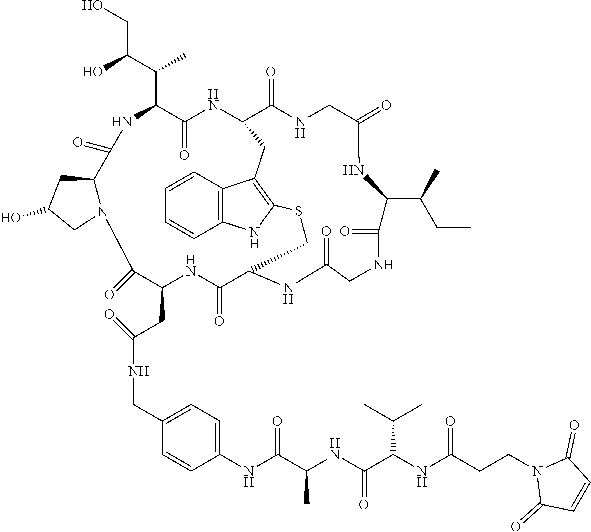

Additional amatoxins that may be used for conjugation to an antibody, or antigen-binding fragment thereof, in accordance with the compositions and methods described herein are described, for example, in WO 2016/142049; WO 2016/071856; WO 2017/046658; and WO2018/115466, the disclosures of each of which are incorporated herein by reference in their entirety.

In some embodiments, Am-L-Z is represented by formula (II), formula IIA, or formula IIB

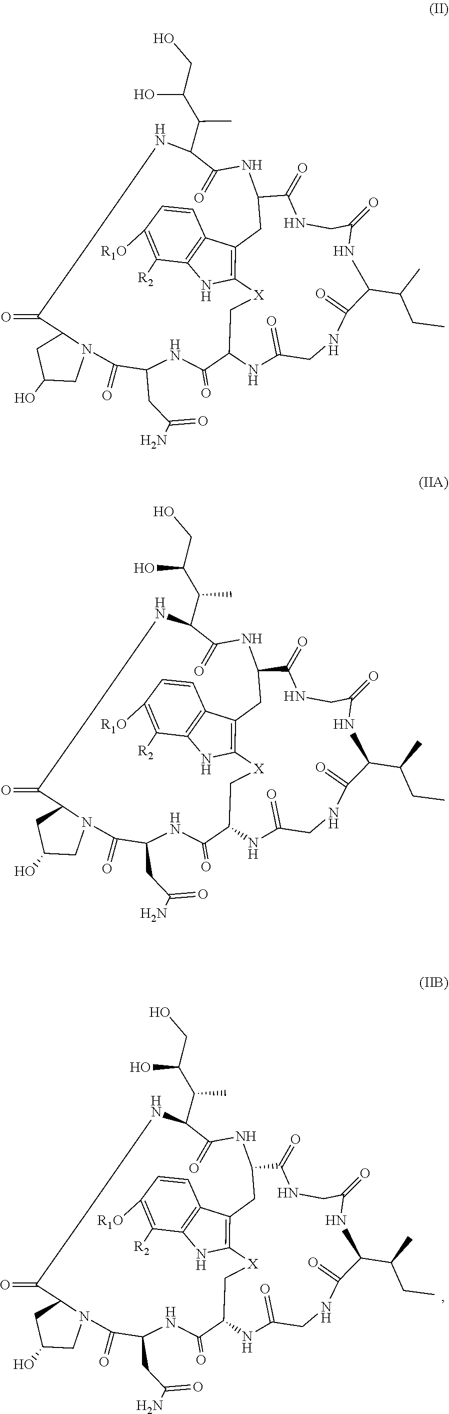

wherein X is S, SO, or SO₂; R₁ is H or a linker covalently bound to the antibody or antigen-binding fragment thereof through a chemical moeity Z, formed from a coupling reaction between a reactive substituent present on the linker and a reactive substituent present within an antibody, or antigen-binding fragment thereof; and R₂ is H or a linker covalently bound to the antibody or antigen-binding fragment thereof through a chemical moeity Z, formed from a coupling reaction between a reactive substituent present on the linker and a reactive substituent present within an antibody, or antigen-binding fragment thereof; wherein when R₁ is H, R₂ is the linker, and when R₂ is H, R₁ is the linker.

In some embodiments, R₁ is the linker and R₂ is H, and the linker and chemical moiety, together as L-Z, is

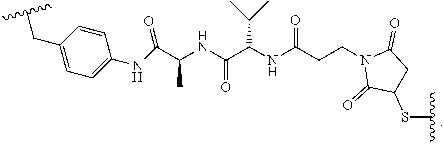

In some embodiments, the linker includes a —(CH₂)ₙ— unit, where n is an integer from 2-6. In some embodiments, R₁ is the linker and R₂ is H, and the linker and chemical moiety, together as L-Z, is

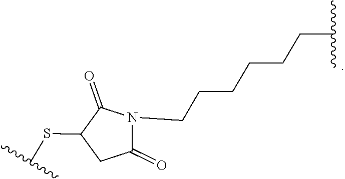 [SMILES: *CCCCCCN1C(=O)CC(S*)C1=O]

In some embodiments, Am-L-Z-Ab is:

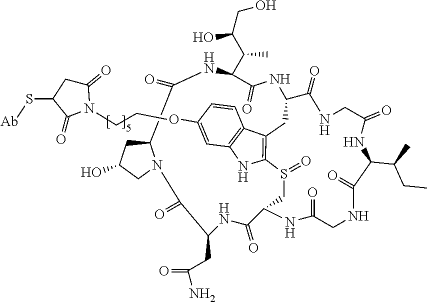

In some embodiments, Am-L-Z-Ab is:

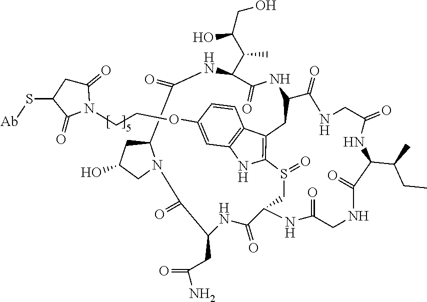

In some embodiments, the Am-L-Z-Ab precursor (i.e., Am-L-Z′) is one of:

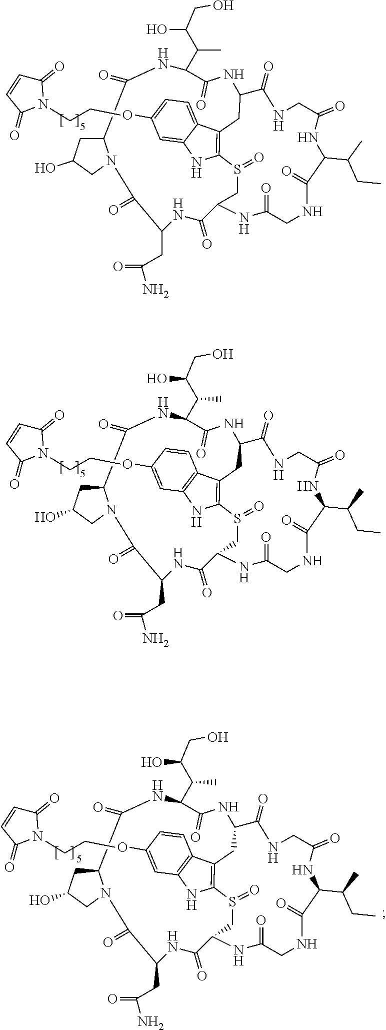

wherein the maleimide reacts with a thiol group found on a cysteine in the antibody.

In some embodiments, the cytotoxin is an α-amanitin. In some embodiments, the α-amanitin is a compound of formula III. In some embodiments, the α-amanitin of formula III is attached to an anti-CD117 antibody via a linker L. The linker L may be attached to the α-amanitin of formula III at any one of several possible positions (e.g., any of R¹-R⁹) to provide an α-amanitin-linker conjugate of formula I, IA, IB, II, IIA, IIB, IV, IVA or IVB. In some embodiments, the linker is attached at position R¹. In some embodiments, the linker is attached at position R². In some embodiments, the linker is attached at position R³. In some embodiments, the linker is attached at position R⁴. In some embodiments, the linker is attached at position R⁵. In some embodiments, the linker is attached at position R⁶. In some embodiments, the linker is attached at position R⁷. In some embodiments, the linker is attached at position R⁸. In some embodiments, the linker is attached at position R⁹. In some embodiments, the linker includes a hydrazine, a disulfide, a thioether or a dipeptide. In some embodiments, the linker includes a dipeptide selected from Val-Ala and Val-Cit. In some embodiments, the linker includes a para-aminobenzyl group (PAB). In some embodiments, the linker includes the moiety PAB-Cit-Val. In some embodiments, the linker includes the moiety PAB-Ala-Val. In some embodiments, the linker includes a —((C═O)(CH₂)ₙ— unit, wherein n is an integer from 1-6.

In some embodiments, the linker includes a —(CH₂)ₙ— unit, where n is an integer from 2-6. In some embodiments, the linker is-PAB-Cit-Val-((C═O)(CH₂)ₙ—. In some embodiments, the linker is-PAB-Ala-Val-((C═O)(CH₂)ₙ—. In some embodiments, the linker L and the chemical moiety Z, taken together as L-Z, is

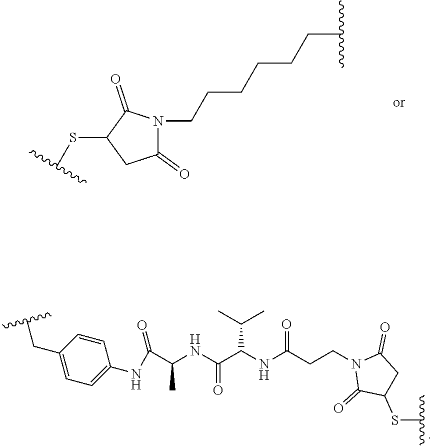

In some embodiments, the cytotoxin is a β-amanitin. In some embodiments, the β-amanitin is a compound of formula III. In some embodiments, the β-amanitin of formula III is attached to an anti-CD117 antibody via a linker L. The linker L may be attached to the β-amanitin of formula III at any one of several possible positions (e.g., any of R¹-R⁹) to provide an β-amanitin-linker conjugate of formula I, IA, IB, II, IIA, or IIB. In some embodiments, the linker is attached at position R¹. In some embodiments, the linker is attached at position R². In some embodiments, the linker is attached at position R³. In some embodiments, the linker is attached at position R⁴. In some embodiments, the linker is attached at position R⁵. In some embodiments, the linker is attached at position R⁶. In some embodiments, the linker is attached at position R⁷. In some embodiments, the linker is attached at position R⁸. In some embodiments, the linker is attached at position R⁹. In some embodiments, the linker includes a hydrazine, a disulfide, a thioether or a dipeptide. In some embodiments, the linker includes a dipeptide selected from Val-Ala and Val-Cit. In some embodiments, the linker includes a para-aminobenzyl group (PAB). In some embodiments, the linker includes the moiety PAB-Cit-Val. In some embodiments, the linker includes the moiety PAB-Ala-Val. In some embodiments, the linker includes a —((C═O)(CH₂)ₙ— unit, wherein n is an integer from 1-6.

In some embodiments, the linker includes a —(CH₂)ₙ— unit, where n is an integer from 2-6. In some embodiments, the linker is-PAB-Cit-Val-((C═O)(CH₂)ₙ—. In some embodiments, the linker is-PAB-Ala-Val-((C═O)(CH₂)ₙ—. In some embodiments, the linker L and the chemical moiety Z, taken together as L-Z, is

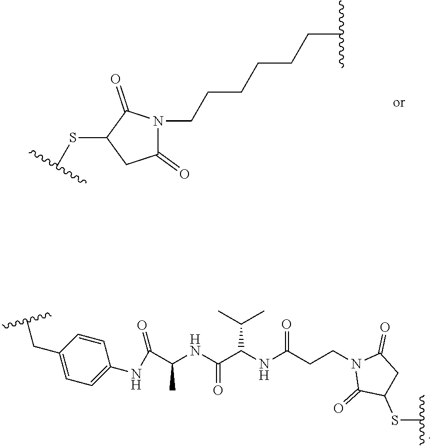

In some embodiments, the cytotoxin is a γ-amanitin. In some embodiments, the γ-amanitin is a compound of formula III. In some embodiments, the γ-amanitin of formula III is attached to an anti-CD117 antibody via a linker L. The linker L may be attached to the γ-amanitin of formula III at any one of several possible positions (e.g., any of R¹-R⁹) to provide an γ-amanitin-linker conjugate of formula I, IA, IB, II, IIA, or IIB. In some embodiments, the linker is attached at position R¹. In some embodiments, the linker is attached at position R². In some embodiments, the linker is attached at position R³. In some embodiments, the linker is attached at position R⁴. In some embodiments, the linker is attached at position R⁵. In some embodiments, the linker is attached at position R⁶. In some embodiments, the linker is attached at position R⁷. In some embodiments, the linker is attached at position R⁸. In some embodiments, the linker is attached at position R⁹. In some embodiments, the linker includes a hydrazine, a disulfide, a thioether or a dipeptide. In some embodiments, the linker includes a dipeptide selected from Val-Ala and Val-Cit. In some embodiments, the linker includes a para-aminobenzyl group (PAB). In some embodiments, the linker includes the moiety PAB-Cit-Val. In some embodiments, the linker includes the moiety PAB-Ala-Val. In some embodiments, the linker includes a —((C═O)(CH₂)ₙ— unit, wherein n is an integer from 1-6.

In some embodiments, the linker includes a —(CH₂)ₙ— unit, where n is an integer from 2-6. In some embodiments, the linker is-PAB-Cit-Val-((C═O)(CH₂)ₙ—. In some embodiments, the linker is-PAB-Ala-Val-((C═O)(CH₂)ₙ—. In some embodiments, the linker L and the chemical moiety Z, taken together as L-Z, is

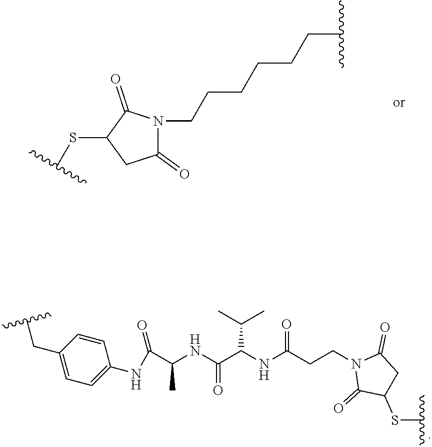

In some embodiments, the cytotoxin is a ε-amanitin. In some embodiments, the ε-amanitin is a compound of formula III. In some embodiments, the ε-amanitin of formula III is attached to an anti-CD117 antibody via a linker L. The linker L may be attached to the ε-amanitin of formula III at any one of several possible positions (e.g., any of R¹-R⁹) to provide an ε-amanitin-linker conjugate of formula I, IA, IB, II, IIA, or IIB. In some embodiments, the linker is attached at position R¹. In some embodiments, the linker is attached at position R². In some embodiments, the linker is attached at position R³. In some embodiments, the linker is attached at position R⁴. In some embodiments, the linker is attached at position R⁵. In some embodiments, the linker is attached at position R⁶. In some embodiments, the linker is attached at position R⁷. In some embodiments, the linker is attached at position R⁸. In some embodiments, the linker is attached at position R⁹. In some embodiments, the linker includes a hydrazine, a disulfide, a thioether or a dipeptide. In some embodiments, the linker includes a dipeptide selected from Val-Ala and Val-Cit. In some embodiments, the linker includes a para-aminobenzyl group (PAB). In some embodiments, the linker includes the moiety PAB-Cit-Val. In some embodiments, the linker includes the moiety PAB-Ala-Val. In some embodiments, the linker includes a —((C═O)(CH₂)ₙ— unit, wherein n is an integer from 1-6.

In some embodiments, the linker includes a —(CH₂)ₙ— unit, where n is an integer from 2-6. In some embodiments, the linker is-PAB-Cit-Val-((C═O)(CH₂)ₙ—. In some embodiments, the linker is-PAB-Ala-Val-((C═O)(CH₂)ₙ—. In some embodiments, the linker L and the chemical moiety Z, taken together as L-Z, is

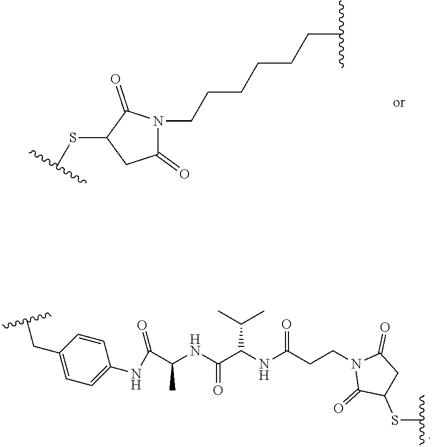

In some embodiments, the cytotoxin is an amanin. In some embodiments, the amanin is a compound of formula III. In some embodiments, the amanin of formula III is attached to an anti-CD117 antibody via a linker L. The linker L may be attached to the amanin of formula III at any one of several possible positions (e.g., any of R¹-R⁹) to provide an amanin-linker conjugate of formula I, IA, IB, II, IIA, or IIB. In some embodiments, the linker is attached at position R¹. In some embodiments, the linker is attached at position R². In some embodiments, the linker is attached at position R³. In some embodiments, the linker is attached at position R⁴. In some embodiments, the linker is attached at position R⁵. In some embodiments, the linker is attached at position R⁶. In some embodiments, the linker is attached at position R⁷. In some embodiments, the linker is attached at position R⁸. In some embodiments, the linker is attached at position R⁹. In some embodiments, the linker includes a hydrazine, a disulfide, a thioether or a dipeptide. In some embodiments, the linker includes a dipeptide selected from Val-Ala and Val-Cit. In some embodiments, the linker includes a para-aminobenzyl group (PAB). In some embodiments, the linker includes the moiety PAB-Cit-Val. In some embodiments, the linker includes the moiety PAB-Ala-Val. In some embodiments, the linker includes a —((C═O)(CH₂)ₙ— unit, wherein n is an integer from 1-6.

In some embodiments, the linker includes a —(CH₂)ₙ— unit, where n is an integer from 2-6. In some embodiments, the linker is-PAB-Cit-Val-((C═O)(CH₂)ₙ—. In some embodiments, the linker is-PAB-Ala-Val-((C═O)(CH₂)ₙ—. In some embodiments, the linker L and the chemical moiety Z, taken together as L-Z, is

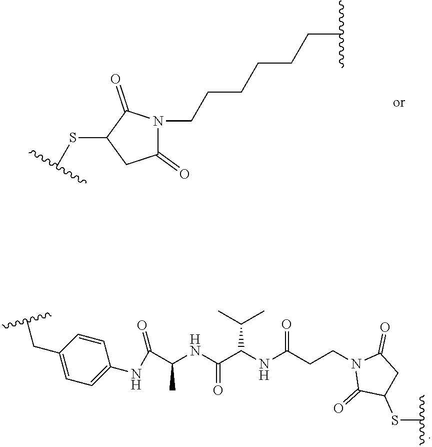

In some embodiments, the cytotoxin is an amaninamide. In some embodiments, the amaninamide is a compound of formula III. In some embodiments, the amaninamide of formula III is attached to an anti-CD117 antibody via a linker L. The linker L may be attached to the amaninamide of formula III at any one of several possible positions (e.g., any of R¹-R⁹) to provide an amaninamide-linker conjugate of formula I, IA, IB, II, IIA, or IIB. In some embodiments, the linker is attached at position R¹. In some embodiments, the linker is attached at position R². In some embodiments, the linker is attached at position R³. In some embodiments, the linker is attached at position R⁴. In some embodiments, the linker is attached at position R⁵. In some embodiments, the linker is attached at position R⁶. In some embodiments, the linker is attached at position R⁷. In some embodiments, the linker is attached at position R⁸. In some embodiments, the linker is attached at position R⁹. In some embodiments, the linker includes a hydrazine, a disulfide, a thioether or a dipeptide. In some embodiments, the linker includes a dipeptide selected from Val-Ala and Val-Cit. In some embodiments, the linker includes a para-aminobenzyl group (PAB). In some embodiments, the linker includes the moiety PAB-Cit-Val. In some embodiments, the linker includes the moiety PAB-Ala-Val. In some embodiments, the linker includes a —((C═O)(CH₂)ₙ— unit, wherein n is an integer from 1-6.

In some embodiments, the linker includes a —(CH₂)ₙ— unit, where n is an integer from 2-6. In some embodiments, the linker is-PAB-Cit-Val-((C═O)(CH₂)ₙ—. In some embodiments, the linker is-PAB-Ala-Val-((C═O)(CH₂)ₙ—. In some embodiments, the linker L and the chemical moiety Z, taken together as L-Z, is

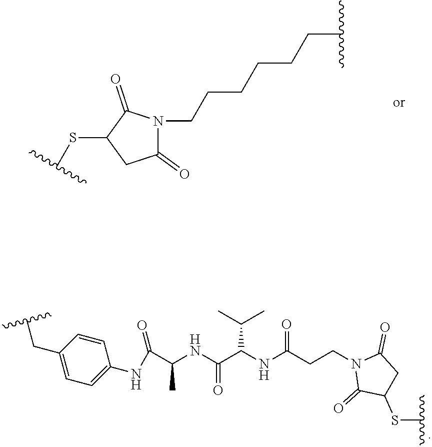

In some embodiments, the cytotoxin is an amanullin. In some embodiments, the amanullin is a compound of formula III. In some embodiments, the amanullin of formula III is attached to an anti-CD117 antibody via a linker L. The linker L may be attached to the amanullin of formula III at any one of several possible positions (e.g., any of R¹-R⁹) to provide an amanullin-linker conjugate of formula I, IA, IB, II, IIA, or IIB. In some embodiments, the linker is attached at position R¹. In some embodiments, the linker is attached at position R². In some embodiments, the linker is attached at position R³. In some embodiments, the linker is attached at position R⁴. In some embodiments, the linker is attached at position R⁵. In some embodiments, the linker is attached at position R⁶. In some embodiments, the linker is attached at position R⁷. In some embodiments, the linker is attached at position R⁸. In some embodiments, the linker is attached at position R⁹. In some embodiments, the linker includes a hydrazine, a disulfide, a thioether or a dipeptide. In some embodiments, the linker includes a dipeptide selected from Val-Ala and Val-Cit. In some embodiments, the linker includes a para-aminobenzyl group (PAB). In some embodiments, the linker includes the moiety PAB-Cit-Val. In some embodiments, the linker includes the moiety PAB-Ala-Val. In some embodiments, the linker includes a —((C═O)(CH₂)ₙ— unit, wherein n is an integer from 1-6. I

In some embodiments, the linker includes a —(CH₂)ₙ— unit, where n is an integer from 2-6. In some embodiments, the linker is-PAB-Cit-Val-((C═O)(CH₂)ₙ—. In some embodiments, the linker is-PAB-Ala-Val-((C═O)(CH₂)ₙ—. In some embodiments, the linker L and the chemical moiety Z, taken together as L-Z, is

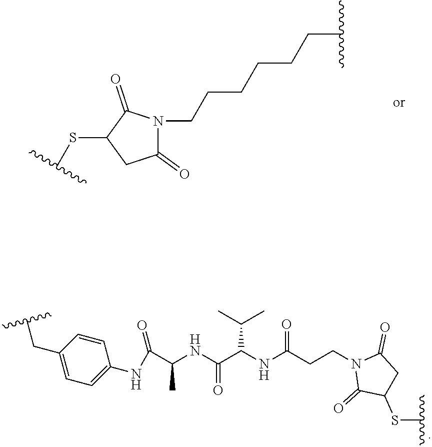

In some embodiments, the cytotoxin is an amanullinic acid. In some embodiments, the amanullinic acid is a compound of formula III. In some embodiments, the amanullinic acid of formula III is attached to an anti-CD117 antibody via a linker L. The linker L may be attached to the amanullinic acid of formula III at any one of several possible positions (e.g., any of R¹-R⁹) to provide an amanullinic acid-linker conjugate of formula I, IA, IB, II, IIA, or IIB. In some embodiments, the linker is attached at position R¹. In some embodiments, the linker is attached at position R². In some embodiments, the linker is attached at position R³. In some embodiments, the linker is attached at position R⁴. In some embodiments, the linker is attached at position R⁵. In some embodiments, the linker is attached at position R⁶. In some embodiments, the linker is attached at position R⁷. In some embodiments, the linker is attached at position R⁸. In some embodiments, the linker is attached at position R⁹. In some embodiments, the linker includes a hydrazine, a disulfide, a thioether or a dipeptide. In some embodiments, the linker includes a dipeptide selected from Val-Ala and Val-Cit. In some embodiments, the linker includes a para-aminobenzyl group (PAB). In some embodiments, the linker includes the moiety PAB-Cit-Val. In some embodiments, the linker includes the moiety PAB-Ala-Val. In some embodiments, the linker includes a —((C═O)(CH₂)ₙ— unit, wherein n is an integer from 1-6.

In some embodiments, the linker includes a —(CH₂)ₙ— unit, where n is an integer from 2-6. In some embodiments, the linker is-PAB-Cit-Val-((C═O)(CH₂)ₙ—. In some embodiments, the linker is-PAB-Ala-Val-((C═O)(CH₂)ₙ—. In some embodiments, the linker L and the chemical moiety Z, taken together as L-Z, is

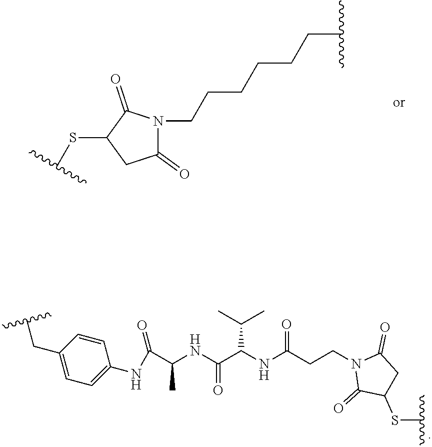

In some embodiments, the cytotoxin is a proamanullin. In some embodiments, the proamanullin is a compound of formula III. In some embodiments, the proamanullin of formula III is attached to an anti-CD117 antibody via a linker L. The linker L may be attached to the proamanullin of formula III at any one of several possible positions (e.g., any of R¹-R⁹) to provide an proamanullin-linker conjugate of formula I, IA, IB, II, IIA, or IIB. In some embodiments, the linker is attached at position R¹. In some embodiments, the linker is attached at position R². In some embodiments, the linker is attached at position R³. In some embodiments, the linker is attached at position R⁴. In some embodiments, the linker is attached at position R⁵. In some embodiments, the linker is attached at position R⁶. In some embodiments, the linker is attached at position R⁷. In some embodiments, the linker is attached at position R⁸. In some embodiments, the linker is attached at position R⁹. In some embodiments, the linker includes a hydrazine, a disulfide, a thioether or a dipeptide. In some embodiments, the linker includes a dipeptide selected from Val-Ala and Val-Cit. In some embodiments, the linker includes a para-aminobenzyl group (PAB). In some embodiments, the linker includes the moiety PAB-Cit-Val. In some embodiments, the linker includes the moiety PAB-Ala-Val. In some embodiments, the linker includes a —((C═O)(CH₂)ₙ— unit, wherein n is an integer from 1-6.

In some embodiments, the linker includes a —(CH₂)ₙ— unit, where n is an integer from 2-6. In some embodiments, the linker is-PAB-Cit-Val-((C═O)(CH₂)ₙ—. In some embodiments, the linker is-PAB-Ala-Val-((C═O)(CH₂)ₙ—. In some embodiments, the linker L and the chemical moiety Z, taken together as L-Z, is

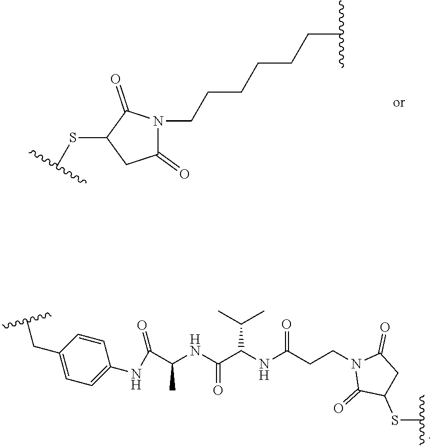

Synthetic methods of making amatoxin are described in U.S. Pat. No. 9,676,702, which is incorporated by reference herein.

Antibodies, or antigen-binding fragments, for use with the compositions and methods described herein can be conjugated to an amatoxin, such as α-amanitin or a variant thereof, using conjugation techniques known in the art or described herein. For instance, antibodies, or antigen-binding fragments thereof, that recognize and bind CD117 (such as GNNK+ CD117) can be conjugated to an amatoxin, such as α-amanitin or a variant thereof, as described in US 2015/0218220, the disclosure of which is incorporated herein by reference as it pertains, for example, to amatoxins, such as α-amanitin and variants thereof, as well as covalent linkers that can be used for covalent conjugation.

Exemplary antibody-drug conjugates useful in conjunction with the methods described herein may be formed by the reaction of an antibody, or antigen-binding fragment thereof, with an amatoxin that is conjugated to a linker containing a substituent suitable for reaction with a reactive residue on the antibody, or antigen-binding fragment thereof. Amatoxins that are conjugated to a linker containing a substituent suitable for reaction with a reactive residue on the antibody, or antigen-binding fragment thereof, described herein include, without limitation, 7′C-(4-(6-(maleimido)hexanoyl)piperazin-1-yl)-amatoxin; 7′C-(4-(6-(maleimido)hexanamido)piperidin-1-yl)-amatoxin; 7′C-(4-(6-(6-(maleimido)hexanamido)hexanoyl)piperazin-1-yl)-amatoxin; 7′C-(4-(4-((maleimido)methyl)cyclohexanecarbonyl)piperazin-1-yl)-amatoxin; 7′C-(4-(6-(4-((maleimido)methyl)cyclohexanecarboxamido)hexanoyl)piperazin-1-yl)-amatoxin; 7′C-(4-(2-(6-(maleimido)hexanamido)ethyl)piperidin-1-yl)-amatoxin; 7′C-(4-(2-(6-(6-(maleimido)hexanamido)hexanamido)ethyl)piperidin-1-yl)-amatoxin; 7′C-(4-(2-(4-((maleimido)methyl)cyclohexanecarboxamido)ethyl)piperidin-1-yl)-amatoxin; 7′C-(4-(2-(6-(4-((maleimido)methyl)cyclohexanecarboxamido)hexanamido)ethyl)piperidin-1-yl)-amatoxin; 7′C-(4-(2-(3-carboxypropanamido)ethyl)piperidin-1-yl)-amatoxin; 7′C-(4-(2-(2-bromoacetamido)ethyl)piperidin-1-yl)-amatoxin; 7′C-(4-(2-(3-(pyridin-2-yldisulfanyl)propanamido)ethyl)piperidin-1-yl)-amatoxin; 7′C-(4-(2-(4-(maleimido)butanamido)ethyl)piperidin-1-yl)-amatoxin; 7′C-(4-(2-(maleimido)acetyl)piperazin-1-yl)-amatoxin; 7′C-(4-(3-(maleimido)propanoyl)piperazin-1-yl)-amatoxin; 7′C-(4-(4-(maleimido)butanoyl)piperazin-1-yl)-amatoxin; 7′C-(4-(2-(6-(4-((maleimido)methyl)cyclohexanecarboxamido)hexanamido)ethyl)piperidin-1-yl)-amatoxin; 7′C-(3-((6-(maleimido)hexanamido)methyl)pyrrolidin-1-yl)-amatoxin; 7′C-(3-((6-(6-(maleimido)hexanamido)hexanamido)methyl)pyrrolidin-1-yl)-amatoxin; 7′C-(3-((4-((maleimido)methyl)cyclohexanecarboxamido)methyl)pyrrolidin-1-yl)-amatoxin; 7′C-(3-((6-((4-(maleimido)methyl)cyclohexanecarboxamido)hexanamido)methyl)pyrrolidin-1-yl)-amatoxin; 7′C-(4-(2-(6-(2-(aminooxy)acetamido)hexanamido)ethyl)piperidin-1-yl)-amatoxin; 7′C-(4-(2-(4-(2-(aminooxy)acetamido)butanamido)ethyl)piperidin-1-yl)-amatoxin; 7′C-(4-(4-(2-(aminooxy)acetamido)butanoyl)piperazin-1-yl)-amatoxin; 7′C-(4-(6-(2-(aminooxy)acetamido)hexanoyl)piperazin-1-yl)-amatoxin; 7′C-((4-(6-(maleimido)hexanamido)piperidin-1-yl)methyl)-amatoxin; 7′C-((4-(2-(6-(maleimido)hexanamido)ethyl)piperidin-1-yl)methyl)-amatoxin; 7′C-((4-(6-(maleimido)hexanoyl)piperazin-1-yl)methyl)-amatoxin; (R)-7′C-((3-((6-(maleimido)hexanamido)methyl)pyrrolidin-1-yl)methyl)-amatoxin; (S)-7′C-((3-((6-(maleimido)hexanamido)methyl)pyrrolidin-1-yl)methyl)-amatoxin; 7′C-((4-(2-(6-(6-(maleimido)hexanamido)hexanamido)ethyl)piperidin-1-yl)methyl)-amatoxin; 7′C-((4-(2-(4-((maleimido)methyl)cyclohexanecarboxamido)ethyl)piperidin-1-yl)methyl)-amatoxin; 7′C-((4-(2-(6-(4-((maleimido)methyl)cyclohexanecarboxamido)hexanamido)ethyl)piperidin-1-yl)methyl)-amatoxin; 7′C-((4-(2-(6-(maleimido)hexanamido)ethyl)piperazin-1-yl)methyl)-amatoxin; 7′C-((4-(2-(6-(6-(maleimido)hexanamido)hexanamido)ethyl)piperazin-1-yl)methyl)-amatoxin; 7′C-((4-(2-(4-((maleimido)methyl)cyclohexanecarboxamido)ethyl)piperazin-1-yl)methyl)-amatoxin; 7′C-((4-(2-(6-(4-((maleimido)methyl)cyclohexanecarboxamido)hexanamido)ethyl)piperazin-1-yl)methyl)-amatoxin; 7′C-((3-((6-(6-(maleimido)hexanamido)hexanamido)-S-methyl)pyrrolidin-1-yl)methyl)-amatoxin; 7′C-((3-((6-(6-(maleimido)hexanamido)hexanamido)-R-methyl)pyrrolidin-1-yl)methyl)-amatoxin; 7′C-((3-((4-((maleimido)methyl)cyclohexanecarboxamido)-S-methyl)pyrrolidin-1-yl)methyl)-amatoxin; 7′C-((3-((4-((maleimido)methyl)cyclohexanecarboxamido)-R-methyl)pyrrolidin-1-yl)methyl)-amatoxin; 7′C-((3-((6-(4-((maleimido)methyl)cyclohexanecarboxamido)hexanamido)methyl)pyrrolidin-1-yl)methyl)-amatoxin; 7′C-((4-(2-(3-carboxypropanamido)ethyl)piperazin-1-yl)methyl)-amatoxin; 7′C-((4-(6-(6-(maleimido)hexanamido)hexanoyl)piperazin-1-yl)methyl)-amatoxin; 7′C-((4-(6-(4-((maleimido)methyl)cyclohexanecarboxamido)hexanoyl)piperazin-1-yl)methyl)-amatoxin; 7′C-((4-(2-(maleimido)acetyl)piperazin-1-yl)methyl)-amatoxin; 7′C-((4-(3-(maleimido)propanoyl)piperazin-1-yl)methyl)-amatoxin; 7′C-((4-(4-(maleimido)butanoyl)piperazin-1-yl)methyl)-amatoxin; 7′C-((4-(2-(2-(maleimido)acetamido)ethyl)piperidin-1-yl)methyl)-amatoxin; 7′C-((4-(2-(4-(maleimido)butanamido)ethyl)piperidin-1-yl)methyl)-amatoxin; 7′C-((4-(2-(6-(4-((maleimido)methyl)cyclohexanecarboxamido)hexanamido)ethyl)piperidin-1-yl)methyl)-amatoxin; 7′C-((3-((6-(maleimido)hexanamido)methyl)azetidin-1-yl)methyl)-amatoxin; 7′C-((3-(2-(6-(maleimido)hexanamido)ethyl)azetidin-1-yl)methyl)-amatoxin; 7′C-((3-((4-((maleimido)methyl)cyclohexanecarboxamido)methyl)azetidin-1-yl)methyl)-amatoxin; 7′C-((3-(2-(4-((maleimido)methyl)cyclohexanecarboxamido)ethyl)azetidin-1-yl)methyl)-amatoxin; 7′C-((3-(2-(6-(4-((maleimido)methyl)cyclohexanecarboxamido)hexanamido)ethyl)azetidin-1-yl)methyl)-amatoxin; 7′C-(((2-(6-(maleimido)-N-methylhexanamido)ethyl)(methyl)amino)methyl)-amatoxin; 7′C-(((4-(6-(maleimido)-N-methylhexanamido)butyl(methyl)amino)methyl)-amatoxin; 7′C-((2-(2-(6-(maleimido)hexanamido)ethyl)aziridin-1-yl)methyl)-amatoxin; 7′C-((2-(2-(6-(4-((maleimido)methyl)cyclohexanecarboxamido)hexanamido)ethyl)aziridin-1-yl)methyl)-amatoxin; 7′C-((4-(6-(6-(2-(aminooxy)acetamido)hexanamido)hexanoyl)piperazin-1-yl)methyl)-amatoxin; 7′C-((4-(1-(aminooxy)-2-oxo-6,9,12,15-tetraoxa-3-azaheptadecan-17-oyl)piperazin-1-yl)methyl)-amatoxin; 7′C-((4-(2-(2-(aminooxy)acetamido)acetyl)piperazin-1-yl)methyl)-amatoxin; 7′C-((4-(3-(2-(aminooxy)acetamido)propanoyl)piperazin-1-yl)methyl)-amatoxin; 7′C-((4-(4-(2-(aminooxy)acetamido)butanoyl)piperazin-1-yl)methyl)-amatoxin; 7′C-((4-(2-(6-(2-(aminooxy)acetamido)hexanamido)ethyl)piperidin-1-yl)methyl)-amatoxin; 7′C-((4-(2-(2-(2-(aminooxy)acetamido)acetamido)ethyl)piperidin-1-yl)methyl)-amatoxin; 7′C-((4-(2-(4-(2-(aminooxy)acetamido)butanamido)ethyl)piperidin-1-yl)methyl)-amatoxin; 7′C-((4-(20-(aminooxy)-4,19-dioxo-6,9,12,15-tetraoxa-3,18-diazaicosyl)piperidin-1-yl)methyl)-amatoxin; 7′C-(((2-(6-(2-(aminooxy)acetamido)-N-methylhexanamido)ethyl)(methyl)amino)methyl)-amatoxin; 7′C-(((4-(6-(2-(aminooxy)acetamido)-N-methylhexanamido)butyl)(methyl)amino)methyl)-amatoxin; 7′C-((3-((6-(4-((maleimido)methyl)cyclohexanecarboxamido)hexanamido)methyl)pyrrolidin-1-yl)-S-methyl)-amatoxin; 7′C-((3-((6-(4-((maleimido)methyl)cyclohexanecarboxamido)hexanamido)-R-methyl)pyrrolidin-1-yl)methyl)-amatoxin; 7′C-((4-(2-(2-bromoacetamido)ethyl)piperazin-1-yl)methyl)-amatoxin; 7′C-((4-(2-(2-bromoacetamido)ethyl)piperidin-1-yl)methyl)-amatoxin; 7′C-((4-(2-(3-(pyridine-2-yldisulfanyl)propanamido)ethyl)piperidin-1-yl)methyl)-amatoxin; 6′O-(6-(6-(maleimido)hexanamido)hexyl)-amatoxin; 6′O-(5-(4-((maleimido)methyl)cyclohexanecarboxamido)pentyl)-amatoxin; 6′O-(2-((6-(maleimido)hexyl)oxy)-2-oxoethyl)-amatoxin; 6′O-((6-(maleimido) hexyl)carbamoyl)-amatoxin; 6′O-((6-(4-((maleimido)methyl)cyclohexanecarboxamido)hexyl)carbamoyl)-amatoxin; 6′O-(6-(2-bromoacetamido)hexyl)-amatoxin; 7′C-(4-(6-(azido)hexanamido)piperidin-1-yl)-amatoxin; 7′C-(4-(hex-5-ynoylamino)piperidin-1-yl)-amatoxin; 7′C-(4-(2-(6-(maleimido)hexanamido)ethyl)piperazin-1-yl)-amatoxin; 7′C-(4-(2-(6-(6-(maleimido)hexanamido)hexanamido)ethyl)piperazin-1-yl)-amatoxin; 6′O-(6-(6-(11,12-didehydro-5,6-dihydro-dibenz[b,f]azocin-5-yl)-6-oxohexanamido)hexyl)-amatoxin; 6′O-(6-(hex-5-ynoylamino)hexyl)-amatoxin; 6′O-(6-(2-(aminooxy)acetylamido)hexyl)-amatoxin; 6′O-((6-aminooxy)hexyl)-amatoxin; and 6′O-(6-(2-iodoacetamido)hexyl)-amatoxin. The foregoing linkers, among others useful in conjunction with the compositions and methods described herein, are described, for example, in US Patent Application Publication No. 2015/0218220, the disclosure of which is incorporated herein by reference in its entirety.

Additional cytotoxins that can be conjugated to antibodies, or antigen-binding fragments thereof, that recognize and bind CD117 (such as GNNK+ CD117 for use in directly treating a cancer, autommine condition, or for conditioning a patient (e.g., a human patient) in preparation for hematopoietic stem cell transplant therapy include, without limitation, 5-ethynyluracil, abiraterone, acylfulvene, adecypenol, adozelesin, aldesleukin, altretamine, ambamustine, amidox, amifostine, aminolevulinic acid, amrubicin, amsacrine, anagrelide, anastrozole, andrographolide, angiogenesis inhibitors, antarelix, anti-dorsalizing morphogenetic protein-1, antiandrogen, prostatic carcinoma, antiestrogen, antineoplaston, antisense oligonucleotides, aphidicolin glycinate, apoptosis gene modulators, apoptosis regulators, apurinic acid, asulacrine, atamestane, atrimustine, axinastatin 1, axinastatin 2, axinastatin 3, azasetron, azatoxin, azatyrosine, baccatin III derivatives, balanol, batimastat, BCR/ABL antagonists, benzochlorins, benzoylstaurosporine, beta lactam derivatives, beta-alethine, betaclamycin B, betulinic acid, bFGF inhibitors, bicalutamide, bisantrene, bisaziridinylspermine, bisnafide, bistratene A, bizelesin, breflate, bleomycin A2, bleomycin B2, bropirimine, budotitane, buthionine sulfoximine, calcipotriol, calphostin C, camptothecin derivatives (e.g., 10-hydroxy-camptothecin), capecitabine, carboxamide-amino-triazole, carboxyamidotriazole, carzelesin, casein kinase inhibitors, castanospermine, cecropin B, cetrorelix, chlorins, chloroquinoxaline sulfonamide, cicaprost, cis-porphyrin, cladribine, clomifene and analogues thereof, clotrimazole, collismycin A, collismycin B, combretastatin A4, combretastatin analogues, conagenin, crambescidin 816, crisnatol, cryptophycin 8, cryptophycin A derivatives, curacin A, cyclopentanthraquinones, cycloplatam, cypemycin, cytarabine ocfosfate, cytolytic factor, cytostatin, dacliximab, decitabine, dehydrodidemnin B, 2′deoxycoformycin (DCF), deslorelin, dexifosfamide, dexrazoxane, dexverapamil, diaziquone, didemnin B, didox, diethylnorspermine, dihydro-5-azacytidine, dihydrotaxol, dioxamycin, diphenyl spiromustine, discodermolide, docosanol, dolasetron, doxifluridine, droloxifene, dronabinol, duocarmycin SA, ebselen, ecomustine, edelfosine, edrecolomab, eflornithine, elemene, emitefur, epothilones, epithilones, epristeride, estramustine and analogues thereof, etoposide, etoposide 4′-phosphate (also referred to as etopofos), exemestane, fadrozole, fazarabine, fenretinide, filgrastim, finasteride, flavopiridol, flezelastine, fluasterone, fludarabine, fluorodaunorunicin hydrochloride, forfenimex, formestane, fostriecin, fotemustine, gadolinium texaphyrin, gallium nitrate, galocitabine, ganirelix, gelatinase inhibitors, gemcitabine, glutathione inhibitors, hepsulfam, homoharringtonine (HHT), hypericin, ibandronic acid, idoxifene, idramantone, ilmofosine, ilomastat, imidazoacridones, imiquimod, immunostimulant peptides, iobenguane, iododoxorubicin, ipomeanol, irinotecan, iroplact, irsogladine, isobengazole, jasplakinolide, kahalalide F, lamellarin-N triacetate, lanreotide, leinamycin, lenograstim, lentinan sulfate, leptolstatin, letrozole, lipophilic platinum compounds, lissoclinamide 7, lobaplatin, lometrexol, lonidamine, losoxantrone, loxoribine, lurtotecan, lutetium texaphyrin, lysofylline, masoprocol, maspin, matrix metalloproteinase inhibitors, menogaril, rnerbarone, meterelin, methioninase, metoclopramide, MIF inhibitor, ifepristone, miltefosine, mirimostim, mithracin, mitoguazone, mitolactol, mitomycin and analogues thereof, mitonafide, mitoxantrone, mofarotene, molgramostim, mycaperoxide B, myriaporone, N-acetyldinaline, N-substituted benzamides, nafarelin, nagrestip, napavin, naphterpin, nartograstim, nedaplatin, nemorubicin, neridronic acid, nilutamide, nisamycin, nitrullyn, octreotide, okicenone, onapristone, ondansetron, oracin, ormaplatin, oxaliplatin, oxaunomycin, paclitaxel and analogues thereof, palauamine, palmitoylrhizoxin, pamidronic acid, panaxytriol, panomifene, parabactin, pazelliptine, pegaspargase, peldesine, pentosan polysulfate sodium, pentostatin, pentrozole, perflubron, perfosfamide, phenazinomycin, picibanil, pirarubicin, piritrexim, podophyllotoxin, porfiromycin, purine nucleoside phosphorylase inhibitors, raltitrexed, rhizoxin, rogletimide, rohitukine, rubiginone B1, ruboxyl, safingol, saintopin, sarcophytol A, sargramostim, sobuzoxane, sonermin, sparfosic acid, spicamycin D, spiromustine, stipiamide, sulfinosine, tallimustine, tegafur, temozolomide, teniposide, thaliblastine, thiocoraline, tirapazamine, topotecan, topsentin, triciribine, trimetrexate, veramine, vinorelbine, vinxaltine, vorozole, zeniplatin, and zilascorb, among others.

### Linkers for Chemical Conjugation

A variety of linkers can be used to conjugate antibodies, or antigen-binding fragments, as described herein (e.g., antibodies, or antigen-binding fragments thereof, that recognize and bind CD117 (such as GNNK+ CD117) with a cytotoxic molecule.

The term “Linker” as used herein means a divalent chemical moiety comprising a covalent bond or a chain of atoms that covalently attaches an antibody or fragment thereof (Ab) to a drug moiety (D) to form antibody-drug conjugates of the present disclosure (ADCs; Ab-Z-L-D, where D is a cytotoxin). Suitable linkers have two reactive termini, one for conjugation to an antibody and the other for conjugation to a cytotoxin. The antibody conjugation reactive terminus of the linker (reactive moiety, Z) is typically a site that is capable of conjugation to the antibody through a cysteine thiol or lysine amine group on the antibody, and so is typically a thiol-reactive group such as a double bond (as in maleimide) or a leaving group such as a chloro, bromo, iodo, or an R-sulfanyl group, or an amine-reactive group such as a carboxyl group; while the antibody conjugation reactive terminus of the linker is typically a site that is capable of conjugation to the cytotoxin through formation of an amide bond with a basic amine or carboxyl group on the cytotoxin, and so is typically a carboxyl or basic amine group. When the term “linker” is used in describing the linker in conjugated form, one or both of the reactive termini will be absent (such as reactive moiety Z, having been converted to chemical moiety Z) or incomplete (such as being only the carbonyl of the carboxylic acid) because of the formation of the bonds between the linker and/or the cytotoxin, and between the linker and/or the antibody or antigen-binding fragment thereof. Such conjugation reactions are described further herein below.

Generally, linkers suitable for the present disclosure may be substantially stable in circulation, but allow for release of the cytotoxin (e.g., an amatoxin as disclosed herein) within or in close proximity to the target cells. In some embodiments, the linker is cleavable under certain intracellular conditions, such that cleavage of the linker may release the drug unit from the antibody in the intracellular environment. In yet other embodiments, the linker unit is not cleavable and the drug may be released, for example, by antibody degradation. Generally, cleavable linkers contain one or more functional groups that is cleaved in response to a physiological environment. For example, a cleavable linker may contain an enzymatic substrate (e.g., valine-alanine) that degrades in the presence of an intracellular enzyme (e.g., cathepsin B), an acid-cleavable group (e.g., a hydrozone) that degrades in the acidic environment of a cellular compartment, or a reducible group (e.g., a disulfide) that degrades in an intracellular reducing environment. By contast, generally, non-cleavable linkers are released from the ADC during degradation (e.g., lysosomal degradation) of the antibody moiety of the ADC inside the target cell. In some embodiments, the linkers may be substantially stable outside the target cell and may be cleaved at some efficacious rate inside the cell. In some embodiments, an effective linker may: (i) maintain the specific binding properties of the antibody; (ii) allow intracellular delivery of the conjugate or drug moiety; (iii) remain stable and intact, i.e. not cleaved, until the conjugate has been delivered or transported to its targeted site; and (iv) maintain a cytotoxic, cell-killing effect or a cytostatic effect of the cytotoxic moiety. Stability of the ADC may be measured by standard analytical techniques such as mass spectroscopy, HPLC, and the separation/analysis technique LC/MS. In some embodiments, covalent attachment of the antibody and the drug moiety requires the linker to have two reactive functional groups, i.e. bivalency in a reactive sense. Bivalent linker reagents which are useful to attach two or more functional or biologically active moieties, such as peptides, nucleic acids, drugs, toxins, antibodies, haptens, and reporter groups are known, and methods have been described their resulting conjugates (Hermanson, G. T. (1996) Bioconjugate Techniques; Academic Press: New York, p. 234-242).

In some embodiments, linkers include those that may be cleaved, for instance, by enzymatic hydrolysis, photolysis, hydrolysis under acidic conditions, hydrolysis under basic conditions, oxidation, disulfide reduction, nucleophilic cleavage, or organometallic cleavage (see, for example, Leriche et al., Bioorg. Med. Chem., 20:571-582, 2012, the disclosure of which is incorporated herein by reference as it pertains to linkers suitable for covalent conjugation).

Linkers hydrolyzable under acidic conditions include, for example, hydrazones, semicarbazones, thiosemicarbazones, cis-aconitic amides, orthoesters, acetals, ketals, or the like. (See, e.g., U.S. Pat. Nos. 5,122,368; 5,824,805; 5,622,929; Dubowchik and Walker, 1999, Pharm. Therapeutics 83:67-123; Neville et al., 1989, Biol. Chem. 264:14653-14661, the disclosure of each of which is incorporated herein by reference in its entirety as it pertains to linkers suitable for covalent conjugation. Such linkers are relatively stable under neutral pH conditions, such as those in the blood, but are unstable at below pH 5.5 or 5.0, the approximate pH of the lysosome.

Linkers cleavable under reducing conditions include, for example, a disulfide. A variety of disulfide linkers are known in the art, including, for example, those that can be formed using SATA (N-succinimidyl-S-acetylthioacetate), SPDP (N-succinimidyl-3-(2-pyridyldithio)propionate), SPDB (N-succinimidyl-3-(2-pyridyldithio)butyrate) and SMPT (N-succinimidyl-oxycarbonyl-alpha-methyl-alpha-(2-pyridyl-dithio)toluene), SPDB and SMPT (See, e.g., Thorpe et al., 1987, Cancer Res. 47:5924-5931; Wawrzynczak et al., In Immunoconjugates: Antibody Conjugates in Radioimagery and Therapy of Cancer (C. W. Vogel ed., Oxford U. Press, 1987. See also U.S. Pat. No. 4,880,935, the disclosure of each of which is incorporated herein by reference in its entirety as it pertains to linkers suitable for covalent conjugation.

Linkers susceptible to enzymatic hydrolysis can be, e.g., a peptide-containing linker that is cleaved by an intracellular peptidase or protease enzyme, including, but not limited to, a lysosomal or endosomal protease. One advantage of using intracellular proteolytic release of the therapeutic agent is that the agent is typically attenuated when conjugated and the serum stabilities of the conjugates are typically high. In some embodiments, the peptidyl linker is at least two amino acids long or at least three amino acids long. Exemplary amino acid linkers include a dipeptide, a tripeptide, a tetrapeptide or a pentapeptide. Examples of suitable peptides include those containing amino acids such as Valine, Alanine, Citrulline (Cit), Phenylalanine, Lysine, Leucine, and Glycine. Amino acid residues which comprise an amino acid linker component include those occurring naturally, as well as minor amino acids and non-naturally occurring amino acid analogs, such as citrulline. Exemplary dipeptides include valine-citrulline (vc or val-cit) and alanine-phenylalanine (af or ala-phe). Exemplary tripeptides include glycine-valine-citrulline (gly-val-cit) and glycine-glycine-glycine (gly-gly-gly). In some embodiments, the linker includes a dipeptide such as Val-Cit, Ala-Val, or Phe-Lys, Val-Lys, Ala-Lys, Phe-Cit, Leu-Cit, Ile-Cit, Phe-Arg, or Trp-Cit. Linkers containing dipeptides such as Val-Cit or Phe-Lys are disclosed in, for example, U.S. Pat. No. 6,214,345, the disclosure of which is incorporated herein by reference in its entirety as it pertains to linkers suitable for covalent conjugation. In some embodiments, the linker includes a dipeptide selected from Val-Ala and Val-Cit. In some embodiments, a dipeptide is used in combination with a self-immolative linker.

Additional linkers suitable for the synthesis of drug-antibody conjugates conjugates as described herein include those capable of releasing a cytotoxin by a 1,6-elimination process (a “self-immolative” group), such as p-aminobenzyl alcohol (PABC), p-aminobenzyl (PAB), 6-maleimidohexanoic acid, pH-sensitive carbonates, and other reagents described in Jain et al., Pharm. Res. 32:3526-3540, 2015, the disclosure of which is incorporated herein by reference in its entirety.

In some embodiments, the linker includes a self-immolative group such as the aforementioned PAB or PABC (para-aminobenzyloxycarbonyl), which are disclosed in, for example, Carl et al., J. Med. Chem. (1981) 24:479-480; Chakravarty et al (1983) J. Med. Chem. 26:638-644; U.S. Pat. No. 6,214,345; US20030130189; US20030096743; U.S. Pat. No. 6,759,509; US20040052793; U.S. Pat. Nos. 6,218,519; 6,835,807; 6,268,488; US20040018194; WO98/13059; US20040052793; U.S. Pat. Nos. 6,677,435; 5,621,002; US20040121940; WO2004/032828). Other such chemical moieties capable of this process (“self-immolative linkers”) include methylene carbamates and heteroaryl groups such as aminothiazoles, aminoimidazoles, aminopyrimidines, and the like. Linkers containing such heterocyclic self-immolative groups are disclosed in, for example, U.S. Patent Publication Nos. 20160303254 and 20150079114, and U.S. Pat. No. 7,754,681; Hay et al. (1999) Bioorg. Med. Chem. Lett. 9:2237; US 2005/0256030; de Groot et al (2001) J. Org. Chem. 66:8815-8830; and U.S. Pat. No. 7,223,837. Linkers suitable for use herein further may include one or more groups selected from C₁-C₆ alkylene, C₁-C₆ heteroalkylene, C₂-C₆ alkenylene, C₂-C₆ heteroalkenylene, C₂-C₆ alkynylene, C₂-C₆ heteroalkynylene, C₃-C₆ cycloalkylene, heterocycloalkylene, arylene, heteroarylene, and combinations thereof, each of which may be optionally substituted. Non-limiting examples of such groups include (CH₂)ₙ, (CH₂CH₂O)ₙ, and —(C═O)(CH₂)ₙ— units, wherein n is an integer from 1-6, independently selected for each occasion.

Suitable linkers may contain groups having solubility enhancing properties. Linkers including the (CH₂CH₂O)ₚ unit (polyethylene glycol, PEG), for example, can enhance solubility, as can alkyl chains substituted with amino, sulfonic acid, phosphonic acid or phosphoric acid residues. Linkers including such moieties are disclosed in, for example, U.S. Pat. Nos. 8,236,319 and 9,504,756, the disclosure of each of which is incorporated herein by reference in its entirety as it pertains to linkers suitable for covalent conjugation. Further solubility enhancing groups include, for example, acyl and carbamoyl sulfamide groups, having the structure:

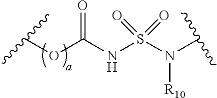

wherein a is 0 or 1; and

R¹⁰ is selected from the group consisting of hydrogen, C₁-C₂₄ alkyl groups, C₃-C₂₄ cycloalkyl groups, C₁-C₂₄ (hetero)aryl groups, C₁-C₂₄ alkyl(hetero)aryl groups and C₁-C₂₄ (hetero)arylalkyl groups, the C₁-C₂₄ alkyl groups, C₃-C₂₄ cycloalkyl groups, C₂-C₂₄ (hetero)aryl groups, C₃-C₂₄ alkyl(hetero)aryl groups and C₃-C₂₄ (hetero)arylalkyl groups, each of which may be optionally substituted and/or optionally interrupted by one or more heteroatoms selected from O, S and NR¹¹R¹² wherein R¹¹ and R¹² are independently selected from the group consisting of hydrogen and C₁-C₄ alkyl groups; or R¹⁰ is a cytotoxin, wherein the cytotoxin is optionally connected to N via a spacer moiety. Linkers containing such groups are described, for example, in U.S. Pat. No. 9,636,421 and U.S. Patent Application Publication No. 2017/0298145, the disclosures of which are incorporated herein by reference in their entirety as they pertain to linkers suitable for covalent conjugation to cytotoxins and antibodies or antigen-binding fragments thereof.

In some embodiments, the linker may include one or more of a hydrazine, a disulfide, a thioether, a dipeptide, a p-aminobenzyl (PAB) group, a heterocyclic self-immolative group, an optionally substituted C₁-C₆ alkyl, an optionally substituted C₁-C₆ heteroalkyl, an optionally substituted C₂-C₆ alkenyl, an optionally substituted C₂-C₆ heteroalkenyl, an optionally substituted C₂-C₆ alkynyl, an optionally substituted C₂-C₆ heteroalkynyl, an optionally substituted C₃-C₆ cycloalkyl, an optionally substituted heterocycloalkyl, an optionally substituted aryl, an optionally substituted heteroaryl, acyl, —(C═O)—, or —(CH₂CH₂O)ₙ— group, wherein n is an integer from 1-6. One of skill in the art will recognize that one or more of the groups listed may be present in the form of a bivalent (diradical) species, e.g., C₁-C₆ alkylene and the like.

In some embodiments, the linker L comprises the moiety *-L₁L₂-**, wherein:
L₁ is absent or is —(CH₂)ₘNR¹³C(═O)—, —(CH₂)ₘNR¹³—, —(CH₂)ₘX₃(CH₂)ₘ—,

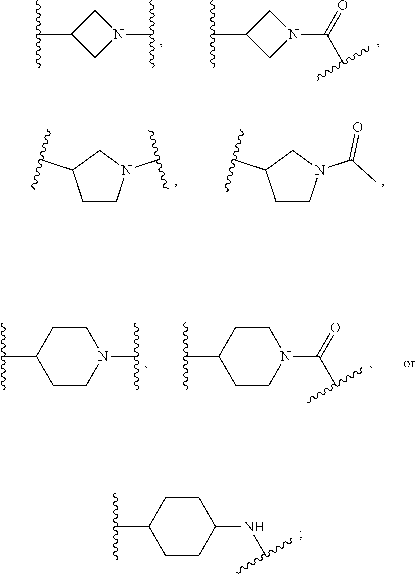

L₂ is absent or is —(CH₂)ₘ—, —NR¹³(CH₂)ₘ—, —(CH₂)ₘNR¹³C(═O)(CH₂)ₘ—, —X₄, —(CH₂)ₘNR¹³C(═O)X₄, —(CH₂)ₘNR¹³C(═O)—, —((CH₂)ₘO)ₙ(CH₂)ₘ—, —((CH₂)ₘO)ₙ(CH₂)ₘX₃(CH₂)ₘ—, —NR¹³((CH₂)ₘO)ₙX₃(CH₂)ₘ—, —NR¹³((CH₂)ₘO)ₙ(CH₂)ₘX₃(CH₂)ₘ—, —X₁X₂C(═O)(CH₂)ₘ—, —(CH₂)ₘ(O(CH₂)ₘ)ₙ—, —(CH₂)ₘNR¹³(CH₂)ₘ—, —(CH₂)ₘNR¹³C(═O)(CH₂)ₘX₃(CH₂)ₘ—, —(CH₂)ₘC(═O)NR¹³(CH₂)ₘNR¹³C(═O)(CH₂)ₘ—, —(CH₂)ₘC(═O)—, —(CH₂)ₘNR¹³(CH₂)ₘC(═O)X₂X₁C(═O)—, —(CH₂)ₘX₃(CH₂)ₘC(═O)X₂X₁C(═O)—, —(CH₂)ₘC(═O)NR¹³(CH₂)ₘ—, —(CH₂)ₘC(═O)NR¹³(CH₂)ₘX₃(CH₂)ₘ—, —(CH₂)ₘX₃(CH₂)ₘNR¹³C(═O)(CH₂)ₘ—, —(CH₂)ₘX₃(CH₂)ₘC(═O)NR¹³(CH₂)ₘ—, —(CH₂)ₘO)ₙ(CH₂)ₘNR¹³C(═O)(CH₂)ₘ—, —(CH₂)ₘC(═O)NR¹³(CH₂)ₘ(O(CH₂)ₘ)ₙ—, —(CH₂)ₘ(O(CH₂)ₘ)ₙC(═O)—, —(CH₂)ₘNR¹³(CH₂)ₘC(═O)—, —(CH₂)ₘC(═O)NR¹³(CH₂)ₘNR¹³C(═O)—, —(CH₂)ₘ(O(CH₂)ₘ)ₙX₃(CH₂)ₘ—, —(CH₂)ₘX₃((CH₂)ₘO)ₙ(CH₂)ₘ—, —(CH₂)ₘX₃(CH₂)ₘC(═O)—, —(CH₂)ₘC(═O)NR¹³(CH₂)ₘO)ₙ(CH₂)ₘX₃(CH₂)ₘ—, —(CH₂)ₘX₃(CH₂)ₘ(O(CH₂)ₘ)ₙNR¹³C(═O)(CH₂)ₘ—, —(CH₂)ₘX₃(CH₂)ₘ(O(CH₂)ₘ)ₙC(═O)—, —(CH₂)ₘX₃(CH₂)ₘ(O(CH₂)ₘ)ₙ—, —(CH₂)ₘC(═O)NR¹³(CH₂)ₘC(═O)—, —(CH₂)ₘC(═O)NR¹³(CH₂)ₘ(O(CH₂)ₘ)ₙC(═O)—, —((CH₂)ₘO)ₙ(CH₂)ₘNR¹³C(═O)(CH₂)ₘ—, —(CH₂)ₘC(═O)NR¹³(CH₂)ₘC(═O)NR¹³(CH₂)ₘ—, —(CH₂)ₘNR¹³C(═O)(CH₂)ₘNR¹³C(═O)(CH₂)ₘ—, —(CH₂)ₘX₃(CH₂)ₘC(═O)NR¹³—, —(CH₂)ₘC(═O)NR¹³—, —(CH₂)ₘX₃—, —C(R¹³)₂(CH₂)ₘ—, —(CH₂)ₘC(R¹³)₂NR¹³—, —(CH₂)ₘC(═O)NR¹³(CH₂)ₘNR¹³—, —(CH₂)ₘC(═O)NR¹³(CH₂)ₘNR¹³C(═O)NR¹³—, —(CH₂)ₘC(═O)X₂X₁C(═O)—, —C(R¹³)₂(CH₂)ₘNR¹³C(═O)(CH₂)ₘ—, —(CH₂)ₘC(═O)NR¹³(CH₂)ₘC(R¹³)₂NR¹³—, —C(R¹³)₂(CH₂)ₘX₃(CH₂)ₘ—, —(CH₂)ₘX₃(CH₂)ₘC(R¹³)₂NR¹³—, —C(R¹³)₂(CH₂)ₘOC(═O)NR¹³(CH₂)ₘ—, —(CH₂)ₘNR¹³C(═O)O(CH₂)ₘC(R¹³)₂NR¹³—, —(CH₂)ₘX₃(CH₂)ₘNR¹³—, —(CH₂)ₘX₃(CH₂)ₘ(O(CH₂)ₘ)ₙNR¹³—, —(CH₂)ₘNR¹³—, —(CH₂)ₘC(═O)NR¹³(CH₂)ₘ(O(CH₂)ₘ)ₙNR¹³—(CH₂)ₘ(O(CH₂)ₘ)ₙNR¹³—, —(CH₂CH₂O)ₙ(CH₂)ₘ—, —(CH₂)ₘ(OCH₂CH₂)ₙ; —(CH₂)ₘO(CH₂)ₘ—, —(CH₂)ₘS(═O)₂—, —(CH₂)ₘC(═O)NR¹³(CH₂)ₘS(═O)₂—, F12)ₘX₃(CH₂)ₘS(═O)₂—, —(CH₂)ₘX₂X₁C(═O)—, —(CH₂)ₘ(O(CH₂)ₘ)ₙC(═O)X₂X₁C(═O)—, —(CH₂)ₘ(O(CH₂)ₘ)ₙX₂X₁C(═O)—, —(CH₂)ₘX₃(CH₂)ₘX₂X₁C(═O)—, —(CH₂)ₘX₃(CH₂)ₘ(O(CH₂)ₘ)ₙX₂X₁C(═O)—, —(CH₂)ₘX₃(CH₂)ₘC(═O)NR¹³(CH₂)ₘNR¹³C(═O)—, —(CH₂)ₘX₃(CH₂)ₘC(═O)NR¹³(CH₂)ₘC(═O)—, —(CH₂)ₘX₃(CH₂)ₘC(═O)NR¹³(CH₂)ₘ(O(CH₂)ₘ)ₙC(═O)—, —(CH₂)ₘC(═O)X₂X₁C(═O)NR¹³(CH₂)ₘ—, —(CH₂)ₘX₃(O(CH₂)ₘ)ₙC(═O)—, —(CH₂)ₘNR¹³C(═O)((CH₂)ₘO)ₙ(CH₂)ₘ—, —(CH₂)ₘ(O(CH₂)ₘ)ₙC(═O)NR¹³(CH₂)ₘ—, —(CH₂)ₘNR¹³C(═O)NR¹³(CH₂)ₘ— or —(CH₂)ₘX₃(CH₂)ₘNR¹³C(═O)—;
wherein

### X₁ is

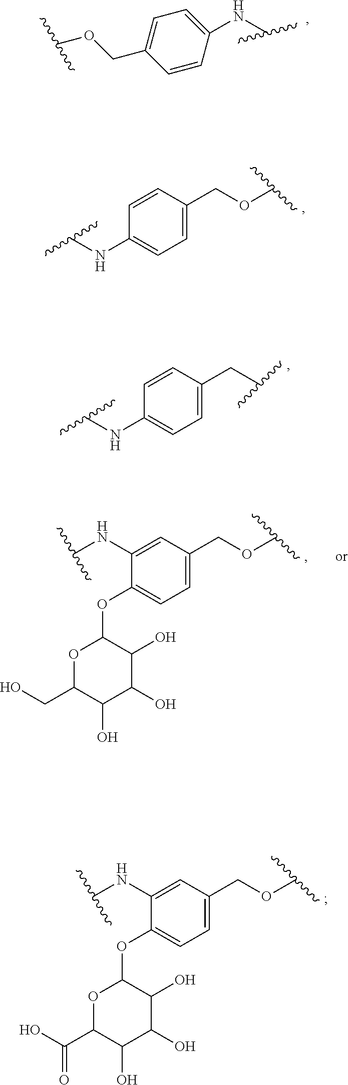 [SMILES: *Nc1ccc(CO*)cc1 ; *Nc1ccc(CO*)cc1 ; *Cc1ccc(N*)cc1 ; *Nc1cc(CO*)ccc1OC1OC(CO)C(O)C(O)C1O ; *Nc1cc(CO*)ccc1OC1OC(C(=O)O)C(O)C(O)C1O]

### X₂ is

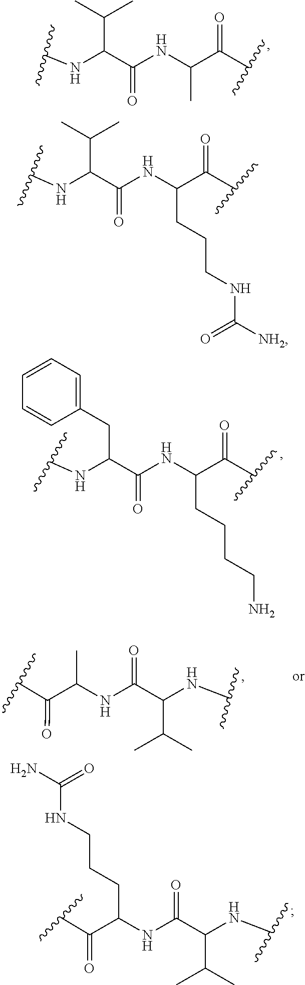

### X₃ is

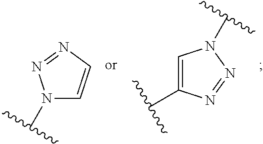 [SMILES: *n1ccnn1 ; *c1cn(*)nn1]

and

### X₄ is

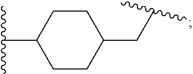

wherein
R¹³ is independently selected for each occasion from H and C₁-C₆ alkyl;
m is independently selected for each occasion from 1, 2, 3, 4, 5, 6, 7, 8, 9 and 10;
n is independently selected for each occasion from 1, 2, 3, 4, 5, 6, 7, 8, 9, 10, 11, 12, 13 and 14; and
wherein the single asterisk (*) indicates the attachment point to the cytotoxin (e.g., an amatoxin), and the double asterisk (**) indicates the attachment point to the reactive substituent Z′ or chemical moiety Z, with the proviso that L₁ and L₂ are not both absent.

In some embodiments, the linker includes a p-aminobenzyl group (PAB). In one embodiment, the p-aminobenzyl group is disposed between the cytotoxic drug and a protease cleavage site in the linker. In one embodiment, the p-aminobenzyl group is part of a p-aminobenzyloxycarbonyl unit. In one embodiment, the p-aminobenzyl group is part of a p-aminobenzylamido unit.

In some embodiments, the linker comprises PAB, Val-Cit-PAB, Val-Ala-PAB, Val-Lys(Ac)-PAB, Phe-Lys-PAB, Phe-Lys(Ac)-PAB, D-Val-Leu-Lys, Gly-Gly-Arg, Ala-Ala-Asn-PAB, or Ala-PAB.

In some embodiments, the linker comprises a combination of one or more of a peptide, oligosaccharide, —(CH₂)ₙ—, —(CH₂CH₂O)ₙ—, PAB, Val-Cit-PAB, Val-Ala-PAB, Val-Lys(Ac)-PAB, Phe-Lys-PAB, Phe-Lys(Ac)-PAB, D-Val-Leu-Lys, Gly-Gly-Arg, Ala-Ala-Asn-PAB, or Ala-PAB.

In some embodiments, the linker comprises a —(C═O)(CH₂)ₙ— unit, wherein n is an integer from 1-6.

In some embodiments, the linker comprises a —(CH₂)ₙ— unit, wherein n is an integer from 2 to 6.

In one specific embodiment, the linker comprises the structure:

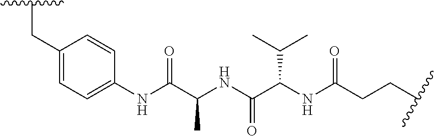 [SMILES: *CCC(=O)N[C@H](C(=O)N[C@@H](C)C(=O)Nc1ccc(C*)cc1)C(C)C]

wherein the wavy lines indicate attachment points to the cytotoxin and the reactive moiety Z′. In another specific embodiment, the linker comprises the structure:

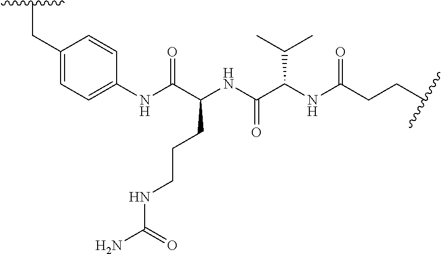 [SMILES: *CCC(=O)N[C@H](C(=O)N[C@@H](CCCNC(N)=O)C(=O)Nc1ccc(C*)cc1)C(C)C]

wherein the wavy lines indicate attachment points to the cytotoxin and the reactive moiety Z′. Such PAB-dipeptide-propionyl linkers are disclosed in, e.g., Patent Application Publication No. WO2017/149077, which is incorporated by reference herein in its entirety. Further, the cytotoxins disclosed in WO2017/149077 are incorporated by reference herein.

In certain embodiments, the linker of the ADC is maleimidocaproyl-Val-Ala-para-aminobenzyl (mc-Val-Ala-PAB).

In certain embodiments, the linker of the ADC is maleimidocaproyl-Val-Cit-para-aminobenzyl (mc-vc-PAB).

In some embodiments, the linker comprises

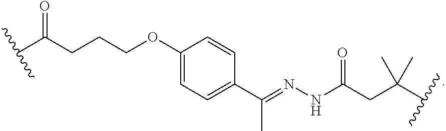 [SMILES: *C(=O)CCCOc1ccc(/C(C)=N/NC(=O)CC(*)(C)C)cc1]

In some embodiments, the linker comprises MCC (4-[N-maleimidomethyl]cyclohexane-1-carboxylate).

Linkers that can be used to conjugate an antibody, or antigen-binding fragment thereof, to a cytotoxic agent include those that are covalently bound to the cytotoxic agent on one end of the linker and, on the other end of the linker, contain a chemical moiety formed from a coupling reaction between a reactive substituent present on the linker and a reactive substituent present within the antibody, or antigen-binding fragment thereof, that binds CD117 (such as GNNK+ CD117). Reactive substituents that may be present within an antibody, or antigen-binding fragment thereof, that binds CD117 (such as GNNK+ CD117) include, without limitation, hydroxyl moieties of serine, threonine, and tyrosine residues; amino moieties of lysine residues; carboxyl moieties of aspartic acid and glutamic acid residues; and thiol moieties of cysteine residues, as well as propargyl, azido, haloaryl (e.g., fluoroaryl), haloheteroaryl (e.g., fluoroheteroaryl), haloalkyl, and haloheteroalkyl moieties of non-naturally occurring amino acids.

Examples of linkers useful for the synthesis of drug-antibody conjugates conjugates include those that contain electrophiles, such as Michael acceptors (e.g., maleimides), activated esters, electron-deficient carbonyl compounds, and aldehydes, among others, suitable for reaction with nucleophilic substituents present within antibodies or antigen-binding fragments, such as amine and thiol moieties. For instance, linkers suitable for the synthesis of drug-antibody conjugates include, without limitation, succinimidyl 4-(N-maleimidomethyl)-cyclohexane-L-carboxylate (SMCC), N-succinimidyl iodoacetate (SIA), sulfo-SMCC, m-maleimidobenzoyl-N-hydroxysuccinimidyl ester (MBS), sulfo-MBS, and succinimidyl iodoacetate, among others described, for instance, Liu et al., 18:690-697, 1979, the disclosure of which is incorporated herein by reference as it pertains to linkers for chemical conjugation. Additional linkers include the non-cleavable maleimidocaproyl linkers, which are particularly useful for the conjugation of microtubule-disrupting agents such as auristatins, are described by Doronina et al., Bioconjugate Chem. 17:14-24, 2006, the disclosure of which is incorporated herein by reference as it pertains to linkers for chemical conjugation.

It will be recognized by one of skill in the art that any one or more of the chemical groups, moieties and features disclosed herein may be combined in multiple ways to form linkers useful for conjugation of the antibodies and cytotoxins as disclosed herein. Further linkers useful in conjunction with the compositions and methods described herein, are described, for example, in U.S. Patent Application Publication No. 2015/0218220, the disclosure of which is incorporated herein by reference in its entirety.

Linkers useful in conjunction with the antibody-drug described herein include, without limitation, linkers containing chemical moieties formed by coupling reactions as depicted in Table 1, below. Curved lines designate points of attachment to the antibody, or antigen-binding fragment, and the cytotoxic molecule, respectively.

TABLE 1
Exemplary chemical moieties Z formed by
coupling reactions in the formation of antibody-drug

| Exemplary | |
|---|---|
| Coupling | |
| Reactions | Chemical Moiety Z Formed by Coupling Reactions |
| [3+2] Cycloaddition | 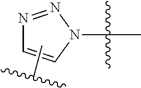 |
| [3+2] Cycloaddition | 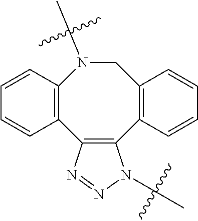 [SMILES: *N1Cc2ccccc2-c2c(nnn2*)-c2ccccc21] |
| [3+2] Cycloaddition, Esterification | 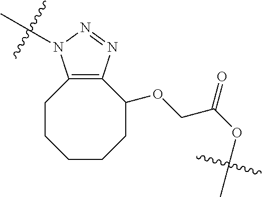 [SMILES: *OC(=O)COC1CCCCCc2c1nnn2*] |
| [3+2] Cycloaddition, Esterification | 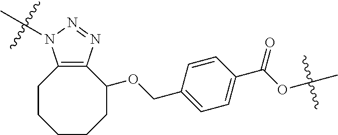 [SMILES: *OC(=O)c1ccc(COC2CCCCCc3c2nnn3*)cc1] |
| [3+2] Cycloaddition, Esterification | 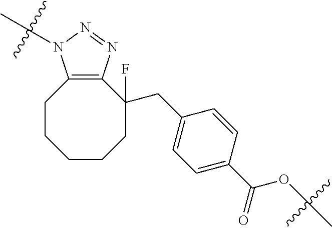 [SMILES: *OC(=O)c1ccc(CC2(F)CCCCCc3c2nnn3*)cc1] |
| [3+2] Cycloaddition, Esterification | 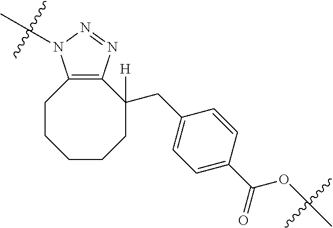 [SMILES: *OC(=O)c1ccc(CC2CCCCCc3c2nnn3*)cc1] |
| [3+2] Cycloaddition, Esterification | 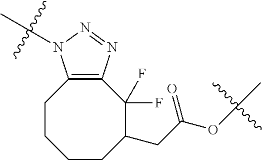 [SMILES: *OC(=O)CC1CCCCc2c(nnn2*)C1(F)F] |
| [3+2] Cycloaddition, Esterification | 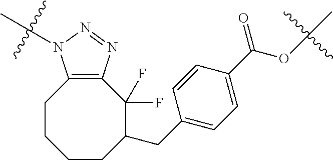 [SMILES: *OC(=O)c1ccc(CC2CCCCc3c(nnn3*)C2(F)F)cc1] |
| [3+2] Cycloaddition, Esterification | 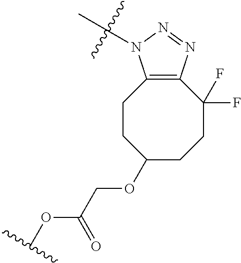 [SMILES: *OC(=O)COC1CCc2c(nnn2*)C(F)(F)CC1] |
| [3+2] Cycloaddition, Esterification | 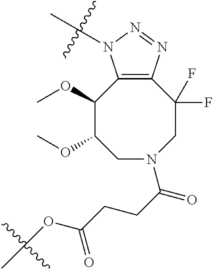 [SMILES: *OC(=O)CCC(=O)N1C[C@H](OC)[C@@H](OC)c2c(nnn2*)C(F)(F)C1] |
| [3+2] Cycloaddition, Esterification | 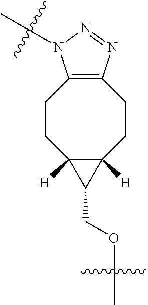 [SMILES: *OC[C@@H]1[C@@H]2CCc3c(nnn3*)CC[C@@H]21] |
| [3+2] Cycloaddition, Esterification | 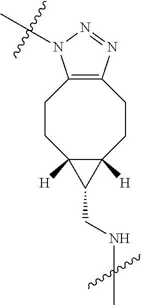 [SMILES: *NC[C@@H]1[C@@H]2CCc3c(nnn3*)CC[C@@H]21] |
| [3+2] Cycloaddition, Esterification | 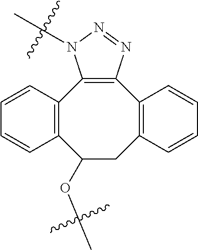 [SMILES: *OC1Cc2ccccc2-c2nnn(*)c2-c2ccccc21] |
| [3+2] Cycloaddition, Etherification | 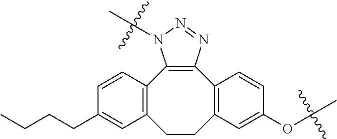 [SMILES: *Oc1ccc2c(c1)CCc1cc(CCCC)ccc1-c1c-2nnn1*] |
| [3+2] Cycloaddition | 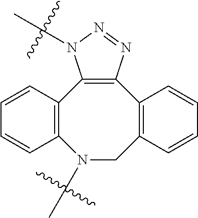 [SMILES: *N1Cc2ccccc2-c2nnn(*)c2-c2ccccc21] |
| Michael addition | 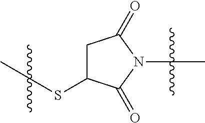 [SMILES: *SC1CC(=O)N(*)C1=O] |
| Michael addition | 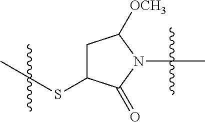 [SMILES: *SC1CC(OC)N(*)C1=O] |
| Imine condensation, Amidation | 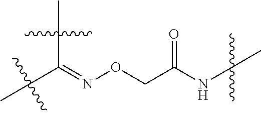 [SMILES: *NC(=O)CON=C(*)*] |
| Imine condensation | 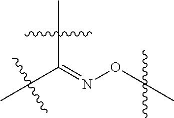 [SMILES: *ON=C(*)*] |
| Disulfide formation | 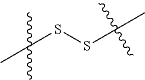 [SMILES: *SS*] |
| Thiol alkylation | 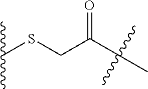 [SMILES: *SCC(*)=O] |
| Condensation, Michael addition | 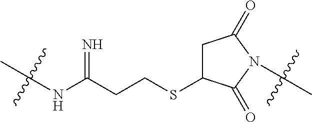 [SMILES: *NC(=N)CCSC1CC(=O)N(*)C1=O] |

One of skill in the art will recognize that a reactive substituent Z′ attached to the linker and a reactive substituent on the antibody or antigen-binding fragment thereof, are engaged in the covalent coupling reaction to produce the chemical moiety Z, and will recognize the reactive substituent Z′. Therefore, antibody-drug conjugates useful in conjunction with the methods described herein may be formed by the reaction of an antibody, or antigen-binding fragment thereof, with a linker or cytotoxin-linker conjugate, as described herein, the linker or cytotoxin-linker conjugate including a reactive substituent Z′, suitable for reaction with a reactive substituent on the antibody, or antigen-binding fragment thereof, to form the chemical moiety Z.

In some embodiments, Z′ is —NR¹³C(═O)CH═CH₂, —N₃, —SH, —S(═O)₂(CH═CH₂), —(CH₂)₂S(═O)₂(CH═CH₂), —NR¹³S(═O)₂(CH═CH₂), —NR¹³C(═O)CH₂R¹⁴, —NR¹³C(═O)CH₂Br, —NR¹³C(═O)CH₂₁, —NHC(═O)CH₂Br, —NHC(═O)CH₂₁, —ONH₂, —C(O)NHNH₂, —CO₂H, —NH₂, —NH(C═O), —NC(═S),

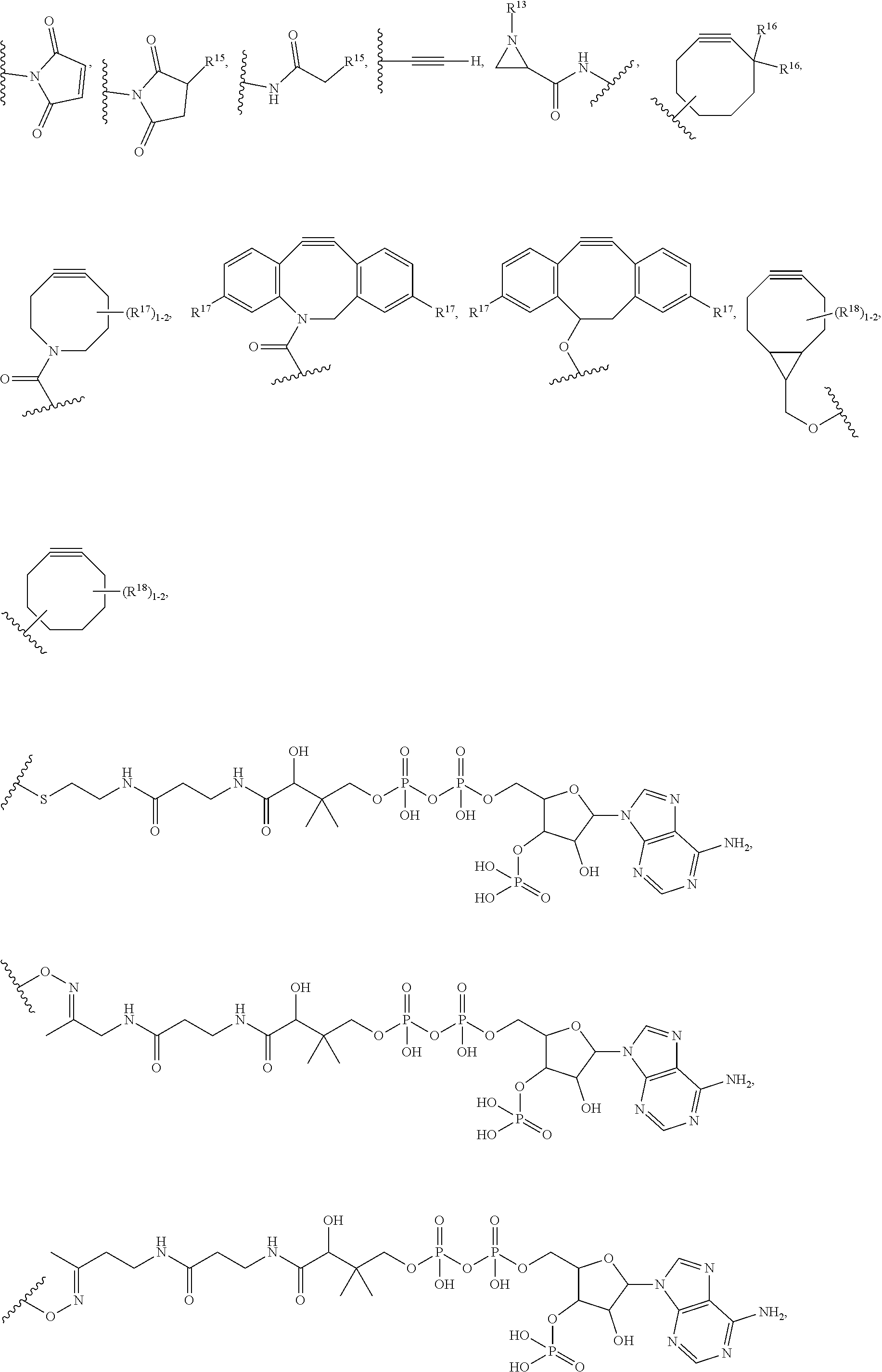

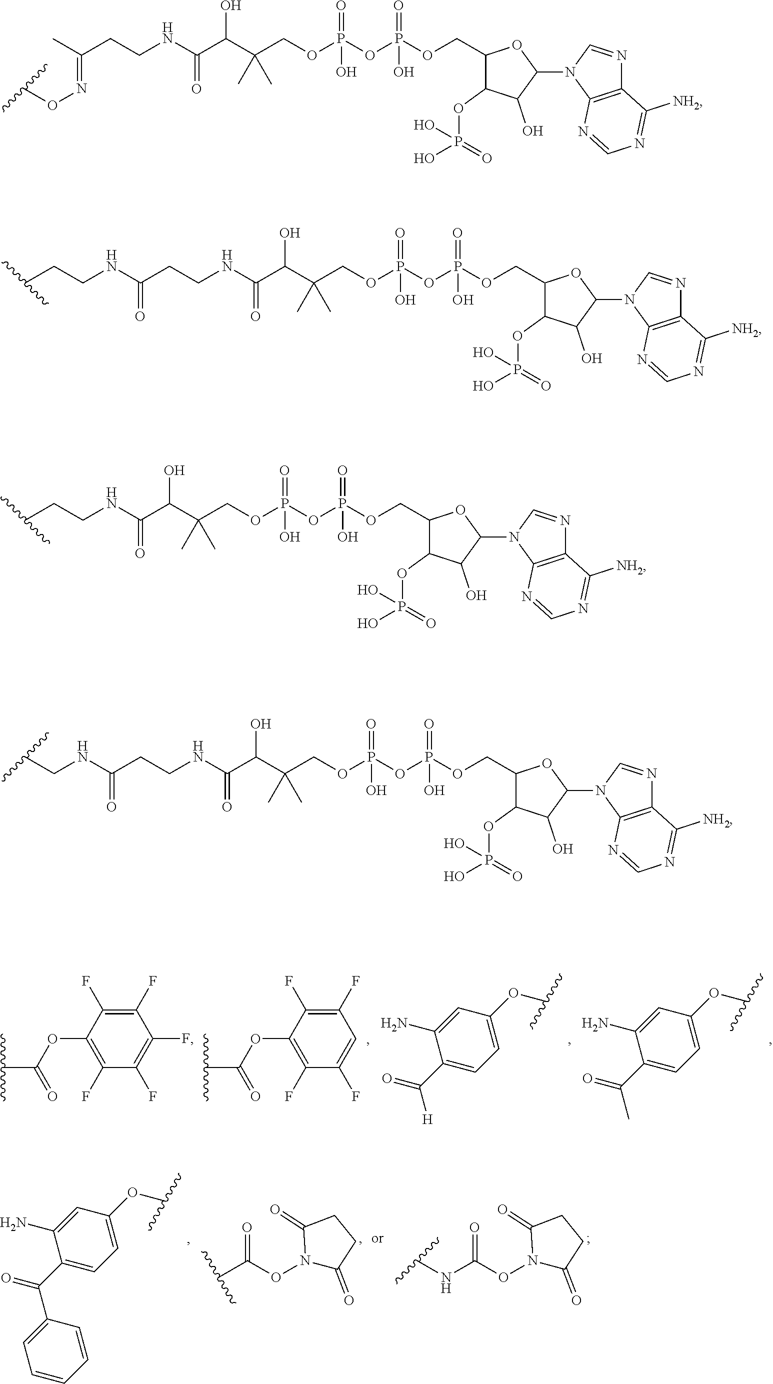

wherein

R¹³ is independently selected for each occasion from H and C₁-C₆ alkyl;

R₁₄ is —S(CH₂)ₙCHR¹⁵NHC(═O)R¹³;

R¹⁵ is R¹³ or —C(═O)OR¹³;

R¹⁶ is independently selected for each occasion from H, C₁-C₆ alkyl, F, Cl, and —OH;

R¹⁷ is independently selected for each occasion from H, C₁-C₆ alkyl, F, Cl, —NH₂, —OCH₃, —OCH₂CH₃, —N(CH₃)₂, —CN, —NO₂ and —OH; and

R¹⁸ is independently selected for each occasion from H, C₁-C₆ alkyl, F, benzyloxy substituted with —C(═O)OH, benzyl substituted with —C(═O)OH, C₁-C₄ alkoxy substituted with —C(═O)OH, and C₁-C₄ alkyl substituted with —C(═O)OH.

As depicted in Table 1, examples of suitably reactive substituents on the linker and antibody or antigen-binding fragment thereof include a nucleophile/electrophile pair (e.g., a thiol/haloalkyl pair, an amine/carbonyl pair, or a thiol/α,β-unsaturated carbonyl pair, and the like), a diene/dienophile pair (e.g., an azide/alkyne pair, or a diene/α,β-unsaturated carbonyl pair, among others), and the like. Coupling reactions between the reactive substitutents to form the chemical moiety Z include, without limitation, thiol alkylation, hydroxyl alkylation, amine alkylation, amine or hydroxylamine condensation, hydrazine formation, amidation, esterification, disulfide formation, cycloaddition (e.g., [4+2] Diels-Alder cycloaddition, [3+2] Huisgen cycloaddition, among others), nucleophilic aromatic substitution, electrophilic aromatic substitution, and other reactive modalities known in the art or described herein. Preferably, the linker contains an electrophilic functional group for reaction with a nucleophilic functional group on the antibody, or antigen-binding fragment thereof.

Reactive substituents that may be present within an antibody, or antigen-binding fragment thereof, as disclosed herein include, without limitation, nucleophilic groups such as (i)N-terminal amine groups, (ii) side chain amine groups, e.g. lysine, (iii) side chain thiol groups, e.g. cysteine, and (iv) sugar hydroxyl or amino groups where the antibody is glycosylated. Reactive substituents that may be present within an antibody, or antigen-binding fragment thereof, as disclosed herein include, without limitation, hydroxyl moieties of serine, threonine, and tyrosine residues; amino moieties of lysine residues; carboxyl moieties of aspartic acid and glutamic acid residues; and thiol moieties of cysteine residues, as well as propargyl, azido, haloaryl (e.g., fluoroaryl), haloheteroaryl (e.g., fluoroheteroaryl), haloalkyl, and haloheteroalkyl moieties of non-naturally occurring amino acids. In some embodiments, the reactive substituents present within an antibody, or antigen-binding fragment thereof as disclosed herein include, are amine or thiol moieties. Certain antibodies have reducible interchain disulfides, i.e. cysteine bridges. Antibodies may be made reactive for conjugation with linker reagents by treatment with a reducing agent such as DTT (dithiothreitol). Each cysteine bridge will thus form, theoretically, two reactive thiol nucleophiles. Additional nucleophilic groups can be introduced into antibodies through the reaction of lysines with 2-iminothiolane (Traut's reagent) resulting in conversion of an amine into a thiol. Reactive thiol groups may be introduced into the antibody (or fragment thereof) by introducing one, two, three, four, or more cysteine residues (e.g., preparing mutant antibodies comprising one or more non-native cysteine amino acid residues). U.S. Pat. No. 7,521,541 teaches engineering antibodies by introduction of reactive cysteine amino acids.

In some embodiments, the reactive moiety Z′ attached to the linker is a nucleophilic group which is reactive with an electrophilic group present on an antibody. Useful electrophilic groups on an antibody include, but are not limited to, aldehyde and ketone carbonyl groups. The heteroatom of a nucleophilic group can react with an electrophilic group on an antibody and form a covalent bond to the antibody. Useful nucleophilic groups include, but are not limited to, hydrazide, oxime, amino, hydroxyl, hydrazine, thiosemicarbazone, hydrazine carboxylate, and arylhydrazide.

In some embodiments, Z is the product of a reaction between reactive nucleophilic substituents present within the antibodies, or antigen-binding fragments thereof, such as amine and thiol moieties, and a reactive electrophilic substituent Z′. For instance, Z′ may be a Michael acceptor (e.g., maleimide), activated ester, electron-deficient carbonyl compound, or an aldehyde, among others. In some embodiments, the reactive substituent Z′ attached to linker L is a maleimide, azide, or alkyne. An example of a maleimide-containing linker is the non-cleavable maleimidocaproyl-based linker, which is particularly useful for the conjugation of microtubule-disrupting agents such as auristatins. In some embodiments, the reactive substituent Z′ is —(C═O)— or —NH(C═O)—, such that the linker may be joined to the antibody, or antigen-binding fragment thereof, by an amide or urea moiety, respectively, resulting from reaction of the —(C═O)— or —NH(C═O)— group with an amino group of the antibody or antigen-binding fragment thereof.

In some embodiments, the ADC comprises an anti-CD117 antibody conjugated to an amatoxin of any of formulae III, IIIA, IIIB, or IIIC as disclosed herein, forming a linker-amatoxin or antibody drug conjugate of any of formulae I, IA, IB, II, IIA, or IIB as disclosed herein, via a linker and a chemical moiety Z, as disclosed herein. In some embodiments, the linker includes a hydrazine, a disulfide, a thioether or a dipeptide. In some embodiments, the linker includes a dipeptide selected from Val-Ala and Val-Cit. In some embodiments, the linker includes a para-aminobenzyl group (PAB). In some embodiments, the linker includes the moiety PAB-Cit-Val. In some embodiments, the linker includes the moiety PAB-Ala-Val. In some embodiments, the linker includes a —((C═O)(CH₂)ₙ— unit, wherein n is an integer from 1-6. In some embodiments, the linker is-PAB-Cit-Val-((C═O)(CH₂)ₙ—.

In some embodiments, the linker includes a —(CH₂)ₙ— unit, where n is an integer from 2-6. In some embodiments, the linker is-PAB-Cit-Val-((C═O)(CH₂)ₙ—. In some embodiments, the linker is —PAB-Ala-Val-((C═O)(CH₂)ₙ—. In some embodiments, the linker is —(CH₂)ₙ—. In some embodiments, the linker is —((CH₂)ₙ—, wherein n is 6.

In some embodiments, the chemical moiety Z is selected from Table 1. In some embodiments, the chemical moiety Z is

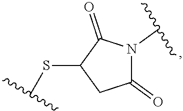

where S is a sulfur atom which represents the reactive substituent present within an antibody, or antigen-binding fragment thereof, that binds CD117 (e.g., from the —SH group of a cysteine residue).

In some embodiments, the linker L and the chemical moiety Z, taken together as L-Z, is

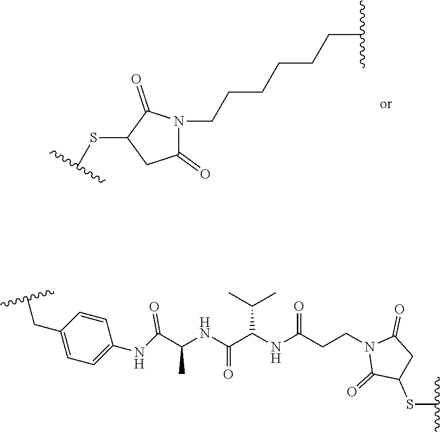

One of skill in the art will recognize the linker-reactive substituent group structure, prior to conjugation with the antibody or antigen binding fragment thereof, includes a maleimide as the group Z′.

In some embodiments, the linker-reactive substituent group structure L-Z′, prior to conjugation with the antibody or antigen binding fragment thereof, is:

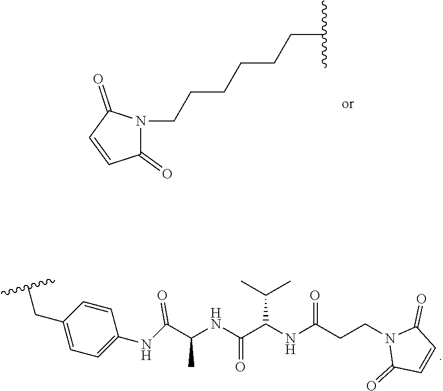

In some embodiments, an amatoxin as disclosed herein is conjugated to a linker-reactive moiety -L-Z′ having the following formula:

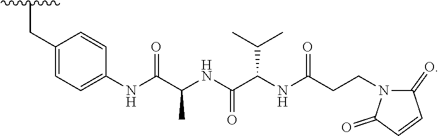 [SMILES: *Cc1ccc(NC(=O)[C@H](C)NC(=O)[C@@H](NC(=O)CCN2C(=O)C=CC2=O)C(C)C)cc1]

In some embodiments, an amatoxin as disclosed herein is conjugated to a linker-reactive moiety -L-Z′ having the following formula:

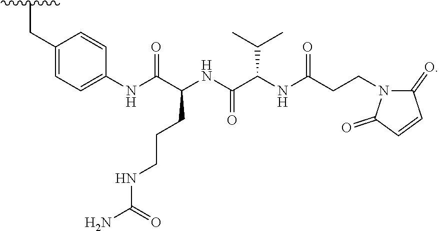 [SMILES: *Cc1ccc(NC(=O)[C@H](CCCNC(N)=O)NC(=O)[C@@H](NC(=O)CCN2C(=O)C=CC2=O)C(C)C)cc1]

The foregoing linker moieties and amatoxin-linker conjugates, among others useful in conjunction with the compositions and methods described herein, are described, for example, in U.S. Patent Application Publication No. 2015/0218220 and Patent Application Publication No. WO2017/149077, the disclosure of each of which is incorporated herein by reference in its entirety.

### Preparation of Antibody-Drug Conjugates

In the ADCs of formula I as disclosed herein, an anti-CD117 antibody or antigen binding fragment thereof is conjugated to one or more cytotoxic drug moieties (D), e.g. about 1 to about 20 drug moieties per antibody, through a linker L and a chemical moiety Z as disclosed herein. The ADCs of the present disclosure may be prepared by several routes, employing organic chemistry reactions, conditions, and reagents known to those skilled in the art, including: (1) reaction of a reactive substituent of an antibody or antigen binding fragment thereof with a bivalent linker reagent to form Ab-Z-L as described herein above, followed by reaction with a drug moiety D; or (2) reaction of a reactive substituent of a drug moiety with a bivalent linker reagent to form D-L-Z, followed by reaction with a reactive substituent of an antibody or antigen binding fragment thereof as described herein above to form an ADC of formula D-L-Z-Ab, such as Am-Z-L-Ab. Additional methods for preparing ADC are described herein.

In another aspect, the anti-CD117 antibody or antigen binding fragment thereof has one or more lysine residues that can be chemically modified to introduce one or more sulfhydryl groups. The ADC is then formed by conjugation through the sulfhydryl group's sulfur atom as described herein above. The reagents that can be used to modify lysine include, but are not limited to, N-succinimidyl S-acetylthioacetate (SATA) and 2-Iminothiolane hydrochloride (Traut's Reagent).

In another aspect, the anti-CD117 antibody or antigen binding fragment thereof can have one or more carbohydrate groups that can be chemically modified to have one or more sulfhydryl groups. The ADC is then formed by conjugation through the sulfhydryl group's sulfur atom as described herein above.

In yet another aspect, the anti-CD117 antibody can have one or more carbohydrate groups that can be oxidized to provide an aldehyde (—CHO) group (see, for e.g., Laguzza, et al., J. Med. Chem. 1989, 32(3), 548-55). The ADC is then formed by conjugation through the corresponding aldehyde as described herein above. Other protocols for the modification of proteins for the attachment or association of cytotoxins are described in Coligan et al., Current Protocols in Protein Science, vol. 2, John Wiley & Sons (2002), incorporated herein by reference.

Methods for the conjugation of linker-drug moieties to cell-targeted proteins such as antibodies, immunoglobulins or fragments thereof are found, for example, in U.S. Pat. Nos. 5,208,020; 6,441,163; WO2005037992; WO2005081711; and WO2006/034488, all of which are hereby expressly incorporated by reference in their entirety.

Alternatively, a fusion protein comprising the anti-CD117 antibody and cytotoxic agent may be made, e.g., by recombinant techniques or peptide synthesis. The length of DNA may comprise respective regions encoding the two portions of the conjugate either adjacent one another or separated by a region encoding a linker peptide which does not destroy the desired properties of the conjugate.

### Methods of Treatment

As described herein, hematopoietic stem cell transplant therapy can be administered to a subject in need of treatment so as to populate or re-populate one or more blood cell types. Hematopoietic stem cells generally exhibit multi-potency, and can thus differentiate into multiple different blood lineages including, but not limited to, granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), monocytes (e.g., monocytes, macrophages), dendritic cells, microglia, osteoclasts, and lymphocytes (e.g., NK cells, B-cells and T-cells). Hematopoietic stem cells are additionally capable of self-renewal, and can thus give rise to daughter cells that have equivalent potential as the mother cell, and also feature the capacity to be reintroduced into a transplant recipient whereupon they home to the hematopoietic stem cell niche and re-establish productive and sustained hematopoiesis.

Hematopoietic stem cells can thus be administered to a patient defective or deficient in one or more cell types of the hematopoietic lineage in order to re-constitute the defective or deficient population of cells in vivo, thereby treating the pathology associated with the defect or depletion in the endogenous blood cell population. The compositions and methods described herein can thus be used to treat a non-malignant hemoglobinopathy (e.g., a hemoglobinopathy selected from the group consisting of sickle cell anemia, thalassemia, Fanconi anemia, aplastic anemia, and Wiskott-Aldrich syndrome). Additionally or alternatively, the compositions and methods described herein can be used to treat an immunodeficiency, such as a congenital immunodeficiency. Additionally or alternatively, the compositions and methods described herein can be used to treat an acquired immunodeficiency (e.g., an acquired immunodeficiency selected from the group consisting of HIV and AIDS). The compositions and methods described herein can be used to treat a metabolic disorder (e.g., a metabolic disorder selected from the group consisting of glycogen storage diseases, mucopolysaccharidoses, Gaucher's Disease, Hurlers Disease, sphingolipidoses, and metachromatic leukodystrophy).

Additionally or alternatively, the compositions and methods described herein can be used to treat a malignancy or proliferative disorder, such as a hematologic cancer, myeloproliferative disease. In the case of cancer treatment, the compositions and methods described herein may be administered to a patient so as to deplete a population of endogenous hematopoietic stem cells prior to hematopoietic stem cell transplantation therapy, in which case the transplanted cells can home to a niche created by the endogenous cell depletion step and establish productive hematopoiesis. This, in turn, can re-constitute a population of cells depleted during cancer cell eradication, such as during systemic chemotherapy. Exemplary hematological cancers that can be treated using the compositions and methods described herein include, without limitation, acute myeloid leukemia, acute lymphoid leukemia, chronic myeloid leukemia, chronic lymphoid leukemia, multiple myeloma, diffuse large B-cell lymphoma, and non-Hodgkin's lymphoma, as well as other cancerous conditions, including neuroblastoma.

Additional diseases that can be treated with the compositions and methods described herein include, without limitation, adenosine deaminase deficiency and severe combined immunodeficiency, hyper immunoglobulin M syndrome, Chediak-Higashi disease, hereditary lymphohistiocytosis, osteopetrosis, osteogenesis imperfecta, storage diseases, thalassemia major, systemic sclerosis, systemic lupus erythematosus, multiple sclerosis, and juvenile rheumatoid arthritis.

The anti-CD117 antibodies, antigen-binding fragments thereof, and conjugates (i.e., ADCs) described herein may be used to induce solid organ transplant tolerance. For instance, the compositions and methods described herein may be used to deplete or ablate a population of cells from a target tissue (e.g., to deplete hematopoietic stem cells from the bone marrow stem cell niche). Following such depletion of cells from the target tissues, a population of stem or progenitor cells from an organ donor (e.g., hematopoietic stem cells from the organ donor) may be administered to the transplant recipient, and following the engraftment of such stem or progenitor cells, a temporary or stable mixed chimerism may be achieved, thereby enabling long-term transplant organ tolerance without the need for further immunosuppressive agents. For example, the compositions and methods described herein may be used to induce transplant tolerance in a solid organ transplant recipient (e.g., a kidney transplant, lung transplant, liver transplant, and heart transplant, among others). The compositions and methods described herein are well-suited for use in connection the induction of solid organ transplant tolerance, for instance, because a low percentage temporary or stable donor engraftment is sufficient to induce long-term tolerance of the transplanted organ.

In addition, the compositions and methods described herein can be used to treat cancers directly, such as cancers characterized by cells that are CD117+. For instance, the compositions and methods described herein can be used to treat leukemia, particularly in patients that exhibit CD117+ leukemic cells. By depleting CD117+ cancerous cells, such as leukemic cells, the compositions and methods described herein can be used to treat various cancers directly. Exemplary cancers that may be treated in this fashion include hematological cancers, such as acute myeloid leukemia, acute lymphoid leukemia, chronic myeloid leukemia, chronic lymphoid leukemia, multiple myeloma, diffuse large B-cell lymphoma, and non-Hodgkin's lymphoma.

Acute myeloid leukemia (AML) is a cancer of the myeloid line of blood cells, characterized by the rapid growth of abnormal white blood cells that build up in the bone marrow and interfere with the production of normal blood cells. AML is the most common acute leukemia affecting adults, and its incidence increases with age. The symptoms of AML are caused by replacement of normal bone marrow with leukemic cells, which causes a drop in red blood cells, platelets, and normal white blood cells. As an acute leukemia, AML progresses rapidly and may be fatal within weeks or months if left untreated. In one embodiment, the anti-CD117 ADCs described herein are used to treat AML in a human patient in need thereof. In certain embodiments the anti-CD117 ADC treatment depletes AML cells in the treated subjects. In some embodiments about 50% or more of the AML cells are depleted. In other embodiments, about 60% or more of the AML cells are depleted, or about 70% or more of the AML cells are depleted, or about 80% of more or about 90% or more, or about 95% or more of the AML cells are depleted. In certain embodiments the anti-CD117 ADC treatments is a single dose treatment. In certain embodiments the single dose anti-CD117 ADC treatment depletes about 60%, about 70%, about 80%, about 90%, about 95% or more of the AML cells.

In addition, the compositions and methods described herein can be used to treat autoimmune disorders. For instance, an anti-CD117 antibody, or antigen-binding fragment thereof, can be administered to a subject, such as a human patient suffering from an autoimmune disorder, so as to kill a CD117+ immune cell. The CD117+ immune cell may be an autoreactive lymphocyte, such as a T-cell that expresses a T-cell receptor that specifically binds, and mounts an immune response against, a self antigen. By depleting self-reactive, CD117+ cells, the compositions and methods described herein can be used to treat autoimmune pathologies, such as those described below. Additionally or alternatively, the compositions and methods described herein can be used to treat an autoimmune disease by depleting a population of endogenous hematopoietic stem cells prior to hematopoietic stem cell transplantation therapy, in which case the transplanted cells can home to a niche created by the endogenous cell depletion step and establish productive hematopoiesis. This, in turn, can re-constitute a population of cells depleted during autoimmune cell eradication.

Autoimmune diseases that can be treated using the compositions and methods described herein include, without limitation, psoriasis, psoriatic arthritis, Type 1 diabetes mellitus (Type 1 diabetes), rheumatoid arthritis (RA), human systemic lupus (SLE), multiple sclerosis (MS), inflammatory bowel disease (IBD), lymphocytic colitis, acute disseminated encephalomyelitis (ADEM), Addison's disease, alopecia universalis, ankylosing spondylitisis, antiphospholipid antibody syndrome (APS), aplastic anemia, autoimmune hemolytic anemia, autoimmune hepatitis, autoimmune inner ear disease (AIED), autoimmune lymphoproliferative syndrome (ALPS), autoimmune oophoritis, Balo disease, Behcet's disease, bullous pemphigoid, cardiomyopathy, Chagas' disease, chronic fatigue immune dysfunction syndrome (CFIDS), chronic inflammatory demyelinating polyneuropathy, Crohn's disease, cicatrical pemphigoid, coeliac sprue-dermatitis herpetiformis, cold agglutinin disease, CREST syndrome, Degos disease, discoid lupus, dysautonomia, endometriosis, essential mixed cryoglobulinemia, fibromyalgia-fibromyositis, Goodpasture's syndrome, Grave's disease, Guillain-Barre syndrome (GBS), Hashimoto's thyroiditis, Hidradenitis suppurativa, idiopathic and/or acute thrombocytopenic purpura, idiopathic pulmonary fibrosis, IgA neuropathy, interstitial cystitis, juvenile arthritis, Kawasaki's disease, lichen planus, Lyme disease, Meniere disease, mixed connective tissue disease (MCTD), myasthenia gravis, neuromyotonia, opsoclonus myoclonus syndrome (OMS), optic neuritis, Ord's thyroiditis, pemphigus vulgaris, pernicious anemia, polychondritis, polymyositis and dermatomyositis, primary biliary cirrhosis, polyarteritis nodosa, polyglandular syndromes, polymyalgia rheumatica, primary agammaglobulinemia, Raynaud phenomenon, Reiter's syndrome, rheumatic fever, sarcoidosis, scleroderma, Sjögren's syndrome, stiff person syndrome, Takayasu's arteritis, temporal arteritis (also known as “giant cell arteritis”), ulcerative colitis, collagenous colitis, uveitis, vasculitis, vitiligo, vulvodynia (“vulvar vestibulitis”), and Wegener's granulomatosis.

### Routes of Administration and Dosing

ADCs, antibodies, or antigen-binding fragments thereof, or described herein can be administered to a patient (e.g., a human patient suffering from cancer, an autoimmune disease, or in need of hematopoietic stem cell transplant therapy) in a variety of dosage forms. For instance, antibodies, or antigen-binding fragments thereof, described herein can be administered to a patient suffering from cancer, an autoimmune disease, or in need of hematopoietic stem cell transplant therapy in the form of an aqueous solution, such as an aqueous solution containing one or more pharmaceutically acceptable excipients. Pharmaceutically acceptable excipients for use with the compositions and methods described herein include viscosity-modifying agents. The aqueous solution may be sterilized using techniques known in the art.

Pharmaceutical formulations comprising anti-CD117 ADCs or antibodies as described herein are prepared by mixing such ADC or anti-CD117 antibody with one or more optional pharmaceutically acceptable carriers (Remington's Pharmaceutical Sciences 16th edition, Osol, A. Ed. (1980)), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

The anti-CD117 ADCs, antibodies, or antigen-binding fragments, described herein may be administered by a variety of routes, such as orally, transdermally, subcutaneously, intranasally, intravenously, intramuscularly, intraocularly, or parenterally. The most suitable route for administration in any given case will depend on the particular antibody, or antigen-binding fragment, administered, the patient, pharmaceutical formulation methods, administration methods (e.g., administration time and administration route), the patient's age, body weight, sex, severity of the diseases being treated, the patient's diet, and the patient's excretion rate.

The effective dose of an anti-CD117 ADC, antibody, or antigen-binding fragment thereof, described herein can range, for example from about 0.001 to about 100 mg/kg of body weight per single (e.g., bolus) administration, multiple administrations, or continuous administration, or to achieve an optimal serum concentration (e.g., a serum concentration of 0.0001-5000 μg/mL) of the antibody, antigen-binding fragment thereof. A dose of the anti-CD117 ADC may be administered one or more times (e.g., 2-10 times) per day, week, or month to a subject (e.g., a human) suffering from cancer, an autoimmune disease, or undergoing conditioning therapy in preparation for receipt of a hematopoietic stem cell transplant. In the case of a conditioning procedure prior to hematopoietic stem cell transplantation, the anti-CD117 ADC, antibody, or antigen-binding fragment thereof, can be administered to the patient at a time that optimally promotes engraftment of the exogenous hematopoietic stem cells, for instance, from about 1 hour to about 1 week (e.g., about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 2 days, about 3 days, about 4 days, about 5 days, about 6 days, or about 7 days) or more prior to administration of the exogenous hematopoietic stem cell transplant.

Using the methods disclosed herein, a physician of skill in the art can administer to a human patient in need of hematopoietic stem cell transplant therapy an anti-CD117 ADC, an antibody or an antigen-binding fragment thereof capable of binding an antigen expressed by hematopoietic stem cells, such as an antibody or antigen-biding fragment thereof that binds CD117 (for example, an antibody or antigen-binding fragment thereof that binds GNNK+ CD117). In this fashion, a population of endogenous hematopoietic stem cells can be depleted prior to administration of an exogenous hematopoietic stem cell graft so as to promote engraftment of the hematopoietic stem cell graft.

As described above, the antibody may be covalently conjugated to a toxin, such as a cytotoxic molecule described herein or known in the art. For instance, an anti-CD117 antibody or antigen-binding fragment thereof (such as an anti-GNNK+ CD117 antibody or antigen-binding fragment thereof) can be covalently conjugated to a cytotoxin, such as Pseudomonas exotoxin A, deBouganin, diphtheria toxin, an amatoxin, such as γ-amanitin, α-amanitin, saporin, maytansine, a maytansinoid, an auristatin, an anthracycline, a calicheamicin, irinotecan, SN-38, a duocarmycin, a shiga toxin, a pyrrolobenzodiazepine, a pyrrolobenzodiazepine dimer, an indolinobenzodiazepine, an indolinobenzodiazepine dimer, or a variant thereof. This conjugation can be performed using covalent bond-forming techniques described herein or known in the art. The antibody, antigen-binding fragment thereof, or drug-antibody conjugate can subsequently be administered to the patient, for example, by intravenous administration, prior to transplantation of exogenous hematopoietic stem cells (such as autologous, syngeneic, or allogeneic hematopoietic stem cells) to the patient.

The anti-CD117 (e.g., anti-GNNK+ CD117) antibody, antigen-binding fragment thereof, or drug-antibody conjugate can be administered in an amount sufficient to reduce the quantity of endogenous hematopoietic stem cells, for example, by about 10%, about 20%, about 30%, about 40%, about 50%, about 60%, about 70%, about 80%, about 90%, about 95%, or more prior to hematopoietic stem cell transplant therapy. The reduction in hematopoietic stem cell count can be monitored using conventional techniques known in the art, such as by FACS analysis of cells expressing characteristic hematopoietic stem cell surface antigens in a blood sample withdrawn from the patient at varying intervals during conditioning therapy. For instance, a physician of skill in the art can withdraw a blood sample from the patient at various time points during conditioning therapy and determine the extent of endogenous hematopoietic stem cell reduction by conducting a FACS analysis to elucidate the relative concentrations of hematopoietic stem cells in the sample using antibodies that bind to hematopoietic stem cell marker antigens. According to some embodiments, when the concentration of hematopoietic stem cells has reached a minimum value in response to conditioning therapy with an anti-CD117 (e.g., anti-GNNK+ CD117) antibody, antigen-binding fragment thereof, or drug-antibody conjugate, the physician may conclude the conditioning therapy, and may begin preparing the patient for hematopoietic stem cell transplant therapy.

The anti-CD117 (e.g., anti-GNNK+ CD117) antibody, antigen-binding fragment thereof, or drug-antibody conjugate can be administered to the patient in an aqueous solution containing one or more pharmaceutically acceptable excipients, such as a viscosity-modifying agent. The aqueous solution may be sterilized using techniques described herein or known in the art. The antibody, antigen-binding fragment thereof, or drug-antibody conjugate can be administered to the patient at a dosage of, for example, from 0.001 mg/kg to 100 mg/kg prior to administration of a hematopoietic stem cell graft to the patient. The antibody, antigen-binding fragment thereof, or drug-antibody conjugate can be administered to the patient at a time that optimally promotes engraftment of the exogenous hematopoietic stem cells, for instance, from about 1 hour to about 1 week (e.g., about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 2 days, about 3 days, about 4 days, about 5 days, about 6 days, or about 7 days) or more prior to administration of the exogenous hematopoietic stem cell transplant.

Following the conclusion of conditioning therapy, the patient may then receive an infusion (e.g., an intravenous infusion) of exogenous hematopoietic stem cells, such as from the same physician that performed the conditioning therapy or from a different physician. The physician may administer the patient an infusion of autologous, syngeneic, or allogeneic hematopoietic stem cells, for instance, at a dosage of from 1×10³ to 1×10⁹ hematopoietic stem cells/kg. The physician may monitor the engraftment of the hematopoietic stem cell transplant, for example, by withdrawing a blood sample from the patient and determining the increase in concentration of hematopoietic stem cells or cells of the hematopoietic lineage (such as megakaryocytes, thrombocytes, platelets, erythrocytes, mast cells, myeloblasts, basophils, neutrophils, eosinophils, microglia, granulocytes, monocytes, osteoclasts, antigen-presenting cells, macrophages, dendritic cells, natural killer cells, T-lymphocytes, and B-lymphocytes) following administration of the transplant. This analysis may be conducted, for example, from about 1 hour to about 6 months, or more, following hematopoietic stem cell transplant therapy (e.g., about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 2 days, about 3 days, about 4 days, about 5 days, about 6 days, about 7 days, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks, about 15 weeks, about 16 weeks, about 17 weeks, about 18 weeks, about 19 weeks, about 20 weeks, about 21 weeks, about 22 weeks, about 23 weeks, about 24 weeks, or more). A finding that the concentration of hematopoietic stem cells or cells of the hematopoietic lineage has increased (e.g., by about 1%, about 2%, about 3%, about 4%, about 5%, about 6%, about 7%, about 8%, about 9%, about 10%, about 20%, about 30%, about 40%, about 50%, about 60%, about 70%, about 80%, about 90%, about 100%, about 200%, about 500%, or more) following the transplant therapy relative to the concentration of the corresponding cell type prior to transplant therapy provides one indication that treatment with the anti-CD117 (e.g., anti-GNNK+ CD117) antibody, antigen-binding fragment thereof, or drug-antibody conjugate has successfully promoted engraftment of the transplanted hematopoietic stem cell graft.

### EXAMPLES

The following examples are put forth so as to provide those of ordinary skill in the art with a description of how the compositions and methods described herein may be used, made, and evaluated, and are intended to be purely exemplary of the present disclosure and are not intended to limit the scope of what the inventors regard as their invention.

### Example 1. Identification of Anti-CD117 Antibody 85 (Ab85)

The identification of anti-CD117 antibody 85 (i.e., Ab85) was created based on a derivative of the human CK6 antibody (i.e., Ab1) as described in PCT/US2018/057180 (the disclosure of which is incorporated herein by reference in its entireity). Antibody 85 is an antagonistic antibody, which can be determined by assaying the ability of Ab85 to block CD117-mediated activation as known to one of skill in the art, comprising the amino acid sequences provided below. Briefly, cells depedendent on the ligand of CD117, SCF1, for proliferation (primary CD34⁺ cells or Mo7e cells) can be incubated in the presence of CD117 binding antibodies to assess their capability to block interactions between CD117 and SCF1, thereby leading to measurable decreases in cellular proliferation. Alternatively, simple binding experiments can be performed to measure antagonism of CD117 and SCF1 binding events.

### Antibody HC-85/LC-85 (Ab 85).

The heavy chain variable region (VH) amino acid sequence of Ab85 is provided below as SEQ ID NO: 2. The VH CDR amino acid sequences of Ab85 underlined below and are as follows: NYWIG (VH CDR1; SEQ ID NO: 3); IINPRDSDTRYRPSFQG (VH CDR2; SEQ ID NO: 4); and HGRGYEGYEGAFDI (VH CDR3; SEQ ID NO: 5).

Ab85 VH sequence

(SEQ ID NO: 2)
EVQLVQSGAEVKKPGESLKISCKGSGYSFTNYWIGWVRQMPGK
GLEWMAIINPRDSDTRYRPSFQGQVTISADKSISTAYLQWSSL
KASDTAMYYCARHGRGYEGYEGAFDIWGQGTLVTVSS

The light chain variable region (VL) amino acid sequence of Ab85 is provided below as SEQ ID NO 6. The VL CDR amino acid sequences of Ab85 underlined below and are as follows:

(VL CDR1; SEQ ID NO: 7)
RSSQGIRSDLG;
(VL CDR2; SEQ ID NO: 8)
DASNLET;
and
(VL CDR3; SEQ ID NO: 9)
QQANGFPLT.

Ab85 VL sequence

(SEQ ID NO: 6)
DIQMTQSPSSLSASVGDRVTITCRSSQGIRSDLGVVYQ
QKPGKAPKLLIYDASNLETGVPSRFSGSGSGTDFTLTI
SSLQPEDFATYYCQQANGFPLTFGGGTKVEIK

A comparison of the amino acid sequences of the heavy and light chain variable regions and CDRs of Ab85 and Ab1 is described in FIGS. 1A and 1B.

### Example 2. Epitope Analysis of Ab85

Chemical Cross-Linking Experiments

Cross-linking experiments allow the direct analysis of non-covalent interaction by High-Mass MALDI mass spectrometry. By mixing a protein sample containing non-covalent interactions with a specially developed cross-linking mixture (Bich, C et al. Anal. Chem., 2010, 82 (1), pp 172-179), it is possible to specifically detect non-covalent complex with high-sensitivity. The covalent binding generated allows the interacting species to survive the sample preparation process and the MALDI ionization. A special High-Mass detection system allows characterizing the interaction in the High-Mass range.

A sample of Ab85 was diluted in distilled water to a concentration of 2.7 mg/mL. 1 μL of the diluted Ab85 was then mixed with 1 μL of a matrix composed of a re-crystallized sinapinic acid matrix (10 mg/mL) in acetonitrile/water (1:1, v/v), TFA (0.1% (K200 MALDI Kit). The mixture was submitted to cross-linking using CovalX's K200 MALDI MS analysis kit. The protein solutions were mixed with 1 μl of K200 Stabilizer reagent (2 mg/ml) and incubated at room temperature. After the incubation time (180 minutes) the samples were prepared for MALDI analysis. The samples were analyzed by High-Mass MALDI MS immediately after crystallization at room temperature.

The MALDI ToF MS analysis has been performed using CovalX's HM4 interaction module with a standard nitrogen laser and focusing on different mass ranges from 0 to 1500 kDa.

Peptide Mass Fingerprint Experiments

In order to characterize CD117, a sample of CD117 was subjected to trypsin, chymotrypsin, Asp-N, elastase and thermolysin proteolysis followed by nLC-LTQ-Orbitrap MS/MS analysis using a nLC Ultimate 3000-RSLC system in line with a LTQ-Orbitrap mass spectrometer (Thermo Scientific).

10 μL of CD117 (6.8 μM) were mixed with 1 μL of DSS d0/d12 (2 mg/mL; DMF) before 180 minutes incubation time at room temperature. After incubation, the reaction was stopped by adding 1 μL of Ammonium Bicarbonate (20 mM final concentration) before 1 h incubation time at room temperature. The solution was then dried using a speedvac before H₂O 8M urea suspension (10 μL). After mixing, 1 μl of DTT (500 mM) was added to the solution. The mixture was then incubated 1 hour at 37° C. After incubation, 1 μl of iodoacetamide (1M) was added before 1 hour incubation time at room temperature, in a dark room. After incubation, 100 μl of the proteolytic buffer were added. The trypsin buffer contains 50 mM Ambic pH 8.5, 5% acetonitrile, the chymotrypsin buffer contains Tris HCl 100 mM, CaCL2 10 mM pH 7.8; The ASP-N buffer contains Phopshate buffer 50 MM pH 7.8; The elastase buffer contains Tris HCl 50 mM pH 8.0 and the thermolysin buffer contains Tris HCl 50 mM, CaCl₂ 0.5 mM pH 9.0.

Trypsin Proteolysis: 100 μl of the reduced/alkyled CD117 were mixed with 1 μl of trypsin (Roche Diagnostic) with the ratio 1/100. The proteolytic mixture was incubated overnight at 37° C.

Chymotrypsin Proteolysis: 100 μl of the reduced/alkyled CD117 were mixed with 0.5 μl of chymotrypsin (Roche Diagnostic) with the ratio 1/200. The proteolytic mixture was incubated overnight at 25° C.

ASP-N Proteolysis: 100 μl of the reduced/alkyled CD117 were mixed with 0.5 μl of ASP-N (Roche Diagnostic) with the ratio 1/200. The proteolytic mixture was incubated overnight at 37° C.

Elastase Proteolysis: 100 μl of the reduced/alkyled CD117 were mixed with 1 μl of elastase (Roche Diagnostic) with the ratio 1/100. The proteolytic mixture was incubated overnight at 37° C.

Thermolysin Proteolysis: 100 μl of the reduced/alkyled CD117 were mixed with 2 μl of thermolysin (Roche Diagnostic) with a ratio 1/50. The proteolytic mixture was incubated overnight at 70° C.

After digestion, formic acid 1% final was added to the solution. After proteolysis, 10 μl of the peptide solution generated by proteolysis were loaded onto a nano-liquid chromatography system (Ultimate 3000-RSLC). The cross-linked peptides were analyzed using Xquest version 2.0 and Stavrox 3.6. software. nLC-LTQ-Orbitrap MS/MS analysis using a nLC Ultimate 3000-RSLC system in line with a LTQ-Orbitrap mass spectrometer (Thermo Scientific) was performed.

Results

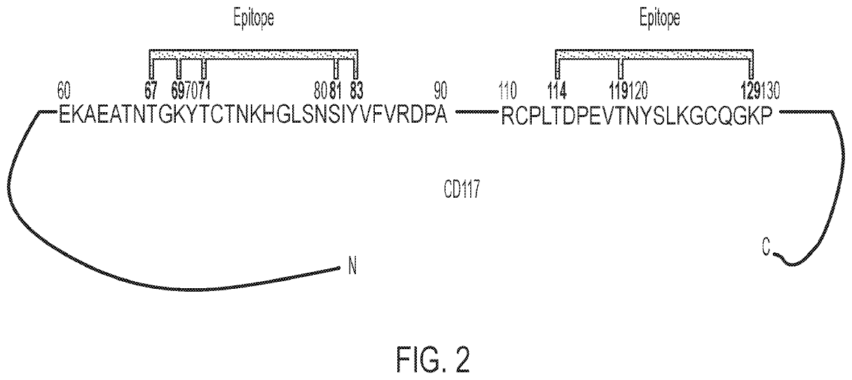

Using High-Mass MALDI mass spectrometry and chemical cross-linking, no non-covalent aggregates of the Ab85 antibody, or multimers of the antigen CD117, were detected. Using chemical cross-linking, High-Mass MALDI mass spectrometry and nLC-Orbitrap mass spectrometry the molecular interface between CD117 and Ab85 was determined. Based on this analysis, the interaction includes the following amino acids on CD117: T67, K69, T71, S81, Y83 and T114, T119, K129 (FIG. 2).

TABLE 2
AMINO ACID SEQUENCE SUMMARY

| Sequence | | |
|---|---|---|
| Identifier | Description | Amino Acid Sequence |
| SEQ ID NO: 1 | Human CD117 | QPSVSPGEPSPPSIHPGKSDLIVRVGDEIRLLCTDPG |
| | antigen | FVKWTFEILDETNENKQNEWITEKAEATNTGKYTCT |
| | | NKHGLSNSIYVFVRDPAKLFLVDRSLYGKEDNDTLV |
| | | RCPLTDPEVTNYSLKGCQGKPLPKDLRFIPDPKAGI |
| | | MIKSVKRAYHRLCLHCSVDQEGKSVLSEKFILKVRPA |
| | | FKAVPVVSVSKASYLLREGEEFTVTCTIKDVSSSVYS |
| | | TWKRENSQTKLQEKYNSWHHGDFNYERQATLTISS |
| | | ARVNDSGVFMCYANNTFGSANVTTTLEVVDKGFINI |
| | | FPMINTTVFVNDGENVDLIVEYEAFPKPEHQQWIYM |
| | | NRTFTDKWEDYPKSENESNIRYVSELHLTRLKGTEG |
| | | GTYTFLVSNSDVNAAIAFNVYVNTKPEILTYDRLVNG |
| | | MLQCVAAGFPEPTIDWYFCPGTEQRCSASVLPVDV |
| | | QTLNSSGPPFGKLVVQSSIDSSAFKHNGTVECKAYN |
| | | DVGKTSAYFNFAFKGNNKEQIHPHTHHHHHH |
| SEQ ID NO: 2 | Heavy chain | EVQLVQSGAEVKKPGESLKISCKGSGYSFTNYWIG |
| | variable region of | WVRQMPGKGLEWMAIINPRDSDTRYRPSFQGQVTI |
| | Ab 85 | SADKSISTAYLQWSSLKASDTAMYYCARHGRGYEG |
| | | YEGAFDIWGQGTLVTVSS |
| SEQ ID NO: 3 | Ab85 CDR-H1 | NYWIG |
| SEQ ID NO: 4 | Ab85 CDR-H2 | IINPRDSDTRYRPSFQG |
| SEQ ID NO: 5 | Ab85 CDR-H3 | HGRGYEGYEGAFDI |
| SEQ ID NO: 6 | Light chain variable | DIQMTQSPSSLSASVGDRVTITCRSSQGIRSDLGWY |
| | region of Ab 85 | QQKPGKAPKLLIYDASNLETGVPSRFSGSGSGTDFT |
| | | LTISSLQPEDFATYYCQQANGFPLTFGGGTKVEIK |
| SEQ ID NO: 7 | Ab85 CDR-L1 | RSSQGIRSDLG |
| SEQ ID NO: 8 | Ab85 CDR-L2 | DASNLET |
| SEQ ID NO: 9 | Ab85 CDR-L3 | QQANGFPLT |
| SEQ ID NO: 10 | Heavy chain | ASTKGPSVFPLAPSSKSTSGGTAALGCLVKDYFPEP |
| | constant region | VTVSWNSGALTSGVHTFPAVLQSSGLYSLSSVVTVP |
| | (Wild type (WT)) | SSSLGTQTYICNVNHKPSNTKVDKKVEPKSCDKTHT |
| | | CPPCPAPELLGGPSVFLFPPKPKDTLMISRTPEVTC |
| | | VVVDVSHEDPEVKFNWYVDGVEVHNAKTKPREEQY |
| | | NSTYRVVSVLTVLHQDWLNGKEYKCKVSNKALPAPI |
| | | EKTISKAKGQPREPQVYTLPPSRDELTKNQVSLTCL |
| | | VKGFYPSDIAVEWESNGQPENNYKTTPPVLDSDGS |
| | | FFLYSKLTVDKSRWQQGNVFSCSVMHEALHNHYTQ |
| | | KSLSLSPGK |
| SEQ ID | Heavy chain | ASTKGPSVFPLAPSSKSTSGGTAALGCLVKDYFPEP |
| NO: 11 | constant region | VTVSWNSGALTSGVHTFPAVLQSSGLYSLSSVVTVP |
| | with L234A, L235A | SSSLGTQTYICNVNHKPSNTKVDKKVEPKSCDKTHT |
| | (LALA) mutations | CPPCPAPEAAGGPSVFLFPPKPKDTLMISRTPEVTC |
| | (mutations in bold)* | VVVDVSHEDPEVKFNWYVDGVEVHNAKTKPREEQY |
| | | NSTYRVVSVLTVLHQDWLNGKEYKCKVSNKALPAPI |
| | | EKTISKAKGQPREPQVYTLPPSRDELTKNQVSLTCL |
| | | VKGFYPSDIAVEWESNGQPENNYKTTPPVLDSDGS |
| | | FFLYSKLTVDKSRWQQGNVFSCSVMHEALHNHYTQ |
| | | KSLSLSPGK |
| SEQ ID | Heavy chain | ASTKGPSVFPLAPSSKSTSGGTAALGCLVKDYFPEP |
| NO: 12 | constant region | VTVSWNSGALTSGVHTFPAVLQSSGLYSLSSVVTVP |
| | with D265C | SSSLGTQTYICNVNHKPSNTKVDKKVEPKSCDKTHT |
| | mutation | CPPCPAPELLGGPSVFLFPPKPKDTLMISRTPEVTCV |
| | (mutation in bold)* | VVCVSHEDPEVKFNWYVDGVEVHNAKTKPREEQYN |
| | | STYRVVSVLTVLHQDWLNGKEYKCKVSNKALPAPIE |
| | | KTISKAKGQPREPQVYTLPPSRDELTKNQVSLTCLV |
| | | KGFYPSDIAVEWESNGQPENNYKTTPPVLDSDGSFF |
| | | LYSKLTVDKSRWQQGNVFSCSVMHEALHNHYTQKS |
| | | LSLSPGK |
| SEQ ID | Heavy chain | ASTKGPSVFPLAPSSKSTSGGTAALGCLVKDYFPEP |
| NO: 13 | constant region | VTVSWNSGALTSGVHTFPAVLQSSGLYSLSSVVTVP |
| | with H435A | SSSLGTQTYICNVNHKPSNTKVDKKVEPKSCDKTHT |
| | mutation | CPPCPAPELLGGPSVFLFPPKPKDTLMISRTPEVTCV |
| | (mutation in bold)* | VVDVSHEDPEVKFNWYVDGVEVHNAKTKPREEQYN |
| | | STYRVVSVLTVLHQDWLNGKEYKCKVSNKALPAPIE |
| | | KTISKAKGQPREPQVYTLPPSRDELTKNQVSLTCLV |
| | | KGFYPSDIAVEWESNGQPENNYKTTPPVLDSDGSFF |
| | | LYSKLTVDKSRWQQGNVFSCSVMHEALHNAYTQKS |
| | | LSLSPGK |
| SEQ ID | Heavy chain | ASTKGPSVFPLAPSSKSTSGGTAALGCLVKDYFPEP |
| NO: 14 | constant region: | VTVSWNSGALTSGVHTFPAVLQSSGLYSLSSVVTVP |
| | modified Fc region | SSSLGTQTYICNVNHKPSNTKVDKKVEPKSCDKTHT |
| | with L234A, L235A, | CPPCPAPEAAGGPSVFLFPPKPKDTLMISRTPEVTC |
| | D265C mutations | VVVCVSHEDPEVKFNWYVDGVEVHNAKTKPREEQY |
| | (mutations in bold)* | NSTYRVVSVLTVLHQDWLNGKEYKCKVSNKALPAPI |
| | | EKTISKAKGQPREPQVYTLPPSRDELTKNQVSLTCL |
| | | VKGFYPSDIAVEWESNGQPENNYKTTPPVLDSDGS |
| | | FFLYSKLTVDKSRWQQGNVFSCSVMHEALHNHYTQ |
| | | KSLSLSPGK |
| SEQ ID | Heavy chain | ASTKGPSVFPLAPSSKSTSGGTAALGCLVKDYFPEP |
| NO: 15 | constant region: | VTVSWNSGALTSGVHTFPAVLQSSGLYSLSSVVTVP |
| | modified Fc region | SSSLGTQTYICNVNHKPSNTKVDKKVEPKSCDKTHT |
| | with L234A, L235A, | CPPCPAPEAAGGPSVFLFPPKPKDTLMISRTPEVTC |
| | D265C, H435A | VVVCVSHEDPEVKFNWYVDGVEVHNAKTKPREEQY |
| | mutations | NSTYRVVSVLTVLHQDWLNGKEYKCKVSNKALPAPI |
| | (mutations in bold)* | EKTISKAKGQPREPQVYTLPPSRDELTKNQVSLTCL |
| | | VKGFYPSDIAVEWESNGQPENNYKTTPPVLDSDGS |
| | | FFLYSKLTVDKSRWQQGNVFSCSVMHEALHNAYTQ |
| | | KSLSLSPGK |
| SEQ ID | Ab85 full length | EVQLVQSGAEVKKPGESLKISCKGSGYSFTNYWIG |
| NO: 16 | heavy chain | WVRQMPGKGLEWMAIINPRDSDTRYRPSFQGQVTI |
| | sequence; constant | SADKSISTAYLQWSSLKASDTAMYYCARHGRGYEG |
| | region underlined | YEGAFDIWGQGTLVTVSSASTKGPSVFPLAPSSKST |
| | | SGGTAALGCLVKDYFPEPVTVSWNSGALTSGVHTF |
| | | PAVLQSSGLYSLSSVVTVPSSSLGTQTYICNVNHKP |
| | | SNTKVDKKVEPKSCDKTHTCPPCPAPELLGGPSVFL |
| | | FPPKPKDTLMISRTPEVTCVVVDVSHEDPEVKFNWY |
| | | VDGVEVHNAKTKPREEQYNSTYRVVSVLTVLHQDW |
| | | LNGKEYKCKVSNKALPAPIEKTISKAKGQPREPQVYT |
| | | LPPSRDELTKNQVSLTCLVKGFYPSDIAVEWESNGQ |
| | | PENNYKTTPPVLDSDGSFFLYSKLTVDKSRWQQGN |
| | | VFSCSVMHEALHNHYTQKSLSLSPGK |
| SEQ ID | Ab85 full length | EVQLVQSGAEVKKPGESLKISCKGSGYSFTNYWIG |
| NO: 17 | heavy chain | WVRQMPGKGLEWMAIINPRDSDTRYRPSFQGQVTI |
| | sequence; constant | SADKSISTAYLQWSSLKASDTAMYYCARHGRGYEG |
| | region underlined; | YEGAFDIWGQGTLVTVSSASTKGPSVFPLAPSSKST |
| | modified Fc region | SGGTAALGCLVKDYFPEPVTVSWNSGALTSGVHTF |
| | with L234A, L235A | PAVLQSSGLYSLSSVVTVPSSSLGTQTYICNVNHKP |
| | mutations | SNTKVDKKVEPKSCDKTHTCPPCPAPEAAGGPSVF |
| | (mutations in bold)* | LFPPKPKDTLMISRTPEVTCVVVDVSHEDPEVKFNW |
| | | YVDGVEVHNAKTKPREEQYNSTYRVVSVLTVLHQD |
| | | WLNGKEYKCKVSNKALPAPIEKTISKAKGQPREPQV |
| | | YTLPPSRDELTKNQVSLTCLVKGFYPSDIAVEWESN |
| | | GQPENNYKTTPPVLDSDGSFFLYSKLTVDKSRWQQ |
| | | GNVFSCSVMHEALHNHYTQKSLSLSPGK |
| SEQ ID | Ab85 full length | EVQLVQSGAEVKKPGESLKISCKGSGYSFTNYWIG |
| NO: 18 | heavy chain | WVRQMPGKGLEWMAIINPRDSDTRYRPSFQGQVTI |
| | sequence: constant | SADKSISTAYLQWSSLKASDTAMYYCARHGRGYEG |
| | region underlined; | YEGAFDIWGQGTLVTVSSASTKGPSVFPLAPSSKST |
| | modified Fc region | SGGTAALGCLVKDYFPEPVTVSWNSGALTSGVHTF |
| | with L234A, L235A, | PAVLQSSGLYSLSSVVTVPSSSLGTQTYICNVNHKP |
| | D265C mutations | SNTKVDKKVEPKSCDKTHTCPPCPAPEAAGGPSVF |
| | (mutations in bold)* | LFPPKPKDTLMISRTPEVTCVVVCVSHEDPEVKFNW |
| | | YVDGVEVHNAKTKPREEQYNSTYRVVSVLTVLHQD |
| | | WLNGKEYKCKVSNKALPAPIEKTISKAKGQPREPQV |
| | | YTLPPSRDELTKNQVSLTCLVKGFYPSDIAVEWESN |
| | | GQPENNYKTTPPVLDSDGSFFLYSKLTVDKSRWQQ |
| | | GNVFSCSVMHEALHNHYTQKSLSLSPGK |
| SEQ ID | Ab85 full length | EVQLVQSGAEVKKPGESLKISCKGSGYSFTNYWIG |
| NO: 19 | heavy chain | WVRQMPGKGLEWMAIINPRDSDTRYRPSFQGQVTI |
| | sequence (LALA- | SADKSISTAYLQWSSLKASDTAMYYCARHGRGYEG |
| | D265C-H435A | YEGAFDIWGQGTLVTVSSASTKGPSVFPLAPSSKST |
| | mutant); constant | SGGTAALGCLVKDYFPEPVTVSWNSGALTSGVHTF |
| | region underlined | PAVLQSSGLYSLSSVVTVPSSSLGTQTYICNVNHKP |
| | | SNTKVDKKVEPKSCDKTHTCPPCPAPEAAGGPSVF |
| | | LFPPKPKDTLMISRTPEVTCVVVCVSHEDPEVKFNW |
| | | YVDGVEVHNAKTKPREEQYNSTYRVVSVLTVLHQD |
| | | WLNGKEYKCKVSNKALPAPIEKTISKAKGQPREPQV |
| | | YTLPPSRDELTKNQVSLTCLVKGFYPSDIAVEWESN |
| | | GQPENNYKTTPPVLDSDGSFFLYSKLTVDKSRWQQ |
| | | GNVFSCSVMHEALHNAYTQKSLSLSPGK |
| SEQ ID | Light chain | RTVAAPSVFIFPPSDEQLKSGTASVVCLLNNFYPREA |
| NO: 20 | constant region | KVQWKVDNALQSGNSQESVTEQDSKDSTYSLSSTL |
| | | TLSKADYEKHKVYACEVTHQGLSSPVTKSFNRGEC |
| SEQ ID | Ab85 full length | DIQMTQSPSSLSASVGDRVTITCRSSQGIRSDLGWY |
| NO: 21 | light chain; | QQKPGKAPKLLIYDASNLETGVPSRFSGSGSGTDFT |
| | constant region | LTISSLQPEDFATYYCQQANGFPLTFGGGTKVEIKRT |
| | underlined | VAAPSVFIFPPSDEQLKSGTASVVCLLNNFYPREAKV |
| | | QWKVDNALQSGNSQESVTEQDSKDSTYSLSSTLTL |
| | | SKADYEKHKVYACEVTHQGLSSPVTKSFNRGEC |
| SEQ ID | Heavy chain | QVQLVQSGAAVKKPGESLKISCKGSGYRFTSYWIG |
| NO: 22 | variable region | WVRQMPGKGLEWMGIIYPGDSDTRYSPSFQGQVTI |
| | amino acid | SAGKSISTAYLQWSSLKASDTAMYYCARHGRGYNG |
| | sequence of CK6 | YEGAFDIWGQGTMVTVSS |
| SEQ ID | CK6 CDR-H1 | SYWIG |
| NO: 23 | | |
| SEQ ID | CK6 CDR-H2 | IIYPGDSDTRYSPSFQG |
| NO: 24 | | |
| SEQ ID | CK6 CDR-H3 | HGRGYNGYEGAFDI |
| NO: 25 | | |
| SEQ ID | Light chain variable | AIQLTQSPSSLSASVGDRVTITCRASQGISSALAWYQ |
| NO: 26 | region amino acid | QKPGKAPKLLIYDASSLESGVPSRFSGSGSGTDFTL |
| | sequence of CK6 | TISSLQPEDFATYYCQQFNSYPLTFGGGTKVEIK |
| SEQ ID | CK6 CDR-L1 | RASQGISSALA |
| NO: 27 | | |
| SEQ ID | CK6 CDR-L2 | DASSLES |
| NO: 28 | | |
| SEQ ID | CK6 CDR-L3 | CQQFNSYPLT |
| NO: 29 | | |
| SEQ ID | Heavy chain | QVQLVQSGAAVKKPGESLKISCKGSGYRFTTYWIG |
| NO: 30 | variable region of | WVRQMPGKGLEWMGIIYPGDSDTRYSPSFQGQVTI |
| | Ab1 | SAGKSISTAYLQWSSLKASDTAMYYCARHGRGYNG |
| | | YEGAFDIWGQGTMVTVSS |
| SEQ ID | Light chain variable | AIQLTQSPSSLSASVGDRVTITCRASQGVSSALAWY |
| NO: 31 | region of Ab1 | QQKPGKAPKLLIYDASSLESGVPSRFSGSGSGTDFT |
| | | LTISSLQPEDFATYYCQQFNSYPLTFGGGTKVEIK |
| SEQ ID | Heavy chain | QVQLVQSGAAVKKPGESLKISCKGSGYRFTTYWIG |
| NO: 32 | variable region of | WVRQMPGKGLEWMGIIYPGDSDTRYSPSFQGQVTI |
| | Ab2 | SAGKSISTAYLQWSSLKASDTAMYYCARHGRGYNG |
| | | YEGAFDIWGQGTMVTVSS |
| SEQ ID | Light chain variable | DIQLTQSPSSLSASVGDRVTITCRASQGIRTDLGWY |
| NO: 33 | region of Ab2 | QQKPGKAPKLLIYDASSLESGVPSRFSGSGSGTDFT |
| | | LTISSLQPEDFATYYCQQFNSYPLTFGGGTKVEIK |
| SEQ ID | Heavy chain | QVQLVQSGAAVKKPGESLKISCKGSGYRFTTYWIG |
| NO: 34 | variable region of | WVRQMPGKGLEWMGIIYPGDSDTRYSPSFQGQVTI |
| | Ab3 | SAGKSISTAYLQWSSLKASDTAMYYCARHGRGYNG |
| | | YEGAFDIWGQGTMVTVSS |
| SEQ ID | Light chain variable | AIRMTQSPSSLSASVGDRVTITCRASQGIRNDLAWY |
| NO: 35 | region of Ab3 | QQKPGKTPKLLIYDASSLESGVPSRFSGSGSGTDFT |
| | | LTISSLQPEDFATYYCQQFNSYPLTFGGGTKVEIK |
| SEQ ID | Heavy chain | QVQLVQSGAAVKKPGESLKISCKGSGYRFTTYWIG |
| NO: 36 | variable region of | WVRQMPGKGLEWMGIIYPGDSDTRYSPSFQGQVTI |
| | Ab4 | SAGKSISTAYLQWSSLKASDTAMYYCARHGRGYNG |
| | | YEGAFDIWGQGTMVTVSS |
| SEQ ID | Light chain variable | AIQMTQSPSSLSASVGDRVTITCRASQGIRNDLGWY |
| NO: 37 | region of Ab4 | QQKPGKAPKLLIYDASSLESGVPSRFSGSGSGTDFT |
| | | LTISSLQPEDFATYYCQQFNSYPLTFGGGTKVDIK |
| SEQ ID | Heavy chain | QVQLVQSGAAVKKPGESLKISCKGSGYRFTTYWIG |
| NO: 38 | variable region of | WVRQMPGKGLEWMGIIYPGDSDTRYSPSFQGQVTI |
| | Ab5 | SAGKSISTAYLQWSSLKASDTAMYYCARHGRGYNG |
| | | YEGAFDIWGQGTMVTVSS |
| SEQ ID | Light chain variable | NIQMTQSPSSLSASVGDRVTITCRASQAISDYLAWF |
| NO: 39 | region of Ab5 | QQKPGKAPKLLIYDASNLETGVPSRFSGSGSGTDFT |
| | | LTISSLQPEDFATYYCQQLNSYPLTFGGGTKVEIK |
| SEQ ID | Heavy chain | QVQLVQSGAAVKKPGESLKISCKGSGYRFTTYWIG |
| NO: 40 | variable region of | WVRQMPGKGLEWMGIIYPGDSDTRYSPSFQGQVTI |
| | Ab6 | SAGKSISTAYLQWSSLKASDTAMYYCARHGRGYNG |
| | | YEGAFDIWGQGTMVTVSS |
| SEQ ID | Light chain variable | AIRMTQSPSSLSASVGDRVIIACRASQGIGGALAWY |
| NO: 41 | region of Ab6 | QQKPGNAPKVLVYDASTLESGVPSRFSGGGSGTDF |
| | | TLTISSLQPEDFATYYCQQFNSYPLTFGGGTKLEIK |
| SEQ ID | Heavy chain | QVQLVQSGAAVKKPGESLKISCKGSGYRFTTYWIG |
| NO: 42 | variable region of | WVRQMPGKGLEWMGIIYPGDSDTRYSPSFQGQVTI |
| | Ab7 | SAGKSISTAYLQWSSLKASDTAMYYCARHGRGYNG |
| | | YEGAFDIWGQGTMVTVSS |
| SEQ ID | Light chain variable | DIAMTQSPPSLSAFVGDRVTITCRASQGIISSLAWYQ |
| NO: 43 | region of Ab7 | QKPGKAPKLLIYDASSLESGVPSRFSGSGSGTDFTL |
| | | TIRSLQPEDFATYYCQQFNSYPLTFGGGTKLEIK |
| SEQ ID | Heavy chain | QVQLVQSGAAVKKPGESLKISCKGSGYRFTTYWIG |
| NO: 44 | variable region of | WVRQMPGKGLEWMGIIYPGDSDTRYSPSFQGQVTI |
| | Ab8 | SAGKSISTAYLQWSSLKASDTAMYYCARHGRGYNG |
| | | YEGAFDIWGQGTMVTVSS |
| SEQ ID | Light chain variable | DIQMTQSPSSLSASVGDRVTITCRASQGISSALAWY |
| NO: 45 | region of Ab8 | QQKAGKAPKVLISDASSLESGVPSRFSGSGSGTDFT |
| | | LSISSLQPEDFATYYCQQFNGYPLTFGGGTKVDIK |
| SEQ ID | Heavy chain | QVQLVQSGAAVKKPGESLKISCKGSGYRFTTYWIG |
| NO: 46 | variable region of | WVRQMPGKGLEWMGIIYPGDSDTRYSPSFQGQVTI |
| | Ab9 | SAGKSISTAYLQWSSLKASDTAMYYCARHGRGYNG |
| | | YEGAFDIWGQGTMVTVSS |
| SEQ ID | Light chain variable | AIRMTQSPSSLSASVGDRVTITCQASQGIRNDLGWY |
| NO: 47 | region of Ab9 | QQKPGKAPKLLIYDASNLETGVPSRFSGSGSGTDFT |
| | | FTISSLOPEDIATYYCQQFNSYPLTFGGGTKLEIK |
| SEQ ID | Heavy chain | QVQLVQSGAAVKKPGESLKISCKGSGYRFTTYWIG |
| NO: 48 | variable region of | WVRQMPGKGLEWMGIIYPGDSDTRYSPSFQGQVTI |
| | Ab10 | SAGKSISTAYLQWSSLKASDTAMYYCARHGRGYNG |
| | | YEGAFDIWGQGTMVTVSS |
| SEQ ID | Light chain variable | NIQMTQSPSSLSTSVGDRVTITCRASQGIGTSLAWY |
| NO: 49 | region of Ab10 | QQKPGKPPKLLIYDASSLESGVPSRLSGSGSGTDFT |
| | | LTISSLOPEDFATYYCQQSNSYPITFGQGTRLEIK |
| SEQ ID | Heavy chain | QVQLVQSGAAVKKPGESLKISCKGSGYRFTTYWIG |
| NO: 50 | variable region of | WVRQMPGKGLEWMGIIYPGDSDTRYSPSFQGQVTI |
| | Ab11 | SAGKSISTAYLQWSSLKASDTAMYYCARHGRGYNG |
| | | YEGAFDIWGQGTMVTVSS |
| SEQ ID | Light chain variable | AIQLTQSPSSLSASVGDRVTITCRASQSIGDYLTWYO |
| NO: 51 | region of Ab11 | QKPGKAPKVLIYGASSLQSGVPPRFSGSGSGTDFTL |
| | | TVSSLOPEDFATYYCQQLNSYPLTFGGGTKLEIK |
| SEQ ID | Heavy chain | QVQLVQSGAAVKKPGESLKISCKGSGYRFTTYWIG |
| NO: 52 | variable region of | WVRQMPGKGLEWMGIIYPGDSDTRYSPSFQGQVTI |
| | Ab12 | SAGKSISTAYLQWSSLKASDTAMYYCARHGRGYNG |
| | | YEGAFDIWGQGTMVTVSS |
| SEQ ID | Light chain variable | DIQLTQSPSSLSASVGDRVTITCRASQGVRSTLAWY |
| NO: 53 | region of Ab12 | QQKPGKAPKLLIYDASILESGVPSRFSGSGSGTDFTL |
| | | TISSLOPEDFATYYCQQFNGYPLTFGQGTRLEIK |
| SEQ ID | Heavy chain | QVQLVQSGAAVKKPGESLKISCKGSGYRFTTYWIG |
| NO: 54 | variable region of | WVRQMPGKGLEWMGIIYPGDSDTRYSPSFQGQVTI |
| | Ab13 | SAGKSISTAYLQWSSLKASDTAMYYCARHGRGYNG |
| | | YEGAFDIWGQGTMVTVSS |
| SEQ ID | Light chain variable | DIVMTOSPSSLSASVGDRVTITCRASQGIRNDLGWY |
| NO: 55 | region of Ab13 | QQKPGKAPKLLIYDASSLESGVPSRFSGSGSGTDFT |
| | | LTISSLOPEDFATYYCQQFNSYPLTFGGGTKLEIK |
| SEQ ID | Heavy chain | QVQLVQSGAAVKKPGESLKISCKGSGYRFTTYWIG |
| NO: 56 | variable region of | WVRQMPGKGLEWMGIIYPGDSDTRYSPSFQGQVTI |
| | Ab14 | SAGKSISTAYLQWSSLKASDTAMYYCARHGRGYNG |
| | | YEGAFDIWGQGTMVTVSS |
| SEQ ID | Light chain variable | DIQLTQSPSSLSASVGDRVTITCRASQGISSFLAWYW |
| NO: 57 | region of Ab14 | QKPGKAPKLLIYDASTLQSGVPSRFSGSASGTDFTL |
| | | TISSLOPEDFATYYCQQLNGYPLTFGGGTKVEIK |
| SEQ ID | Heavy chain | QVQLVQSGAAVKKPGESLKISCKGSGYRFTTYWIG |
| NO: 58 | variable region of | WVRQMPGKGLEWMGIIYPGDSDTRYSPSFQGQVTI |
| | Ab15 | SAGKSISTAYLQWSSLKASDTAMYYCARHGRGYNG |
| | | YEGAFDIWGQGTMVTVSS |
| SEQ ID | Light chain variable | AIQLTQSPSSLSASVGDRVTITCRASQGIGSALAWYQ |
| NO: 59 | region of Ab15 | QKPGIGPKLLIYDASTLESGVPARFSGSGSRTDFTLTI |
| | | TSLQPEDFATYYCQQFNGYPLTFGGGTKLEIK |
| SEQ ID | Heavy chain | QVQLVQSGAAVKKPGESLKISCKGSGYRFTTYWIG |
| NO: 60 | variable region of | WVRQMPGKGLEWMGIIYPGDSDTRYSPSFQGQVTI |
| | Ab16 | SAGKSISTAYLQWSSLKASDTAMYYCARHGRGYNG |
| | | YEGAFDIWGQGTMVTVSS |
| SEQ ID | Light chain variable | AIQLTQSPSSLSASVGDRVTITCRASQGITSALAWYQ |
| NO: 61 | region of Ab16 | EKPGKAPNLLIYDASSLESGVPSRFSGSGYGTDFTL |
| | | TISSLOPEDFATYYCQQLNSYPLTFGGGTKVDIK |
| SEQ ID | Heavy chain | QVQLVQSGAEVKKPGASVKVSCKASGYTFTDYYIH |
| NO: 62 | variable region of | WVRQAPGQGLEWMGWMNPHSGDTGYAQKFQGR |
| | Ab29 | VTMTRDTSTSTVYMELSSLRSEDTAVYYCARHGRG |
| | | YNGYEGAFDIWGQGTLVTVSSAS |
| SEQ ID | Light chain variable | DIQMTQSPSSLSASVGDRVTITCRASQGIGNELGWY |
| NO: 63 | region of Ab29 | QQKPGKAPKLLIYAASNLQSGVPSRFSGSGSGTDFT |
| | | LTISSLQPEDFATYYCQQYDNLPLTFGQGTKVEIK |
| SEQ ID | Heavy chain | QVQLVQSGAEVKKPGASVKVSCKASGYTFTGYYLH |
| NO: 64 | variable region of | WVRQAPGQGLEWMGWINPNSGDTNYAQNFQGRV |
| | Ab30 | TMTRDTSTSTVYMELSSLRSEDTAVYYCARHGRGY |
| | | NGYEGAFDIWGQGTLVTVSSAS |
| SEQ ID | Light chain variable | DIQMTQSPSSLSASVGDRVTITCRASQGIRNDLGWY |
| NO: 65 | region of Ab30 | QQKPGKAPKLLIYDASSLESGVPSRFSGSGSGTDFT |
| | | LTISSLQPEDFATYYCQQLNGYPLTFGGGTKVEIK |
| SEQ ID | Heavy chain | QVQLVQSGAEVKKPGASVKVSCKASGYTFTGYYLH |
| NO: 66 | variable region of | WVRQAPGQGLEWMGWINPNSGGTNYAQKFQGRV |
| | Ab31 | TMTRDTSTSTVYMELSSLRSEDTAVYYCARHGRGY |
| | | EGYEGAFDIWGQGTLVTVSSAS |
| SEQ ID | Light chain variable | DIQMTQSPSSLSASVGDRVTITCRASQGIRNDLGWY |
| NO: 67 | region of Ab31 | QQKPGKAPKLLIYDASELETGVPSRFSGSGSGTDFT |
| | | LTISSLQPEDFATYYCQQLNGYPITFGQGTKVEIK |
| SEQ ID | Heavy chain | QVQLVQSGAEVKKPGASVKVSCKASGYTFTSYYIH |
| NO: 68 | variable region of | WVRQAPGQGLEWMGWLNPSGGGTSYAQKFQGRV |
| | Ab32 | TMTRDTSTSTVYMELSSLRSEDTAVYYCARHGRGY |
| | | DGYEGAFDIWGQGTLVTVSSAS |
| SEQ ID | Light chain variable | DIQMTQSPSSLSASVGDRVTITCRASQGIRNDLGWY |
| NO: 69 | region of Ab32 | QQKPGKAPKLLIYDASNLETGVPSRFSGSGSGTDFT |
| | | LTISSLQPEDFATYYCQQLNGYPLTFGGGTKVEIK |
| SEQ ID | Heavy chain | QVQLVQSGAEVKKPGASVKVSCKASGYTFSTYYMH |
| NO: 70 | variable region of | WVRQAPGQGLEWMGIINPSGGSTSYAQKFQGRVT |
| | Ab33 | MTRDTSTSTVYMKLSSLRSEDTAVYYCARHGRGYE |
| | | GYEGAFDIWGQGTLVTVSSAS |
| SEQ ID | Light chain variable | DIQMTQSPSSLSASVGDRVTITCRASQGIRDDLGWY |
| NO: 71 | region of Ab33 | QQKPGKAPKLLIYDASNLETGVPSRFSGSGSGTDFT |
| | | LTISSLQPEDFATYYCQQANGFPLTFGGGTKVEIK |
| SEQ ID | Heavy chain | QVQLVQSGAEVKKPGASVKVSCKASGYTFTGYYIH |
| NO: 72 | variable region of | WVRQAPGQGLEWMGIINPSGGNTNYAQNFQGRVT |
| | Ab34 | MTRDTSTSTVYMELSSLRSEDTAVYYCARHGRGYN |
| | | AYEGAFDIWGQGTLVTVSSAS |
| SEQ ID | Light chain variable | DIQMTQSPSSLSASVGDRVTITCRASQGIRNDLGWY |
| NO: 73 | region of Ab34 | QQKPGKAPKLLIYDASNLETGVPSRFSGSGSGTDFT |
| | | LTISSLQPEDFATYYCQQVNGYPLTFGGGTKVEIK |
| SEQ ID | Heavy chain | QVQLVQSGAEVKKPGASVKVSCKASGGTFSSYAIS |
| NO: 74 | variable region of | WVRQAPGQGLEWMGVINPTVGGANYAQKFQGRVT |
| | Ab35 | MTRDTSTSTVYMELSSLRSEDTAVYYCARHGRGYN |
| | | EYEGAFDIWGQGTLVTVSSAS |
| SEQ ID | Light chain variable | DIQMTQSPSSLSASVGDRVTITCQASQDISDYLNWY |
| NO: 75 | region of Ab35 | QQKPGKAPKLLIYDASNLETGVPSRFSGSGSGTDFT |
| | | LTISSLQPEDFATYYCQQGNSFPLTFGGGTKLEIK |
| SEQ ID | Heavy chain | QVQLVQSGAEVKKLGASVKVSCKASGYTFSSYYMH |
| NO: 76 | variable region of | WVRQAPGQGLEWMGVINPNGAGTNFAQKFQGRVT |
| | Ab36 | MTRDTSTSTVYMELSSLRSEDTAVYYCARHGRGYE |
| | | GYEGAFDIWGQGTLVTVSSAS |
| SEQ ID | Light chain variable | DIQMTQSPSSLSASVGDRVTITCRASQGIRNDLGWY |
| NO: 77 | region of Ab36 | QQKPGKAPKLLIYDASSLESGVPSRFSGSGSGTDFT |
| | | LTISSLQPEDFATYYCQQLNGYPLTFGGGTKVEIK |
| SEQ ID | Heavy chain | QVQLVQSGAEVKKPGASVKVSCKASGYTFTTYYMH |
| NO: 78 | variable region of | WVRQAPGQGLEWMGWINPTGGGTNYAQNFQGRV |
| | Ab37 | TMTRDTSTSTVYMELSSLRSEDTAVYYCARHGRGY |
| | | EGYEGAFDIWGQGTLVTVSSAS |
| SEQ ID | Light chain variable | DIQMTQSPSSLSASVGDRVTITCRASQGIRNDVSWY |
| NO: 79 | region of Ab37 | QQKPGKAPKLLIYDASNLETGVPSRFSGSGSGTDFT |
| | | LTISSLQPEDFATYYCQQLSGYPITFGQGTKLEIK |
| SEQ ID | Heavy chain | QVQLVQSGAEVKKPGASVKVSCKASGYTFTSYYIH |
| NO: 80 | variable region of | WVRQAPGQGLEWMGMINPSGGSTNYAQKFQGRVT |
| | A38 | MTRDTSTSTVYMELSSLRSEDTAVYYCARHGRGYN |
| | | DYEGAFDIWGQGTLVTVSSAS |
| SEQ ID | Light chain variable | DIQMTQSPSSLSASVGDRVTITCRASQSISDWLAWY |
| NO: 81 | region of Ab38 | QQKPGKAPKLLIYEASNLEGGVPSRFSGSGSGTDFT |
| | | LTISSLQPEDFATYYCQQANSFPYTFGQGTKVEIK |
| SEQ ID | Heavy chain | QVQLVQSGAEVKKPGASVKVSCKASGYIFSAYYIHW |
| NO: 82 | variable region of | VRQAPGQGLEWMGIINPSGGSTRYAQKFQGRVTMT |
| | Ab39 | RDTSTSTVYMELSSLRSEDTAVYYCARHGRGYGGY |
| | | EGAFDIWDQGTLVTVSSAS |
| SEQ ID | Light chain variable | DIQMTQSPSSLSASVGDRVTITCRASQGIGDYVAWY |
| NO: 83 | region of Ab39 | QQKPGKAPKLLIYDASNLETGVPSRFSGSGSGTDFT |
| | | LTISSLQPEDFATYYCQQLNGYPITFGQGTRLEIK |
| SEQ ID | Heavy chain | EVQLVQSGAEVKKPGESLKISCKGSGYRFTSYWIG |
| NO: 84 | variable region of | WVRQMPGKGLEWMGIIYPDDSDTRYSPSFQGQVTI |
| | Ab40 | SVDKSNSTAYLQWSSLKASDTAMYYCARHGRGYN |
| | | GYEGAFDIWGQGTLVTVSSAS |
| SEQ ID | Light chain variable | DIQMTQSPSSLSASVGDRVTITCRASQGISSYLAWY |
| NO: 85 | region of Ab40 | QQKPGKAPKLLIYDASNLETGVPSRFSGSGSGTYFT |
| | | LTISSLQPEDFATYYCQQGASFPITFGQGTKVEIK |
| SEQ ID | Heavy chain | EVQLVQSGAEVKKPGESLKISCKGSGSSFPNSWIAW |
| NO: 86 | variable region of | VRQMPGKGLEWMGIIYPSDSDTRYSPSFQGQVTISA |
| | Ab41 | DKSISTAYLQWSSLEASDTAMYYCARHGRGYNGYE |
| | | GAFDIWGQGTLVTVSSAS |
| SEQ ID | Light chain variable | DIQMTQSPSSLSASVGDRVTITCRASQGIRNYLAWY |
| NO: 87 | region of Ab41 | QQKPGKAPKLLIYDASSLQSGVPSRFSGSGSGTDFT |
| | | LTISSLQPEDFATYYCQQLNSYPLTFGGGTKVEIK |
| SEQ ID | Heavy chain | EVQLVQSGAEVKKPGESLKISCKGSGYSFDSYWIG |
| NO: 88 | variable region of | WVRQMPGKGLEWMGIMYPGDSDTRYSPSFQGQVT |
| | Ab42 | ISADKSISTAYLQWSSLKASDTAMYYCARHGRGYNA |
| | | YEGAFDIWGQGTLVTVSSAS |
| SEQ ID | Light chain variable | DIQMTQSPSSLSASVGDRVTITCRASQSINNWLAWY |
| NO: 89 | region of Ab42 | QQKPGKAPKLLIYDAFILQSGVPSRFSGSGSGTDFTL |
| | | TISSLQPEDFATYYCLQLNSYPLTFGPGTKVDIK |
| SEQ ID | Heavy chain | EVQLVQSGAEVKKPGESLKISCKGSGYSFTNWIAW |
| NO: 90 | variable region of | RQMPGKGLEWMGIIYPGDSETRYSPSFQGQVTISAD |
| | Ab43 | KSISTAYLQWSSLKASDTAMYYCARHGRGYYGYEG |
| | | AFDIWGQGTLVTVSSAS |
| SEQ ID | Light chain variable | DIQMTQSPSSLSASVGDRVTITCRASQGISDNLNWY |
| NO: 91 | region of Ab43 | QQKPGKAPKLLIYDASNLETGVPSRFSGSGSGTDFT |
| | | LTISSLQPEDFATYYCQQAISFPLTFGQGTKVEIK |
| SEQ ID | Heavy chain | EVQLVQSGAEVKKPGESLKISCKGSGYNFTSYWIG |
| NO: 92 | variable region of | WVRQMPGKGLEWMGVIYPDDSETRYSPSFQGQVTI |
| | Ab44 | SADKSISTAYLQWSSLKASDTAMYYCARHGRGYNG |
| | | YEGAFDIWGQGTLVTVSSAS |
| SEQ ID | Light chain variable | DIQMTQSPSSLSASVGDRVTITCRASRDIRDDLGWY |
| NO: 93 | region of Ab44 | QQKPGKAPKLLIYDASNLETGVPSRFSGSGSGTDFT |
| | | LTISSLQPEDFATYYCQQANSFPLTFGGGTKVEIK |
| SEQ ID | Heavy chain | EVQLVQSGAEVKKPGESLKISCKGSGYTFNTYIGWV |
| NO: 94 | variable region of | RQMPGKGLEWMGIIYPGDSGTRYSPSFQGQVTISA |
| | Ab45 | DKAISTAYLQWSSLKASDTAMYYCARHSRGYNGYE |
| | | GAFDIWGQGTLVTVSSAS |
| SEQ ID | Light chain variable | DIQMTQSPSSLSASVGDRVTITCRASQGISNYLAWY |
| NO: 95 | region of Ab45 | QQKPGKAPKLLIYDASNLETGVPSRFSGSGSGTDFT |
| | | LTISSLQPEDFATYYCQQANSFPVTFGQGTKVEIK |
| SEQ ID | Heavy chain | EVQLVQSGAEVKKPGESLKISCKGSGYNFTTYWIGW |
| NO: 96 | variable region of | VRQMPGKGLEWMGIIHPADSDTRYNPSFQGQVTISA |
| | Ab46 | DKSISTAYLQWSSLKASDTAMYYCARHGRGYNGYE |
| | | GAFDIWGQGTLVTVSSAS |
| SEQ ID | Light chain variable | DIQMTQSPSSLSASVGDRVTITCRVSQGISSYLAWY |
| NO: 97 | region of Ab46 | QQKPGKAPKLLIYDASNLETGVPSRFSGSGSGTDFT |
| | | LTISSLQPEDFATYYCQQANSFPLTFGGGTKVEIK |
| SEQ ID | Heavy chain | EVQLVQSGAEVKKPGESLKISCKGSGYRFSNYWIA |
| NO: 98 | variable region of | WVRQMPGKGLEWMGIIYPDNSDTRYSPSFQGQVTI |
| | Ab47 | SADKSISTAYLQWSSLKASDTAMYYCARHGRGYDG |
| | | YEGAFDIWGQGTLVTVSSAS |
| SEQ ID | Light chain variable | DIQMTQSPSSLSASVGDRVTITCRASQGIRSDLAWY |
| NO: 99 | region of Ab47 | QQKPGKAPKLLIYGASSLQSGVPSRFSGSGSGTDFT |
| | | LTISSLQPEDFATYYCQQANSFPLSFGQGTKVEIK |
| SEQ ID | Heavy chain | EVQLVQSGAEVKKPGESLKISCKGSGYRFASYWIG |
| NO: 100 | variable region of | WVRQMPGKGLEWMGITYPGDSETRYNPSQGQVTIS |
| | Ab48 | ADKSISTAYLQWSSLKASDTAMYYCARHGRGYGGY |
| | | EGAFDIWGQGTLVTVSSAS |
| SEQ ID | Light chain variable | DIQMTQSPSSLSASVGDRVTITCRASQGIRNDLGWY |
| NO: 101 | region of Ab48 | QQKPGKAPKLLIYDASNLETGVPSRFSGSGSGTDFT |
| | | LTISSLQPEDFATYYCQQANSFPLTFGGGTKVEIK |
| SEQ ID | Heavy chain | EVQLVQSGAEVKKPGESLKISCKGSGYSFTSYWIGW |
| NO: 102 | variable region of | VRQMPGKGLEWMGIIYPGDSDTRYSPSFQGQVTISA |
| | Ab49 | DKSISTAYLQWSSLKASDTAMYYCARHGRGYNGYE |
| | | GAFDIWGQGTLVTVSSAS |
| SEQ ID | Light chain variable | DIQMTQSPSSLSASVGDRVTITCRASQSISNWLAWY |
| NO: 103 | region of Ab49 | QQKPGKAPKLLIYDASNLETGVPSRFSGSGSGTDFT |
| | | LTISSLQPEDFATYYCQQTNSFPLTFGQGTRLEIK |
| SEQ ID | Heavy chain | QVQLVQSGAAVKKPGESLKISCKGSGYRFTTYWIG |
| NO: 104 | variable region of | WVRQMPGKGLEWMGIIYPGDSDTRYSPSFQGQVTI |
| | Ab74 | SAGKSISTAYLQWSSLKASDTAMYYCARHGRGYNG |
| | | YEGAFDIWGQGTMVTVSS |
| SEQ ID | Light chain variable | DIQLTQSPSSLSASVGDRVTITCRASQGVISALAWYQ |
| NO: 105 | region of Ab74 | QKPGKAPKLLIYDASSLESGVPSRFSGSGSGTDFTL |
| | | TISSLQPEDFATYYCQQFNSYPLTFGGGTKVEIK |
| SEQ ID | Heavy chain | QVQLVQSGAAVKKPGESLKISCKGSGYRFTTYWIG |
| NO: 106 | variable region of | WVRQMPGKGLEWMGIIYPGDSDTRYSPSFQGQVTI |
| | Ab75 | SAGKSISTAYLQWSSLKASDTAMYYCARHGRGYNG |
| | | YEGAFDIWGQGTMVTVSS |
| SEQ ID | Light chain variable | DIQLTQSPSSLSASVGDRVTITCRASQGIRSALAWYQ |
| NO: 107 | region of Ab75 | QKPGKAPKLLIYDASSLESGVPSRFSGSGSGTDFTL |
| | | TISSLQPEDFATYYCQQFNSYPLTFGGGTKVEIK |
| SEQ ID | Heavy chain | QVQLVQSGAAVKKPGESLKISCKGSGYRFTTYWIG |
| NO: 108 | variable region of | WVRQMPGKGLEWMGIIYPGDSDTRYSPSFQGQVTI |
| | Ab76 | SAGKSISTAYLQWSSLKASDTAMYYCARHGRGYNG |
| | | YEGAFDIWGQGTMVTVSS |
| SEQ ID | Light chain variable | DIQLTQSPSSLSASVGDRVTITCRASQGVGSALAWY |
| NO: 109 | region of Ab76 | QQKPGKAPKLLIYDASSLESGVPSRFSGSGSGTDFT |
| | | LTISSLQPEDFATYYCQQFNSYPLTFGGGTKVEIK |
| SEQ ID | Heavy chain | QVQLVQSGAAVKKPGESLKISCKGSGYRFTTYWIG |
| NO: 110 | variable region of | WVRQMPGKGLEWMGIIYPGDSDTRYSPSFQGQVTI |
| | Ab77 | SAGKSISTAYLQWSSLKASDTAMYYCARHGRGYNG |
| | | YEGAFDIWGQGTMVTVSS |
| SEQ ID | Light chain variable | DIQLTQSPSSLSASVGDRVTITCRASQGVISALAWYQ |
| NO: 111 | region of Ab77 | QKPGKAPKLLIYDASILESGVPSRFSGSGSGTDFTLTI |
| | | SSLQPEDFATYYCQQFNSYPLTFGGGTKVEIK |
| SEQ ID | Heavy chain | QVQLVQSGAAVKKPGESLKISCKGSGYRFTTYWIG |
| NO: 112 | variable region of | WVRQMPGKGLEWMGIIYPGDSDTRYSPSFQGQVTI |
| | Ab78 | SAGKSISTAYLQWSSLKASDTAMYYCARHGRGYNG |
| | | YEGAFDIWGQGTMVTVSS |
| SEQ ID | Light chain variable | DIQLTQSPSSLSASVGDRVTITCRASQGIRSALAWYQ |
| NO: 113 | region of Ab78 | QKPGKAPKLLIYDASILESGVPSRFSGSGSGTDFTLTI |
| | | SSLQPEDFATYYCQQFNSYPLTFGGGTKVEIK |
| SEQ ID | Heavy chain | QVQLVQSGAAVKKPGESLKISCKGSGYRFTTYWIG |
| NO: 114 | variable region of | WVRQMPGKGLEWMGIIYPGDSDTRYSPSFQGQVTI |
| | Ab79 | SAGKSISTAYLQWSSLKASDTAMYYCARHGRGYNG |
| | | YEGAFDIWGQGTMVTVSS |
| SEQ ID | Light chain variable | DIQLTQSPSSLSASVGDRVTITCRASQGVGSALAWY |
| NO: 115 | region of Ab79 | QQKPGKAPKLLIYDASILESGVPSRFSGSGSGTDFTL |
| | | TISSLQPEDFATYYCQQFNSYPLTFGGGTKVEIK |
| SEQ ID | Heavy chain | QVQLVQSGAAVKKPGESLKISCKGSGYRFTTYWIG |
| NO: 116 | variable region of | WVRQMPGKGLEWMGIIYPGDSDTRYSPSFQGQVTI |
| | Ab80 | SAGKSISTAYLQWSSLKASDTAMYYCARHGRGYNG |
| | | YEGAFDIWGQGTMVTVSS |
| SEQ ID | Light chain variable | DIQLTQSPSSLSASVGDRVTITCRASQGISSALAWYQ |
| NO: 117 | region of Ab80 | QKPGKAPKLLIYDASILESGVPSRFSGSGSGTDFTLTI |
| | | SSLQPEDFATYYCQQFNSYPLTFGGGTKVEIK |
| SEQ ID | Heavy chain | QVQLVQSGAAVKKPGESLKISCKGSGYRFTTYWIG |
| NO: 118 | variable region of | WVRQMPGKGLEWMGIIYPGDSDTRYSPSFQGQVTI |
| | Ab81 | SAGKSISTAYLQWSSLKASDTAMYYCARHGRGYNG |
| | | YEGAFDIWGQGTMVTVSS |
| SEQ ID | Light chain variable | DIQLTQSPSSLSASVGDRVTITCRASQGVISALAWYQ |
| NO: 119 | region of Ab81 | QKPGKAPKLLIYDASTLESGVPSRFSGSGSGTDFTL |
| | | TISSLQPEDFATYYCQQFNSYPLTFGGGTKVEIK |
| SEQ ID | Heavy chain | QVQLVQSGAAVKKPGESLKISCKGSGYRFTTYWIG |
| NO: 120 | variable region of | WVRQMPGKGLEWMGIIYPGDSDTRYSPSFQGQVTI |
| | Ab82 | SAGKSISTAYLQWSSLKASDTAMYYCARHGRGYNG |
| | | YEGAFDIWGQGTMVTVSS |
| SEQ ID | Light chain variable | DIQLTQSPSSLSASVGDRVTITCRASQGIRSALAWYQ |
| NO: 121 | region of Ab82 | QKPGKAPKLLIYDASTLESGVPSRFSGSGSGTDFTL |
| | | TISSLQPEDFATYYCQQFNSYPLTFGGGTKVEIK |
| SEQ ID | Heavy chain | QVQLVQSGAAVKKPGESLKISCKGSGYRFTTYWIG |
| NO: 122 | variable region of | WVRQMPGKGLEWMGIIYPGDSDTRYSPSFQGQVTI |
| | Ab83 | SAGKSISTAYLQWSSLKASDTAMYYCARHGRGYNG |
| | | YEGAFDIWGQGTMVTVSS |
| SEQ ID | Light chain variable | DIQLTQSPSSLSASVGDRVTITCRASQGVGSALAWY |
| NO: 123 | region of Ab83 | QQKPGKAPKLLIYDASTLESGVPSRFSGSGSGTDFT |
| | | LTISSLQPEDFATYYCQQFNSYPLTFGGGTKVEIK |
| SEQ ID | Heavy chain | QVQLVQSGAAVKKPGESLKISCKGSGYRFTTYWIG |
| NO: 124 | variable region of | WVRQMPGKGLEWMGIIYPGDSDTRYSPSFQGQVTI |
| | Ab84 | SAGKSISTAYLQWSSLKASDTAMYYCARHGRGYNG |
| | | YEGAFDIWGQGTMVTVSS |
| SEQ ID | Light chain variable | DIQLTQSPSSLSASVGDRVTITCRASQGVGSALAWY |
| NO: 125 | region of Ab84 | QQKPGKAPKLLIYDASTLESGVPSRFSGSGSGTDFT |
| | | LTISSLQPEDFATYYCQQFNSYPLTFGGGTKVEIK |
| SEQ ID | Heavy chain | EVQLVQSGAEVKKPGESLKISCKGSGYSFTNYWIG |
| NO: 126 | variable region of | WVRQMPGKGLEWMGIIYPGDSDIRYSPSLQGQVTIS |
| | Ab86 | VDTSTSTAYLQWNSLKPSDTAMYYCARHGRGYNGY |
| | | EGAFDIWGQGTLVTVSS |
| SEQ ID | Light chain variable | DIQMTQSPSSLSASVGDRVTITCRASQGIGDSLAWY |
| NO: 127 | region of Ab86 | QQKPGKAPKLLIYDASNLETGVPSRFSGSGSGTDFT |
| | | LTISSLQPEDFATYYCQQLNGYPITFGQGTKVEIK |
| SEQ ID | Heavy chain | EVQLVQSGAEVKKPGESLKISCKGSGYSFTNYWIG |
| NO: 128 | variable region of | WVRQMPGKGLEWMAIINPRDSDTRYRPSFQGQVTI |
| | Ab87 | SADKSISTAYLQWSSLKASDTAMYYCARHGRGYEG |
| | | YEGAFDIWGQGTLVTVSS |
| SEQ ID | Light chain variable | DIQMTQSPSSLSASVGDRVTITCRASQGIRNDLGWY |
| NO: 129 | region of Ab87 | QQKPGKAPKLLIYDASSLESGVPSRFSGSGSGTDFT |
| | | LTISSLQPEDFATYYCQQLNGYPITFGQGTKVEIK |
| SEQ ID | Heavy chain | EVQLVQSGAEVKKPGESLKISCKGSGYSFTNYWIG |
| NO: 130 | variable region of | WVRQMPGKGLEWMGIIYPGDSLTRYSPSFQGQVTI |
| | Ab88 | SADKSISTAYLQWSSLKASDTAMYYCARHGRGYNG |
| | | YEGAFDIWGQGTLVTVSS |
| SEQ ID | Light chain variable | DIQMTQSPSSLSASVGDRVTITCRASQGIRNDLGWY |
| NO: 131 | region of Ab88 | QQKPGKAPKLLIYDASSLESGVPSRFSGSGSGTDFT |
| | | LTISSLQPEDFATYYCQQLNGYPITFGQGTKVEIK |
| SEQ ID | Heavy chain | EVQLVQSGAEVKKPGESLKISCKGSGYSFTNYWIG |
| NO: 132 | variable region of | WVRQMPGKGLEWMGIIYPGDSDTRYSPSFQGQVTI |
| | Ab89 | SADKSISTAYLQWSSLKASDTAMYYCARHGRGYNG |
| | | YEGAFDIWGQGTLVTVSS |
| SEQ ID | Light chain variable | DIQMTQSPSSLSASVGDRVTITCRASQGIGDSLAWY |
| NO: 133 | region of Ab89 | QQKPGKAPKLLIYDASNLETGVPSRFSGSGSGTDFT |
| | | LTISSLQPEDFATYYCQQLNGYPITFGQGTKVEIK |
| SEQ ID | Heavy chain | EVQLVQSGAEVKKPGESLKISCKGSGYRFTTSWIGW |
| NO: 134 | variable region of | VRQMPGKGLEWMGIIYPGDSDTRYSPSFQGQVTISA |
| | Ab245 | DKSISTAYLQWSSLKASDTAMYYCARHGLGYNGYE |
| | | GAFDIWGQGTLVTVSS |
| SEQ ID | Light chain variable | DIQMTQSPSSLSASVGDRVTITCRASQGIGSALAWY |
| NO: 135 | region of Ab245 | QQKPGKAPKLLIYDASTLESGVPSRFSGSGSGTDFT |
| | | LTISSLQPEDFATYYCQQFNGYPLTFGQGTRLEIK |
| SEQ ID | Heavy chain | QVQLVQSGAAVKKPGESLKISCKGSGYRFTTYWIG |
| NO: 136 | variable region of | WVRQMPGKGLEWMGIIYPGDSDTRYSPSFQGQVTI |
| | Ab246 | SAGKSISTAYLQWSSLKASDTAMYYCARHGRGYNG |
| | | YEGAFDIWGQGTMVTVSS |
| SEQ ID | Light chain variable | DIQMTQSPSSLSASVGDRVTITCRASQGIGSALAWY |
| NO: 137 | region of Ab246 | QQKPGKAPKLLIYDASTLESGVPSRFSGSGSGTDFT |
| | | LTISSLQPEDFATYYCQQFNGYPLTFGQGTRLEIK |
| SEQ ID | Heavy chain | QVQLVQSGAAVKKPGESLKISCKGSGYRFTTYWIG |
| NO: 138 | variable region of | WVRQMPGKGLEWMGIIYPGDSDTRYSPSFQGQVTI |
| | Ab247 | SAGKSISTAYLQWSSLKASDTAMYYCARHGRGYNG |
| | | YEGAFDIWGQGTMVTVSS |
| SEQ ID | Light chain variable | DIQMTQSPSSLSASVGDRVTITCRASRGISDYLAWY |
| NO: 139 | region of Ab247 | QQKPGKAPKLLIYDASNLETGVPSRFSGSGSGTDFT |
| | | LTISSLQPEDFATYYCQQANSFPITFGQGTRLEIK |
| SEQ ID | Heavy chain | EVQLVQSGAEVKKPGESLKISCKGSGYRFTTSWIGW |
| NO: 140 | variable region of | VRQMPGKGLEWMGIIYPGDSDTRYSPSFQGQVTISA |
| | Ab248 | DKSISTAYLQWSSLKASDTAMYYCARHGLGYNGYE |
| | | GAFDIWGQGTLVTVSS |
| SEQ ID | Light chain variable | DIQMTQSPSSLSASVGDRVTITCRASQGIGSALAWY |
| NO: 141 | region of Ab248 | QQKPGKAPKLLIYDASTLESGVPSRFSGSGSGTDFT |
| | | LTISSLQPEDFATYYCQQLNGYPLTFGQGTRLEIK |
| SEQ ID | Heavy chain | EVQLVQSGAEVKKPGESLKISCKGSGYRFTTSWIGW |
| NO: 142 | variable region of | VRQMPGKGLEWMGIIYPGDSDTRYSPSFQGQVTISA |
| | Ab249 | DKSISTAYLQWSSLKASDTAMYYCARHGLGYNGYE |
| | | GAFDIWGQGTLVTVSS |
| SEQ ID | Light chain variable | DIQMTQSPSSLSASVGDRVTITCRASQGIGSALAWY |
| NO: 143 | region of Ab249 | QQKPGKAPKLLIYDASNLETGVPSRFSGSGSGTDFT |
| | | LTISSLQPEDFATYYCQQLNGYPLTFGQGTRLEIK |

### Other Embodiments

All publications, patents, and patent applications mentioned in this specification are incorporated herein by reference to the same extent as if each independent publication or patent application was specifically and individually indicated to be incorporated by reference.

While the present disclosure has been described in connection with specific embodiments thereof, it will be understood that it is capable of further modifications and this application is intended to cover any variations, uses, or adaptations of the present disclosure following, in general, the principles of the present disclosure and including such departures from the present disclosure that come within known or customary practice within the art to which the present disclosure pertains and may be applied to the essential features hereinbefore set forth, and follows in the scope of the claims.

Other embodiments are within the claims.

1. An isolated anti-CD117 antibody, or antigen-binding fragment thereof, which binds to human CD117 at least at two of the following amino acid residues T67, K69, T71, S81, Y83, T114, T119, or K129 of CD117 listed in SEQ ID NO: 1.

2. The isolated anti-CD117 antibody, or antigen-binding fragment thereof, of claim 1, wherein the antibody, or antigen-binding fragment thereof, binds to at least three of the following amino acid residues T67, K69, T71, S81, Y83, T114, T119, or K129 of CD117 listed in SEQ ID NO: 1.

3. The isolated anti-CD117 antibody, or antigen-binding fragment thereof, of claim 1, wherein the antibody, or antigen-binding fragment thereof, binds to at least four of the following amino acid residues T67, K69, T71, S81, Y83, T114, T119, or K129 of CD117 listed in SEQ ID NO: 1.

4. The isolated anti-CD117 antibody, or antigen-binding fragment thereof, of claim 1, wherein the antibody, or antigen-binding fragment thereof, binds to at least five of the following amino acid residues T67, K69, T71, S81, Y83, T114, T119, or K129 of CD117 listed in SEQ ID NO: 1.

5. The isolated anti-CD117 antibody, or antigen-binding fragment thereof, of claim 1, wherein the antibody, or antigen-binding fragment thereof, binds to at least six of the following amino acid residues T67, K69, T71, S81, Y83, T114, T119, or K129 of CD117 listed in SEQ ID NO: 1.

6. The isolated anti-CD117 antibody, or antigen-binding fragment thereof, of claim 1, wherein the antibody, or antigen-binding fragment thereof, binds to at least seven of the following amino acid residues T67, K69, T71, S81, Y83, T114, T119, or K129 of CD117 listed in SEQ ID NO: 1.

7. The isolated anti-CD117 antibody, or antigen-binding fragment thereof, of claim 1, wherein the antibody, or antigen-binding fragment thereof, binds to each of the following amino acid residues T67, K69, T71, S81, Y83, T114, T119, or K129 of CD117 listed in SEQ ID NO: 1.

8. The isolated anti-CD117 antibody, or antigen-binding fragment thereof, of claim 1, wherein the antibody, or antigen-binding fragment thereof, comprises a heavy chain variable region having an amino acid sequence that is about 95% identical to SEQ ID NO: 2 and a light chain variable region having an amino acid sequence that is about 95% identical to the SEQ ID NO: 6.

9. The isolated anti-CD117 antibody, or antigen-binding fragment thereof, of claim 1, wherein the antibody, or antigen-binding fragment thereof, comprises a heavy chain variable region having an amino acid sequence that is at least 96% identical to SEQ ID NO: 2 and a light chain variable region having an amino acid sequence that is at least 96% identical to the SEQ ID NO: 6.

10. The isolated anti-CD117 antibody, or antigen-binding fragment thereof, of claim 1, wherein the antibody, or antigen-binding fragment thereof, comprises a heavy chain variable region having an amino acid sequence that is about 97% identical to SEQ ID NO: 2 and a light chain variable region having an amino acid sequence that is about 97% identical to the SEQ ID NO: 6.

11. The isolated anti-CD117 antibody, or antigen-binding fragment thereof, of claim 1, wherein the antibody, or antigen-binding fragment thereof, comprises a heavy chain variable region having an amino acid sequence that is about 99% identical to SEQ ID NO: 2 and a light chain variable region having an amino acid sequence that is about 99% identical to the SEQ ID NO: 6.

12. An isolated anti-CD117 antibody, or antigen-binding fragment thereof, capable of binding CD117 that binds to an epitope having residues within at least amino acids 67-83 and 114-129 of SEQ ID NO: 1.

13. (canceled)

14. The antibody of claim 1, wherein the anti-CD117 antibody, or antigen-binding fragment thereof, does not comprise the CDRs or the variable regions set forth in SEQ ID NO: 2 and SEQ ID NO: 6.

15. The antibody of claim 1, wherein the anti-CD117 antibody, or antigen-binding fragment thereof, is antagonistic.

16. A pharmaceutical composition comprising an isolated anti-CD117 antibody, or antigen-binding fragment thereof, wherein, when bound to CD117, the antibody, or antigen-binding fragment thereof, binds to at least two of the following amino acid residues T67, K69, T71, S81, Y83, T114, T119, or K129 of CD117 listed in SEQ ID NO: 1 and blocks or inhibits the biological activity of CD117.

17. The pharmaceutical composition of claim 16 wherein the antibody, or antigen-binding fragment thereof, binds to at least three of the following amino acid residues T67, K69, T71, S81, Y83, T114, T119, or K129 of CD117 listed in SEQ ID NO: 1 and blocks or inhibits the biological activity of CD117.

18. The pharmaceutical composition of claim 16 wherein the antibody, or antigen-binding fragment thereof, binds to at least four of the following amino acid residues T67, K69, T71, S81, Y83, T114, T119, or K129 of CD117 listed in SEQ ID NO: 1 and blocks or inhibits the biological activity of CD117.

19. The pharmaceutical composition of claim 16 wherein the antibody, or antigen-binding fragment thereof, binds to at least five of the following amino acid residues T67, K69, T71, S81, Y83, T114, T119, or K129 of CD117 listed in SEQ ID NO: 1 and blocks or inhibits the biological activity of CD117.

20. The pharmaceutical composition of claim 16 wherein the antibody, or antigen-binding fragment thereof, binds to at least six of the following amino acid residues T67, K69, T71, S81, Y83, T114, T119, or K129 of CD117 listed in SEQ ID NO: 1 and blocks or inhibits the biological activity of CD117.

21. The pharmaceutical composition of claim 16 wherein the antibody, or antigen-binding fragment thereof, binds to at least seven of the following amino acid residues T67, K69, T71, S81, Y83, T114, T119, or K129 of CD117 listed in SEQ ID NO: 1 and blocks or inhibits the biological activity of CD117.

22. The pharmaceutical composition of claim 16 wherein the antibody, or antigen-binding fragment thereof, binds to each of the following amino acid residues T67, K69, T71, S81, Y83, T114, T119, or K129 of CD117 listed in SEQ ID NO: 1 and blocks or inhibits the biological activity of CD117.
